(19) United States
(12) Patent Application Publication
(10) Pub. No.: US 2022/0379130 A1
(43) Pub. Date: Dec. 1, 2022

(54) ELECTRICAL SYSTEM FOR TREATMENT OF A SUBJECT

(71) Applicant: TheraBionic Inc., Winston-Salem, NC (US)

(72) Inventor: Alexandre Barbault, Colmar (FR)

(73) Assignee: TheraBionic Inc., Winston-Salem, NC (US)

(21) Appl. No.: 17/755,118

(22) PCT Filed: Oct. 20, 2020
(86) PCT No.: PCT/US20/56427

§ 371 Date: Apr. 21, 2022

Related U.S. Application Data
(60) Provisional application No. 62/934,212, filed on Nov. 12, 2019.
(60) Provisional application No. 62/923,908, filed on Oct. 21, 2019.

(51) Int. Cl. A61N 1/40

(52) U.S. Cl. CPC A61N 1/40

(57) ABSTRACT

The systems and methods of this disclosure are directed to the treatment of disease states, and particularly different types of cancer, by application of low energy emission therapy. The device and method provide treatments of disease states in a patient, and particularly of cancer types, by the application to the patient of particular and disease-specific low energy high frequency radiation. The device uses a high precision frequency synthesizer to generate radio frequency radiation that is amplitude-modulated at identified tumor-specific frequencies for application to the patient during therapy.

### CROSS-REFERENCE TO RELATED APPLICATIONS

This application claims priority under 35 U.S.C. § 119(e) to U.S. Provisional Patent Application Ser. No. 62/923,908, filed on Oct. 21, 2019 and U.S. Provisional Patent Application Ser. No. 62/934,212, filed on Nov. 12, 2019, and which are hereby incorporated by reference in their entirety.

### FIELD OF INVENTION

This invention relates to systems and methods for treatment of a subject using low energy high frequency radiation. The electronic system and programmed control thereof has therapeutic application for influencing cellular functions (or malfunctions), including directly or indirectly influencing cancerous cell growth or proliferation thereof in warm-blooded mammalian subjects. The device and method of the invention provide treatments of different cancer types by the application to the patient of electromagnetic fields that are amplitude-modulated at particular and cancer-specific frequencies.

### BACKGROUND OF INVENTION

Reference is made to European Patent EP 0 592 851 B1 and corresponding patents and patent applications and to the various publications referred to therein, related to effects of very low energy electromagnetic fields on subjects. A number of further publications related to effects of very low energy electromagnetic fields on patients suffering from insomnia and/or anxiety disorders have taken place and are as follows:
Koziol, J. A., Erman, M., Pasche B., Hajdukovic R., Mitler, M. M., (1993), “Assessing a changepoint in a sequence of repeated measurements with application to a low-energy emission therapy sleep study”. J. Applied Statistics 20: 393-400;
Amato, D., Pasche, B., (1993), “An evaluation of the safety of low energy emission therapy”. Compr Ther 19: 242-247;
Higgs, L., Reite, M., Barbault, A., Lebet, J. P., Rossel, C., Amato, D., Dafni, U., Pasche. B., (1994), “Subjective and Objective Relaxation Effects of Low Energy Emission Therapy”. Stress Medicine 10: 5-13;
Reite, M., Higgs, L, Lebet, J. P., Barbault, A., Rossel, C., Kuster, N., Dafni, U., Amato, D., Pasche, B., (1994), “Sleep Inducing Effect of Low Energy Emission Therapy”. Bioelectromagnetics 15: 67-75.
Lebet, J. P., Barbault, A., Rossel, C., Tomic, Z., Reite, M., Higgs, L., Dafni, U., Amato, D., Pasche, B., (1996), “Electroencephalographic changes following low energy emission therapy”. Ann Biomed Eng 24: 424-429;
Pasche, B., Erman, M., Hayduk, R., Mitler, M., Reite, M., Higgs, L., Dafni, U., Amato, D., Rossel, C., Kuster, N., Barbault, A., Lebet, J. P., (1996), “Effects of Low Energy Emission Therapy in chronic psychophysiological insomnia”. Sleep 19: 327-336;
Kelly, T. L., Kripke, D. F., Hayduk, R., Ryman, D., Pasche, B., Barbault, A., (1997), “Bright light and LEET effects on circadian rhythms, sleep and cognitive performance”. Stress Medicine 13: 251-258; and
Pasche, B., Barbault, A., (2003), “Low-Energy Emission Therapy Current Status and Future Directions. In Bioelectromagnetic Medicine”, Rosch, P. J., Markov, M. S. (eds.), pages 321-327, Marcel Dekker, Inc.: New York, N.Y.

The above publications are related to an earlier device, system and use thereof described in said EP 0 592 851 B1.

Electromagnetic energy generating devices and use of electromagnetic energies for treating living mammalian subjects harboring cancerous cells described in the literature include: U.S. Pat. No. 5,908,441 issued Jun. 1, 1999 to James E. Bare and the references cited therein and so-called “NovoCure technology” involving in vivo implantation of electrodes to either side of tumorous growths. The method of this patent is invasive as it involves implantation of electrodes into the patient. This may also render the method unsuitable for treatment of some tumor types. This patent does not contemplate very low energy emissions of electromagnetic energy involving amplitude-modulated high frequency carrier signals as required in terms of the present invention.

U.S. Pat. No. 5,690,692 issued Nov. 25, 1997 entitled “Bio-Active Frequency Generator and Method” describes a programmable control which instructs a frequency synthesizer to enable generation of an electrical current at a specific precise frequency signal or at a series of specific precise frequency signals having a square wave form to within an accuracy of 0.001 Hz. This patent contemplates amplifying the voltage of the generated signals and applying the signals to a subject at the specific precise frequency or sequentially at the series of specific precise frequencies by means of electrodes held by or otherwise connected to the subject (which may be a mammal or a food). This patent also does not contemplate very low energy emissions of electromagnetic energy involving amplitude-modulated high frequency carrier signals as required in terms of the present invention.

U.S. Pat. No. 8,977,365 issued Mar. 10, 2015 entitled “Electronic System For Influencing Cellular Functions In A Warm-Blooded Mammalian Subject” describes an electronic system activatable by electrical power is useful for influencing cellular functions or malfunctions in a warm-blooded mammalian subject. The system includes one or more controllable low energy HF (High Frequency) carrier signal generator circuits, one or more data processors for receiving control information, one or more amplitude modulation control generators and one or more amplitude modulation frequency control generators. The amplitude modulation frequency control generators are adapted to accurately control the frequency of the amplitude modulations to within an accuracy of at least 1000 ppm, most preferably to within about 1 ppm, relative to one or more determined or predetermined reference amplitude modulation frequencies. While the improved electronic system and programmed control thereof in accordance with U.S. Pat. No. 8,977,365, were shown to directly or indirectly influence cancerous cell growth or proliferation thereof in warm-blooded mammalian subjects, this patent utilized an analog synthesizer for generating a carrier signal and lead to a classy output signal.

The device and method of the instant invention is generally non-invasive, without the need of implanted electrodes. The method and device provided herein applies very low energy emissions of electromagnetic energy with precisely determined amplitude-modulated frequencies to a patient, particularly for the treatment of cancer.

There is a great need for improved cancer therapies to expand the treatment options for cancer. The need is particularly acute for the treatment of cancers that are difficult to treat or are resistant to treatment, such as refractory cancers, cancers in which other treatment options have lost effectiveness, and metastasized cancers and particularly in metastasis to the bone and brain. The systems and methods of the current disclosure provide treatments of cancer which fulfill these and other needs as discussed in detail below.

### SUMMARY OF THE INVENTION

The systems and methods of the current disclosure are directed to the treatment of different types of cancer by application of low energy emission therapy. Such systems and methods have been determined to find therapeutic application not only for influencing cellular functions (or malfunctions) leading to central nervous system (CNS) disorders, but more particularly for influencing other cellular functions (or malfunctions), including directly or indirectly influencing cancerous cell growth or proliferation thereof in warm-blooded mammalian subjects. The direct or indirect influence on cancerous cell growth may involve, but is not necessarily limited to, any of prophylactic avoidance of cancerous cell formation, influencing of cell functions such as for example influencing leukocyte cell functions which can lead to inhibition of cancerous cell growth or proliferation thereof, and/or killing of cancerous cells harbored by a warm-blooded mammalian subject.

In one aspect, the invention provides an apparatus for treating a subject suffering from cancer, the apparatus comprising (i) a conductive applicator configured to apply to the subject low energy high frequency radiation, wherein the low energy high frequency radiation comprises one or more amplitude-modulated output signals; and (ii) a frequency synthesizer coupled to the conductive applicator and configured to generate the one or more amplitude-modulated output signals by generating a carrier frequency signal having a carrier frequency from about 1 KHz to 5000 MHz; and one or more amplitude modulation frequency signals having amplitude modulation frequencies from about 0.1 Hz to about 150,000 Hz, wherein the amplitude modulation frequencies are selected to be cancer-specific frequencies.

The frequency synthesizer may be a digital frequency synthesizer comprising (i) a digital carrier frequency synthesizer configured to output the carrier frequency signal; (ii) a digital modulation frequency synthesizer configured to output the one or more amplitude modulation frequency signals; (iii) an arithmetic logic unit (ALU) configured to numerically compute one or more digital modulated signals from the carrier frequency signal and a modulation frequency digital signal in real-time; and (iv) a digital to analog converter (DAC) configured to convert the one or more digital modulated signals to the one or more amplitude-modulated output signals.

In another aspect, the invention, provides a method of treating a subject suffering from cancer, the method comprising exposing the subject to low energy high frequency radiation, wherein the low energy high frequency radiation comprises one or more amplitude-modulated output signals, wherein the one or more amplitude-modulated output signal(s) have a carrier frequency from about 1 KHz to 5000 MHz; and have amplitude modulation frequencies from about 0.1 Hz to about 150,000 Hz, and wherein the amplitude modulation frequencies are selected to be cancer-specific frequencies; and wherein the subject is treated with one or more, or 10 or more, 15 or more, or 20 or more, 25 or more, or 30 or more, 35 or more, or 40 or more, 45 or more, or 50 or more, 60 or more, 70 or more, 80 or more, 90 or more, or 100 or more of cancer-specific amplitude modulation frequencies disclosed herein.

In another aspect, the devices and methods disclosed herein use a specific absorption rate (SAR) of the low energy high frequency radiation absorbed by the patient that is from about 1 microWatt per kilogram of tissue to about 50 Watts per kilogram of tissue, is from about 100 microWatts per kilogram of tissue to about 10 Watts per kilogram of tissue, or is from about 0.02 milliWatt per kilogram of tissue to about 400 milliWatts per kilogram of tissue. The low energy high frequency radiation may be applied to the subject undergoing treatment via an electrically conductive probe, which may be configured for contact with a mucosa of the subject or with the skin of the subject.

In another aspect the frequency synthesizer is a digital frequency synthesizer comprising a digital carrier frequency synthesizer configured to output the carrier frequency signal; a digital modulation frequency synthesizer configured to output the one or more amplitude modulation frequency signals; an arithmetic logic unit (ALU) configured to numerically compute one or more digital modulated signals from the carrier frequency signal and a modulation frequency digital signal in real-time; and a digital to analog converter (DAC) configured to convert the one or more digital modulated signals to the one or more amplitude-modulated output signals.

The methods and devices disclosed herein may be used for the treatment of a wide variety of cancers, including breast cancer, neuroendocrine tumors, non-Hodgkin lymphoma, adenocarcinoma, head and neck cancer, gastric cancer, glioblastoma, squamous cell carcinoma, hepatocellular carcinoma, cholangiocarcinoma, mesothelioma, thyroid cancer, prostate cancer, rhabdomyosarcoma, lung cancer, kidney cancer, ovarian cancer, bladder cancer, leiomyosarcoma, myeloma, lymphoma, leukemia, chronic lymphoid cancer, brain cancer, and colorectal cancer.

### BRIEF DESCRIPTION OF THE DRAWINGS

FIG. 1 shows an example system for application of a low energy electromagnetic radiation to a subject.

FIG. 2 illustrates a block diagram of an example emission device for application of a low energy electromagnetic radiation to a subject.

FIG. 3 illustrates a block diagram of an example digital frequency synthesizer of the emission device of FIG. 2.

FIG. 4 illustrates a block diagram of a controller of the emission device of FIG. 2.

FIG. 5 illustrates an example amplitude-modulated output signal.

FIG. 6 illustrates a block diagram of internal hardware included in any of the electronic components of the emission device of FIG. 2.

### DETAILED DESCRIPTION OF THE PREFERRED EMBODIMENTS

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such “first” and “second” are used to modify a noun or phrase, such use is simply intended to distinguish one item from another, and is not intended to require a sequential order unless specifically stated. The term “about” when used in connection with a numeric value, is intended to include values that are close to, but not exactly, the number. For example, in some embodiments, the term “about” may include values that are within +/−10 percent of the value.

The present disclosure relates generally to a method and apparatus for using low energy emission therapy, which involves application of low energy radio frequency (RF) electromagnetic fields to a warm-blooded mammalian subject for the treatment of various forms of cancer, using particular frequencies of radio frequency (RF) radiation. The disclosures of EP 0 592 851 B1 and U.S. Pat. No. 8,977,365 (“the '365 patent”) are incorporated herein in entirety by reference.

Terminology that is relevant to this document includes:

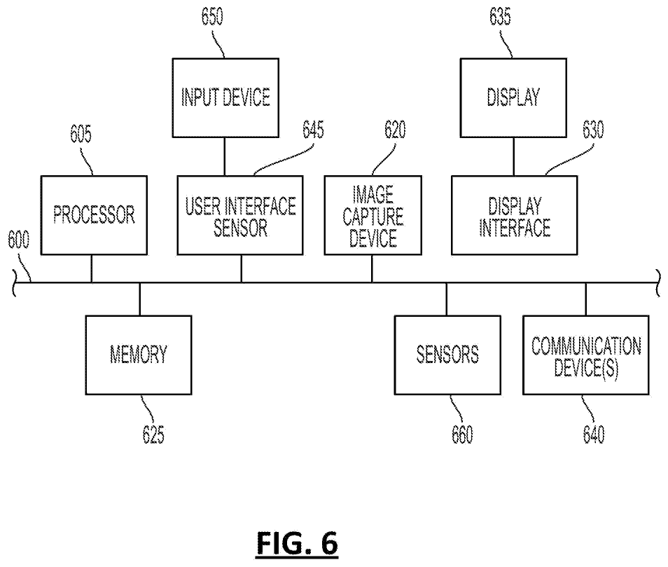

An “electronic device” or a “computing device” refers to a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, gaming systems, televisions, digital home assistants and mobile electronic devices such as smartphones, fitness tracking devices, and wearable virtual reality devices. Electronic devices also may include Internet-connected wearables such as smart watches, smart clothing and smart eyewear. Electronic devices also may be embedded in products that are designed to be used by a human during treatment, such as a spoon shaped probe, and may be connected to a subject by any means that provides an adequate transmission of the frequencies to the subject, including but not limited to devices in direct contact with a mucosa of a subject such as a spoon shaped or other shaped probe, devices in contact with the subject's skin such as a band or patch or a means to transmit the frequencies without direct contact with the subject. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion below, a client device, server device, virtual machine or container may be referred to simply as a “device” for brevity. Additional elements that may be included in electronic devices will be discussed below in the context of FIG. 6.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “data store,” “data storage facility” and the like each refer to a non-transitory device on which computer-readable data, programming instructions or both are stored. Except where specifically stated otherwise, the terms “memory,” “memory device,” “data store,” “data storage facility” and the like are intended to include single device embodiments, embodiments in which multiple memory devices together or collectively store a set of data or instructions, as well as individual sectors within such devices.

As used herein, the term “treat”, “treating” or “stimulating” refers to any process, action, application, therapy, or the like, wherein a subject (or patient), including a human being, is provided medical aid with the object of improving the subject's condition, directly or indirectly, or slowing the progression of a condition or disorder in the subject, or ameliorating at least one symptom of the disease or disorder under treatment.

The term “patient” or “subject”, as used herein, refers to any animal, preferably a mammal, and more preferably a human, and includes, but is not limited to, domestic and farm animals, primates, and humans, for example, human beings, non-human primates, cows, horses, pigs, sheep, goats, dogs, cats, or rodents like rats and mice.

The device and method of the invention provide treatments of disease states in a patient, and particularly of cancer types, by the application to the patient of particular and disease-specific low energy high frequency radiation. The device uses a high precision frequency synthesizer to generate radio frequency EMF that is amplitude-modulated at identified tumor-specific frequencies for application to the patient during therapy.

The system of the current disclosure is employed to influence cellular functions or malfunctions in a warm-blooded mammalian subject. The system comprises one or more controllable low energy electromagnetic energy generator circuits for generating one or more radio frequency output signals. One or more controller circuits comprising or communicating with the one or more generator circuits are provided, which are also for receiving control information from a source of programmed control information. The one or more generator circuits may include a programmable field-programmable gate array (FPGA) configured for digital synthesis of a complete modulated digital signal using one or more direct digital synthesizers (DDS). FPGA generates the modulated digital signal by numerical computation based on a carrier frequency digital signal and a modulation frequency digital signal. The complete modulated digital signal generated by the FPGA is, in terms of an important improvement of the present invention, preferably adapted to accurately control the frequency of the amplitude modulations to within an accuracy of 1 part per 10,000, more preferably within an accuracy of 1 part per 100,000 and most preferably within an accuracy of 1 parts per million (ppm), with a long term stability of +/−5.3 ppm, relative to one or more determined or predetermined reference amplitude modulation frequencies selected from within a range of 0.1 Hz to 150,000 Hz, more preferably within 100 Hz to 99,000 Hz. The FPGA synthesis of complete modulated digital signal also allows for other types of modulation and combination of different modulation frequencies simultaneously. Moreover, such a digital synthesis also allows the resolution to be independent of the actual modulation frequency. The complete modulated digital signal may be converted to an analog RF output signal using a suitable digital to analog (DAC) converter. The system furthermore comprises a connection or coupling position for connection or coupling to or being connected or coupled to an electrically conductive applicator for applying to the warm-blooded mammalian subject the one or more amplitude-modulated low energy emissions at said accurately controlled modulation frequencies.

While the current disclosure describes the use of modulated digital signals, analog form and analog modulation signals may also be used, as described in the '365 patent.

As used herein, the term, “accurately controlled” means that the modulated low energy electromagnetic emissions preferably should be modulated to within a resolution of at most about 0.1 Hz, more preferably, about 0.05 Hz, most preferably about 3 to about 5 milli Hz (0.003-0.005 Hz) of intended determined or predetermined modulation frequencies. For example, if one of the one or more determined or predetermined modulation frequencies to be applied to the warm-blooded mammalian subject is about 2000 Hz, the accurate control should lead to such modulated low energy emission being generated at a frequency of between about 1999.995-1999.997 and about 2000.003-2000.005 Hz.

Of importance is the condition for emissions to be at a very low and safe energy level and result in low levels of absorption. The reason believed to be that physiological exchanges or flow of electrical impulses within warm-blooded animals (which are to be affected by application of the emissions of the present invention) are similarly at very low energy levels. In any event, in the region (at or near to the position of contact or close-by induction of the electrically conductive applicator with a subject receiving treatment), the specific absorption rate (SAR) should be between about 0.02 and about 400 milliW/kg.

Furthermore of importance to achieve the intended biological therapeutic effect is that the stability of the emissions be maintained during emission, and that such stability should preferably be at least about 10⁻⁵, more preferably at least about 10⁻⁶, and most preferably at least about 10⁻⁷, stability being determined as the relative deviation of frequency divided by the desired frequency, e.g., 0.01 Hz (deviation)/1,000 Hz (desired freq.)=10⁻⁵.

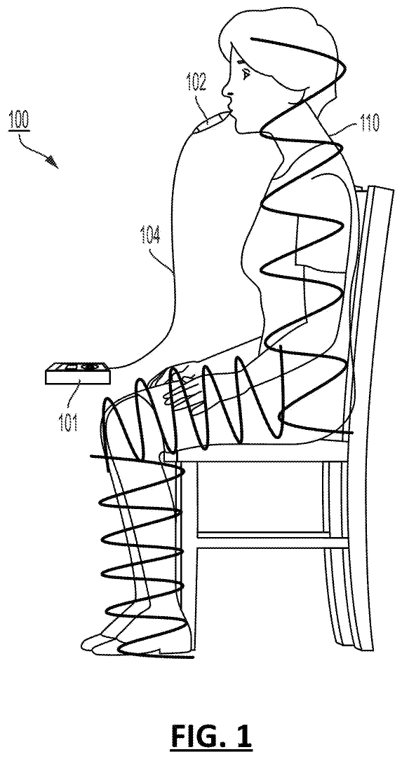

Referring now to FIG. 1, a system 100 for treatment of cancer in a subject may include an emission device 101 in communication with a subject 110. The emission device 101 may be configured to provide a modulated RF electromagnetic radiation (hereinafter “output signal”) at desired frequencies to the subject 110 via a probe 102. In certain embodiments, the output signal has various control parameters such as, without limitation, duration of a session, sequence of applied frequencies in a session, and duration of each applied frequency. The applied frequencies and their corresponding duration may be determined based on the type of tumor being treated. Specifically, the selection of such output signals is based on output signals predetermined to provide beneficial therapeutic effects in multiple subjects or patients pre-diagnosed with an identified poor health condition. The above-mentioned output signals may be provided by the emission device 101 adapted to generate EMF output signals at certain predetermined modulation frequencies. Different types of modulation may be used such as, without limitation, amplitude modulation, frequency modulation, and phase modulation. Preferably, the output signals are generated at tumor-specific amplitude modulation (AM) frequencies.

In certain embodiments, the probe 102 may be an electrically conductive applicator for applying one or more electromagnetic emissions to the warm-blooded mammalian subject via, for example, a conductive, inductive, capacitive, radiated coupling, or combinations thereof. One form of applicator may comprise an electrically conductive probe, for example, a mouthpiece which is inserted into the mouth of a subject undergoing treatment. The probe may be adapted for application to any mucosa of the subject (such as by being located within oral, nasal, optical, urethral, anal, and/or vaginal cavities or surfaces), adapted for physical contact with the skin of the patient (e.g., an insulated probe in contact with the ears, head, neck, etc. of a subject), or any other means.

In other embodiments, the EMF output signals are applied to the subject through an antenna which is not in direct physical contact with the subject. In such embodiments, the subject may be in a chamber exposed to the EMF output signals or the subject may be kept in proximity to the broadcasting antenna for the EMF output signals during the duration of the treatment(s).

The probe 102 may be connected to the emission device 101 through, for example, a coaxial cable 104.

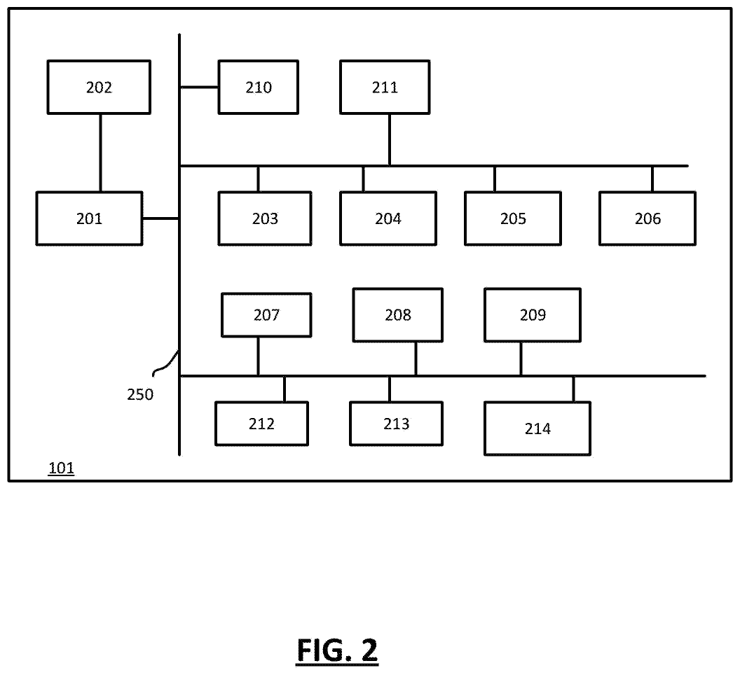

Referring now to FIG. 2, a block diagram illustrating an example emission device 101 is shown. As shown in FIG. 2, the emission device 101 includes a controller 201, a memory 202, an oscillator 203, a digital frequency synthesizer 204, a digital-to-analog (DAC) converter 205, an amplifier 206, a filter 207, a directional coupler 208, impedance transformer 209, a power source 210, and a communications interface 211. Optionally, the emission device 101 may also include a display 212, a user interface 213, and other output component(s) 214 (e.g., LEDs, speaker, etc.).

In certain embodiments, the controller 201 may operate as a controller for the emission device 101 to control one or more operations of one or more components of the emission device 101. The controller 201 may be communicatively coupled to various components of the emission device 101 via, for example, an address bus, a data bus, and input/output lines, or the like (not shown here). The timing for the controller 201 may be provided by a system clock (not shown here) that runs at any suitable clock frequency for the type of processor. In general, controller 201 is configured to control one or more operations of one or more components of the emission device 101 to produce a desired form of modulated low energy electromagnetic emission for application to a subject through probe 102.

The level of power applied is preferably controlled by the controller 201 to cause the specific absorption rate (SAR) of energy absorbed by the patient to be from about 1 microWatt per kilogram of tissue to about 50 Watts per kilogram of tissue. Preferably, the power level is controlled to cause an SAR of from about 100 microWatts per kilogram of tissue to about 10 Watts per kilogram of tissue. Most preferably, the power level is controlled to cause an SAR of from about 0.02 milliWatt per kilogram of tissue to about 400 milliWatts per kilogram of tissue. These SARs may be in any tissue of the patient.

The frequencies of electromagnetic radiation may be tumor-specific and are described below in more detail.

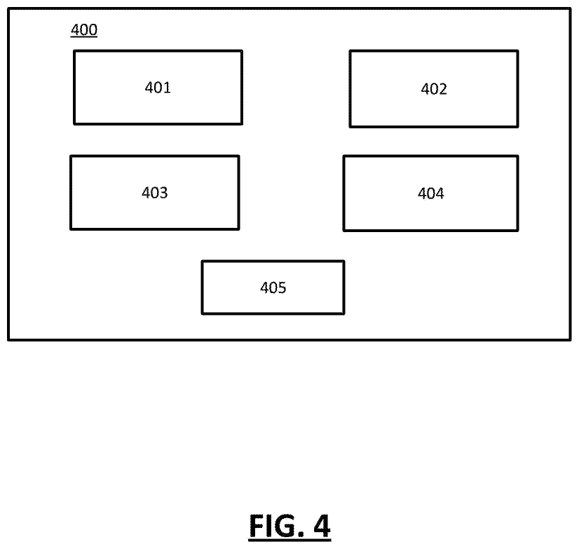

Referring now to FIG. 4, a typical controller 400 is shown with a processor 402, Random Access Memory (RAM) 403, nonvolatile memory 404, device specific circuits 401, and input output (I/O) interface 405. Alternatively, the RAM 403 and/or nonvolatile memory 404 may be contained in the processor 402 as could the device specific circuits 401 and I/O interface 405. The processor 402 may comprise, for example, an off-the-shelf microprocessor, custom processor, Field Programmable Gate Array (FPGA), Application Specific Integrated Circuit (ASIC), discrete logic, etc. The RAM 403 is typically used to hold variable data, stack data, executable instructions, etc.

According to various approaches, the nonvolatile memory 404 may comprise any type of nonvolatile memory such as, but not limited to, Electrically Erasable Programmable Read Only Memory (EEPROM), flash Programmable Read Only Memory (PROM), battery backup RAM, hard disk drives, etc. However, the nonvolatile memory 404 is typically used to hold the executable firmware and any nonvolatile data containing programming instructions that can be executed to cause the processor 402 to perform certain functions.

In some embodiments, the I/O interface 405 may include a communication interface that allows the processor 402 to communicate with devices external to the controller. Examples of the communication interface may comprise, but are not limited to, serial interfaces such as RS-232, USB (Universal Serial Bus), Small Computer Systems Interface (SCSI), Ethernet, RS-422 or a wireless communication interface such as Wi-Fi, Bluetooth, near-field communication (NFC) or other wireless interfaces. The controller 400 may communicate with an external device via the communication interface 405 in any communication protocols such as Automation/Drive Interface (ADI).

According to various approaches described and/or suggested herein, a controller may have a single processor or controller, or it may include multiple processors or controllers, or multiple cores in a processor chip.

The memory 202 may be any storage device capable of storing information for later retrieval, and may be configured to store data for the operation of the emission device 101. For example, memory 202 may be a magnetic media based storage device (such as a card, tape, disk, or drum), a semiconductor memory-based storage device (such as an erasable programmable read only memory (EPROM), an electrical erasable programmable read only memory (EEPROM) or a non-volatile random access memory (RAM)), a mechanical information storage device (such as a punched card, cam, or the like), and/or an optical storage device (such as a compact disk read only memory (CD ROM)).

The memory 202 may include control information that specifies various controllable parameters (to be used by the controller 201) of the modulated low energy radiation to be applied to a subject through probe 102. Such controllable parameters include, for example, but are not necessarily limited to, the frequency and amplitude of the output signal, the duration of the emission, the power level of the emission, the duty cycle of the emission (i.e., the ratio of on time to off time of pulsed emissions applied during a treatment), the sequence of application of different modulation frequencies for a particular application, and the total number of treatments and duration of each treatment prescribed for a particular subject, and combinations thereof.

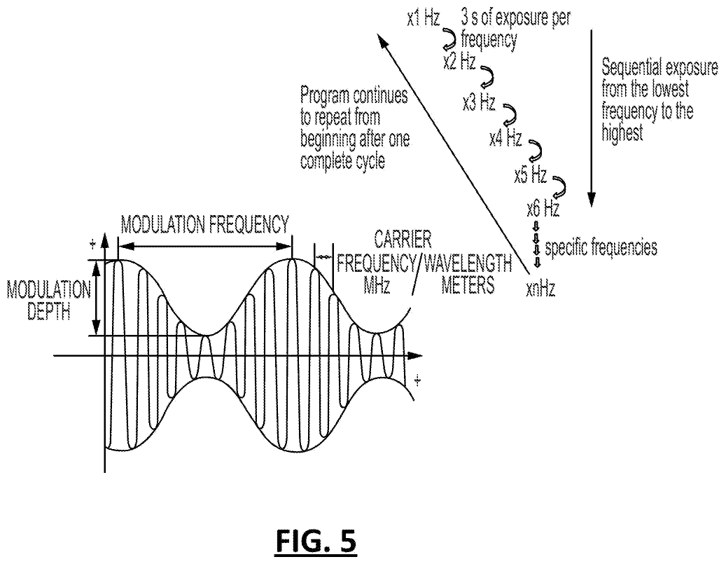

For example, FIG. 5 illustrates an example amplitude-modulated output signal that may be applied to a subject in a sequence of application of different modulation frequencies, ranging from the lowest frequency to the highest frequency, with each modulation frequency applied for a predetermined time duration. As discussed above, while the current disclosure describes the use of digitals signals (carrier frequency signal and/or modulation frequency signal), use of analog signals for generating the modulation frequencies of this disclosure are within the scope of this disclosure. Methods and systems for generation of analog signals is described in the '365 patent.

In certain embodiments, the carrier frequency digital signal and the modulation frequency digital signal (described below) may be selected to drive the probe 102 with an amplitude-modulated output signal. The carrier frequency digital signal may be from about 1 KHz to 5000 MHz, or from about 0.1-1000 MHz, or from about 1-500 MHz, or from about 1-100 MHz, or from about 5-50 MHz, or from about 10 MHz to about 40 MHz, or from about 15 MHz to about 30 MHz, or any other frequency that is capable of leveraging a subject's body as an antenna (e.g., 27 MHz). The one or more modulation frequencies may be simultaneously emitted or sequenced to form the modulation signal. The modulation frequency digital signal may be about 0.1 Hz to 150,000 Hz, more preferably within 100 Hz to 99,000 Hz, and may be determined and selected based on the type of disease state or cancer to be treated (as described below).

In certain embodiments, the memory 202 may also store event data, user information (e.g., authentication data, medical information, etc.), troubleshooting instructions, or the like, corresponding to the emission device 101. Examples of event data may include, without limitation, error logs, use history and associated data, treatment information, battery information, etc. Such event data may be used to monitor patient compliance, detect problems with the functions and the handling of the device, or the like. The information or data stored in the memory 202 may be retrieved using the communications interface 211 directly and/or indirectly (e.g., via a docking station for charging the emission device 101). Such information may then be used by a physician or other clinician to assess patient treatment compliance and effect. Treatment information may include, for example: the number of treatments applied for a given time period; the actual time and date of each treatment; the number of attempted treatments; the treatment compliance (i.e., whether the probe was in place or not in place during the treatment session); and the cumulative dose of a particular modulation frequency.

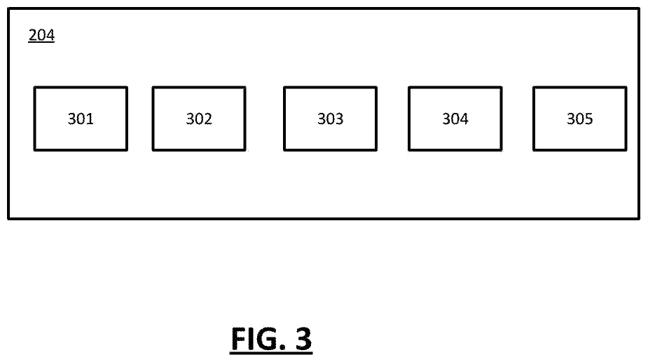

The digital frequency synthesizer 204 may be a programmable logic device (PLD) such as a field programmable gate array (FPGA), and may be configured to provide digital synthesis of a complete modulated output signal. Those skilled in the art will appreciate that the digital synthesis implementations could be comparably used when implemented on other PLDs. FIG. 3 illustrates a block diagram of an example digital frequency synthesizer 204.

As shown in FIG. 3, the digital frequency synthesizer 204 may include a carrier frequency direct digital synthesizer (DDS) 301, a modulation frequency DDS 302, one or more control registers 303, a phase-locked loop (PLL) frequency multiplier 304, and an arithmetic logic unit (ALU) 305.

Direct digital synthesis (DDS) is an increasingly popular technique for the generation of radio frequency (RF) signals due to its high performance and low cost. A DDS creates RF signals by using a reference clock supplied from an external source and data programmed into registers of the DDS. The DDS uses the reference clock to create an internal system clock as a multiple of the reference clock. The DDS uses the system clock to read the data programmed into the registers to create a digital signal output. The DDS also generates a synchronization clock to be used by external hardware to synchronize external hardware with the internal system clock of the DDS. The carrier frequency DDS 301 and the modulation frequency DDS 302 may include a core architecture consisting of a phase accumulator, which uses the data programmed into control registers 303 to set the output frequency and phase offset, and a phase to amplitude sine or cosine lookup table at the output eliminates high frequency sampling images to output a pure sine wave digital signal. In certain embodiments, carrier frequency DDS 301 may be an M-bit DDS (e.g., a 32-bit sinus DDS), and the modulation frequency DDS 302 may be an N-bit DDS (e.g., a 32-bit sinus DDS). Digital signal forms that have been successfully employed include square wave forms, sinusoidal wave forms, rectified sinusoidal, triangular, or other wave forms and/or combinations thereof.

The control registers 303 may receive data and/or instructions for setting the output frequency and phase offset of the output digital signals from the controller 201.

The carrier frequency DDS 301 may generate an M-bit carrier frequency digital signal, and the modulation frequency DDS 302 may generate an N-bit modulation frequency digital signal. The N-bit modulation frequency digital signal may be modified to control the modulation factor of the modulation frequency digital signal before being input into the ALU 305. In an embodiment, the ALU 305 may numerically compute a modulated digital output signal from the carrier frequency digital signal and the modulation frequency digital signal in real-time. For example, the digital frequency synthesizer 204 may output a K-bit (e.g., 12-bit) parallel modulated digital output signal. It should be noted the ALU 305 may be configured to numerically compute a digital output signal for providing amplitude modulation, frequency modulation, and/or phase modulation.

The modulated digital output signal may optionally undergo further amplitude modulation in a digital multiplier (not shown here) before conversion to an analog RF output.

The digital frequency synthesizer 204 also includes a PLL frequency multiplier configured to generate the internal system clock for the carrier frequency DDS 301 and the modulation frequency DDS 302, using a reference clock generated by a clock oscillator 203. The clock oscillator 203 may be a precision temperature compensated crystal oscillator with an initial accuracy of +/−1 ppm. In an exemplary embodiment, the internal system clock may be set to 128 MHz and reference clock may be set to 16 MHz.

It should be noted that while FIG. 3 illustrates one modulation frequency DDS 302, the disclosure is not so limiting, and the digital frequency synthesizer may include one or more modulation frequency generators to allow for transmitting multiple modulation frequency digital signals simultaneously.

Referring back to FIG. 2, the modulated digital output signal from the digital frequency synthesizer 204 may be input into DAC converter 205 to generate an analog RF signal. The RF signal from the DAC converter 205 passes through an amplifier 206 (e.g., a linear RF power amplifier in bridge configuration), an optional transformer (e.g., a balun) (not shown here), a filter circuit 207 (e.g., a 5^{th} order elliptic cauer filter), followed by output to the probe 102. The filter circuit 207 may be connected to probe 102 via a coaxial cable 104 and impedance transformer 209 (configured to substantially match the impedance of the subject with the impedance of the output of the emission device 101. It has been determined through impedance measurements that when probe 102 is applied to the mouth of a patient, the probe/patient combination exhibits a complex impedance on the order of 150+j₂₀₀ Ohms. Impedance transformer 209 serves to match this complex impedance with the impedance of coaxial cable, and therefore the output impedance of the filter circuit 207. This promotes power transmission, and minimizes reflections. In a further example, a conductive, isolated probe 102 has been used at a frequency around 433 MHz coupling to the outer ear channel. Due to the different probe design in such a frequency band and with this coupling method, the values of matching elements would be different or could even be omitted. Probe 102 may then be regarded as a capacitive coupler or as an antenna matched to the capacitive load.

In certain embodiments, the output from the filter circuit 207 may pass through a directional coupler 208. The output signals from the directional coupler 208 may be fed into two ADC inputs of the controller 201, where the amplitude and the ratio of the two signals allow the controller 201 to continuously monitor both the output power/frequency of the signal in the probe 102 and the quality of the probe's contact to the subject. This information may be used by the controller 201 to determine whether electromagnetic emissions at the desired frequency and power are present. Controller 201 then takes appropriate action, for example, display an error message on display 212, provide appropriate corrective signals to one or more components of the emission device 101, determine and control the amount of power applied to the subject, assess patient treatment compliance, record indicia of the patient treatment compliance on memory 202 for later analysis and assessment by a physician or other clinician.

The directional coupler 208 may operate to couple a portion of the energy emitted by filter circuit 207 through an output connector into detecting circuits. The output connector is connected to a primary input of the directional coupler, and co-axial cable is connected to a primary output of directional coupler. Direction coupler includes two secondary outputs, each of which is connected to a detecting circuit. First detecting circuit functions to detect the amount of power applied to the patient, and second detecting circuit functions to detect the amount of power reflected from the patient. First detecting circuit is connected through a resistive divider to the positive input of a differential amplifier. Second detecting circuit is connected through the resistive divider to the negative input of differential amplifier. The output of differential amplifier is indicative of the difference between the power transmitted to the patient by filter circuit, and the power reflected from the patient, and thus is indicative of an amount of power absorbed by the patient. The output of differential amplifier is applied to an analog to digital converter (ADC) or comparator, the output of which connected to controller 201.

The fully digital synthesis of the modulated digital signal in the digital frequency synthesizer 204 combined with the linear output stage allows for modulation of the applied radiation using other modulation types in addition to amplitude modulation.

The emission device 101 may also include a power source 210 for supplying power to one or more components of the emission device 101. The power source 210 may be an energy storage system like a battery, a super capacitor, lithium ion cell, fuel cell or another energy storage. The power source 210 may be recharged using direct or inductive charging from a power source such as an AC power outlet, a docking station, or the like. In certain embodiments, the controller 201 may monitor the power level of the power source 210, and may provide an alert to a user when the power level reaches a threshold level. In addition, the controller 201 may perform actions such as switch off (and/or initiate low power mode) the emission device 101, or one or more of its components of the emission device 101 based on the power level.

Optionally, a docking station may be included in the system (not shown here) configured to recharge the power source 210. The docking station may include components such as a communications interface, a user authentication module, an activation module (for activating the emission device 101, or any other now or hereafter known components.

The emission device 101 may also include a communications interface 211 that allows the controller 201 to communicate with devices external to the emission device 101 such as a server, an electronic device, a docking station, etc. Examples of the communication interfaces may comprise, but are not limited to, serial interfaces such as RS-232, Universal Serial Bus (USB), Small Computer Systems Interface (SCSI), Ethernet, RS-422 or a wireless communication interface such as Wi-Fi, Bluetooth, near-field communication (NFC) or other wireless interfaces. The controller 201 may communicate with an external device via the communication interface 211 in any communication protocols such as Automation/Drive Interface (ADI).

As discussed above, the emission device 101 may also include optional components such as a display 212 (which can display various indications/alerts of the operation of emission device 101), a user interface 213 (e.g., a keyboard, microphone, touch interface, etc. configured to user instructions), and other output component(s) 214 (e.g., LEDs, speakers, or the like).

In certain embodiments, the emission device 101 may include an activation module (not shown here) for activating the emission device 101 for a particular treatment session. The treatment session may be user specific and determined based on authentication information received from a user. For example, the treatment session may be activated for a user depending upon the disease state of the user. Various parameters of a treatment session may include, without limitation, the total duration of the emission for a treatment session, the power level of the emission, the duty cycle of the emission (i.e., the ratio of on time to off time of pulsed emissions applied during a treatment), the sequence of application of different modulation frequencies for a particular application, and the total number of treatments and duration of each treatment prescribed for a particular subject, and combinations thereof.

The activation module may receive authentication information from a user, via for example, the user interface (e.g., fingerprint, retina scan, login credentials, etc.), an activation card (e.g., an RFID chip card, an ISO chip card, a contact less (NFC) card, etc.) in communication with an activation interface of the emission device 101 and/or an external device in communication with the emission device 101 such as the docking station.

The controller 201 of the emission device 101 may operate to analyze the information provided by the directional coupler to determine and control the amount of power applied to the patient, to assess patient treatment compliance, and to possibly to record indicia of the patient treatment compliance on memory 202 for later analysis and assessment by a physician or other clinician.

Exemplary of treatments performed on patients have included brain, bladder, colorectal, kidney, mesothelium, neuroendocrine, liver, biliary tract, lung, breast, ovary, pancreas, prostate and thyroid tumor types. The treatments involved applying carrier signal, amplitude-modulated at specifically defined frequencies ranging from about 0.1 to about 150,000 Hz at very high precision and stability. The carrier signal may be from about 1 KHz to 5000 MHz, or from about 0.1-1000 MHz, or from about 1-500 MHz, or from about 1-100 MHz, or from about 5-50 MHz, or from about 10 MHz to about 40 MHz, or from about 15 MHz to about 30 MHz, or any other frequency that is capable of leveraging a subject's body as an antenna (e.g., 27 MHz). The one or more modulation frequencies may be simultaneously emitted or sequenced to form the modulation signal. The modulation frequency digital signal may be about 0.1 Hz to about 150,000 Hz, more preferably within about 100 Hz to about 99,000 Hz. The one or more modulation frequencies may be determined and selected based on the type of disease state or cancer to be treated. Further examples of treatment modes (at specific accurately controlled AM frequencies) for specified types of tumors are described in detail below.

FIG. 6 depicts an example of internal hardware that may be included in any of the electronic components of the emission device 101. An electrical bus 600 serves as an information highway interconnecting the other illustrated components of the hardware. Processor 605 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. Read only memory (ROM), random access memory (RAM), flash memory, hard drives and other devices capable of storing electronic data constitute examples of memory devices 625. A memory device may include a single device or a collection of devices across which data and/or instructions are stored.

An optional display interface 630 may permit information from the bus 600 to be displayed on a display device 635 in visual, graphic or alphanumeric format. An audio interface and audio output (such as a speaker) also may be provided. Communication with external devices may occur using various communication devices 640 such as a wireless antenna, an RFID tag and/or short-range or near-field communication transceiver, each of which may optionally communicatively connect with other components of the device via one or more communication system. The communication device 640 may be configured to be communicatively connected to a communications network, such as the Internet, a local area network or a cellular telephone data network.

The hardware may also include a user interface sensor 645 that allows for receipt of data from input devices 650 such as a keyboard, a mouse, a joystick, a touchscreen, a touch pad, a remote control, a pointing device and/or microphone. Digital image frames also may be received from a camera 620 that can capture video and/or still images. The hardware may also include one or more sensors 660 such as position sensors (global positioning systems), temperature sensors, pulse rate sensors, heart pressure monitors, resistance sensors, or the like.

Frequencies that are effective for treatment of a particular disease state or cancer may be discovered using any now or hereafter known methods. Frequency discovery may include exposing one or more subjects pre-diagnosed with a particular health condition (for example, a particular cancer type) to precise modulation frequencies that are applied to the subject or subjects and measuring variations in one or more physiological responses of the patient(s). The frequencies determined in this manner may also be screened against healthy subjects to determine disease-specific modulation frequencies. Control measurements may be obtained by exposing one or more subjects that do not suffer from a particular health condition (for example, a particular cancer type) to modulation frequencies that are applied to the subject or subjects and measuring variations in one or more physiological responses of the patient(s). For example, frequency discovery may include measuring variations in skin electrical resistance, pulse amplitude and/or blood pressure of a subject while being exposed to modulation frequencies generated using the methods and systems disclosed herein. For frequency discovery, the subject may be exposed to modulation frequencies from about 0.1 Hz to about 150,000 Hz incrementally (e.g., using increments of about 50 Hz to about 150 Hz, preferably about 75 Hz to about 125 Hz), and variations in skin electrical resistance, pulse amplitude and/or blood pressure may be measured. Whenever a change is observed in the measured skin electrical resistance (threshold change), pulse amplitude (for about 1-2 beats), and/or blood pressure (threshold change), exposure to the corresponding modulation frequencies may be repeated using increasingly smaller increments (e.g., 10⁻⁴-10⁻³ Hz). Frequencies eliciting the best biofeedback responses are selected as tumor-specific frequencies. For example, if the variations in the amplitude of the pulse are used, best biofeedback responses may be defined by the magnitude of increased amplitude and/or the number of beats with increased amplitude. Such frequency detection may be performed in subjects at different disease stages: disease progression, stable disease and/or partial response. The selection of specific frequencies may be performed according to the method described in Barbault et al. 2009 J. Exp. Clin. Cancer Res. 28(1): 51.

Using the above methods, it has been determined that the methods and systems of this disclosure provide a safe and promising novel treatment modality for multiple types of cancer such as, without limitation, breast cancer, pancreatic cancer, non-small cell and small cell lung cancer, neuroendocrine tumors, non-Hodgkin lymphoma, adenocarcinoma, head and neck cancer, gastric cancer, glioblastoma, squamous cell carcinoma, hepatocellular carcinoma, cholangiocarcinoma, mesothelioma, thyroid cancer, prostate cancer, rhabdomyosarcoma, multiple myeloma, leukemia, and colorectal cancer.

Amplitude-modulated frequencies discovered to be effective in treatment of different types of cancer are listed below. In general, it may be preferred that all frequencies determined to be specific to a particular cancer-type be applied in the treatment of subjects suffering from the indicated form of cancer. However, a limited number of the determined frequencies also leads to beneficial effects, for example 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed frequencies.

TABLE A

| | |
|---|---|
| 262-262.7 | Hz |
| 266.3-267 | Hz |
| 295-295.6 | Hz |
| 334-334.7 | Hz |
| 374-374.7 | Hz |
| 408-408.7 | Hz |
| 415-415.7 | Hz |
| 418-418.7 | Hz |
| 419.8-420.4 | Hz |
| 420-420.7 | Hz |
| 420.4-420.8 | Hz |
| 429-429.7 | Hz |
| 429.7-430.3 | Hz |
| 439-439.7 | Hz |
| 441-441.7 | Hz |
| 442-442.7 | Hz |
| 465-465.7 | Hz |
| 485-485.7 | Hz |
| 527-527.7 | Hz |
| 556-556.7 | Hz |
| 561-561.7 | Hz |
| 568-568.7 | Hz |
| 572-572.7 | Hz |
| 581-581.7 | Hz |
| 594-594.7 | Hz |
| 605-605.7 | Hz |
| 611-611.7 | Hz |
| 612-612.7 | Hz |
| 624.32-624.8 | Hz |
| 626-626.7 | Hz |
| 627-627.7 | Hz |
| 631-631.7 | Hz |
| 633-633.7 | Hz |
| 638-638.7 | Hz |
| 677.4-678 | Hz |
| 679-679.7 | Hz |
| 709-709.7 | Hz |
| 717-717.7 | Hz |
| 727-727.7 | Hz |
| 733-733.7 | Hz |
| 737-737.7 | Hz |
| 759-759.7 | Hz |
| 761-761.7 | Hz |
| 810-810.7 | Hz |
| 811-811.7 | Hz |
| 817-817.7 | Hz |
| 826-826.7 | Hz |
| 829-829.7 | Hz |
| 830-830.7 | Hz |
| 834-834.7 | Hz |
| 836-836.7 | Hz |
| 883-883.7 | Hz |
| 959-959.7 | Hz |
| 961-961.7 | Hz |
| 963.8-964.25 | Hz |
| 964.5-965 | Hz |
| 966-966.7 | Hz |
| 969-969.7 | Hz |
| 972-972.7 | Hz |
| 974.4-975 | Hz |
| 977-977.7 | Hz |
| 996-996.7 | Hz |
| 1027-1027.7 | Hz |
| 1045-1045.7 | Hz |
| 1157-1157.7 | Hz |
| 1179.2-1179.8 | Hz |
| 1195-1195.7 | Hz |
| 1213-1213.7 | Hz |
| 1220-1220.7 | Hz |
| 1252-1252.7 | Hz |
| 1314-1314.7 | Hz |
| 1336-1336.7 | Hz |
| 1352-1352.7 | Hz |
| 1369-1369.7 | Hz |
| 1436-1436.7 | Hz |
| 1467-1467.7 | Hz |
| 1622-1622.7 | Hz |
| 1652-1652.7 | Hz |
| 1668-1668.7 | Hz |
| 1728-1728.7 | Hz |
| 1759-1759.7 | Hz |
| 1771.7-1772.3 | Hz |
| 1772.3-1772.8 | Hz |
| 1785-1785.7 | Hz |
| 1808-1808.6 | Hz |
| 1815-1815.7 | Hz |
| 1823-1823.7 | Hz |
| 1858-1858.7 | Hz |
| 1868-1868.7 | Hz |
| 1881-1881.7 | Hz |
| 1897-1897.7 | Hz |
| 1903-1903.7 | Hz |
| 1914-1914.7 | Hz |
| 1928-1928.7 | Hz |
| 1963-1963.7 | Hz |
| 1966-1966.7 | Hz |
| 1967-1967.7 | Hz |
| 1973-1973.7 | Hz |
| 1989-1989.7 | Hz |
| 2021-2021.7 | Hz |
| 2023-2023.7 | Hz |
| 2035-2035.7 | Hz |
| 2044-2044.7 | Hz |
| 2065-2065.7 | Hz |
| 2094-2094.7 | Hz |
| 2111-2111.7 | Hz |
| 2128-2128.7 | Hz |
| 2189-2189.7 | Hz |
| 2284-2284.7 | Hz |
| 2287-2287.7 | Hz |
| 2313-2313.7 | Hz |
| 2321-2321.7 | Hz |
| 2328-2328.7 | Hz |
| 2346-2346.7 | Hz |
| 2359-2359.7 | Hz |
| 2372.3-2372.8 | Hz |
| 2373-2373.7 | Hz |
| 2380-2380.7 | Hz |
| 2429-2429.7 | Hz |
| 2439.7-2440.3 | Hz |
| 2455-2455.7 | Hz |
| 2459-2459.7 | Hz |
| 2469.2-2469.7 | Hz |
| 2483-2483.7 | Hz |
| 2513-2513.7 | Hz |
| 2518-2518.7 | Hz |
| 2612-2612.7 | Hz |
| 2627-2627.7 | Hz |
| 2629-2629.7 | Hz |
| 2632-2632.7 | Hz |
| 2636-2636.7 | Hz |
| 2647-2647.7 | Hz |
| 2648-2648.7 | Hz |
| 2653.3-2654 | Hz |
| 2653.7-2654.4 | Hz |
| 2679-2679.7 | Hz |
| 2697-2697.7 | Hz |
| 2706-2706.7 | Hz |
| 2707-2707.7 | Hz |
| 2714-2714.7 | Hz |
| 2720.3-2721 | Hz |
| 2726-2726.7 | Hz |
| 2738-2738.7 | Hz |
| 2747-2747.7 | Hz |
| 2754-2754.7 | Hz |
| 2759-2759.7 | Hz |
| 2762-2762.7 | Hz |
| 2774-2774.7 | Hz |
| 2778-2778.7 | Hz |
| 2807-2807.7 | Hz |
| 2811-2811.7 | Hz |
| 2824-2824.7 | Hz |
| 2825-2825.7 | Hz |
| 2826-2826.7 | Hz |
| 2830-2830.7 | Hz |
| 2834-2834.7 | Hz |
| 2836-2836.7 | Hz |
| 2840-2840.7 | Hz |
| 2842-2842.7 | Hz |
| 2854-2854.7 | Hz |
| 2854-2854.6 | Hz |
| 2858.8-2859.35 | Hz |
| 2860-2860.7 | Hz |
| 2864-2864.7 | Hz |
| 2865-2865.7 | Hz |
| 2878-2878.7 | Hz |
| 2890-2890.7 | Hz |
| 2893-2893.7 | Hz |
| 2940-2940.7 | Hz |
| 2971-2971.6 | Hz |
| 2975-2975.7 | Hz |
| 2981-2981.7 | Hz |
| 3030-3030.7 | Hz |
| 3056-3056.7 | Hz |
| 3085.7-3086.3 | Hz |
| 3121-3121.7 | Hz |
| 3133-3133.7 | Hz |
| 3135-3135.7 | Hz |
| 3143-3143.7 | Hz |
| 3171-3171.7 | Hz |
| 3188-3188.7 | Hz |
| 3207-3207.7 | Hz |
| 3209-3209.7 | Hz |
| 3213-3213.7 | Hz |
| 3218-3218.7 | Hz |
| 3222-3222.7 | Hz |
| 3227.3-3228 | Hz |
| 3228-3228.7 | Hz |
| 3234-3234.7 | Hz |
| 3244-3244.7 | Hz |
| 3245-3245.7 | Hz |
| 3250.3-3250.8 | Hz |
| 3251-3251.7 | Hz |
| 3261-3261.7 | Hz |
| 3267-3267.7 | Hz |
| 3269-3269.7 | Hz |
| 3272-3272.7 | Hz |
| 3279.3-3280 | Hz |
| 3280.6-3281.3 | Hz |
| 3281.6-3282.1 | Hz |
| 3322-3322.7 | Hz |
| 3360-3360.7 | Hz |
| 3371-3371.7 | Hz |
| 3428-3428.7 | Hz |
| 3446-3446.7 | Hz |
| 3466-3466.7 | Hz |
| 3522-3522.7 | Hz |
| 3529-3529.7 | Hz |
| 3537-3537.7 | Hz |
| 3548-3548.7 | Hz |
| 3556-3556.7 | Hz |
| 3562-3562.7 | Hz |
| 3571-3571.7 | Hz |
| 3595-3595.7 | Hz |
| 3609-3609.7 | Hz |
| 3618-3618.7 | Hz |
| 3621-3621.7 | Hz |
| 3622.5-3623 | Hz |
| 3627-3627.7 | Hz |
| 3630-3630.7 | Hz |
| 3631-3631.7 | Hz |
| 3633-3633.7 | Hz |
| 3634-3634.7 | Hz |
| 3637.7-3638.3 | Hz |
| 3644-3644.7 | Hz |
| 3646-3646.7 | Hz |
| 3646.4-3647 | Hz |
| 3656-3656.7 | Hz |
| 3661-3661.7 | Hz |
| 3663-3663.7 | Hz |
| 3667-3667.7 | Hz |
| 3678-3678.7 | Hz |
| 3681.7-3682.3 | Hz |
| 3687-3687.7 | Hz |
| 3694-3694.7 | Hz |
| 3731-3731.7 | Hz |
| 3743-3743.7 | Hz |
| 3749.3-3750 | Hz |
| 3756-3756.7 | Hz |
| 3762-3762.7 | Hz |
| 3787-3787.7 | Hz |
| 3817-3817.7 | Hz |
| 3823-3823.7 | Hz |
| 3833-3833.7 | Hz |
| 3839-3839.7 | Hz |
| 3842.4-3843 | Hz |
| 3854-3854.7 | Hz |
| 3877-3877.7 | Hz |
| 3908.7-3909.22 | Hz |
| 3910-3910.7 | Hz |
| 3915-3915.7 | Hz |
| 3922-3922.7 | Hz |
| 3936-3936.7 | Hz |
| 3940-3940.7 | Hz |
| 3946-3946.7 | Hz |
| 3947-3947.7 | Hz |
| 3948-3948.7 | Hz |
| 3954-3954.7 | Hz |
| 3958-3958.6 | Hz |
| 3962-3962.7 | Hz |
| 3981-3981.7 | Hz |
| 4007-4007.7 | Hz |
| 4013-4013.7 | Hz |
| 4027-4027.7 | Hz |
| 4048-4048.7 | Hz |
| 4055-4055.7 | Hz |
| 4135-4135.7 | Hz |
| 4148-4148.7 | Hz |
| 4152-4152.7 | Hz |
| 4155-4155.7 | Hz |
| 4190-4190.7 | Hz |
| 4198-4198.7 | Hz |
| 4224-4224.7 | Hz |
| 4233-4233.7 | Hz |
| 4249-4249.7 | Hz |
| 4281-4281.7 | Hz |
| 4283-4283.7 | Hz |
| 4323-4323.7 | Hz |
| 4368.3-4369 | Hz |
| 4386.4-4387 | Hz |
| 4409-4409.7 | Hz |
| 4410-4410.7 | Hz |
| 4418-4418.7 | Hz |
| 4425-4425.7 | Hz |
| 4425.7-4426.22 | Hz |
| 4430-4430.7 | Hz |
| 4438.7-4439.2 | Hz |
| 4441-4441.7 | Hz |
| 4459-4459.7 | Hz |
| 4465-4465.7 | Hz |
| 4476-4476.7 | Hz |
| 4477.4-4478 | Hz |
| 4523-4523.7 | Hz |
| 4548-4548.7 | Hz |
| 4580.11-4580.7 | Hz |
| 4584-4584.7 | Hz |
| 4621-4621.7 | Hz |
| 4632-4632.7 | Hz |
| 4637-4637.7 | Hz |
| 4657-4657.6 | Hz |
| 4707-4707.7 | Hz |
| 4708-4708.7 | Hz |
| 4713-4713.7 | Hz |
| 4714-4714.7 | Hz |
| 4722-4722.7 | Hz |
| 4723-4723.7 | Hz |
| 4725-4725.7 | Hz |
| 4731-4731.7 | Hz |
| 4733-4733.7 | Hz |
| 4748-4748.7 | Hz |
| 4776-4776.7 | Hz |
| 4792-4792.7 | Hz |
| 4805.3-4806 | Hz |
| 4806-4806.7 | Hz |
| 4809-4809.7 | Hz |
| 4812-4812.7 | Hz |
| 4813-4813.7 | Hz |
| 4821-4821.7 | Hz |
| 4822-4822.7 | Hz |
| 4823-4823.7 | Hz |
| 4833-4833.7 | Hz |
| 4841-4841.7 | Hz |
| 4844-4844.7 | Hz |
| 4850-4850.7 | Hz |
| 4866-4866.7 | Hz |
| 4870-4870.7 | Hz |
| 4874-4874.7 | Hz |
| 4890-4890.7 | Hz |
| 4924-4924.7 | Hz |
| 4941-4941.7 | Hz |
| 4946-4946.7 | Hz |
| 4948-4948.7 | Hz |
| 4997-4997.7 | Hz |
| 4998-4998.7 | Hz |
| 5034-5034.7 | Hz |
| 5063-5063.7 | Hz |
| 5089-5089.7 | Hz |
| 5116-5116.7 | Hz |
| 5139-5139.7 | Hz |
| 5142-5142.7 | Hz |
| 5152-5152.7 | Hz |
| 5154-5154.7 | Hz |
| 5156-5156.7 | Hz |
| 5165.4-5166 | Hz |
| 5205-5205.7 | Hz |
| 5209-5209.7 | Hz |
| 5209.4-5210 | Hz |
| 5221-5221.7 | Hz |
| 5229-5229.7 | Hz |
| 5232-5232.7 | Hz |
| 5239-5239.7 | Hz |
| 5243-5243.7 | Hz |
| 5259-5259.7 | Hz |
| 5265-5265.7 | Hz |
| 5270-5270.7 | Hz |
| 5286-5286.7 | Hz |
| 5322-5322.7 | Hz |
| 5336-5336.7 | Hz |
| 5378.2-5378.7 | Hz |
| 5407.3-5408 | Hz |
| 5414-5414.7 | Hz |
| 5424-5424.7 | Hz |
| 5427-5427.7 | Hz |
| 5438-5438.7 | Hz |
| 5484.4-5485 | Hz |
| 5510-5510.7 | Hz |
| 5533-5533.7 | Hz |
| 5618-5618.7 | Hz |
| 5640.4-5641 | Hz |
| 5671-5671.7 | Hz |
| 5714-5714.7 | Hz |
| 5716-5716.7 | Hz |
| 5741-5741.7 | Hz |
| 5742-5742.7 | Hz |
| 5752-5752.7 | Hz |
| 5766-5766.7 | Hz |
| 5881-5881.7 | Hz |
| 5884-5884.7 | Hz |
| 5976-5976.7 | Hz |
| 5996-5996.7 | Hz |
| 6010-6010.7 | Hz |
| 6024-6024.7 | Hz |
| 6058-6058.7 | Hz |
| 6070-6070.7 | Hz |
| 6074-6074.7 | Hz |
| 6094-6094.7 | Hz |
| 6112-6112.7 | Hz |
| 6117-6117.7 | Hz |
| 6159-6159.7 | Hz |
| 6167-6167.7 | Hz |
| 6179-6179.7 | Hz |
| 6220.3-6221 | Hz |
| 6252-6252.7 | Hz |
| 6297-6297.7 | Hz |
| 6298.4-6299 | Hz |
| 6317-6317.7 | Hz |
| 6330-6330.7 | Hz |
| 6333-6333.7 | Hz |
| 6334-6334.7 | Hz |
| 6346-6346.7 | Hz |
| 6380.4-6381 | Hz |
| 6392-6392.7 | Hz |
| 6413-6413.7 | Hz |
| 6414-6414.7 | Hz |
| 6416.3-6417 | Hz |
| 6419.3-6420 | Hz |
| 6423-6423.7 | Hz |
| 6432-6432.7 | Hz |
| 6436-6436.7 | Hz |
| 6438-6438.7 | Hz |
| 6441-6441.7 | Hz |
| 6443-6443.7 | Hz |
| 6444-6444.7 | Hz |
| 6468-6468.7 | Hz |
| 6469-6469.7 | Hz |
| 6479.4-6480 | Hz |
| 6488-6488.7 | Hz |
| 6492.3-6493 | Hz |
| 6513-6513.7 | Hz |
| 6537-6537.7 | Hz |
| 6648-6648.7 | Hz |
| 6717-6717.7 | Hz |
| 6727.4-6728 | Hz |
| 6730-6730.7 | Hz |
| 6748-6748.7 | Hz |
| 6751-6751.7 | Hz |
| 6781-6781.7 | Hz |
| 6782-6782.7 | Hz |
| 6808-6808.7 | Hz |
| 6809-6809.6 | Hz |
| 6825-6825.7 | Hz |
| 6832-6832.7 | Hz |
| 6840-6840.7 | Hz |
| 6841-6841.7 | Hz |
| 6847-6847.7 | Hz |
| 6864-6864.7 | Hz |
| 6869-6869.7 | Hz |
| 6897-6897.7 | Hz |
| 6911.3-6912 | Hz |
| 6912-6912.7 | Hz |
| 6914-6914.7 | Hz |
| 6968.4-6969 | Hz |
| 6974-6974.7 | Hz |
| 6982-6982.7 | Hz |
| 6986-6986.7 | Hz |
| 7013-7013.7 | Hz |
| 7037-7037.7 | Hz |
| 7042-7042.7 | Hz |
| 7080-7080.7 | Hz |
| 7098-7098.7 | Hz |
| 7109-7109.7 | Hz |
| 7110-7110.7 | Hz |
| 7112-7112.7 | Hz |
| 7117-7117.7 | Hz |
| 7136-7136.7 | Hz |
| 7137-7137.7 | Hz |
| 7145.4-7146 | Hz |
| 7149-7149.7 | Hz |
| 7151-7151.7 | Hz |
| 7158-7158.7 | Hz |
| 7160-7160.7 | Hz |
| 7169-7169.7 | Hz |
| 7191.7-7192.3 | Hz |
| 7222.4-7223 | Hz |
| 7225-7225.7 | Hz |
| 7234-7234.7 | Hz |
| 7239-7239.7 | Hz |
| 7246-7246.7 | Hz |
| 7253-7253.7 | Hz |
| 7268-7268.7 | Hz |
| 7274-7274.7 | Hz |
| 7283-7283.7 | Hz |
| 7338-7338.7 | Hz |
| 7367-7367.7 | Hz |
| 7378-7378.7 | Hz |
| 7404.4-7405 | Hz |
| 7424-7424.7 | Hz |
| 7440-7440.7 | Hz |
| 7481-7481.7 | Hz |
| 7510-7510.7 | Hz |
| 7526-7526.6 | Hz |
| 7531-7531.7 | Hz |
| 7546-7546.7 | Hz |
| 7551-7551.7 | Hz |
| 7560-7560.7 | Hz |
| 7646-7646.7 | Hz |
| 7652-7652.7 | Hz |
| 7655-7655.7 | Hz |
| 7673-7673.7 | Hz |
| 7676-7676.7 | Hz |
| 7719-7719.7 | Hz |
| 7748-7748.7 | Hz |
| 7787-7787.7 | Hz |
| 7808-7808.7 | Hz |
| 7811-7811.7 | Hz |
| 7855-7855.7 | Hz |
| 7857-7857.7 | Hz |
| 7865-7865.7 | Hz |
| 7879-7879.7 | Hz |
| 7906-7906.7 | Hz |
| 7910-7910.7 | Hz |
| 7913-7913.7 | Hz |
| 7923-7923.7 | Hz |
| 7927-7927.7 | Hz |
| 7930-7930.7 | Hz |
| 7936-7936.7 | Hz |
| 7951-7951.7 | Hz |
| 7970-7970.7 | Hz |
| 7981-7981.7 | Hz |
| 7994-7994.7 | Hz |
| 8012.3-8013 | Hz |
| 8013-8013.7 | Hz |
| 8024-8024.7 | Hz |
| 8026-8026.7 | Hz |
| 8032-8032.7 | Hz |
| 8039-8039.7 | Hz |
| 8043-8043.7 | Hz |
| 8074-8074.7 | Hz |
| 8079-8079.7 | Hz |
| 8097-8097.7 | Hz |
| 8125-8125.7 | Hz |
| 8145-8145.7 | Hz |
| 8158-8158.7 | Hz |
| 8217.4-8218 | Hz |
| 8251-8251.7 | Hz |
| 8314-8314.7 | Hz |
| 8332-8332.7 | Hz |
| 8347-8347.7 | Hz |
| 8355-8355.7 | Hz |
| 8373-8373.7 | Hz |
| 8386-8386.7 | Hz |
| 8388-8388.7 | Hz |
| 8412-8412.7 | Hz |
| 8413-8413.7 | Hz |
| 8421-8421.7 | Hz |
| 8427-8427.7 | Hz |
| 8445-8445.7 | Hz |
| 8447-8447.7 | Hz |
| 8467-8467.7 | Hz |
| 8478.3-8479 | Hz |
| 8480-8480.7 | Hz |
| 8484-8484.7 | Hz |
| 8509.4-8510 | Hz |
| 8522-8522.7 | Hz |
| 8546-8546.7 | Hz |
| 8562-8562.7 | Hz |
| 8576-8576.7 | Hz |
| 8698-8698.7 | Hz |
| 8731-8731.7 | Hz |
| 8734-8734.7 | Hz |
| 8740-8740.7 | Hz |
| 8755-8755.7 | Hz |
| 8756-8756.7 | Hz |
| 8757-8757.7 | Hz |
| 8765-8765.7 | Hz |
| 8771-8771.7 | Hz |
| 8789.3-8790 | Hz |
| 8809-8809.7 | Hz |
| 8810-8810.7 | Hz |
| 8814-8814.7 | Hz |
| 8823-8823.7 | Hz |
| 8824-8824.7 | Hz |
| 8827.4-8828 | Hz |
| 8829-8829.7 | Hz |
| 8837-8837.7 | Hz |
| 8841-8841.7 | Hz |
| 8843-8843.7 | Hz |
| 8845-8845.7 | Hz |
| 8846-8846.7 | Hz |
| 8853-8853.7 | Hz |
| 8859.3-8860 | Hz |
| 8872-8872.7 | Hz |
| 8877-8877.7 | Hz |
| 8881-8881.7 | Hz |
| 8889-8889.7 | Hz |
| 8926-8926.7 | Hz |
| 8935-8935.6 | Hz |
| 8949-8949.7 | Hz |
| 9023-9023.7 | Hz |
| 9071-9071.7 | Hz |
| 9075-9075.7 | Hz |
| 9078-9078.7 | Hz |
| 9084-9084.7 | Hz |
| 9106-9106.7 | Hz |
| 9109-9109.7 | Hz |
| 9112-9112.7 | Hz |
| 9122-9122.7 | Hz |
| 9123-9123.7 | Hz |
| 9133-9133.7 | Hz |
| 9136-9136.7 | Hz |
| 9146-9146.7 | Hz |
| 9155.7-9156.2 | Hz |
| 9157-9157.7 | Hz |
| 9161-9161.7 | Hz |
| 9169-9169.7 | Hz |
| 9174-9174.7 | Hz |
| 9179-9179.7 | Hz |
| 9194-9194.7 | Hz |
| 9230-9230.7 | Hz |
| 9236-9236.7 | Hz |
| 9247-9247.7 | Hz |
| 9273-9273.7 | Hz |
| 9506-9506.7 | Hz |
| 9512-9512.7 | Hz |
| 9573-9573.7 | Hz |
| 9588-9588.7 | Hz |
| 9635-9635.7 | Hz |
| 9656-9656.7 | Hz |
| 9681-9681.7 | Hz |
| 9695-9695.7 | Hz |
| 9731-9731.7 | Hz |
| 9846-9846.7 | Hz |
| 9851-9851.7 | Hz |
| 9886-9886.7 | Hz |
| 9921-9921.7 | Hz |
| 9929-9929.7 | Hz |
| 9935-9935.7 | Hz |
| 9947-9947.7 | Hz |
| 9961-9961.7 | Hz |
| 9970-9970.7 | Hz |
| 10013-10013.7 | Hz |
| 10064-10064.7 | Hz |
| 10073-10073.7 | Hz |
| 10137-10137.7 | Hz |
| 10140-10140.7 | Hz |
| 10159-10159.7 | Hz |
| 10339.8-10340.4 | Hz |
| 10342-10342.6 | Hz |
| 10345-10345.7 | Hz |
| 10352-10352.7 | Hz |
| 10362-10362.7 | Hz |
| 10378-10378.7 | Hz |
| 10384-10384.7 | Hz |
| 10387-10387.7 | Hz |
| 10395-10395.7 | Hz |
| 10427-10427.7 | Hz |
| 10436-10436.7 | Hz |
| 10439-10439.7 | Hz |
| 10484-10484.7 | Hz |
| 10488-10488.7 | Hz |
| 10541-10541.7 | Hz |
| 10566-10566.7 | Hz |
| 10566.4-10567 | Hz |
| 10633-10633.7 | Hz |
| 10641-10641.7 | Hz |
| 10645-10645.7 | Hz |
| 10661-10661.7 | Hz |
| 10665.4-10666 | Hz |
| 10671-10671.7 | Hz |
| 10673-10673.7 | Hz |
| 10690-10690.7 | Hz |
| 10708-10708.7 | Hz |
| 10727-10727.7 | Hz |
| 10731-10731.7 | Hz |
| 10735-10735.7 | Hz |
| 10740-10740.7 | Hz |
| 10745-10745.7 | Hz |
| 10748-10748.7 | Hz |
| 10762-10762.7 | Hz |
| 10768-10768.7 | Hz |
| 10776-10776.7 | Hz |
| 10781-10781.7 | Hz |
| 10788-10788.7 | Hz |
| 10802-10802.7 | Hz |
| 10805-10805.7 | Hz |
| 10809-10809.7 | Hz |
| 10813.2-10813.8 | Hz |
| 10830-10830.7 | Hz |
| 10831-10831.7 | Hz |
| 10831.7-10832.1 | Hz |
| 10834-10834.7 | Hz |
| 10844-10844.7 | Hz |
| 10845-10845.7 | Hz |
| 10848.7-10849.3 | Hz |
| 10851-10851.7 | Hz |
| 10854-10854.7 | Hz |
| 10859-10859.7 | Hz |
| 10870-10870.7 | Hz |
| 10873-10873.7 | Hz |
| 10876-10876.7 | Hz |
| 10878-10878.7 | Hz |
| 10889-10889.7 | Hz |
| 10891-10891.7 | Hz |
| 10894-10894.7 | Hz |
| 10906-10906.7 | Hz |
| 10947-10947.7 | Hz |
| 10974-10974.7 | Hz |
| 10993-10993.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11081-11081.7 | Hz |
| 11083-11083.7 | Hz |
| 11109.3-11110 | Hz |
| 11110-11110.7 | Hz |
| 11117-11117.7 | Hz |
| 11124-11124.7 | Hz |
| 11134-11134.7 | Hz |
| 11155-11155.7 | Hz |
| 11159-11159.7 | Hz |
| 11163-11163.7 | Hz |
| 11164-11164.7 | Hz |
| 11174-11174.7 | Hz |
| 11209-11209.7 | Hz |
| 11219-11219.7 | Hz |
| 11225-11225.7 | Hz |
| 11234-11234.7 | Hz |
| 11243-11243.7 | Hz |
| 11247-11247.7 | Hz |
| 11253-11253.7 | Hz |
| 11255-11255.7 | Hz |
| 11260-11260.7 | Hz |
| 11264-11264.7 | Hz |
| 11267-11267.7 | Hz |
| 11275-11275.7 | Hz |
| 11277.3-11278 | Hz |
| 11284-11284.7 | Hz |
| 11295-11295.7 | Hz |
| 11336-11336.7 | Hz |
| 11381-11381.7 | Hz |
| 11387-11387.7 | Hz |
| 11414-11414.7 | Hz |
| 11416-11416.7 | Hz |
| 11429-11429.7 | Hz |
| 11438-11438.7 | Hz |
| 11469-11469.7 | Hz |
| 11524-11524.7 | Hz |
| 11530-11530.7 | Hz |
| 11533-11533.7 | Hz |
| 11551.3-11552 | Hz |
| 11556-11556.7 | Hz |
| 11569-11569.7 | Hz |
| 11576-11576.7 | Hz |
| 11582-11582.7 | Hz |
| 11590-11590.7 | Hz |
| 11619-11619.7 | Hz |
| 11621-11621.7 | Hz |
| 11628-11628.7 | Hz |
| 11630-11630.7 | Hz |
| 11637-11637.7 | Hz |
| 11638-11638.7 | Hz |
| 11681-11681.7 | Hz |
| 11699-11699.7 | Hz |
| 11719-11719.7 | Hz |
| 11730.6-11731.1 | Hz |
| 11745-11745.7 | Hz |
| 11779-11779.7 | Hz |
| 11785-11785.7 | Hz |
| 11809-11809.7 | Hz |
| 11831-11831.7 | Hz |
| 11867-11867.7 | Hz |
| 11872-11872.7 | Hz |
| 11905-11905.7 | Hz |
| 11911-11911.7 | Hz |
| 11913-11913.7 | Hz |
| 11929-11929.7 | Hz |
| 11931-11931.7 | Hz |
| 11934-11934.7 | Hz |
| 11946-11946.7 | Hz |
| 11949-11949.7 | Hz |
| 11956-11956.7 | Hz |
| 11974-11974.7 | Hz |
| 11993-11993.7 | Hz |
| 11994-11994.7 | Hz |
| 12118-12118.7 | Hz |
| 12120.2-12120.7 | Hz |
| 12135-12135.7 | Hz |
| 12137-12137.7 | Hz |
| 12144-12144.7 | Hz |
| 12152-12152.7 | Hz |
| 12165-12165.7 | Hz |
| 12182-12182.7 | Hz |
| 12184-12184.7 | Hz |
| 12302-12302.7 | Hz |
| 12309-12309.7 | Hz |
| 12318-12318.7 | Hz |
| 12330-12330.7 | Hz |
| 12342-12342.7 | Hz |
| 12356-12356.7 | Hz |
| 12364-12364.7 | Hz |
| 12373-12373.7 | Hz |
| 12391-12391.7 | Hz |
| 12498-12498.7 | Hz |
| 12557-12557.7 | Hz |
| 12572-12572.7 | Hz |
| 12575-12575.7 | Hz |
| 12588-12588.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 12649-12649.7 | Hz |
| 12682-12682.7 | Hz |
| 12711-12711.7 | Hz |
| 12752-12752.7 | Hz |
| 12783-12783.7 | Hz |
| 12880-12880.7 | Hz |
| 12929-12929.7 | Hz |
| 13117-13117.7 | Hz |
| 13127-13127.7 | Hz |
| 13215-13215.7 | Hz |
| 13222-13222.7 | Hz |
| 13241-13241.7 | Hz |
| 13252-13252.7 | Hz |
| 13307-13307.7 | Hz |
| 13329-13329.7 | Hz |
| 13330.7-13331.2 | Hz |
| 13373-13373.7 | Hz |
| 13375-13375.7 | Hz |
| 13377-13377.7 | Hz |
| 13386-13386.7 | Hz |
| 13408-13408.7 | Hz |
| 13418-13418.7 | Hz |
| 13434-13434.7 | Hz |
| 13475-13475.7 | Hz |
| 13562-13562.6 | Hz |
| 13571-13571.7 | Hz |
| 13609-13609.7 | Hz |
| 13611-13611.7 | Hz |
| 13615-13615.7 | Hz |
| 13617-13617.7 | Hz |
| 13636.2-13636.7 | Hz |
| 13644-13644.7 | Hz |
| 13661-13661.7 | Hz |
| 13670-13670.7 | Hz |
| 13679-13679.7 | Hz |
| 13686.4-13687 | Hz |
| 13694-13694.7 | Hz |
| 13713-13713.7 | Hz |
| 13714-13714.7 | Hz |
| 13734-13734.7 | Hz |
| 13736-13736.7 | Hz |
| 13738.3-13739 | Hz |
| 13742-13742.7 | Hz |
| 13746-13746.7 | Hz |
| 13747-13747.7 | Hz |
| 13751-13751.7 | Hz |
| 13755-13755.7 | Hz |
| 13760-13760.7 | Hz |
| 13761-13761.7 | Hz |
| 13767-13767.7 | Hz |
| 13773-13773.7 | Hz |
| 13774-13774.7 | Hz |
| 13775-13775.7 | Hz |
| 13780-13780.7 | Hz |
| 13805-13805.7 | Hz |
| 13819-13819.7 | Hz |
| 13828-13828.7 | Hz |
| 13834-13834.7 | Hz |
| 13847-13847.7 | Hz |
| 13856-13856.7 | Hz |
| 13857.4-13858 | Hz |
| 13858-13858.7 | Hz |
| 13871-13871.7 | Hz |
| 13903-13903.7 | Hz |
| 13947-13947.7 | Hz |
| 13978.3-13979 | Hz |
| 13981-13981.7 | Hz |
| 14005-14005.7 | Hz |
| 14009-14009.7 | Hz |
| 14016.4-14017 | Hz |
| 14027-14027.7 | Hz |
| 14034-14034.7 | Hz |
| 14052-14052.7 | Hz |
| 14070-14070.7 | Hz |
| 14078-14078.7 | Hz |
| 14111-14111.7 | Hz |
| 14114-14114.7 | Hz |
| 14116-14116.7 | Hz |
| 14153-14153.7 | Hz |
| 14156-14156.7 | Hz |
| 14160-14160.7 | Hz |
| 14164-14164.7 | Hz |
| 14170.7-14171.3 | Hz |
| 14174-14174.7 | Hz |
| 14182-14182.7 | Hz |
| 14249-14249.7 | Hz |
| 14279-14279.7 | Hz |
| 14281-14281.7 | Hz |
| 14285-14285.7 | Hz |
| 14308-14308.7 | Hz |
| 14333.4-14333.9 | Hz |
| 14350-14350.7 | Hz |
| 14356-14356.7 | Hz |
| 14376-14376.7 | Hz |
| 14417-14417.7 | Hz |
| 14422-14422.7 | Hz |
| 14432-14432.7 | Hz |
| 14441-14441.7 | Hz |
| 14443-14443.7 | Hz |
| 14456-14456.7 | Hz |
| 14462-14462.7 | Hz |
| 14479-14479.7 | Hz |
| 14483-14483.7 | Hz |
| 14489-14489.7 | Hz |
| 14511-14511.7 | Hz |
| 14523-14523.7 | Hz |
| 14524-14524.7 | Hz |
| 14531-14531.7 | Hz |
| 14532-14532.7 | Hz |
| 14540-14540.7 | Hz |
| 14545-14545.7 | Hz |
| 14553-14553.7 | Hz |
| 14558-14558.7 | Hz |
| 14564-14564.7 | Hz |
| 14570-14570.7 | Hz |
| 14574-14574.7 | Hz |
| 14585-14585.7 | Hz |
| 14589-14589.7 | Hz |
| 14621-14621.7 | Hz |
| 14630-14630.7 | Hz |
| 14670.4-14671 | Hz |
| 14671-14671.7 | Hz |
| 14674-14674.7 | Hz |
| 14690-14690.7 | Hz |
| 14716-14716.7 | Hz |
| 14729-14729.7 | Hz |
| 14756-14756.7 | Hz |
| 14796-14796.7 | Hz |
| 14813-14813.7 | Hz |
| 14835-14835.7 | Hz |
| 14858-14858.7 | Hz |
| 14863-14863.7 | Hz |
| 14867-14867.7 | Hz |
| 14877-14877.7 | Hz |
| 14885-14885.7 | Hz |
| 14911-14911.7 | Hz |
| 14912-14912.7 | Hz |
| 14913.4-14914 | Hz |
| 14922-14922.7 | Hz |
| 14940-14940.7 | Hz |
| 14940.4-14941 | Hz |
| 14957-14957.7 | Hz |
| 14961-14961.7 | Hz |
| 14974-14974.7 | Hz |
| 14982-14982.7 | Hz |
| 15006-15006.7 | Hz |
| 15033-15033.7 | Hz |
| 15035-15035.6 | Hz |
| 15056-15056.7 | Hz |
| 15067-15067.7 | Hz |
| 15071-15071.7 | Hz |
| 15109-15109.7 | Hz |
| 15112-15112.7 | Hz |
| 15126-15126.7 | Hz |
| 15133-15133.7 | Hz |
| 15154-15154.7 | Hz |
| 15173-15173.7 | Hz |
| 15179-15179.7 | Hz |
| 15195-15195.7 | Hz |
| 15223-15223.7 | Hz |
| 15224-15224.7 | Hz |
| 15225-15225.6 | Hz |
| 15226-15226.7 | Hz |
| 15230-15230.7 | Hz |
| 15233-15233.7 | Hz |
| 15236-15236.7 | Hz |
| 15244-15244.7 | Hz |
| 15250-15250.7 | Hz |
| 15253-15253.7 | Hz |
| 15263-15263.7 | Hz |
| 15269-15269.7 | Hz |
| 15278-15278.7 | Hz |
| 15329-15329.7 | Hz |
| 15432-15432.7 | Hz |
| 15435-15435.7 | Hz |
| 15460-15460.7 | Hz |
| 15515-15515.7 | Hz |
| 15521-15521.7 | Hz |
| 15528-15528.7 | Hz |
| 15531-15531.7 | Hz |
| 15536-15536.7 | Hz |
| 15539-15539.7 | Hz |
| 15545-15545.7 | Hz |
| 15546.3-15547 | Hz |
| 15550.4-15551 | Hz |
| 15563-15563.7 | Hz |
| 15576-15576.7 | Hz |
| 15589-15589.7 | Hz |
| 15615-15615.7 | Hz |
| 15621-15621.7 | Hz |
| 15624-15624.7 | Hz |
| 15642-15642.7 | Hz |
| 15647-15647.7 | Hz |
| 15663-15663.7 | Hz |
| 15669-15669.7 | Hz |
| 15678-15678.7 | Hz |
| 15698-15698.7 | Hz |
| 15711-15711.7 | Hz |
| 15724-15724.7 | Hz |
| 15729-15729.7 | Hz |
| 15740-15740.7 | Hz |
| 15749-15749.7 | Hz |
| 15773-15773.7 | Hz |
| 15817-15817.7 | Hz |
| 15823-15823.7 | Hz |
| 15824-15824.7 | Hz |
| 15825-15825.7 | Hz |
| 15835-15835.7 | Hz |
| 15836-15836.7 | Hz |
| 15857-15857.7 | Hz |
| 15867-15867.7 | Hz |
| 15897-15897.7 | Hz |
| 15919-15919.7 | Hz |
| 15942-15942.7 | Hz |
| 15982-15982.7 | Hz |
| 16071-16071.7 | Hz |
| 16082-16082.7 | Hz |
| 16096-16096.7 | Hz |
| 16143-16143.7 | Hz |
| 16176-16176.7 | Hz |
| 16274-16274.7 | Hz |
| 16309-16309.7 | Hz |
| 16329-16329.7 | Hz |
| 16347-16347.7 | Hz |
| 16381-16381.7 | Hz |
| 16515-16515.7 | Hz |
| 16679-16679.7 | Hz |
| 16804-16804.7 | Hz |
| 16821-16821.7 | Hz |
| 17095-17095.7 | Hz |
| 17106-17106.7 | Hz |
| 17123-17123.7 | Hz |
| 17167-17167.7 | Hz |
| 17214-17214.7 | Hz |
| 17229-17229.7 | Hz |
| 17246-17246.7 | Hz |
| 17268-17268.7 | Hz |
| 17271-17271.7 | Hz |
| 17283-17283.7 | Hz |
| 17333-17333.7 | Hz |
| 17379-17379.7 | Hz |
| 17417-17417.7 | Hz |
| 17418-17418.7 | Hz |
| 17435-17435.7 | Hz |
| 17455-17455.7 | Hz |
| 17472-17472.7 | Hz |
| 17473-17473.7 | Hz |
| 17474-17474.7 | Hz |
| 17485-17485.7 | Hz |
| 17515-17515.7 | Hz |
| 17556-17556.7 | Hz |
| 17578.4-17579 | Hz |
| 17581-17581.7 | Hz |
| 17621-17621.7 | Hz |
| 17636-17636.7 | Hz |
| 17655-17655.7 | Hz |
| 17673-17673.7 | Hz |
| 17737-17737.7 | Hz |
| 17739-17739.7 | Hz |
| 17749-17749.7 | Hz |
| 17757-17757.7 | Hz |
| 17808-17808.7 | Hz |
| 17825-17825.7 | Hz |
| 17832-17832.7 | Hz |
| 17944-17944.7 | Hz |
| 17962-17962.7 | Hz |
| 17974-17974.7 | Hz |
| 17978-17978.7 | Hz |
| 18021-18021.7 | Hz |
| 18040-18040.7 | Hz |
| 18087-18087.7 | Hz |
| 18090-18090.7 | Hz |
| 18126-18126.7 | Hz |
| 18140-18140.7 | Hz |
| 18157-18157.7 | Hz |
| 18171-18171.7 | Hz |
| 18182-18182.7 | Hz |
| 18211-18211.7 | Hz |
| 18221-18221.7 | Hz |
| 18222-18222.7 | Hz |
| 18246-18246.7 | Hz |
| 18260-18260.7 | Hz |
| 18274-18274.7 | Hz |
| 18279-18279.7 | Hz |
| 18294-18294.7 | Hz |
| 18372-18372.7 | Hz |
| 18375-18375.7 | Hz |
| 18396-18396.7 | Hz |
| 18411-18411.7 | Hz |
| 18454-18454.7 | Hz |
| 18462-18462.7 | Hz |
| 18521-18521.7 | Hz |
| 18533-18533.7 | Hz |
| 18567-18567.7 | Hz |
| 18585-18585.7 | Hz |
| 18596-18596.7 | Hz |
| 18618-18618.7 | Hz |
| 18635-18635.7 | Hz |
| 18641-18641.7 | Hz |
| 18655-18655.7 | Hz |
| 18670.3-18671 | Hz |
| 18672-18672.7 | Hz |
| 18678-18678.7 | Hz |
| 18678.7-18679.3 | Hz |
| 18686-18686.7 | Hz |
| 18742-18742.7 | Hz |
| 18831-18831.7 | Hz |
| 18842-18842.7 | Hz |
| 18884-18884.7 | Hz |
| 18895-18895.7 | Hz |
| 18897-18897.7 | Hz |
| 18922-18922.7 | Hz |
| 19026-19026.7 | Hz |
| 19059-19059.7 | Hz |
| 19125-19125.7 | Hz |
| 19269-19269.7 | Hz |
| 19342-19342.7 | Hz |
| 19365-19365.7 | Hz |
| 19384-19384.7 | Hz |
| 19393-19393.7 | Hz |
| 19419-19419.7 | Hz |
| 19426-19426.7 | Hz |
| 19433-19433.7 | Hz |
| 19437-19437.7 | Hz |
| 19446-19446.7 | Hz |
| 19447-19447.7 | Hz |
| 19453-19453.7 | Hz |
| 19464-19464.7 | Hz |
| 19476-19476.7 | Hz |
| 19489.3-19490 | Hz |
| 19510-19510.7 | Hz |
| 19577-19577.7 | Hz |
| 19686-19686.4 | Hz |
| 19715-19715.7 | Hz |
| 19724-19724.7 | Hz |
| 19744-19744.7 | Hz |
| 19782-19782.7 | Hz |
| 19817-19817.7 | Hz |
| 19819-19819.7 | Hz |
| 19823-19823.7 | Hz |
| 19827-19827.7 | Hz |
| 19829-19829.7 | Hz |
| 19861-19861.7 | Hz |
| 19867-19867.7 | Hz |
| 19889-19889.7 | Hz |
| 20097-20097.7 | Hz |
| 20123-20123.7 | Hz |
| 20128-20128.7 | Hz |
| 20144-20144.7 | Hz |
| 20149.3-20150 | Hz |
| 20173-20173.7 | Hz |
| 20219-20219.7 | Hz |
| 20229-20229.7 | Hz |
| 20237-20237.7 | Hz |
| 20254-20254.7 | Hz |
| 20256-20256.7 | Hz |
| 20278.4-20279 | Hz |
| 20284-20284.7 | Hz |
| 20286-20286.7 | Hz |
| 20324-20324.7 | Hz |
| 20499-20499.7 | Hz |
| 20513-20513.7 | Hz |
| 20634-20634.7 | Hz |
| 20747-20747.7 | Hz |
| 20818-20818.7 | Hz |
| 20825-20825.7 | Hz |
| 20908-20908.7 | Hz |
| 20948-20948.7 | Hz |
| 21119-21119.7 | Hz |
| 21167-21167.7 | Hz |
| 21291.4-21292 | Hz |
| 21372-21372.7 | Hz |
| 21389-21389.7 | Hz |
| 21430-21430.7 | Hz |
| 21445-21445.7 | Hz |
| 21466-21466.7 | Hz |
| 21467-21467.7 | Hz |
| 21490-21490.7 | Hz |
| 21546-21546.7 | Hz |
| 21567-21567.7 | Hz |
| 21616-21616.7 | Hz |
| 21636-21636.7 | Hz |
| 21663-21663.7 | Hz |
| 21664-21664.7 | Hz |
| 21708-21708.7 | Hz |
| 21714-21714.7 | Hz |
| 21720-21720.7 | Hz |
| 21778-21778.7 | Hz |
| 21833-21833.7 | Hz |
| 21840-21840.7 | Hz |
| 21844-21844.7 | Hz |
| 21854-21854.7 | Hz |
| 21865-21865.7 | Hz |
| 21870-21870.7 | Hz |
| 21876-21876.7 | Hz |
| 21887-21887.7 | Hz |
| 21890-21890.7 | Hz |
| 21894-21894.7 | Hz |
| 21963-21963.7 | Hz |
| 22020-22020.7 | Hz |
| 22073-22073.7 | Hz |
| 22119-22119.7 | Hz |
| 22167-22167.7 | Hz |
| 22312-22312.7 | Hz |
| 22338-22338.7 | Hz |
| 22344-22344.7 | Hz |
| 22350-22350.7 | Hz |
| 22353-22353.7 | Hz |
| 22355-22355.7 | Hz |
| 22361-22361.7 | Hz |
| 22411-22411.7 | Hz |
| 22419-22419.7 | Hz |
| 22422-22422.7 | Hz |
| 22423.4-22424 | Hz |
| 22425-22425.7 | Hz |
| 22430-22430.7 | Hz |
| 22437-22437.7 | Hz |
| 22443-22443.7 | Hz |
| 22444-22444.7 | Hz |
| 22454-22454.7 | Hz |
| 22459-22459.7 | Hz |
| 22484-22484.7 | Hz |
| 22491-22491.7 | Hz |
| 22519-22519.7 | Hz |
| 22535-22535.7 | Hz |
| 22538-22538.7 | Hz |
| 22548-22548.7 | Hz |
| 22559.3-22560 | Hz |
| 22582-22582.7 | Hz |
| 22597-22597.7 | Hz |
| 22709-22709.7 | Hz |
| 22730-22730.6 | Hz |
| 22742-22742.7 | Hz |
| 22743-22743.7 | Hz |
| 22763-22763.7 | Hz |
| 22822-22822.7 | Hz |
| 22832-22832.7 | Hz |
| 22840.3-22841 | Hz |
| 22846-22846.7 | Hz |
| 22859-22859.7 | Hz |
| 22863-22863.7 | Hz |
| 22867-22867.7 | Hz |
| 22879-22879.7 | Hz |
| 22932-22932.7 | Hz |
| 22973-22973.7 | Hz |
| 22983-22983.7 | Hz |
| 23012-23012.7 | Hz |
| 23044-23044.7 | Hz |
| 23111-23111.7 | Hz |
| 23117-23117.7 | Hz |
| 23137-23137.7 | Hz |
| 23216-23216.7 | Hz |
| 23237-23237.7 | Hz |
| 23246-23246.7 | Hz |
| 23249-23249.7 | Hz |
| 23258-23258.7 | Hz |
| 23259-23259.7 | Hz |
| 23261-23261.7 | Hz |
| 23284-23284.7 | Hz |
| 23344-23344.7 | Hz |
| 23412-23412.7 | Hz |
| 23444-23444.7 | Hz |
| 23509-23509.7 | Hz |
| 23526-23526.7 | Hz |
| 23548-23548.7 | Hz |
| 23560-23560.7 | Hz |
| 23589-23589.7 | Hz |
| 23624-23624.7 | Hz |
| 23652-23652.7 | Hz |
| 23659-23659.7 | Hz |
| 23667-23667.7 | Hz |
| 23686-23686.7 | Hz |
| 23730-23730.7 | Hz |
| 23921-23921.7 | Hz |
| 23946-23946.7 | Hz |
| 23957-23957.7 | Hz |
| 24014-24014.7 | Hz |
| 24060-24060.7 | Hz |
| 24073-24073.7 | Hz |
| 24085-24085.7 | Hz |
| 24112-24112.7 | Hz |
| 24117-24117.7 | Hz |
| 24142-24142.7 | Hz |
| 24146-24146.7 | Hz |
| 24174-24174.7 | Hz |
| 24196-24196.7 | Hz |
| 24198-24198.7 | Hz |
| 24228-24228.7 | Hz |
| 24275-24275.7 | Hz |
| 24413-24413.7 | Hz |
| 24508-24508.7 | Hz |
| 24609.3-24610 | Hz |
| 24789-24789.7 | Hz |
| 24811-24811.7 | Hz |
| 24841-24841.7 | Hz |
| 24869-24869.7 | Hz |
| 25199-25199.7 | Hz |
| 25449-25449.7 | Hz |
| 25454-25454.7 | Hz |
| 26044-26044.7 | Hz |
| 26073-26073.7 | Hz |
| 26113-26113.7 | Hz |
| 26142-26142.7 | Hz |
| 26163-26163.7 | Hz |
| 26164-26164.7 | Hz |
| 26212-26212.7 | Hz |
| 26343-26343.7 | Hz |
| 26403-26403.7 | Hz |
| 26465-26465.7 | Hz |
| 26534-26534.7 | Hz |
| 26722-26722.7 | Hz |
| 26861-26861.7 | Hz |
| 26954-26954.7 | Hz |
| 27114-27114.7 | Hz |
| 27214-27214.7 | Hz |
| 27291-27291.7 | Hz |
| 27328-27328.6 | Hz |
| 27478-27478.7 | Hz |
| 27713-27713.7 | Hz |
| 28212-28212.7 | Hz |
| 28222-28222.7 | Hz |
| 28272-28272.7 | Hz |
| 28361-28361.7 | Hz |
| 28537-28537.7 | Hz |
| 28653-28653.7 | Hz |
| 28744-28744.7 | Hz |
| 30075-30075.7 | Hz |
| 30304-30304.7 | Hz |
| 30373-30373.7 | Hz |
| 30463.3-30464 | Hz |
| 30666-30666.7 | Hz |
| 30713-30713.7 | Hz |
| 30759-30759.7 | Hz |
| 30942-30942.7 | Hz |
| 31051-31051.7 | Hz |
| 31572-31572.7 | Hz |
| 31633-31633.7 | Hz |
| 31685-31685.7 | Hz |
| 31713-31713.7 | Hz |
| 31847-31847.7 | Hz |
| 32143-32143.7 | Hz |
| 32493-32493.7 | Hz |
| 32591.3-32592 | Hz |
| 32866-32866.7 | Hz |
| 32870-32870.7 | Hz |
| 33366-33366.7 | Hz |
| 33414-33414.7 | Hz |
| 33536-33536.7 | Hz |
| 33546-33546.7 | Hz |
| 33553-33553.7 | Hz |
| 33765-33765.7 | Hz |
| 34405-34405.7 | Hz |
| 34557.3-34558 | Hz |
| 34562-34562.7 | Hz |
| 34614-34614.7 | Hz |
| 34854-34854.7 | Hz |
| 34984-34984.6 | Hz |
| 35296-35296.7 | Hz |
| 35324-35324.7 | Hz |
| 35543-35543.7 | Hz |
| 35826-35826.7 | Hz |
| 36343-36343.7 | Hz |
| 36410-36410.7 | Hz |
| 37527-37527.7 | Hz |
| 37546-37546.7 | Hz |
| 37680-37680.7 | Hz |
| 38144-38144.7 | Hz |
| 38216-38216.7 | Hz |
| 38483-38483.7 | Hz |
| 38659-38659.7 | Hz |
| 38682-38682.7 | Hz |
| 39035-39035.7 | Hz |
| 39053-39053.7 | Hz |
| 39153-39153.7 | Hz |
| 39183-39183.7 | Hz |
| 39223-39223.7 | Hz |
| 39423-39423.7 | Hz |
| 39510-39510.7 | Hz |
| 39586-39586.7 | Hz |
| 39729-39729.7 | Hz |
| 40111-40111.7 | Hz |
| 40133-40133.7 | Hz |
| 40394-40394.7 | Hz |
| 40489-40489.7 | Hz |
| 40595-40595.7 | Hz |
| 40693-40693.7 | Hz |
| 40734-40734.7 | Hz |
| 40762-40762.7 | Hz |
| 40896-40896.7 | Hz |
| 40899-40899.7 | Hz |
| 41062-41062.7 | Hz |
| 41172-41172.7 | Hz |
| 41244-41244.7 | Hz |
| 41279-41279.7 | Hz |
| 41325-41325.7 | Hz |
| 41424-41424.7 | Hz |
| 41473-41473.7 | Hz |
| 41534-41534.7 | Hz |
| 41557-41557.7 | Hz |
| 41561-41561.7 | Hz |
| 41574-41574.7 | Hz |
| 41623-41623.7 | Hz |
| 41735-41735.7 | Hz |
| 41859-41859.7 | Hz |
| 41861-41861.7 | Hz |
| 41883-41883.7 | Hz |
| 42146-42146.7 | Hz |
| 42293-42293.7 | Hz |
| 42402-42402.7 | Hz |
| 42544-42544.7 | Hz |
| 42692.3-42693 | Hz |
| 42713-42713.7 | Hz |
| 42812-42812.7 | Hz |
| 42813-42813.7 | Hz |
| 42913-42913.7 | Hz |
| 42923-42923.7 | Hz |
| 43162-43162.7 | Hz |
| 43166-43166.7 | Hz |
| 43172-43172.7 | Hz |
| 43243-43243.7 | Hz |
| 43325-43325.7 | Hz |
| 43379-43379.7 | Hz |
| 43413-43413.7 | Hz |
| 43542-43542.7 | Hz |
| 43603-43603.7 | Hz |
| 43633-43633.7 | Hz |
| 43692-43692.7 | Hz |
| 43851-43851.7 | Hz |
| 44162-44162.7 | Hz |
| 44517-44517.7 | Hz |
| 44531-44531.7 | Hz |
| 44575.3-44576 | Hz |
| 44652-44652.7 | Hz |
| 44692-44692.7 | Hz |
| 44742-44742.7 | Hz |
| 44773-44773.7 | Hz |
| 44855-44855.7 | Hz |
| 44913-44913.7 | Hz |
| 45163-45163.7 | Hz |
| 45362-45362.7 | Hz |
| 45393-45393.7 | Hz |
| 45474-45474.7 | Hz |
| 45490-45490.7 | Hz |
| 45526-45526.7 | Hz |
| 46112-46112.7 | Hz |
| 46143-46143.7 | Hz |
| 46282-46282.7 | Hz |
| 46285-46285.7 | Hz |
| 46340-46340.7 | Hz |
| 46662-46662.7 | Hz |
| 46666-46666.7 | Hz |
| 46672.3-46673 | Hz |
| 46673-46673.7 | Hz |
| 46689-46689.6 | Hz |
| 46746-46746.7 | Hz |
| 46793-46793.7 | Hz |
| 47036-47036.7 | Hz |
| 47297-47297.7 | Hz |
| 47693.4-47694 | Hz |
| 48103-48103.7 | Hz |
| 48173-48173.7 | Hz |
| 48204-48204.7 | Hz |
| 48314-48314.7 | Hz |
| 48535-48535.7 | Hz |
| 48583-48583.7 | Hz |
| 48653-48653.7 | Hz |
| 48683-48683.7 | Hz |
| 48793-48793.7 | Hz |
| 49434-49434.7 | Hz |
| 50072-50072.7 | Hz |
| 50229-50229.7 | Hz |
| 50243-50243.7 | Hz |
| 50254-50254.7 | Hz |
| 50292-50292.7 | Hz |
| 51008-51008.7 | Hz |
| 51592-51592.7 | Hz |
| 51946-51946.7 | Hz |
| 52063-52063.7 | Hz |
| 52097-52097.7 | Hz |
| 52752-52752.7 | Hz |
| 52786-52786.7 | Hz |
| 52812-52812.7 | Hz |
| 52912-52912.7 | Hz |
| 53079-53079.7 | Hz |
| 53947-53947.7 | Hz |
| 54293-54293.7 | Hz |
| 54297-54297.7 | Hz |
| 55072-55072.7 | Hz |
| 55592-55592.7 | Hz |
| 55723-55723.7 | Hz |
| 56093-56093.7 | Hz |
| 57927-57927.7 | Hz |
| 58094-58094.7 | Hz |
| 59089-59089.7 | Hz |
| 59288-59288.7 | Hz |
| 59907-59907.7 | Hz |
| 59997-59997.7 | Hz |
| 61287-61287.7 | Hz |
| 61789-61789.7 | Hz |
| 65586-65586.7 | Hz |
| 66553-66553.7 | Hz |
| 66969-66969.7 | Hz |
| 67683-67683.7 | Hz |
| 67697-67697.6 | Hz |
| 69481-69481.7 | Hz |
| 71204-71204.7 | Hz |
| 71447-71447.7 | Hz |
| 71635-71635.7 | Hz |
| 74017-74017.7 | Hz |
| 76227-76227.7 | Hz |
| 76537-76537.7 | Hz |
| 76659-76659.7 | Hz |
| 78229-78229.7 | Hz |
| 78477.4-78478 | Hz |
| 79658-79658.7 | Hz |
| 79867-79867.7 | Hz |
| 80715-80715.7 | Hz |
| 80993.3-80994 | Hz |
| 81999-81999.7 | Hz |
| 82024-82024.7 | Hz |
| 82845-82845.7 | Hz |
| 83612-83612.7 | Hz |
| 84086-84086.7 | Hz |
| 85810-85810.7 | Hz |
| 86521-86521.7 | Hz |
| 87507-87507.7 | Hz |
| 87798.3-87799 | Hz |
| 88281-88281.7 | Hz |
| 88898-88898.7 | Hz |
| 89748-89748.7 | Hz |
| 91245-91245.7 | Hz |
| 91271-91271.7 | Hz |
| 91877-91877.7 | Hz |
| 92182-92182.7 | Hz |
| 92361.3-92362 | Hz |
| 92787-92787.7 | Hz |
| 93332-93332.7 | Hz |
| 93530-93530.7 | Hz |
| 94494-94494.7 | Hz |
| 94968.3-94969 | Hz |
| 95245-95245.7 | Hz |
| 95742-95742.7 | Hz |
| 99281-99281.7 | Hz |
| 99846-99846.7 | Hz |

In certain embodiments, the cancer specific AM frequencies provided in Table A may also be used in combination with one or more of the AM frequencies disclosed in the '365 patent. One or more of the AM frequencies of Table A may be combined with one or more of the frequencies as provided below in Tables 2, 4, 7, 13, 16 18, 20 and 23-32. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of cancer, which frequencies are selected from Table A and, optionally from Tables 2, 4, 7, 13, 16 18, 20 and 23-32, provided that at least one or more frequencies are selected from Table A.

The device and method described herein for the treatment of different cancer types by the application to the patient of electromagnetic fields that are amplitude-modulated at particular and cancer-specific frequencies may be used as a stand-alone cancer therapy or use in conjunction with other now or hereafter known cancer treatment modalities. The device and method described herein, may be used in combination with surgical intervention, radiotherapy and/or chemotherapy.

When used in combination with other cancer therapies, the treatments methods described in this disclosure may be used as an adjuvant or neoadjuvant therapy in the treatment of cancer. For neoadjuvant therapy, the treatment methods described in this disclosure are administered to the subject before a main treatment. Such neoadjuvant therapy may be applied to reduce the extent and/or size of the cancer prior to the use of a more radical treatment intervention, or to lower the risk of re-occurrence and/or metastasis following the main treatment. The use of the disclosed methods as a neoadjuvant therapy may result in the main treatment being easier and more likely to succeed, thereby reducing the side-effects and/or improving the outcome of the more extensive treatment. The main treatment may be a surgical intervention, radiotherapy and/or chemotherapy.

The treatments methods described in this disclosure may be used as an adjuvant therapy, which is administered in addition to the main therapy to improve its effectiveness. The main therapy may be surgery, radiotherapy and/or chemotherapy. The adjuvant therapy may be administered concurrently with the main therapy and/or after the main therapy. The adjuvant therapy may be used to reduce or prevent recurrence of the cancer after a subject is treated by a main therapy, such as surgery, radiotherapy and/or chemotherapy.

Treatment of Breast Cancer

Breast cancer may be treated according to the methods disclosed herein by the application of breast cancer specific amplitude-modulated frequencies. Breast cancer specific frequencies are provided below in Table 1. A patient suffering from breast cancer may be treated with any number of the frequencies provided in Table 1, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed breast cancer specific frequencies may be used in the treatment of breast cancer. It should be noted that while the table below provides certain frequencies for treatment of breast cancer, the disclosure is not so limiting, and other frequencies discovered to be effective in the treatment of breast cancer are within the scope of this disclosure. Furthermore, each of the frequencies listed below include values that are close to (i.e., within +/−0.1 Hz), but not exactly, the number or range.

TABLE 1
Amplitude-modulated frequencies discovered to
be effective for treatment of breast cancer.

| | |
|---|---|
| 374-374.7 | Hz |
| 419.8-420.4 | Hz |
| 420.4-420.8 | Hz |
| 429-429.7 | Hz |
| 1336-1336.7 | Hz |
| 1369-1369.7 | Hz |
| 1728-1728.7 | Hz |
| 1771.8-1772.3 | Hz |
| 1772.3-1772.8 | Hz |
| 2811-2811.7 | Hz |
| 2824-2824.7 | Hz |
| 2840-2840.7 | Hz |
| 2858.8-2859.35 | Hz |
| 2860-2860.7 | Hz |
| 2878-2878.7 | Hz |
| 2890-2890.7 | Hz |
| 3209-3209.7 | Hz |
| 3322-3322.7 | Hz |
| 3360-3360.7 | Hz |
| 3663-3663.7 | Hz |
| 3839-3839.7 | Hz |
| 3842.4-3843 | Hz |
| 3854-3854.7 | Hz |
| 3936-3936.7 | Hz |
| 4323-4323.7 | Hz |
| 4329-4329.7 | Hz |
| 4416-4416.7 | Hz |
| 4430-4430.7 | Hz |
| 4477.4-4478 | Hz |
| 4480.9-4481.35 | Hz |
| 4714-4714.7 | Hz |
| 4805.3-4806 | Hz |
| 4813-4813.7 | Hz |
| 4819-4819.7 | Hz |
| 4822-4822.7 | Hz |
| 4823-4823.7 | Hz |
| 4833-4833.7 | Hz |
| 4890-4890.7 | Hz |
| 5154-5154.7 | Hz |
| 5243-5243.7 | Hz |
| 5407.3-5408 | Hz |
| 5716-5716.7 | Hz |
| 5976-5976.7 | Hz |
| 6024-6024.7 | Hz |
| 6070-6070.7 | Hz |
| 6094-6094.7 | Hz |
| 6414-6414.7 | Hz |
| 6717-6717.7 | Hz |
| 6751-6751.7 | Hz |
| 6968.4-6969 | Hz |
| 7013-7013.7 | Hz |
| 7042-7042.7 | Hz |
| 7080-7080.7 | Hz |
| 7145.4-7146 | Hz |
| 7151-7151.7 | Hz |
| 7158-7158.7 | Hz |
| 7225-7225.7 | Hz |
| 8332-8332.7 | Hz |
| 8824-8824.7 | Hz |
| 8837-8837.7 | Hz |
| 9935-9935.7 | Hz |
| 9961-9961.7 | Hz |
| 10013-10013.7 | Hz |
| 10064-10064.7 | Hz |
| 10339.8-10340.4 | Hz |
| 10384-10384.7 | Hz |
| 10745-10745.7 | Hz |
| 10788-10788.7 | Hz |
| 10834-10834.7 | Hz |
| 10854-10854.7 | Hz |
| 10873-10873.7 | Hz |
| 10894-10894.7 | Hz |
| 11163-11163.7 | Hz |
| 11414-11414.7 | Hz |
| 11421-11421.7 | Hz |
| 11429-11429.7 | Hz |
| 11719-11719.7 | Hz |
| 11745-11745.7 | Hz |
| 11931-11931.7 | Hz |
| 11974-11974.7 | Hz |
| 11993-11993.7 | Hz |
| 12118-12118.7 | Hz |
| 12137-12137.7 | Hz |
| 12144-12144.7 | Hz |
| 12318-12318.7 | Hz |
| 12342-12342.7 | Hz |
| 12364-12364.7 | Hz |
| 12557-12557.7 | Hz |
| 12588-12588.7 | Hz |
| 13329-13329.7 | Hz |
| 13377-13377.7 | Hz |
| 13386-13386.7 | Hz |
| 13644-13644.7 | Hz |
| 13661-13661.7 | Hz |
| 13713-13713.7 | Hz |
| 14164-14164.7 | Hz |
| 14308-14308.7 | Hz |
| 14333.4-14333.9 | Hz |
| 14432-14432.7 | Hz |
| 14524-14524.7 | Hz |
| 14589-14589.7 | Hz |
| 14729-14729.7 | Hz |
| 14813-14813.7 | Hz |
| 14835-14835.7 | Hz |
| 14858-14858.7 | Hz |
| 14885-14885.7 | Hz |
| 15035-15035.6 | Hz |
| 15067-15067.7 | Hz |
| 15126-15126.7 | Hz |
| 15195-15195.7 | Hz |
| 15269-15269.7 | Hz |
| 15432-15432.7 | Hz |
| 15432-15432.7 | Hz |
| 15563-15563.7 | Hz |
| 15621-15621.7 | Hz |
| 15624-15624.7 | Hz |
| 15642-15642.7 | Hz |
| 15647-15647.7 | Hz |
| 15669-15669.7 | Hz |
| 15698-15698.7 | Hz |
| 15823-15823.7 | Hz |
| 16347-16347.7 | Hz |
| 17832-17832.7 | Hz |
| 18221-18221.7 | Hz |
| 18260-18260.7 | Hz |
| 19433-19433.7 | Hz |
| 19446-19446.7 | Hz |
| 19489.3-19490 | Hz |
| 19782-19782.7 | Hz |
| 19827-19827.7 | Hz |
| 21291.4-21292 | Hz |
| 21887-21887.7 | Hz |
| 22020-22020.7 | Hz |
| 22419-22419.7 | Hz |
| 22422-22422.7 | Hz |
| 22425-22425.7 | Hz |
| 22444-22444.7 | Hz |
| 22491-22491.7 | Hz |
| 22840.3-22841 | Hz |
| 22859-22859.7 | Hz |
| 22973-22973.7 | Hz |
| 22983-22983.7 | Hz |
| 23258-23258.7 | Hz |
| 23652-23652.7 | Hz |
| 24174-24174.7 | Hz |
| 24811-24811.7 | Hz |
| 26954-26954.7 | Hz |
| 27114-27114.7 | Hz |
| 30463.3-30464 | Hz |
| 34557.3-34558 | Hz |
| 34854-34854.7 | Hz |
| 38144-38144.7 | Hz |
| 38682-38682.7 | Hz |
| 41561-41561.7 | Hz |
| 41623-41623.7 | Hz |
| 41735-41735.7 | Hz |
| 42713-42713.7 | Hz |
| 44575.3-44576 | Hz |
| 46662-46662.7 | Hz |
| 48173-48173.7 | Hz |
| 50292-50292.7 | Hz |

In certain embodiments, the frequencies listed above may also be used in combination with one or more of the frequencies disclosed in the '365 patent. Specifically, treatment modalities for treatment of breast cancer in accordance with this disclosure may include application of one, some or all of the above listed frequencies with one or more of the frequencies disclosed in Table 2 for breast cancer. Alternatively and/or additionally, the frequencies disclosed in the '365 patent may be used for treatment of breast cancer using the methods and systems described herein.

TABLE 2
Amplitude-modulated frequencies for treatment of breast cancer.

| | |
|---|---|
| 78.76 | Hz |
| 181.821 | Hz |
| 331.3 | Hz |
| 414.817 | Hz |
| 430.439 | Hz |
| 440.933 | Hz |
| 0449.323 | Hz |
| 618.8 | Hz |
| 628.431 | Hz |
| 655.435 | Hz |
| 677.972 | Hz |
| 721.313 | Hz |
| 752.933 | Hz |
| 813.205 | Hz |
| 818.342 | Hz |
| 825.145 | Hz |
| 839.521 | Hz |
| 841.211 | Hz |
| 843.312 | Hz |
| 891.901 | Hz |
| 929.095 | Hz |
| 929.1 | Hz |
| 929.131 | Hz |
| 958.929 | Hz |
| 1021 | Hz |
| 1021.311 | Hz |
| 1156.79 | Hz |
| 1372.207 | Hz |
| 1372.934 | Hz |
| 1555.282 | Hz |
| 1588.721 | Hz |
| 1624.802 | Hz |
| 1670.699 | Hz |
| 1821.729 | Hz |
| 1836.219 | Hz |
| 2193.937 | Hz |
| 2221.323 | Hz |
| 2278.312 | Hz |
| 2332.949 | Hz |
| 2357.832 | Hz |
| 2381.443 | Hz |
| 2417.323 | Hz |
| 2423.292 | Hz |
| 2431.334 | Hz |
| 2450.332 | Hz |
| 2551.313 | Hz |
| 2556.221 | Hz |
| 2598.853 | Hz |
| 2621.322 | Hz |
| 2740.191 | Hz |
| 2823.428 | Hz |
| 2831.386 | Hz |
| 2851.347 | Hz |
| 2885.322 | Hz |
| 2919.273 | Hz |
| 3074.333 | Hz |
| 3115.188 | Hz |
| 3239.212 | Hz |
| 3249.529 | Hz |
| 3405.182 | Hz |
| 3432.274 | Hz |
| 3434.693 | Hz |
| 3594.231 | Hz |
| 3647.619 | Hz |
| 3657.931 | Hz |
| 3742.957 | Hz |
| 3753.382 | Hz |
| 3830.732 | Hz |
| 3855.823 | Hz |
| 3916.321 | Hz |
| 3935.218 | Hz |
| 3975.383 | Hz |
| 3993.437 | Hz |
| 4153.192 | Hz |
| 4194.968 | Hz |
| 4241.321 | Hz |
| 4243.393 | Hz |
| 4253.432 | Hz |
| 4314.444 | Hz |
| 4318.222 | Hz |
| 4375.962 | Hz |
| 4393.419 | Hz |
| 4394.134 | Hz |
| 4417.243 | Hz |
| 4481.463 | Hz |
| 4482.223 | Hz |
| 4495.138 | Hz |
| 4549.808 | Hz |
| 4558.306 | Hz |
| 4751.908 | Hz |
| 4779.451 | Hz |
| 4838.674 | Hz |
| 4871.513 | Hz |
| 4878.687 | Hz |
| 4895.296 | Hz |
| 4962.213 | Hz |
| 4969.224 | Hz |
| 4979.321 | Hz |
| 5027.231 | Hz |
| 5059.792 | Hz |
| 5118.094 | Hz |
| 5176.287 | Hz |
| 5365.222 | Hz |
| 5376.392 | Hz |
| 5426.323 | Hz |
| 5431.542 | Hz |
| 5521.621 | Hz |
| 5536.242 | Hz |
| 5739.422 | Hz |
| 5745.218 | Hz |
| 5821.975 | Hz |
| 6037.432 | Hz |
| 6044.333 | Hz |
| 6086.256 | Hz |
| 6208.932 | Hz |
| 6212.808 | Hz |
| 6231.031 | Hz |
| 6280.321 | Hz |
| 6329.391 | Hz |
| 6476.896 | Hz |
| 6477.098 | Hz |
| 6497.319 | Hz |
| 6504.983 | Hz |
| 6651.276 | Hz |
| 6657.913 | Hz |
| 6757.901 | Hz |
| 6758.321 | Hz |
| 6855.286 | Hz |
| 6858.121 | Hz |
| 6898.489 | Hz |
| 6915.886 | Hz |
| 7092.219 | Hz |
| 7120.218 | Hz |
| 7127.311 | Hz |
| 7156.489 | Hz |
| 7208.821 | Hz |
| 7224.197 | Hz |
| 7282.169 | Hz |
| 7285.693 | Hz |
| 7376.329 | Hz |
| 7488.742 | Hz |
| 7541.319 | Hz |
| 7577.421 | Hz |
| 7621.085 | Hz |
| 7627.207 | Hz |
| 7650.939 | Hz |
| 7668.231 | Hz |
| 7691.212 | Hz |
| 7842.184 | Hz |
| 7849.231 | Hz |
| 7915.423 | Hz |
| 7932.482 | Hz |
| 7949.196 | Hz |
| 7967.311 | Hz |
| 8021.229 | Hz |
| 8070.181 | Hz |
| 8114.032 | Hz |
| 8149.922 | Hz |
| 8194.19 | Hz |
| 8245.801 | Hz |
| 8328.322 | Hz |
| 8330.534 | Hz |
| 8355.987 | Hz |
| 8408.121 | Hz |
| 8431.184 | Hz |
| 8452.119 | Hz |
| 8548.324 | Hz |
| 8749.383 | Hz |
| 8782.421 | Hz |
| 8784.424 | Hz |
| 8887.182 | Hz |
| 8894.222 | Hz |
| 8923.1 | Hz |
| 8923.361 | Hz |
| 8935.752 | Hz |
| 8936.1 | Hz |
| 9012.282 | Hz |
| 9012.896 | Hz |
| 9060.323 | Hz |
| 9072.409 | Hz |
| 9131.419 | Hz |
| 9199.232 | Hz |
| 9245.927 | Hz |
| 9270.322 | Hz |
| 9279.193 | Hz |
| 9393.946 | Hz |
| 10227.242 | Hz |
| 10340.509 | Hz |
| 10363.313 | Hz |
| 10456.383 | Hz |
| 10468.231 | Hz |
| 10470.456 | Hz |
| 10472.291 | Hz |
| 10689.339 | Hz |
| 10832.222 | Hz |
| 11525.121 | Hz |
| 11541.915 | Hz |
| 11812.328 | Hz |
| 11812.419 | Hz |
| 11840.323 | Hz |
| 11925.089 | Hz |
| 12123.281 | Hz |
| 12267.281 | Hz |
| 12294.283 | Hz |
| 12334.419 | Hz |
| 12611.288 | Hz |
| 12629.222 | Hz |
| 12633.372 | Hz |
| 12648.221 | Hz |
| 13315.335 | Hz |
| 13331.358 | Hz |
| 13735.241 | Hz |
| 13826.325 | Hz |
| 13853.232 | Hz |
| 13915.231 | Hz |
| 13990.123 | Hz |
| 14122.942 | Hz |
| 14162.332 | Hz |
| 14519.232 | Hz |
| 14543.128 | Hz |
| 15651.323 | Hz |
| 17352.085 | Hz |
| 17970.122 | Hz |
| 18524.419 | Hz |
| 18619.331 | Hz |
| 18662.112 | Hz |
| 18679.492 | Hz |
| 18785.463 | Hz |
| 19385.893 | Hz |
| 19406.211 | Hz |
| 22479.333 | Hz |
| 30182.932 | Hz |

One or more of the AM frequencies of Table 1 may be used in combination with one or more of the frequencies provided in Table 2. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of breast cancer, which frequencies are selected from Table 1 and, optionally from Table 2, provided that at least one or more frequencies are selected from Table 1.

Treatment of Neuroendocrine Tumors

Neuroendocrine tumors may be treated according to the methods disclosed herein by the application of neuroendocrine tumor specific amplitude-modulated frequencies. Neuroendocrine tumors specific frequencies are provided below in Table 3. A patient suffering from neuroendocrine tumors may be treated with any number of the frequencies provided in Table 3, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed neuroendocrine tumor specific frequencies may be used in the treatment of neuroendocrine tumors. It should be noted that while the table below provides certain frequencies for treatment of neuroendocrine tumors, the disclosure is not so limiting, and other frequencies discovered to be effective in the treatment of neuroendocrine tumors are within the scope of this disclosure. Furthermore, each of the frequencies listed below include values that are close to (i.e., within +/−0.1 Hz), but not exactly, the number or range.

TABLE 3
Amplitude-modulated frequencies discovered to be
effective for treatment of neuroendocrine tumors.

| | |
|---|---|
| 843-843.7 | Hz |
| 1656-1656.7 | Hz |
| 2423-2423.7 | Hz |
| 2480-2480.7 | Hz |
| 2707-2707.7 | Hz |
| 2890-2890.7 | Hz |
| 3080.3-3081 | Hz |
| 3661-3661.7 | Hz |
| 3678-3678.7 | Hz |
| 3976.4-3977 | Hz |
| 4048-4048.7 | Hz |
| 4055-4055.7 | Hz |
| 4368.3-4369 | Hz |
| 4459-4459.7 | Hz |
| 4621-4621.7 | Hz |
| 4837-4837.7 | Hz |
| 4866-4866.7 | Hz |
| 5165.4-5166 | Hz |
| 5265-5265.7 | Hz |
| 5270-5270.7 | Hz |
| 6436-6436.7 | Hz |
| 7037-7037.7 | Hz |
| 8008-8008.7 | Hz |
| 8024-8024.7 | Hz |
| 8097-8097.7 | Hz |
| 8427-8427.7 | Hz |
| 8478.3-8479 | Hz |
| 8480-8480.7 | Hz |
| 8492-8492.7 | Hz |
| 8853-8853.7 | Hz |
| 8877-8877.7 | Hz |
| 9122-9122.7 | Hz |
| 9131-9131.7 | Hz |
| 9174-9174.7 | Hz |
| 10073-10073.7 | Hz |
| 10848.7-10849.3 | Hz |
| 11267-11267.7 | Hz |
| 11336-11336.7 | Hz |
| 11524-11524.7 | Hz |
| 11551.3-11552 | Hz |
| 11927.4-11928 | Hz |
| 12309-12309.7 | Hz |
| 13617-13617.7 | Hz |
| 13674-13674.7 | Hz |
| 13774-13774.7 | Hz |
| 14350-14350.7 | Hz |
| 14456-14456.7 | Hz |
| 14479-14479.7 | Hz |
| 14540-14540.7 | Hz |
| 14553-14553.7 | Hz |
| 14756-14756.7 | Hz |
| 14922-14922.7 | Hz |
| 14961-14961.7 | Hz |
| 15226-15226.7 | Hz |
| 15817-15817.7 | Hz |
| 15825-15825.7 | Hz |
| 15835-15835.7 | Hz |
| 15836-15836.7 | Hz |
| 15897-15897.7 | Hz |
| 16071-16071.7 | Hz |
| 17417-17417.7 | Hz |
| 17418-17418.7 | Hz |
| 17473-17473.7 | Hz |
| 17474-17474.7 | Hz |
| 17621-17621.7 | Hz |
| 17655-17655.7 | Hz |
| 18655-18655.7 | Hz |
| 18672-18672.7 | Hz |
| 18678-18678.7 | Hz |
| 19393-19393.7 | Hz |
| 19426-19426.7 | Hz |
| 19817-19817.7 | Hz |
| 19819-19819.7 | Hz |
| 19861-19861.7 | Hz |
| 19867-19867.7 | Hz |
| 20818-20818.7 | Hz |
| 21708-21708.7 | Hz |
| 21720-21720.7 | Hz |
| 21865-21865.7 | Hz |
| 21876-21876.7 | Hz |
| 22119-22119.7 | Hz |
| 22454-22454.7 | Hz |
| 24073-24073.7 | Hz |
| 30304-30304.7 | Hz |
| 31685-31685.7 | Hz |
| 43633-43633.7 | Hz |
| 44773-44773.7 | Hz |
| 46662-46662.7 | Hz |
| 46793-46793.7 | Hz |
| 48103-48103.7 | Hz |
| 50243-50243.7 | Hz |

In certain embodiments, the frequencies listed above may also be used in combination with one or more of the frequencies disclosed in the '365 patent. Specifically, treatment modalities for treatment of neuroendocrine tumors in accordance with this disclosure may include application of some or all of the above listed frequencies with one or more of the frequencies disclosed in the Table 4 for neuroendocrine cancer. Alternatively and/or additionally, the frequencies disclosed in the '365 patent may be used for treatment of neuroendocrine tumors using the methods and systems described herein.

TABLE 4
Amplitude-modulated frequencies for
treatment of neuroendocrine tumors.

| | |
|---|---|
| 1766.335 | Hz |
| 2408.225 | Hz |
| 2441.502 | Hz |
| 2647.938 | Hz |
| 2741.261 | Hz |
| 3020.212 | Hz |
| 3128.822 | Hz |
| 3238.742 | Hz |
| 3296.431 | Hz |
| 3348.783 | Hz |
| 3360.971 | Hz |
| 3440.212 | Hz |
| 3533.328 | Hz |
| 3666.283 | Hz |
| 4079.282 | Hz |
| 4243.393 | Hz |
| 4426.387 | Hz |
| 5245.818 | Hz |
| 5536.242 | Hz |
| 5548.879 | Hz |
| 5739.422 | Hz |
| 5849.241 | Hz |
| 6291.631 | Hz |
| 6406.891 | Hz |
| 6780.679 | Hz |
| 7151.264 | Hz |
| 7482.245 | Hz |
| 7575.393 | Hz |
| 8359.932 | Hz |
| 9073.418 | Hz |

One or more of the AM frequencies of Table 3 may be combined with one or more of the frequencies as provided in Table 4. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of breast cancer, which frequencies are selected from Table 3 and, optionally from Table 4, provided that at least one or more frequencies are selected from Table 3.

Treatment of Non-Hodgkin Lymphoma

Non-Hodgkin lymphoma may be treated according to the methods disclosed herein by the application of non-Hodgkin lymphoma specific amplitude-modulated frequencies. Non-Hodgkin lymphoma tumor specific frequencies are provided below in Table 5. A patient suffering from non-Hodgkin lymphoma may be treated with any number of the frequencies provided in Table 5, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more of the listed non-Hodgkin lymphoma specific frequencies may be used in the treatment of non-Hodgkin lymphoma tumors. It should be noted that while the table below provides certain frequencies for treatment of non-Hodgkin lymphoma tumors, the disclosure is not so limiting, and other frequencies discovered to be effective in the treatment of non-Hodgkin lymphoma tumors are within the scope of this disclosure. Furthermore, each of the frequencies listed below include values that are close to (i.e., within +/−0.1 Hz), but not exactly, the number or range.

TABLE 5
Amplitude-modulated frequencies discovered to be
effective for treatment of non-Hodgkin lymphoma.

| | |
|---|---|
| 3222-3222.7 | Hz |
| 3228-3228.7 | Hz |
| 3272-3272.7 | Hz |
| 3618-3618.7 | Hz |
| 4523-4523.7 | Hz |
| 4556-4556.7 | Hz |
| 4584-4584.7 | Hz |
| 5116-5116.7 | Hz |
| 5142-5142.7 | Hz |
| 6413-6413.7 | Hz |
| 6419.3-6420 | Hz |
| 6441-6441.7 | Hz |
| 6474-6474.7 | Hz |
| 6911.3-6912 | Hz |
| 6912-6912.7 | Hz |
| 6943-6943.7 | Hz |
| 7551-7551.7 | Hz |
| 7811-7811.7 | Hz |
| 7857-7857.7 | Hz |
| 7865-7865.7 | Hz |
| 8125-8125.7 | Hz |
| 9106-9106.7 | Hz |
| 9161-9161.7 | Hz |
| 9179-9179.7 | Hz |
| 9230-9230.7 | Hz |
| 9236-9236.7 | Hz |
| 9247-9247.7 | Hz |
| 11730.6-11731.1 | Hz |
| 11785-11785.7 | Hz |
| 13222-13222.7 | Hz |
| 13751-13751.7 | Hz |
| 13780-13780.7 | Hz |
| 15006-15006.7 | Hz |
| 15033-15033.7 | Hz |
| 15056-15056.7 | Hz |
| 15729-15729.7 | Hz |
| 15773-15773.7 | Hz |
| 17229-17229.7 | Hz |
| 17246-17246.7 | Hz |
| 17268-17268.7 | Hz |
| 20256-20256.7 | Hz |
| 22444-22444.7 | Hz |
| 30666-30666.7 | Hz |
| 30713-30713.7 | Hz |
| 30843-30843.7 | Hz |
| 38216-38216.7 | Hz |

Treatment of Adenocarcinoma of Pancreas

Adenocarcinoma of pancreas may be treated according to the methods disclosed herein by the application of adenocarcinoma of pancreas specific amplitude-modulated frequencies. Adenocarcinoma of pancreas specific frequencies are provided below in Table 6. A patient suffering from adenocarcinoma of pancreas may be treated with any number of the frequencies provided in Table 6, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed specific frequencies may be used in the treatment of adenocarcinoma of pancreas. It should be noted that while the table below provides certain frequencies for treatment of adenocarcinoma of pancreas, the disclosure is not so limiting, and other frequencies discovered to be effective in the treatment of adenocarcinoma of pancreas are within the scope of this disclosure. Furthermore, each of the frequencies listed below include values that are close to (i.e., within +/−0.1 Hz), but not exactly, the number or range.

TABLE 6
Amplitude-modulated frequencies discovered to be effective
for treatment of adenocarcinoma of the pancreas.

| | |
|---|---|
| 403-403.7 | Hz |
| 441-441.7 | Hz |
| 527-527.7 | Hz |
| 624.32-624.8 | Hz |
| 633-633.7 | Hz |
| 727-727.7 | Hz |
| 759-759.7 | Hz |
| 785-785.7 | Hz |
| 811-811.7 | Hz |
| 829-829.7 | Hz |
| 841-841.7 | Hz |
| 962-962.7 | Hz |
| 964.5-965 | Hz |
| 972-972.7 | Hz |
| 974.4-975 | Hz |
| 977-977.7 | Hz |
| 1179.2-1179.8 | Hz |
| 1250.3-1251 | Hz |
| 1314-1314.7 | Hz |
| 1352-1352.7 | Hz |
| 1622-1622.7 | Hz |
| 1652-1652.7 | Hz |
| 1656-1656.7 | Hz |
| 1670.3-1671 | Hz |
| 1914-1914.7 | Hz |
| 2346-2346.7 | Hz |
| 2362-2362.7 | Hz |
| 2380-2380.7 | Hz |
| 2469.2-2469.7 | Hz |
| 2627-2627.7 | Hz |
| 2706-2706.7 | Hz |
| 2720.3-2721 | Hz |
| 2726-2726.7 | Hz |
| 2754-2754.7 | Hz |
| 2778-2778.7 | Hz |
| 2842-2842.7 | Hz |
| 2864-2864.7 | Hz |
| 2940-2940.7 | Hz |
| 2988-2988.7 | Hz |
| 3234-3234.7 | Hz |
| 3280.6-3281.3 | Hz |
| 3366-3366.7 | Hz |
| 3428-3428.7 | Hz |
| 3446-3446.7 | Hz |
| 3466-3466.7 | Hz |
| 3523-3523.7 | Hz |
| 3605-3605.7 | Hz |
| 3622.5-3623 | Hz |
| 3627-3627.7 | Hz |
| 3631-3631.7 | Hz |
| 3656-3656.7 | Hz |
| 3663-3663.7 | Hz |
| 3667-3667.7 | Hz |
| 3687-3687.7 | Hz |
| 4152-4152.7 | Hz |
| 4233-4233.7 | Hz |
| 4249-4249.7 | Hz |
| 4281-4281.7 | Hz |
| 4368.3-4369 | Hz |
| 4417-4417.7 | Hz |
| 4425-4425.7 | Hz |
| 4440.4-4441 | Hz |
| 4465-4465.7 | Hz |
| 4480.9-4481.35 | Hz |
| 4580.11-4580.7 | Hz |
| 4621-4621.7 | Hz |
| 4637-4637.7 | Hz |
| 4657-4657.6 | Hz |
| 4725-4725.7 | Hz |
| 4733-4733.7 | Hz |
| 4749-4749.7 | Hz |
| 4841-4841.7 | Hz |
| 4924-4924.7 | Hz |
| 4946-4946.7 | Hz |
| 5063-5063.7 | Hz |
| 5089-5089.7 | Hz |
| 5378.2-5378.7 | Hz |
| 5424-5424.7 | Hz |
| 5438-5438.7 | Hz |
| 6112-6112.7 | Hz |
| 6159-6159.7 | Hz |
| 6161.3-6162 | Hz |
| 6167-6167.7 | Hz |
| 6179-6179.7 | Hz |
| 6220.3-6221 | Hz |
| 6346-6346.7 | Hz |
| 6380.4-6381 | Hz |
| 6808-6808.7 | Hz |
| 6825-6825.7 | Hz |
| 6847-6847.7 | Hz |
| 6850-6850.7 | Hz |
| 6878-6878.7 | Hz |
| 7036-7036.7 | Hz |
| 7037-7037.7 | Hz |
| 7118-7118.7 | Hz |
| 7160-7160.7 | Hz |
| 7432-7432.7 | Hz |
| 7541-7541.7 | Hz |
| 7676-7676.7 | Hz |
| 7936-7936.7 | Hz |
| 8024-8024.7 | Hz |
| 8032-8032.7 | Hz |
| 8043-8043.7 | Hz |
| 8447-8447.7 | Hz |
| 8734-8734.7 | Hz |
| 8756-8756.7 | Hz |
| 8757-8757.7 | Hz |
| 8809-8809.7 | Hz |
| 8823-8823.7 | Hz |
| 8841-8841.7 | Hz |
| 8845-8845.7 | Hz |
| 8846-8846.7 | Hz |
| 8872-8872.7 | Hz |
| 9133-9133.7 | Hz |
| 9155.7-9156.2 | Hz |
| 9520-9520.7 | Hz |
| 9859-9859.7 | Hz |
| 10342-10342.6 | Hz |
| 10352-10352.7 | Hz |
| 10378-10378.7 | Hz |
| 10436-10436.7 | Hz |
| 10566.4-10567 | Hz |
| 10633-10633.7 | Hz |
| 10641-10641.7 | Hz |
| 10671-10671.7 | Hz |
| 10708-10708.7 | Hz |
| 10859-10859.7 | Hz |
| 10876-10876.7 | Hz |
| 11174-11174.7 | Hz |
| 11220-11220.7 | Hz |
| 11247-11247.7 | Hz |
| 11264-11264.7 | Hz |
| 11284-11284.7 | Hz |
| 11630-11630.7 | Hz |
| 11994-11994.7 | Hz |
| 12120.2-12120.7 | Hz |
| 12135-12135.7 | Hz |
| 12152-12152.7 | Hz |
| 12184-12184.7 | Hz |
| 12711-12711.7 | Hz |
| 13375-13375.7 | Hz |
| 13571-13571.7 | Hz |
| 13636.2-13636.7 | Hz |
| 13738.3-13739 | Hz |
| 13947-13947.7 | Hz |
| 14116-14116.7 | Hz |
| 14279-14279.7 | Hz |
| 14376-14376.7 | Hz |
| 14462-14462.7 | Hz |
| 14519-14519.7 | Hz |
| 14558-14558.7 | Hz |
| 14729-14729.7 | Hz |
| 14940.4-14941 | Hz |
| 14957-14957.7 | Hz |
| 15179-15179.7 | Hz |
| 15225-15225.6 | Hz |
| 15536-15536.7 | Hz |
| 15576-15576.7 | Hz |
| 15597-15597.7 | Hz |
| 15663-15663.7 | Hz |
| 15678-15678.7 | Hz |
| 15711-15711.7 | Hz |
| 15724-15724.7 | Hz |
| 15740-15740.7 | Hz |
| 15749-15749.7 | Hz |
| 15824-15824.7 | Hz |
| 15867-15867.7 | Hz |
| 16143-16143.7 | Hz |
| 17214-17214.7 | Hz |
| 17253-17253.7 | Hz |
| 17271-17271.7 | Hz |
| 17283-17283.7 | Hz |
| 17739-17739.7 | Hz |
| 17944-17944.7 | Hz |
| 17962-17962.7 | Hz |
| 18279-18279.7 | Hz |
| 18678.7-18679.3 | Hz |
| 18921-18921.7 | Hz |
| 18922-18922.7 | Hz |
| 19059-19059.7 | Hz |
| 19464-19464.7 | Hz |
| 19686-19686.4 | Hz |
| 19724-19724.7 | Hz |
| 20123-20123.7 | Hz |
| 20128-20128.7 | Hz |
| 20144-20144.7 | Hz |
| 20149.3-20150 | Hz |
| 20219-20219.7 | Hz |
| 20254-20254.7 | Hz |
| 21546-21546.7 | Hz |
| 21616-21616.7 | Hz |
| 21664-21664.7 | Hz |
| 21840-21840.7 | Hz |
| 21876-21876.7 | Hz |
| 21894-21894.7 | Hz |
| 22355-22355.7 | Hz |
| 22430-22430.7 | Hz |
| 22597-22597.7 | Hz |
| 22742-22742.7 | Hz |
| 22822-22822.7 | Hz |
| 22846-22846.7 | Hz |
| 22867-22867.7 | Hz |
| 23237-23237.7 | Hz |
| 23284-23284.7 | Hz |
| 24228-24228.7 | Hz |
| 24275-24275.7 | Hz |
| 24508-24508.7 | Hz |
| 24609.3-24610 | Hz |
| 24841-24841.7 | Hz |
| 27478-27478.7 | Hz |
| 32143-32143.7 | Hz |
| 32493-32493.7 | Hz |
| 33546-33546.7 | Hz |
| 40133-40133.7 | Hz |
| 40595-40595.7 | Hz |
| 44913-44913.7 | Hz |
| 45474-45474.7 | Hz |
| 46672.3-46673 | Hz |
| 48204-48204.7 | Hz |
| 48314-48314.7 | Hz |
| 50072-50072.7 | Hz |
| 50254-50254.7 | Hz |
| 52812-52812.7 | Hz |
| 79867-79867.7 | Hz |
| 80993.3-80994 | Hz |
| 91877-91877.7 | Hz |
| 95742-95742.7 | Hz. |

In certain embodiments, the frequencies listed above may also be used in combination with one or more of the frequencies disclosed in the '365 patent. Specifically, treatment modalities for treatment of adenocarcinoma of the pancreas in accordance with this disclosure may include application of some or all of the above listed frequencies with one or more of the frequencies disclosed in the Table 7. Alternatively and/or additionally, the frequencies disclosed in the '365 patent may be used for treatment of adenocarcinoma of pancreas state using the methods and systems described herein.

TABLE 7
Amplitude-modulated frequencies for
treatment of pancreatic cancer.

| | |
|---|---|
| 331.3 | Hz |
| 331.365 | Hz |
| 436.3 | Hz |
| 436.332 | Hz |
| 447.942 | Hz |
| 476.127 | Hz |
| 559.292 | Hz |
| 589.187 | Hz |
| 624.218 | Hz |
| 727 | Hz |
| 734.921 | Hz |
| 809.313 | Hz |
| 845.309 | Hz |
| 870.4 | Hz |
| 963.221 | Hz |
| 1156.79 | Hz |
| 1157 | Hz |
| 1179 | Hz |
| 1360.133 | Hz |
| 1372.207 | Hz |
| 1372.934 | Hz |
| 1804.126 | Hz |
| 1816.221 | Hz |
| 1873.477 | Hz |
| 1967.211 | Hz |
| 1990.482 | Hz |
| 2278.312 | Hz |
| 2315.921 | Hz |
| 2320.315 | Hz |
| 2334.178 | Hz |
| 2381.443 | Hz |
| 2469 | Hz |
| 2477.919 | Hz |
| 2542.221 | Hz |
| 2598.853 | Hz |
| 2647.938 | Hz |
| 2685.081 | Hz |
| 2716.095 | Hz |
| 2721.331 | Hz |
| 2732.231 | Hz |
| 2809.849 | Hz |
| 2823.428 | Hz |
| 2835.332 | Hz |
| 3134.313 | Hz |
| 3241.461 | Hz |
| 3255.219 | Hz |
| 3263.432 | Hz |
| 3286.255 | Hz |
| 3330.935 | Hz |
| 3373.892 | Hz |
| 3438.109 | Hz |
| 3449.219 | Hz |
| 3535.219 | Hz |
| 3549.215 | Hz |
| 3564.419 | Hz |
| 3619.412 | Hz |
| 3622.312 | Hz |
| 3638.432 | Hz |
| 3696.424 | Hz |
| 3943.214 | Hz |
| 3976.929 | Hz |
| 4014.889 | Hz |
| 4041.219 | Hz |
| 4044.195 | Hz |
| 4056.384 | Hz |
| 4085.971 | Hz |
| 4144.592 | Hz |
| 4153.192 | Hz |
| 4161.889 | Hz |
| 4243.393 | Hz |
| 4332.498 | Hz |
| 4341.423 | Hz |
| 4355.327 | Hz |
| 4417.885 | Hz |
| 4422.322 | Hz |
| 4451.297 | Hz |
| 4486.384 | Hz |
| 4558.306 | Hz |
| 4580 | Hz |
| 4685.082 | Hz |
| 4839.589 | Hz |
| 5151.402 | Hz |
| 5209.911 | Hz |
| 5262.282 | Hz |
| 5271.312 | Hz |
| 5387.73 | Hz |
| 5494.928 | Hz |
| 5521.221 | Hz |
| 5573.209 | Hz |
| 5609.382 | Hz |
| 5929.616 | Hz |
| 5948.897 | Hz |
| 5966.112 | Hz |
| 5976.825 | Hz |
| 6064.197 | Hz |
| 6086.256 | Hz |
| 6157.253 | Hz |
| 6215.298 | Hz |
| 6333.917 | Hz |
| 6365.242 | Hz |
| 6558.342 | Hz |
| 6568.278 | Hz |
| 6823.194 | Hz |
| 6853.391 | Hz |
| 6855.286 | Hz |
| 7213.204 | Hz |
| 7228.528 | Hz |
| 7238.232 | Hz |
| 7277.921 | Hz |
| 7280.422 | Hz |
| 7320.494 | Hz |
| 7366.412 | Hz |
| 7534.221 | Hz |
| 7548.713 | Hz |
| 7567.127 | Hz |
| 7620.851 | Hz |
| 7663.209 | Hz |
| 7725.203 | Hz |
| 7852.233 | Hz |
| 7920.879 | Hz |
| 7985.122 | Hz |
| 8008.323 | Hz |
| 8013.312 | Hz |
| 8045.484 | Hz |
| 8242.332 | Hz |
| 8351.622 | Hz |
| 8408.121 | Hz |
| 8455.894 | Hz |
| 8551.231 | Hz |
| 8743.321 | Hz |
| 8789.631 | Hz |
| 8868.809 | Hz |
| 9012.241 | Hz |
| 9028.994 | Hz |
| 9131.232 | Hz |
| 9658.296 | Hz |
| 9663.495 | Hz |
| 9680.737 | Hz |
| 9824.442 | Hz |
| 9942.321 | Hz |
| 10279.122 | Hz |
| 10388.49 | Hz |
| 10438.495 | Hz |
| 10518.311 | Hz |
| 10528.239 | Hz |
| 10582.095 | Hz |
| 10926.111 | Hz |
| 10948.411 | Hz |
| 10955.558 | Hz |
| 11538.193 | Hz |
| 11904.741 | Hz |
| 12255.229 | Hz |
| 12613.341 | Hz |
| 12819.942 | Hz |
| 13674.482 | Hz |
| 13731.322 | Hz |
| 14525.312 | Hz |
| 14537.218 | Hz |
| 14549.331 | Hz |
| 14845.453 | Hz |
| 14944.989 | Hz |
| 15246.315 | Hz |
| 18668.239 | Hz |
| 19321.231 | Hz |
| 19347.208 | Hz |
| 30182.932 | Hz |

One or more of the AM frequencies of Table 6 may be used in combination with one or more of the frequencies provided in Table 7. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of adenocarcinoma of the pancreas, which frequencies are selected from Table 6 and, optionally from Table 7, provided that at least one or more frequencies are selected from Table 6.

Treatment of Head and Neck Cancer

Head and neck cancer may be treated according to the methods disclosed herein by the application of head and neck cancer specific amplitude-modulated frequencies. Head and neck cancer specific frequencies are provided below in Table 8. A patient suffering from head and neck cancer may be treated with any number of the frequencies provided in Table 8, however it is generally preferable to employ as many of the frequencies as is practical for the treatment of the patient. Accordingly, 10 or more, 15 or more of the listed head and neck cancer specific frequencies may be used in the treatment of head and neck cancer. It should be noted that while the table below provides certain frequencies for treatment of head and neck cancer, the disclosure is not so limiting, and other frequencies discovered to be effective in the treatment of head and neck cancer are within the scope of this disclosure. Furthermore, each of the frequencies listed below include values that are close to (i.e., within +/−0.1 Hz), but not exactly, the number or range.

TABLE 8
Amplitude-modulated frequencies discovered to be
effective for treatment of head and neck cancer.

| | |
|---|---|
| 1858-1858.7 | Hz |
| 2893-2893.7 | Hz |
| 5152-5152.7 | Hz |
| 6897-6897.7 | Hz |
| 7080-7080.7 | Hz |
| 7136-7136.7 | Hz |
| 7274-7274.7 | Hz |
| 8755-8755.7 | Hz |
| 9573-9573.7 | Hz |
| 9656-9656.7 | Hz |
| 10439-10439.7 | Hz |
| 10484-10484.7 | Hz |
| 10645-10645.7 | Hz |
| 10673-10673.7 | Hz |
| 11559-11559.7 | Hz |
| 14052-14052.7 | Hz |
| 17379-17379.7 | Hz |
| 19269-19269.7 | Hz |

Treatment of Gastric Cancer

Gastric cancer may be treated according to the methods disclosed herein by the application of gastric cancer specific amplitude-modulated frequencies. Gastric cancer specific frequencies are provided below in Table 9. A patient suffering from gastric cancer may be treated with any number of the frequencies provided in Table 9, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed gastric cancer specific frequencies may be used in the treatment of gastric cancer. It should be noted that while the table below provides certain frequencies for treatment of gastric cancer, the disclosure is not so limiting, and other frequencies discovered to be effective in the treatment of gastric cancer are within the scope of this disclosure. Furthermore, each of the frequencies listed below include values that are close to (i.e., within +/−0.1 Hz), but not exactly, the number or range.

TABLE 9
Amplitude-modulated frequencies discovered to
be effective for treatment of gastric cancer.

| | |
|---|---|
| 374-374.7 | Hz |
| 560-560.7 | Hz |
| 628-628.7 | Hz |
| 642.4-643 | Hz |
| 708.3-709 | Hz |
| 749-749.7 | Hz |
| 806-806.6 | Hz |
| 825-825.7 | Hz |
| 843-843.7 | Hz |
| 914-914.7 | Hz |
| 959-959.7 | Hz |
| 969-969.7 | Hz |
| 1157-1157.7 | Hz |
| 1436-1436.7 | Hz |
| 1656-1656.7 | Hz |
| 2379.3-2380 | Hz |
| 2417-2417.7 | Hz |
| 2436-2436.7 | Hz |
| 2455-2455.7 | Hz |
| 2478-2478.7 | Hz |
| 2747-2747.7 | Hz |
| 2762-2762.7 | Hz |
| 2774-2774.7 | Hz |
| 2856.4-2857 | Hz |
| 2891-2891.7 | Hz |
| 2981-2981.7 | Hz |
| 3104.3-3105 | Hz |
| 3133-3133.7 | Hz |
| 3522-3522.7 | Hz |
| 3537-3537.7 | Hz |
| 3549-3549.7 | Hz |
| 3556-3556.7 | Hz |
| 3595-3595.7 | Hz |
| 3927-3927.7 | Hz |
| 4154-4154.7 | Hz |
| 4548-4548.7 | Hz |
| 4723-4723.7 | Hz |
| 5286-5286.7 | Hz |
| 5494.4-5495 | Hz |
| 5548.3-5549 | Hz |
| 5742-5742.7 | Hz |
| 6010-6010.7 | Hz |
| 6064-6064.7 | Hz |
| 6329-6329.7 | Hz |
| 6444-6444.7 | Hz |
| 6469-6469.7 | Hz |
| 6488-6488.7 | Hz |
| 7222.4-7223 | Hz |
| 7646-7646.7 | Hz |
| 7748-7748.7 | Hz |
| 7855-7855.7 | Hz |
| 7927-7927.7 | Hz |
| 7951-7951.7 | Hz |
| 7970-7970.7 | Hz |
| 7979-7979.7 | Hz |
| 8039-8039.7 | Hz |
| 8055.4-8056 | Hz |
| 8926-8926.7 | Hz |
| 9109-9109.7 | Hz |
| 9123-9123.7 | Hz |
| 9136-9136.7 | Hz |
| 9146-9146.7 | Hz |
| 9157-9157.7 | Hz |
| 9194-9194.7 | Hz |
| 10137-10137.7 | Hz |
| 10345-10345.7 | Hz |
| 10727-10727.7 | Hz |
| 10802-10802.7 | Hz |
| 11533-11533.7 | Hz |
| 12165-12165.7 | Hz |
| 13241-13241.7 | Hz |
| 13307-13307.7 | Hz |
| 13330.7-13331.2 | Hz |
| 13408-13408.7 | Hz |
| 13434-13434.7 | Hz |
| 13714-13714.7 | Hz |
| 13746-13746.7 | Hz |
| 14016.4-14017 | Hz |
| 14160-14160.7 | Hz |
| 14483-14483.7 | Hz |
| 14511-14511.7 | Hz |
| 14670.4-14671 | Hz |
| 16309-16309.7 | Hz |
| 17418-17418.7 | Hz |
| 17455-17455.7 | Hz |
| 17485-17485.7 | Hz |
| 17581-17581.7 | Hz |
| 19125-19125.7 | Hz |
| 21430-21430.7 | Hz |
| 21714-21714.7 | Hz |
| 23946-23946.7 | Hz |
| 39586-39586.7 | Hz |
| 40734-40734.7 | Hz |
| 41172-41172.7 | Hz |
| 42146-42146.7 | Hz |
| 46143-46143.7 | Hz |

Treatment of Glioblastoma

Glioblastoma may be treated according to the methods disclosed herein by the application of glioblastoma specific amplitude-modulated frequencies. Glioblastoma specific frequencies are provided below in Table 10. A patient suffering from glioblastoma may be treated with any number of the frequencies provided in Table 10, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed glioblastoma specific frequencies may be used in the treatment of glioblastoma. It should be noted that while the table below provides certain frequencies for treatment of glioblastoma, the disclosure is not so limiting, and other frequencies discovered to be effective in the treatment of glioblastoma are within the scope of this disclosure. Furthermore, each of the frequencies listed below include values that are close to (i.e., within +/−0.1 Hz), but not exactly, the number or range.

TABLE 10
Amplitude-modulated frequencies discovered to
be effective for treatment of glioblastoma.

| | |
|---|---|
| 262-262.7 | Hz |
| 295-295.6 | Hz |
| 415-415.7 | Hz |
| 418-418.7 | Hz |
| 423-423.7 | Hz |
| 434-434.7 | Hz |
| 440.4-441 | Hz |
| 476-476.7 | Hz |
| 556-556.7 | Hz |
| 608-608.7 | Hz |
| 612-612.7 | Hz |
| 627-627.7 | Hz |
| 656-656.7 | Hz |
| 694-694.7 | Hz |
| 708.3-709 | Hz |
| 741-741.7 | Hz |
| 752.4-753 | Hz |
| 757-757.6 | Hz |
| 759-759.7 | Hz |
| 776-776.7 | Hz |
| 796.3-797 | Hz |
| 836-836.7 | Hz |
| 941-941.7 | Hz |
| 1027-1027.7 | Hz |
| 1045-1045.7 | Hz |
| 1195-1195.7 | Hz |
| 1868-1868.7 | Hz |
| 1897-1897.7 | Hz |
| 2044-2044.7 | Hz |
| 2439-2439.7 | Hz |
| 2612-2612.7 | Hz |
| 2632-2632.7 | Hz |
| 2653.3-2654 | Hz |
| 2686-2686.7 | Hz |
| 2690-2690.7 | Hz |
| 2697-2697.7 | Hz |
| 2738-2738.7 | Hz |
| 3157-3157.7 | Hz |
| 3218-3218.7 | Hz |
| 3227.3-3228 | Hz |
| 3249-3249.7 | Hz |
| 3277.3-3278 | Hz |
| 3281.6-3282.1 | Hz |
| 3436-3436.7 | Hz |
| 3633-3633.7 | Hz |
| 3656-3656.7 | Hz |
| 3962-3962.7 | Hz |
| 4148-4148.7 | Hz |
| 4241-4241.7 | Hz |
| 4410-4410.7 | Hz |
| 4416-4416.7 | Hz |
| 4454-4454.7 | Hz |
| 4619-4619.7 | Hz |
| 4632-4632.7 | Hz |
| 4853-4853.7 | Hz |
| 5139-5139.7 | Hz |
| 5156-5156.7 | Hz |
| 5996-5996.7 | Hz |
| 6292-6292.7 | Hz |
| 6297-6297.7 | Hz |
| 6459-6459.7 | Hz |
| 6474-6474.7 | Hz |
| 6492.3-6493 | Hz |
| 6626.3-6627 | Hz |
| 6727.4-6728 | Hz |
| 6809-6809.6 | Hz |
| 6841-6841.7 | Hz |
| 6915.3-6916 | Hz |
| 6943-6943.7 | Hz |
| 7110-7110.7 | Hz |
| 7182.4-7183 | Hz |
| 7194.3-7195 | Hz |
| 7268-7268.7 | Hz |
| 7323-7323.7 | Hz |
| 7480-7480.7 | Hz |
| 7663-7663.7 | Hz |
| 7796-7796.7 | Hz |
| 7936-7936.7 | Hz |
| 8070-8070.7 | Hz |
| 8128.4-8129 | Hz |
| 8145-8145.7 | Hz |
| 8158-8158.7 | Hz |
| 8314-8314.7 | Hz |
| 8355-8355.7 | Hz |
| 8484-8484.7 | Hz |
| 9138.3-9139 | Hz |
| 9155.7-9156.2 | Hz |
| 9921-9921.7 | Hz |
| 10427-10427.7 | Hz |
| 10468-10468.7 | Hz |
| 10478-10478.7 | Hz |
| 10719-10719.7 | Hz |
| 10802-10802.7 | Hz |
| 10809-10809.7 | Hz |
| 10851-10851.7 | Hz |
| 11556-11556.7 | Hz |
| 11913-11913.7 | Hz |
| 12828.3-12829 | Hz |
| 13686.4-13687 | Hz |
| 14249-14249.7 | Hz |
| 14515.4-14516 | Hz |
| 14586.3-14587 | Hz |
| 14796-14796.7 | Hz |
| 15067-15067.7 | Hz |
| 15112-15112.7 | Hz |
| 17095-17095.7 | Hz |
| 17167-17167.7 | Hz |
| 18090-18090.7 | Hz |
| 18670.3-18671 | Hz |
| 18895-18895.7 | Hz |
| 19026-19026.7 | Hz |
| 20237-20237.7 | Hz |
| 20254-20254.7 | Hz |
| 20286-20286.7 | Hz |
| 24196-24196.7 | Hz |
| 25199-25199.7 | Hz |
| 26073-26073.7 | Hz |
| 26163-26163.7 | Hz |
| 26164-26164.7 | Hz |
| 30759-30759.7 | Hz |
| 32591.3-32592 | Hz |
| 36343-36343.7 | Hz |
| 41883-41883.7 | Hz |
| 44517-44517.7 | Hz |
| 44531-44531.7 | Hz |
| 45490-45490.7 | Hz |
| 47297-47297.7 | Hz |
| 49434-49434.7 | Hz |
| 50229-50229.7 | Hz |
| 69481-69481.7 | Hz |
| 78477.4-78478 | Hz |
| 80993.3-80994 | Hz |
| 81999-81999.7 | Hz |
| 85810-85810.7 | Hz |
| 89748-89748.7 | Hz |
| 92182-92182.7 | Hz |
| 95245-95245.7 | Hz |

Treatment of Squamous Cell Carcinoma of the Anal Canal

Squamous cell carcinoma of the anal canal may be treated according to the methods disclosed herein by the application of squamous cell carcinoma of the anal canal specific amplitude-modulated frequencies. Squamous cell carcinoma of the anal canal specific frequencies are provided below in Table 11. A patient suffering from squamous cell carcinoma of the anal canal may be treated with any number of the frequencies provided in Table 11, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more of the listed squamous cell carcinoma of the anal canal specific frequencies may be used in the treatment of squamous cell carcinoma of the anal canal. It should be noted that while the table below provides certain frequencies for treatment of squamous cell carcinoma of the anal canal, the disclosure is not so limiting, and other frequencies discovered to be effective in the treatment of squamous cell carcinoma of the anal canal are within the scope of this disclosure. Furthermore, each of the frequencies listed below include values that are close to (i.e., within +/−0.1 Hz), but not exactly, the number or range.

TABLE 11
Amplitude-modulated frequencies discovered to be effective
for treatment of squamous cell carcinoma of the anal canal.

| | |
|---|---|
| 295-295.6 | Hz |
| 304-304.7 | Hz |
| 436-436.7 | Hz |
| 581-581.7 | Hz |
| 679-679.7 | Hz |
| 694.3-695 | Hz |
| 708.3-709 | Hz |
| 796.3-797 | Hz |
| 1759-1759.7 | Hz |
| 1858-1858.7 | Hz |
| 1873-1873.7 | Hz |
| 2442-2442.7 | Hz |
| 2522-2522.7 | Hz |
| 2648-2648.7 | Hz |
| 2893-2893.7 | Hz |
| 2975-2975.7 | Hz |
| 2981-2981.7 | Hz |
| 3238-3238.7 | Hz |
| 3249.3-3250 | Hz |
| 3250.3-3250.8 | Hz |
| 3461-3461.7 | Hz |
| 3531-3531.7 | Hz |
| 3535-3535.7 | Hz |
| 3681-3681.7 | Hz |
| 4279-4279.7 | Hz |
| 4440.4-4441 | Hz |
| 4718-4718.7 | Hz |
| 4876-4876.7 | Hz |
| 5034-5034.7 | Hz |
| 5068-5068.7 | Hz |
| 5151-5151.7 | Hz |
| 5249-5249.7 | Hz |
| 5286-5286.7 | Hz |
| 5869-5869.7 | Hz |
| 5881-5881.7 | Hz |
| 6298.4-6299 | Hz |
| 6851-6851.7 | Hz |
| 6879-6879.7 | Hz |
| 6882.4-6883 | Hz |
| 6897-6897.7 | Hz |
| 6980.3-6981 | Hz |
| 7136-7136.7 | Hz |
| 7274-7274.7 | Hz |
| 7673-7673.7 | Hz |
| 7930-7930.7 | Hz |
| 7981-7981.7 | Hz |
| 8328-8328.7 | Hz |
| 8432-8432.7 | Hz |
| 8755-8755.7 | Hz |
| 8949-8949.7 | Hz |
| 9656-9656.7 | Hz |
| 10439-10439.7 | Hz |
| 10484-10484.7 | Hz |
| 11145-11145.7 | Hz |
| 11159-11159.7 | Hz |
| 11253-11253.7 | Hz |
| 11260-11260.7 | Hz |
| 11277.3-11278 | Hz |
| 11559-11559.7 | Hz |
| 11699-11699.7 | Hz |
| 11905-11905.7 | Hz |
| 12139-12139.7 | Hz |
| 13819-13819.7 | Hz |
| 13847-13847.7 | Hz |
| 13903-13903.7 | Hz |
| 17379-17379.7 | Hz |
| 18618-18618.7 | Hz |
| 18742-18742.7 | Hz |
| 21445-21445.7 | Hz |
| 21467-21467.7 | Hz |
| 22763-22763.7 | Hz |
| 33536-33536.7 | Hz |
| 37527-37527.7 | Hz |
| 37546-37546.7 | Hz |
| 53079-53079.7 | Hz |
| 58094-58094.7 | Hz |
| 59288-59288.7 | Hz |
| 59997-59997.7 | Hz |
| 88281-88281.7 | Hz |

Treatment of Hepatocellular Carcinoma

Hepatocellular carcinoma may be treated according to the methods disclosed herein by the application of hepatocellular carcinoma specific amplitude-modulated frequencies. Hepatocellular carcinoma specific frequencies are provided below in Table 12. A patient suffering from hepatocellular carcinoma may be treated with any number of the frequencies provided in Table 12, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed hepatocellular carcinoma specific frequencies may be used in the treatment of hepatocellular carcinoma. It should be noted that while the table below provides certain frequencies for treatment of hepatocellular carcinoma, the disclosure is not so limiting, and other frequencies discovered to be effective in the treatment of hepatocellular carcinoma are within the scope of this disclosure. Furthermore, each of the frequencies listed below include values that are close to (i.e., within +/−0.1 Hz), but not exactly, the number or range.

TABLE 12
Amplitude-modulated frequencies discovered to be effective
for treatment of hepatocellular carcinoma.

| | |
|---|---|
| 439-439.7 | Hz |
| 594-594.7 | Hz |
| 737-737.7 | Hz |
| 817-817.7 | Hz |
| 966-966.7 | Hz |
| 996-996.7 | Hz |
| 1785-1785.7 | Hz |
| 1823-1823.7 | Hz |
| 1881-1881.7 | Hz |
| 1963-1963.7 | Hz |
| 1973-1973.7 | Hz |
| 2189-2189.7 | Hz |
| 2321-2321.7 | Hz |
| 2372.3-2372.8 | Hz |
| 2459-2459.7 | Hz |
| 2647-2647.7 | Hz |
| 2825-2825.7 | Hz |
| 2836-2836.7 | Hz |
| 2860-2860.7 | Hz |
| 2971-2971.6 | Hz |
| 3171-3171.7 | Hz |
| 3188-3188.7 | Hz |
| 3749-3749.7 | Hz |
| 3762-3762.7 | Hz |
| 3823-3823.7 | Hz |
| 4007-4007.7 | Hz |
| 4013-4013.7 | Hz |
| 4454-4454.7 | Hz |
| 4997-4997.7 | Hz |
| 5151-5151.7 | Hz |
| 5239-5239.7 | Hz |
| 5322-5322.7 | Hz |
| 6317-6317.7 | Hz |
| 6330-6330.7 | Hz |
| 6334-6334.7 | Hz |
| 6432-6432.7 | Hz |
| 6840-6840.7 | Hz |
| 7098-7098.7 | Hz |
| 7169-7169.7 | Hz |
| 7239-7239.7 | Hz |
| 7338-7338.7 | Hz |
| 7378-7378.7 | Hz |
| 7481-7481.7 | Hz |
| 7510-7510.7 | Hz |
| 7719-7719.7 | Hz |
| 7748-7748.7 | Hz |
| 7787-7787.7 | Hz |
| 8388-8388.7 | Hz |
| 8412-8412.7 | Hz |
| 9588-9588.7 | Hz |
| 8522-8522.7 | Hz |
| 8845-8845.7 | Hz |
| 8935-8935.6 | Hz |
| 8949-8949.7 | Hz |
| 9273-9273.7 | Hz |
| 9695-9695.7 | Hz |
| 10387-10387.7 | Hz |
| 10641-10641.7 | Hz |
| 10731-10731.7 | Hz |
| 10740-10740.7 | Hz |
| 10762-10762.7 | Hz |
| 10768-10768.7 | Hz |
| 10776-10776.7 | Hz |
| 10805-10805.7 | Hz |
| 10831-10831.7 | Hz |
| 10889-10889.7 | Hz |
| 11209-11209.7 | Hz |
| 11243-11243.7 | Hz |
| 11247-11247.7 | Hz |
| 11275-11275.7 | Hz |
| 11295-11295.7 | Hz |
| 11569-11569.7 | Hz |
| 11590-11590.7 | Hz |
| 11946-11946.7 | Hz |
| 12498-12498.7 | Hz |
| 12572-12572.7 | Hz |
| 12649-12649.7 | Hz |
| 13215-13215.7 | Hz |
| 13252-13252.7 | Hz |
| 13735-13735.7 | Hz |
| 13856-13856.7 | Hz |
| 14114-14114.7 | Hz |
| 14170.7-14171.3 | Hz |
| 14532-14532.7 | Hz |
| 14574-14574.7 | Hz |
| 15521-15521.7 | Hz |
| 15528-15528.7 | Hz |
| 15546.3-15547 | Hz |
| 15589-15589.7 | Hz |
| 15982-15982.7 | Hz |
| 16381-16381.7 | Hz |
| 17556-17556.7 | Hz |
| 17749-17749.7 | Hz |
| 17978-17978.7 | Hz |
| 18030-18030.7 | Hz |
| 18246-18246.7 | Hz |
| 18567-18567.7 | Hz |
| 18686-18686.7 | Hz |
| 18842-18842.7 | Hz |
| 18897-18897.7 | Hz |
| 19510-19510.7 | Hz |
| 20324-20324.7 | Hz |
| 20825-20825.7 | Hz |
| 20908-20908.7 | Hz |
| 21616-21616.7 | Hz |
| 21778-21778.7 | Hz |
| 21876-21876.7 | Hz |
| 22353-22353.7 | Hz |
| 22459-22459.7 | Hz |
| 22535-22535.7 | Hz |
| 22709-22709.7 | Hz |
| 23509-23509.7 | Hz |
| 23526-23526.7 | Hz |
| 23560-23560.7 | Hz |
| 23589-23589.7 | Hz |
| 23957-23957.7 | Hz |
| 24014-24014.7 | Hz |
| 24117-24117.7 | Hz |
| 24869-24869.7 | Hz |
| 25449-25449.7 | Hz |
| 25454-25454.7 | Hz |
| 26722-26722.7 | Hz |
| 26861-26861.7 | Hz |
| 30942-30942.7 | Hz |
| 31713-31713.7 | Hz |
| 32866-32866.7 | Hz |
| 32870-32870.7 | Hz |
| 33765-33765.7 | Hz |
| 34405-34405.7 | Hz |
| 34562-34562.7 | Hz |
| 34984-34984.6 | Hz |
| 36410-36410.7 | Hz |
| 38483-38483.7 | Hz |
| 38659-38659.7 | Hz |
| 39035-39035.7 | Hz |
| 39223-39223.7 | Hz |
| 39510-39510.7 | Hz |
| 39729-39729.7 | Hz |
| 40693-40693.7 | Hz |
| 41244-41244.7 | Hz |
| 41574-41574.7 | Hz |
| 42913-42913.7 | Hz |
| 42923-42923.7 | Hz |
| 43243-43243.7 | Hz |
| 43413-43413.7 | Hz |
| 43542-43542.7 | Hz |
| 43603-43603.7 | Hz |
| 44742-44742.7 | Hz |
| 47693.4-47694 | Hz |
| 51592-51592.7 | Hz |
| 53947-53947.7 | Hz |
| 55072-55072.7 | Hz |
| 56093-56093.7 | Hz |
| 59089-59089.7 | Hz |
| 66969-66969.7 | Hz |
| 67683-67683.7 | Hz |
| 76537-76537.7 | Hz |
| 78229-78229.7 | Hz |
| 82845-82845.7 | Hz |
| 83612-83612.7 | Hz |
| 84086-84086.7 | Hz |
| 91245-91245.7 | Hz |
| 92361.3-92362 | Hz |
| 93332-93332.7 | Hz |
| 93530-93530.7 | Hz |
| 94968.3-94969 | Hz |
| 99281-99281.7 | Hz |
| 99846-99846.7 | Hz |

In certain embodiments, the frequencies listed above may also be used in combination with one or more of the frequencies disclosed in the '365 patent. Specifically, treatment modalities for treatment of hepatocellular carcinoma in accordance with this disclosure may include application of some or all of the above listed frequencies with one or more of the frequencies disclosed in Table 13 for hepatocellular carcinoma. Alternatively and/or additionally, the frequencies disclosed in the '365 patent may be used for treatment of hepatocellular carcinoma state using the methods and systems described herein.

TABLE 13
Amplitude-modulated frequencies for treatment
of hepatocellular carcinoma.

| | |
|---|---|
| 380.293 | Hz |
| 410.231 | Hz |
| 423.321 | Hz |
| 427.062 | Hz |
| 434.332 | Hz |
| 470.181 | Hz |
| 560.32 | Hz |
| 642.932 | Hz |
| 655.435 | Hz |
| 657.394 | Hz |
| 668.209 | Hz |
| 677.972 | Hz |
| 728.232 | Hz |
| 806.021 | Hz |
| 811.924 | Hz |
| 842.311 | Hz |
| 843.22 | Hz |
| 845.208 | Hz |
| 891.901 | Hz |
| 914.219 | Hz |
| 920.321 | Hz |
| 964.394 | Hz |
| 1250.504 | Hz |
| 1755.402 | Hz |
| 1814.223 | Hz |
| 1851.202 | Hz |
| 1873.477 | Hz |
| 1924.702 | Hz |
| 1975.196 | Hz |
| 2017.962 | Hz |
| 2053.396 | Hz |
| 2083.419 | Hz |
| 2190.731 | Hz |
| 2221.323 | Hz |
| 2308.294 | Hz |
| 2315.208 | Hz |
| 2324.393 | Hz |
| 2338.221 | Hz |
| 2353.478 | Hz |
| 2362.309 | Hz |
| 2379.571 | Hz |
| 2419.309 | Hz |
| 2425.222 | Hz |
| 2430.219 | Hz |
| 2431.094 | Hz |
| 2471.328 | Hz |
| 2478.331 | Hz |
| 2480.191 | Hz |
| 2522.328 | Hz |
| 2743.995 | Hz |
| 2744.211 | Hz |
| 2831.951 | Hz |
| 2843.283 | Hz |
| 2859.891 | Hz |
| 2873.542 | Hz |
| 2886.232 | Hz |
| 3009.332 | Hz |
| 3020.286 | Hz |
| 3042.012 | Hz |
| 3044.213 | Hz |
| 3051.218 | Hz |
| 3076.892 | Hz |
| 3078.983 | Hz |
| 3086.443 | Hz |
| 3104.854 | Hz |
| 3127.232 | Hz |
| 3160.942 | Hz |
| 3161.331 | Hz |
| 3167.22 | Hz |
| 3206.315 | Hz |
| 3255.219 | Hz |
| 3267.433 | Hz |
| 3269.321 | Hz |
| 3281.432 | Hz |
| 3457.291 | Hz |
| 3505.229 | Hz |
| 3516.296 | Hz |
| 3530.188 | Hz |
| 3531.296 | Hz |
| 3546.323 | Hz |
| 3572.106 | Hz |
| 3576.189 | Hz |
| 3669.513 | Hz |
| 3923.221 | Hz |
| 3927.331 | Hz |
| 4013.932 | Hz |
| 4071.121 | Hz |
| 4079.951 | Hz |
| 4123.953 | Hz |
| 4161.889 | Hz |
| 4222.821 | Hz |
| 4238.402 | Hz |
| 4256.321 | Hz |
| 4289.296 | Hz |
| 4312.947 | Hz |
| 4375.962 | Hz |
| 4426.387 | Hz |
| 4428.185 | Hz |
| 4435.219 | Hz |
| 4471.188 | Hz |
| 4483.889 | Hz |
| 4486.384 | Hz |
| 4556.322 | Hz |
| 4629.941 | Hz |
| 4715.222 | Hz |
| 4732.211 | Hz |
| 4767.185 | Hz |
| 4873.333 | Hz |
| 4876.218 | Hz |
| 5086.281 | Hz |
| 5124.084 | Hz |
| 5133.121 | Hz |
| 5247.142 | Hz |
| 5270.834 | Hz |
| 5340.497 | Hz |
| 5520.218 | Hz |
| 5570.234 | Hz |
| 5882.292 | Hz |
| 5926.512 | Hz |
| 6037.311 | Hz |
| 6180.334 | Hz |
| 6329.195 | Hz |
| 6350.333 | Hz |
| 6361.321 | Hz |
| 6364.928 | Hz |
| 6383.321 | Hz |
| 6461.175 | Hz |
| 6661.109 | Hz |
| 6711.392 | Hz |
| 6733.331 | Hz |
| 6758.232 | Hz |
| 6779.482 | Hz |
| 6856.222 | Hz |
| 6877.183 | Hz |
| 6915.886 | Hz |
| 6980.525 | Hz |
| 7019.235 | Hz |
| 7041.321 | Hz |
| 7043.209 | Hz |
| 7078.307 | Hz |
| 7130.323 | Hz |
| 7144.142 | Hz |
| 7210.223 | Hz |
| 7232.343 | Hz |
| 7291.21 | Hz |
| 7482.245 | Hz |
| 7510.92 | Hz |
| 7529.233 | Hz |
| 7549.212 | Hz |
| 7650.028 | Hz |
| 7680.518 | Hz |
| 7692.522 | Hz |
| 7829.231 | Hz |
| 7862.209 | Hz |
| 7932.482 | Hz |
| 7935.423 | Hz |
| 7947.392 | Hz |
| 7979.308 | Hz |
| 8025.322 | Hz |
| 8028.339 | Hz |
| 8055.942 | Hz |
| 8072.134 | Hz |
| 8141.174 | Hz |
| 8208.285 | Hz |
| 8328.312 | Hz |
| 8336.383 | Hz |
| 8394.793 | Hz |
| 8432.181 | Hz |
| 8452.119 | Hz |
| 8460.944 | Hz |
| 8475.221 | Hz |
| 8492.193 | Hz |
| 8542.311 | Hz |
| 8779.229 | Hz |
| 8818.104 | Hz |
| 8852.329 | Hz |
| 8853.444 | Hz |
| 8858.179 | Hz |
| 8915.221 | Hz |
| 8939.212 | Hz |
| 8953.231 | Hz |
| 8993.239 | Hz |
| 9278.889 | Hz |
| 9332.397 | Hz |
| 9381.221 | Hz |
| 9520.333 | Hz |
| 9719.314 | Hz |
| 9740.219 | Hz |
| 9768.331 | Hz |
| 9773.111 | Hz |
| 9797.294 | Hz |
| 9819.511 | Hz |
| 9845.319 | Hz |
| 10015.419 | Hz |
| 10043.293 | Hz |
| 10317.499 | Hz |
| 10438.495 | Hz |
| 10443.311 | Hz |
| 10456.383 | Hz |
| 10579.425 | Hz |
| 10863.209 | Hz |
| 10866.382 | Hz |
| 11067.418 | Hz |
| 11149.935 | Hz |
| 11163.895 | Hz |
| 11195.509 | Hz |
| 11421.219 | Hz |
| 11802.821 | Hz |
| 11953.424 | Hz |
| 12024.502 | Hz |
| 12223.329 | Hz |
| 12228.369 | Hz |
| 12247.233 | Hz |
| 12260.933 | Hz |
| 12265.295 | Hz |
| 12267.233 | Hz |
| 12267.296 | Hz |
| 12274.219 | Hz |
| 12623.191 | Hz |
| 12633.372 | Hz |
| 12685.231 | Hz |
| 12721.423 | Hz |
| 12785.342 | Hz |
| 13433.323 | Hz |
| 13457.388 | Hz |
| 14085.222 | Hz |
| 14212.122 | Hz |
| 14226.313 | Hz |
| 14333.209 | Hz |
| 14537.331 | Hz |
| 14542.432 | Hz |
| 14655.03 | Hz |
| 14736.223 | Hz |
| 14828.234 | Hz |
| 15149.213 | Hz |
| 15237.489 | Hz |
| 15560.908 | Hz |
| 15717.221 | Hz |
| 16110.932 | Hz |
| 16144.343 | Hz |
| 17153.322 | Hz |
| 17660.109 | Hz |
| 18121.184 | Hz |
| 18265.238 | Hz |
| 18283.323 | Hz |
| 18863.292 | Hz |
| 18930.995 | Hz |
| 19970.311 | Hz |
| 20330.294 | Hz |
| 20365.284 | Hz |
| 22321.331 | Hz |
| 24119.295 | Hz |
| 24181.221 | Hz |

One or more of the AM frequencies of Table 12 may be used in combination with one or more of the frequencies provided in Table 13. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of hepatocellular carcinoma, which frequencies are selected from Table 12 and, optionally from Table 13, provided that at least one or more frequencies are selected from Table 12.

Treatment of Cholangiocarcinoma

Cholangiocarcinoma may be treated according to the methods disclosed herein by the application of cholangiocarcinoma specific amplitude-modulated frequencies. Cholangiocarcinoma specific frequencies are provided below in Table 14. A patient suffering from cholangiocarcinoma may be treated with any number of the frequencies provided in Table 14, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed cholangiocarcinoma specific frequencies may be used in the treatment of cholangiocarcinoma. It should be noted that while the table below provides certain frequencies for treatment of cholangiocarcinoma, the disclosure is not so limiting, and other frequencies discovered to be effective in the treatment of cholangiocarcinoma are within the scope of this disclosure. Furthermore, each of the frequencies listed below include values that are close to (i.e., within +/−0.1 Hz), but not exactly, the number or range.

TABLE 14
Amplitude-modulated frequencies discovered to be
effective for treatment of cholangiocarcinoma.

| | |
|---|---|
| 334-334.7 | Hz |
| 380-380.7 | Hz |
| 410-410.7 | Hz |
| 427-427.6 | Hz |
| 434-434.7 | Hz |
| 476-476.7 | Hz |
| 527-527.7 | Hz |
| 560-560.7 | Hz |
| 624-624.7 | Hz |
| 642.4-643 | Hz |
| 657-657.7 | Hz |
| 677.4-678 | Hz |
| 728-728.7 | Hz |
| 806-806.6 | Hz |
| 809-809.7 | Hz |
| 845-845.7 | Hz |
| 920-920.7 | Hz |
| 961-961.7 | Hz |
| 963.8-964.25 | Hz |
| 964.3-964.8 | Hz |
| 974.4-975 | Hz |
| 1156.3-1157 | Hz |
| 1157-1157.7 | Hz |
| 1755-1755.7 | Hz |
| 1873-1873.7 | Hz |
| 1967-1967.7 | Hz |
| 2017.4-2018 | Hz |
| 2021-2021.7 | Hz |
| 2023-2023.7 | Hz |
| 2035-2035.7 | Hz |
| 2053-2053.7 | Hz |
| 2065-2065.7 | Hz |
| 2083-2083.7 | Hz |
| 2094-2094.7 | Hz |
| 2111-2111.7 | Hz |
| 2128-2128.7 | Hz |
| 2278-2278.7 | Hz |
| 2284-2284.7 | Hz |
| 2308-2308.7 | Hz |
| 2324-2324.7 | Hz |
| 2338-2338.7 | Hz |
| 2362-2362.7 | Hz |
| 2381-2381.7 | Hz |
| 2419-2419.7 | Hz |
| 2425-2425.7 | Hz |
| 2430-2430.7 | Hz |
| 2431-2431.6 | Hz |
| 2471-2471.7 | Hz |
| 2480-2480.7 | Hz |
| 2522-2522.7 | Hz |
| 2636-2636.7 | Hz |
| 2669-2669.7 | Hz |
| 2679-2679.7 | Hz |
| 2685-2685.6 | Hz |
| 2716-2716.6 | Hz |
| 2743.4-2744 | Hz |
| 2835-2835.7 | Hz |
| 2854-2854.7 | Hz |
| 2865-2865.7 | Hz |
| 2873.3-2874 | Hz |
| 2886-2886.7 | Hz |
| 3009-3009.7 | Hz |
| 3020-3020.7 | Hz |
| 3042-3042.6 | Hz |
| 3076.3-3077 | Hz |
| 3104.3-3105 | Hz |
| 3135-3135.7 | Hz |
| 3143-3143.7 | Hz |
| 3160.4-3161 | Hz |
| 3167-3167.7 | Hz |
| 3206-3206.7 | Hz |
| 3255-3255.7 | Hz |
| 3267-3267.7 | Hz |
| 3269-3269.7 | Hz |
| 3330.4-3331 | Hz |
| 3516-3516.7 | Hz |
| 3531-3531.7 | Hz |
| 3549-3549.7 | Hz |
| 3564-3564.7 | Hz |
| 3572-3572.7 | Hz |
| 3576-3576.7 | Hz |
| 3619-3619.7 | Hz |
| 3630-3630.7 | Hz |
| 3637.7-3638.3 | Hz |
| 3644-3644.7 | Hz |
| 3696-3696.7 | Hz |
| 3749.3-3750 | Hz |
| 3947-3947.7 | Hz |
| 3954-3954.7 | Hz |
| 4013.4-4014 | Hz |
| 4079.4-4080 | Hz |
| 4123.4-4124 | Hz |
| 4281-4281.7 | Hz |
| 4289-4289.7 | Hz |
| 4312.4-4313 | Hz |
| 4418-4418.7 | Hz |
| 4426-4426.7 | Hz |
| 4441-4441.7 | Hz |
| 4685-4685.6 | Hz |
| 4707-4707.7 | Hz |
| 4715-4715.7 | Hz |
| 4732-4732.7 | Hz |
| 4732-4732.7 | Hz |
| 4748-4748.7 | Hz |
| 4767-4767.7 | Hz |
| 4812-4812.7 | Hz |
| 4821-4821.7 | Hz |
| 4873-4873.7 | Hz |
| 4874-4874.7 | Hz |
| 4876-4876.7 | Hz |
| 4941-4941.7 | Hz |
| 5209.4-5210 | Hz |
| 5229-5229.7 | Hz |
| 5243-5243.7 | Hz |
| 5247-5247.7 | Hz |
| 5262-5262.7 | Hz |
| 5271-5271.7 | Hz |
| 5387.3-5388 | Hz |
| 5573-5573.7 | Hz |
| 5671-5671.7 | Hz |
| 5679-5679.7 | Hz |
| 5966-5966.7 | Hz |
| 5976.3-5977 | Hz |
| 6064-6064.7 | Hz |
| 6086-6086.7 | Hz |
| 6180-6180.7 | Hz |
| 6479.4-6480 | Hz |
| 6513-6513.7 | Hz |
| 6711-6711.7 | Hz |
| 6779-6779.7 | Hz |
| 6915.3-6916 | Hz |
| 7019-7019.7 | Hz |
| 7043-7043.7 | Hz |
| 7130-7130.7 | Hz |
| 7213-7213.7 | Hz |
| 7228.3-7229 | Hz |
| 7234-7234.7 | Hz |
| 7246-7246.7 | Hz |
| 7268-7268.7 | Hz |
| 7367-7367.7 | Hz |
| 7482-7482.7 | Hz |
| 7510.4-7511 | Hz |
| 7680.3-7681 | Hz |
| 7862-7862.7 | Hz |
| 7935-7935.7 | Hz |
| 8012.3-8013 | Hz |
| 8013-8013.7 | Hz |
| 8026-8026.7 | Hz |
| 8028-8028.7 | Hz |
| 8028-8028.7 | Hz |
| 8055.4-8056 | Hz |
| 8072-8072.7 | Hz |
| 8074-8074.7 | Hz |
| 8079-8079.7 | Hz |
| 8141-8141.7 | Hz |
| 8336-8336.7 | Hz |
| 8394.3-8395 | Hz |
| 8421-8421.7 | Hz |
| 8432-8432.7 | Hz |
| 8452-8452.7 | Hz |
| 8492-8492.7 | Hz |
| 8509.4-8510 | Hz |
| 8542-8542.7 | Hz |
| 8779-8779.7 | Hz |
| 8789.3-8790 | Hz |
| 8818-8818.7 | Hz |
| 8852-8852.7 | Hz |
| 8993-8993.7 | Hz |
| 9023-9023.7 | Hz |
| 9112-9112.7 | Hz |
| 9332-9332.7 | Hz |
| 9753-9753.7 | Hz |
| 9846-9846.7 | Hz |
| 10140-10140.7 | Hz |
| 10317-10317.7 | Hz |
| 10362-10362.7 | Hz |
| 10488-10488.7 | Hz |
| 10661-10661.7 | Hz |
| 10665.4-10666 | Hz |
| 10690-10690.7 | Hz |
| 10866-10866.7 | Hz |
| 11081-11081.7 | Hz |
| 11109.3-11110 | Hz |
| 11219-11219.7 | Hz |
| 11582-11582.7 | Hz |
| 11619-11619.7 | Hz |
| 11637-11637.7 | Hz |
| 11802.3-11803 | Hz |
| 11809-11809.7 | Hz |
| 11831-11831.7 | Hz |
| 11872-11872.7 | Hz |
| 11904.3-11905 | Hz |
| 12024.3-12025 | Hz |
| 12228-12228.7 | Hz |
| 12247-12247.7 | Hz |
| 12260.4-12261 | Hz |
| 12274-12274.7 | Hz |
| 12682-12682.7 | Hz |
| 12752-12752.7 | Hz |
| 12783-12783.7 | Hz |
| 12785-12785.7 | Hz |
| 13127-13127.7 | Hz |
| 13457-13457.7 | Hz |
| 13609-13609.7 | Hz |
| 13734-13734.7 | Hz |
| 13747-13747.7 | Hz |
| 13755-13755.7 | Hz |
| 13760-13760.7 | Hz |
| 13761-13761.7 | Hz |
| 13767-13767.7 | Hz |
| 13981-13981.7 | Hz |
| 14005-14005.7 | Hz |
| 14027-14027.7 | Hz |
| 14070-14070.7 | Hz |
| 14078-14078.7 | Hz |
| 14356-14356.7 | Hz |
| 14531-14531.7 | Hz |
| 14545-14545.7 | Hz |
| 14564-14564.7 | Hz |
| 14828-14828.7 | Hz |
| 14867-14867.7 | Hz |
| 14913.4-14914 | Hz |
| 14940-14940.7 | Hz |
| 15173-15173.7 | Hz |
| 15224-15224.7 | Hz |
| 15244-15244.7 | Hz |
| 15432-15432.7 | Hz |
| 15460-15460.7 | Hz |
| 15515-15515.7 | Hz |
| 15539-15539.7 | Hz |
| 15550.4-15551 | Hz |
| 15857-15857.7 | Hz |
| 16082-16082.7 | Hz |
| 16515-16515.7 | Hz |
| 17636-17636.7 | Hz |
| 17673-17673.7 | Hz |
| 18087-18087.7 | Hz |
| 18222-18222.7 | Hz |
| 18521-18521.7 | Hz |
| 18585-18585.7 | Hz |
| 19347-19347.7 | Hz |
| 19889-19889.7 | Hz |
| 21466-21466.7 | Hz |
| 21567-21567.7 | Hz |
| 21844-21844.7 | Hz |
| 21870-21870.7 | Hz |
| 21894-21894.7 | Hz |
| 22073-22073.7 | Hz |
| 22119-22119.7 | Hz |
| 22411-22411.7 | Hz |
| 22437-22437.7 | Hz |
| 22479-22479.7 | Hz |
| 22582-22582.7 | Hz |
| 22730-22730.6 | Hz |
| 22832-22832.7 | Hz |
| 22863-22863.7 | Hz |
| 23237-23237.7 | Hz |
| 23261-23261.7 | Hz |
| 23444-23444.7 | Hz |
| 26212-26212.7 | Hz |
| 27291-27291.7 | Hz |
| 31051-31051.7 | Hz |
| 35296-35296.7 | Hz |
| 37680-37680.7 | Hz |
| 39053-39053.7 | Hz |
| 39183-39183.7 | Hz |
| 39423-39423.7 | Hz |
| 40111-40111.7 | Hz |
| 41557-41557.7 | Hz |
| 42293-42293.7 | Hz |
| 43172-43172.7 | Hz |
| 43379-43379.7 | Hz |
| 44855-44855.7 | Hz |
| 45163-45163.7 | Hz |
| 45362-45362.7 | Hz |
| 46666-46666.7 | Hz |
| 46673-46673.7 | Hz |
| 46689-46689.6 | Hz |
| 51008-51008.7 | Hz |
| 52063-52063.7 | Hz |
| 52786-52786.7 | Hz |
| 52912-52912.7 | Hz |
| 55723-55723.7 | Hz |
| 61287-61287.7 | Hz |
| 61789-61789.7 | Hz |
| 71635-71635.7 | Hz |
| 86521-86521.7 | Hz |
| 87798.3-87799 | Hz |
| 88898-88898.7 | Hz |
| 91271-91271.7 | Hz |
| 92787-92787.7 | Hz. |

Treatment of Mesothelioma

Mesothelioma may be treated according to the methods disclosed herein by the application of mesothelioma specific amplitude-modulated frequencies. Mesothelioma specific frequencies are provided below in Table 15. A patient suffering from mesothelioma may be treated with any number of the frequencies provided in Table 15, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed mesothelioma specific frequencies may be used in the treatment of mesothelioma. It should be noted that while the table below provides certain frequencies for treatment of mesothelioma, the disclosure is not so limiting, and other frequencies discovered to be effective in the treatment of mesothelioma are within the scope of this disclosure. Furthermore, each of the frequencies listed below include values that are close to (i.e., within +/−0.1 Hz), but not exactly, the number or range.

TABLE 15
Amplitude-modulated frequencies discovered to
be effective for treatment of mesothelioma.

| | |
|---|---|
| 262-262.7 | Hz |
| 412-412.7 | Hz |
| 418-418.7 | Hz |
| 429-429.7 | Hz |
| 441-441.7 | Hz |
| 442-442.7 | Hz |
| 465-465.7 | Hz |
| 485-485.7 | Hz |
| 517-517.7 | Hz |
| 556-556.7 | Hz |
| 581-581.7 | Hz |
| 612-612.7 | Hz |
| 626-626.7 | Hz |
| 638-638.7 | Hz |
| 664-664.7 | Hz |
| 694.3-695 | Hz |
| 708.3-709 | Hz |
| 734.4-735 | Hz |
| 761-761.7 | Hz |
| 764-764.7 | Hz |
| 776-776.7 | Hz |
| 806-806.6 | Hz |
| 831-831.7 | Hz |
| 883-883.7 | Hz |
| 1191-1191.7 | Hz |
| 1591-1591.7 | Hz |
| 2475.4-2476 | Hz |
| 2581-2581.7 | Hz |
| 2749-2749.7 | Hz |
| 2988-2988.7 | Hz |
| 3042-3042.6 | Hz |
| 3131-3131.7 | Hz |
| 3193-3193.7 | Hz |
| 3240-3240.7 | Hz |
| 3335-3335.7 | Hz |
| 3371-3371.7 | Hz |
| 3518-3518.7 | Hz |
| 3523-3523.7 | Hz |
| 3530-3530.7 | Hz |
| 3556-3556.7 | Hz |
| 3621-3621.7 | Hz |
| 3656-3656.7 | Hz |
| 3674-3674.7 | Hz |
| 3681.7-3682.3 | Hz |
| 3817-3817.7 | Hz |
| 3833-3833.7 | Hz |
| 3877-3877.7 | Hz |
| 3948-3948.7 | Hz |
| 3958-3958.6 | Hz |
| 4044-4044.7 | Hz |
| 4190-4190.7 | Hz |
| 4261-4261.7 | Hz |
| 4330-4330.7 | Hz |
| 5496-5496.7 | Hz |
| 5640.4-5641 | Hz |
| 6416.3-6417 | Hz |
| 6441-6441.7 | Hz |
| 6717-6717.7 | Hz |
| 6758-6758.7 | Hz |
| 6783-6783.7 | Hz |
| 7110-7110.7 | Hz |
| 7140-7140.7 | Hz |
| 7154-7154.7 | Hz |
| 7156-7156.7 | Hz |
| 7253-7253.7 | Hz |
| 7283-7283.7 | Hz |
| 7546-7546.7 | Hz |
| 7725-7725.7 | Hz |
| 8386-8386.7 | Hz |
| 8562.4-8563 | Hz |
| 8810-8810.7 | Hz |
| 8827.4-8828 | Hz |
| 8843-8843.7 | Hz |
| 8859.3-8860 | Hz |
| 8863-8863.7 | Hz |
| 8877-8877.7 | Hz |
| 8881-8881.7 | Hz |
| 9075-9075.7 | Hz |
| 9929-9929.7 | Hz |
| 9947-9947.7 | Hz |
| 9970-9970.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11083-11083.7 | Hz |
| 11124-11124.7 | Hz |
| 11540-11540.7 | Hz |
| 11831-11831.7 | Hz |
| 11929-11929.7 | Hz |
| 11956-11956.7 | Hz |
| 12267-12267.7 | Hz |
| 12929-12929.7 | Hz |
| 13674-13674.7 | Hz |
| 13742-13742.7 | Hz |
| 14285-14285.7 | Hz |
| 14947-14947.7 | Hz |
| 15531-15531.7 | Hz |
| 17737-17737.7 | Hz |
| 17757-17757.7 | Hz |
| 18121-18121.7 | Hz |
| 18126-18126.7 | Hz |
| 18140-18140.7 | Hz |
| 18921-18921.7 | Hz |
| 19406-19406.7 | Hz |
| 19476-19476.7 | Hz |
| 19577-19577.7 | Hz |
| 20634-20634.7 | Hz |
| 21372-21372.7 | Hz |
| 22411-22411.7 | Hz |
| 22519-22519.7 | Hz |
| 22538-22538.7 | Hz |
| 22548-22548.7 | Hz |
| 23111-23111.7 | Hz |
| 23921-23921.7 | Hz |
| 24112-24112.7 | Hz |
| 24146-24146.7 | Hz |
| 39153-39153.7 | Hz |

In certain embodiments, the frequencies listed above may also be used in combination with one or more of the frequencies disclosed in the '365 patent. Specifically, treatment modalities for treatment of mesothelioma in accordance with this disclosure may include application of some or all of the above listed frequencies with one or more of the frequencies disclosed in the Table 16 for mesothelioma. Alternatively and/or additionally, the frequencies disclosed in the '365 patent may be used for treatment of mesothelioma using the methods and systems described herein.

TABLE 16
Amplitude-modulated frequencies effective
for treatment of mesothelioma.

| | |
|---|---|
| 958.929 | Hz |
| 1713.913 | Hz |
| 1736.782 | Hz |
| 2334.178 | Hz |
| 2607.193 | Hz |
| 3112.974 | Hz |
| 3319.945 | Hz |
| 3449.219 | Hz |
| 3622.312 | Hz |
| 5151.402 | Hz |
| 5887.022 | Hz |
| 5965.922 | Hz |
| 6516.793 | Hz |
| 7224.197 | Hz |
| 9471.152 | Hz |
| 14617.393 | Hz |

One or more of the AM frequencies of Table 15 may be used in combination with one or more of the frequencies provided in Table 16. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of mesothelioma, which frequencies are selected from Table 15 and, optionally from Table 16, provided that at least one or more frequencies are selected from Table 15.

Treatment of Thyroid Cancer

Thyroid cancer may be treated according to the methods disclosed herein by the application of thyroid cancer specific amplitude-modulated frequencies. Thyroid cancer specific frequencies are provided below in Table 17. A patient suffering from thyroid cancer may be treated with any number of the frequencies provided in Table 17, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed thyroid cancer specific frequencies may be used in the treatment of thyroid cancer. It should be noted that while the table below provides certain frequencies for treatment of thyroid cancer, the disclosure is not so limiting, and other frequencies discovered to be effective in the treatment of thyroid cancer are within the scope of this disclosure. Furthermore, each of the frequencies listed below include values that are close to (i.e., within +/−0.1 Hz), but not exactly, the number or range.

TABLE 17
Amplitude-modulated frequencies discovered to
be effective for treatment of thyroid cancer.

| | |
|---|---|
| 834-834.7 | Hz |
| 836-836.7 | Hz |
| 845-845.7 | Hz |
| 1213-1213.7 | Hz |
| 1220-1220.7 | Hz |
| 1252-1252.7 | Hz |
| 1815-1815.7 | Hz |
| 1966-1966.7 | Hz |
| 2050-2050.7 | Hz |
| 2313-2313.7 | Hz |
| 2318-2318.7 | Hz |
| 2320-2320.7 | Hz |
| 2328-2328.7 | Hz |
| 2336-2336.7 | Hz |
| 2359-2359.7 | Hz |
| 2373-2373.7 | Hz |
| 2380-2380.7 | Hz |
| 2425-2425.7 | Hz |
| 2429-2429.7 | Hz |
| 2436-2436.7 | Hz |
| 2513-2513.7 | Hz |
| 2629-2629.7 | Hz |
| 2653.7-2654.4 | Hz |
| 2830-2830.7 | Hz |
| 2834-2834.7 | Hz |
| 3030-3030.7 | Hz |
| 3056-3056.7 | Hz |
| 3609-3609.7 | Hz |
| 3646-3646.7 | Hz |
| 3674-3674.7 | Hz |
| 3694-3694.7 | Hz |
| 3908.7-3909.22 | Hz |
| 3981-3981.7 | Hz |
| 4027-4027.7 | Hz |
| 4386.4-4387 | Hz |
| 4425.7-4426.22 | Hz |
| 4792-4792.7 | Hz |
| 4844-4844.7 | Hz |
| 4870-4870.7 | Hz |
| 5259-5259.7 | Hz |
| 5510-5510.7 | Hz |
| 5533-5533.7 | Hz |
| 5714-5714.7 | Hz |
| 5741-5741.7 | Hz |
| 5742-5742.7 | Hz |
| 5752-5752.7 | Hz |
| 5766-5766.7 | Hz |
| 6392-6392.7 | Hz |
| 6423-6423.7 | Hz |
| 6781-6781.7 | Hz |
| 6832-6832.7 | Hz |
| 6864-6864.7 | Hz |
| 7404.4-7405 | Hz |
| 7526-7526.6 | Hz |
| 7646-7646.7 | Hz |
| 7652-7652.7 | Hz |
| 7913-7913.7 | Hz |
| 8217.4-8218 | Hz |
| 8251-8251.7 | Hz |
| 8347-8347.7 | Hz |
| 8731-8731.7 | Hz |
| 8740-8740.7 | Hz |
| 8749-8749.7 | Hz |
| 8814-8814.7 | Hz |
| 9169-9169.7 | Hz |
| 9506-9506.7 | Hz |
| 10159-10159.7 | Hz |
| 10395-10395.7 | Hz |
| 10541-10541.7 | Hz |
| 10830-10830.7 | Hz |
| 10844-10844.7 | Hz |
| 10878-10878.7 | Hz |
| 10993-10993.7 | Hz |
| 11117-11117.7 | Hz |
| 11779-11779.7 | Hz |
| 11867-11867.7 | Hz |
| 12302-12302.7 | Hz |
| 12356-12356.7 | Hz |
| 13117-13117.7 | Hz |
| 13373-13373.7 | Hz |
| 13679-13679.7 | Hz |
| 13694-13694.7 | Hz |
| 14111-14111.7 | Hz |
| 14116-14116.7 | Hz |
| 14156-14156.7 | Hz |
| 14174-14174.7 | Hz |
| 14281-14281.7 | Hz |
| 14523-14523.7 | Hz |
| 14585-14585.7 | Hz |
| 14690-14690.7 | Hz |
| 14716-14716.7 | Hz |
| 14912-14912.7 | Hz |
| 14974-14974.7 | Hz |
| 15223-15223.7 | Hz |
| 15236-15236.7 | Hz |
| 15329-15329.7 | Hz |
| 15615-15615.7 | Hz |
| 15867-15867.7 | Hz |
| 15919-15919.7 | Hz |
| 15942-15942.7 | Hz |
| 16096-16096.7 | Hz |
| 18040-18040.7 | Hz |
| 18182-18182.7 | Hz |
| 18211-18211.7 | Hz |
| 18246-18246.7 | Hz |
| 18274-18274.7 | Hz |
| 18411-18411.7 | Hz |
| 18454-18454.7 | Hz |
| 18462-18462.7 | Hz |
| 18596-18596.7 | Hz |
| 19365-19365.7 | Hz |
| 19419-19419.7 | Hz |
| 19715-19715.7 | Hz |
| 19744-19744.7 | Hz |
| 19823-19823.7 | Hz |
| 20229-20229.7 | Hz |
| 20284-20284.7 | Hz |
| 20513-20513.7 | Hz |
| 20747-20747.7 | Hz |
| 21963-21963.7 | Hz |
| 22338-22338.7 | Hz |
| 22344-22344.7 | Hz |
| 22350-22350.7 | Hz |
| 22422-22422.7 | Hz |
| 22484-22484.7 | Hz |
| 22932-22932.7 | Hz |
| 23012-23012.7 | Hz |
| 23044-23044.7 | Hz |
| 23216-23216.7 | Hz |
| 23659-23659.7 | Hz |
| 23730-23730.7 | Hz |
| 24060-24060.7 | Hz |
| 24085-24085.7 | Hz |
| 26044-26044.7 | Hz |
| 26343-26343.7 | Hz |
| 27214-27214.7 | Hz |
| 28361-28361.7 | Hz |
| 33366-33366.7 | Hz |
| 33414-33414.7 | Hz |
| 33553-33553.7 | Hz |
| 34614-34614.7 | Hz |
| 35826-35826.7 | Hz |
| 40133-40133.7 | Hz |
| 40394-40394.7 | Hz |
| 40896-40896.7 | Hz |
| 40899-40899.7 | Hz |
| 41424-41424.7 | Hz |
| 41534-41534.7 | Hz |
| 41859-41859.7 | Hz |
| 41861-41861.7 | Hz |
| 42402-42402.7 | Hz |
| 43851-43851.7 | Hz |
| 44162-44162.7 | Hz |
| 44692-44692.7 | Hz |
| 45393-45393.7 | Hz |
| 45526-45526.7 | Hz |
| 46282-46282.7 | Hz |
| 46285-46285.7 | Hz |
| 47036-47036.7 | Hz |
| 48314-48314.7 | Hz |
| 48535-48535.7 | Hz |
| 52097-52097.7 | Hz |
| 54293-54293.7 | Hz |
| 54297-54297.7 | Hz |
| 55592-55592.7 | Hz |
| 57927-57927.7 | Hz |
| 59907-59907.7 | Hz |
| 65586-65586.7 | Hz |
| 67697-67697.6 | Hz |
| 71204-71204.7 | Hz |
| 71447-71447.7 | Hz |
| 74017-74017.7 | Hz |
| 76227-76227.7 | Hz |
| 76659-76659.7 | Hz |
| 82024-82024.7 | Hz |

In certain embodiments, the frequencies listed above may also be used in combination with one or more of the frequencies disclosed in the '365 patent. Specifically, treatment modalities for treatment of thyroid cancer in accordance with this disclosure may include application of some or all of the above listed frequencies with one or more of the frequencies disclosed in Table 18 for thyroid cancer. Alternatively and/or additionally, the frequencies disclosed in the '365 patent may be used for treatment of thyroid cancer using the methods and systems described herein.

TABLE 18
Amplitude-modulated frequencies for treatment of thyroid cancer.

| | |
|---|---|
| 410.231 | Hz |
| 412.209 | Hz |
| 479.222 | Hz |
| 493.442 | Hz |
| 517.202 | Hz |
| 556.233 | Hz |
| 617.313 | Hz |
| 618.407 | Hz |
| 618.813 | Hz |
| 618.927 | Hz |
| 621.321 | Hz |
| 628.321 | Hz |
| 648.252 | Hz |
| 658.191 | Hz |
| 663.407 | Hz |
| 694.689 | Hz |
| 777.432 | Hz |
| 812.512 | Hz |
| 814.251 | Hz |
| 820.907 | Hz |
| 821.202 | Hz |
| 831.223 | Hz |
| 874.341 | Hz |
| 914.429 | Hz |
| 941.311 | Hz |
| 942.331 | Hz |
| 983.429 | Hz |
| 1127.239 | Hz |
| 1191.341 | Hz |
| 1380.828 | Hz |
| 1552.123 | Hz |
| 1587.811 | Hz |
| 1614.409 | Hz |
| 1723.389 | Hz |
| 1771.402 | Hz |
| 2155.311 | Hz |
| 2179.231 | Hz |
| 2185.282 | Hz |
| 2221.323 | Hz |
| 2228.832 | Hz |
| 2315.888 | Hz |
| 2341.312 | Hz |
| 2445.123 | Hz |
| 2454.232 | Hz |
| 2723.302 | Hz |
| 2740.384 | Hz |
| 2749.323 | Hz |
| 2856.253 | Hz |
| 2856.921 | Hz |
| 2859.495 | Hz |
| 2871.795 | Hz |
| 2886.232 | Hz |
| 2928.911 | Hz |
| 2988.212 | Hz |
| 3021.122 | Hz |
| 3078.275 | Hz |
| 3080.592 | Hz |
| 3186.331 | Hz |
| 3198.323 | Hz |
| 3248.321 | Hz |
| 3271.329 | Hz |
| 3284.192 | Hz |
| 3335.332 | Hz |
| 3432.343 | Hz |
| 3434.911 | Hz |
| 3440.212 | Hz |
| 3475.216 | Hz |
| 3509.522 | Hz |
| 3533.328 | Hz |
| 3610.203 | Hz |
| 3637.085 | Hz |
| 3682.489 | Hz |
| 3789.288 | Hz |
| 3822.392 | Hz |
| 3909.333 | Hz |
| 3917.211 | Hz |
| 4023.33 | Hz |
| 4028.204 | Hz |
| 4043.332 | Hz |
| 4046.321 | Hz |
| 4154.301 | Hz |
| 4207.322 | Hz |
| 4226.263 | Hz |
| 4236.945 | Hz |
| 4243.393 | Hz |
| 4261.228 | Hz |
| 4330.289 | Hz |
| 4340.833 | Hz |
| 4347.125 | Hz |
| 4358.333 | Hz |
| 4366.294 | Hz |
| 4426.387 | Hz |
| 4440.962 | Hz |
| 4458.339 | Hz |
| 4478.443 | Hz |
| 4479.113 | Hz |
| 4486.193 | Hz |
| 4744.424 | Hz |
| 4827.642 | Hz |
| 4854.318 | Hz |
| 4865.421 | Hz |
| 4897.212 | Hz |
| 5323.192 | Hz |
| 5324.123 | Hz |
| 5548.879 | Hz |
| 5711.283 | Hz |
| 5730.432 | Hz |
| 5754.332 | Hz |
| 5881.295 | Hz |
| 5924.221 | Hz |
| 6455.131 | Hz |
| 6558.342 | Hz |
| 6620.132 | Hz |
| 6666.839 | Hz |
| 6675.951 | Hz |
| 6714.189 | Hz |
| 6745.333 | Hz |
| 6766.281 | Hz |
| 6779.088 | Hz |
| 6780.679 | Hz |
| 6884.432 | Hz |
| 6917.194 | Hz |
| 6946.928 | Hz |
| 7036.122 | Hz |
| 7083.191 | Hz |
| 7230.838 | Hz |
| 7323.209 | Hz |
| 7355.378 | Hz |
| 7432.143 | Hz |
| 7495.763 | Hz |
| 7505.282 | Hz |
| 7534.221 | Hz |
| 7577.421 | Hz |
| 7623.184 | Hz |
| 7626.332 | Hz |
| 7725.339 | Hz |
| 7726.085 | Hz |
| 7920.879 | Hz |
| 8013.953 | Hz |
| 8019.912 | Hz |
| 8021.331 | Hz |
| 8040.231 | Hz |
| 8078.955 | Hz |
| 8082.173 | Hz |
| 8147.1 | Hz |
| 8281.259 | Hz |
| 8309.752 | Hz |
| 8311.371 | Hz |
| 8435.094 | Hz |
| 8442.293 | Hz |
| 8505.312 | Hz |
| 8521.311 | Hz |
| 8525.789 | Hz |
| 8537.321 | Hz |
| 8540.329 | Hz |
| 8543.211 | Hz |
| 8553.329 | Hz |
| 8744.527 | Hz |
| 8881.819 | Hz |
| 9009.329 | Hz |
| 9068.311 | Hz |
| 9070.809 | Hz |
| 9085.911 | Hz |
| 9535.393 | Hz |
| 9720.412 | Hz |
| 10020.521 | Hz |
| 10039.109 | Hz |
| 10127.279 | Hz |
| 10134.161 | Hz |
| 10257.324 | Hz |
| 10498.339 | Hz |
| 10765.224 | Hz |
| 10849.412 | Hz |
| 10924.342 | Hz |
| 10976.321 | Hz |
| 11030.418 | Hz |
| 11360.332 | Hz |
| 11537.292 | Hz |
| 11559.292 | Hz |
| 11812.119 | Hz |
| 11913.222 | Hz |
| 11927.934 | Hz |
| 11955.949 | Hz |
| 11960.179 | Hz |
| 12120.049 | Hz |
| 12139.222 | Hz |
| 12146.335 | Hz |
| 12489.233 | Hz |
| 12984.462 | Hz |
| 13425.229 | Hz |
| 13636.082 | Hz |
| 13654.272 | Hz |
| 13677.211 | Hz |
| 14014.941 | Hz |
| 14228.295 | Hz |
| 14445.214 | Hz |
| 14540.932 | Hz |
| 14823.325 | Hz |
| 14826.334 | Hz |
| 14910.894 | Hz |
| 15180.492 | Hz |
| 15561.322 | Hz |
| 15597.284 | Hz |
| 16023.119 | Hz |
| 16048.391 | Hz |
| 16080.831 | Hz |
| 16129.321 | Hz |
| 16539.532 | Hz |
| 17222.225 | Hz |
| 17253.222 | Hz |
| 17323.196 | Hz |
| 17461.504 | Hz |
| 17577.221 | Hz |
| 17671.321 | Hz |
| 17881.709 | Hz |
| 17911.323 | Hz |
| 17913.286 | Hz |
| 17937.203 | Hz |
| 17948.264 | Hz |
| 18036.921 | Hz |
| 18715.412 | Hz |
| 19859.429 | Hz |
| 21425.321 | Hz |
| 21452.445 | Hz |

One or more of the AM frequencies of Table 17 may be used in combination with one or more of the frequencies provided in Table 18. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of thyroid cancer, which frequencies are selected from Table 17 and, optionally from Table 18, provided that at least one or more frequencies are selected from Table 17.

Treatment of Prostate Cancer

Prostate cancer may be treated according to the methods disclosed herein by the application of prostate cancer specific amplitude-modulated frequencies. Prostate cancer specific frequencies are provided below in Table 19. A patient suffering from prostate cancer may be treated with any number of the frequencies provided in Table 19, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed prostate cancer specific frequencies may be used in the treatment of prostate cancer. It should be noted that while the table below provides certain frequencies for treatment of prostate cancer, the disclosure is not so limiting, and other frequencies discovered to be effective in the treatment of prostate cancer are within the scope of this disclosure. Furthermore, each of the frequencies listed below include values that are close to (i.e., within +/−0.1 Hz), but not exactly, the number or range.

TABLE 19
Amplitude-modulated frequencies discovered to
be effective for treatment of prostate cancer.

| | |
|---|---|
| 408-408.7 | Hz |
| 429.7-430.3 | Hz |
| 561-561.7 | Hz |
| 568-568.7 | Hz |
| 572-572.7 | Hz |
| 631-631.7 | Hz |
| 709-709.7 | Hz |
| 717-717.7 | Hz |
| 733-733.7 | Hz |
| 810-810.7 | Hz |
| 817-817.7 | Hz |
| 830-830.7 | Hz |
| 1668-1668.7 | Hz |
| 1808-1808.6 | Hz |
| 2483-2483.7 | Hz |
| 2518-2518.7 | Hz |
| 2824-2824.7 | Hz |
| 3207-3207.7 | Hz |
| 3245-3245.7 | Hz |
| 3251-3251.7 | Hz |
| 3266.3-3267 | Hz |
| 3279.3-3280 | Hz |
| 3529-3529.7 | Hz |
| 3562-3562.7 | Hz |
| 3571-3571.7 | Hz |
| 3627-3627.7 | Hz |
| 3661-3661.7 | Hz |
| 3731-3731.7 | Hz |
| 3743-3743.7 | Hz |
| 3756-3756.7 | Hz |
| 3787-3787.7 | Hz |
| 3962-3962.7 | Hz |
| 4152-4152.7 | Hz |
| 4409-4409.7 | Hz |
| 4476-4476.7 | Hz |
| 4806-4806.7 | Hz |
| 4809-4809.7 | Hz |
| 4823-4823.7 | Hz |
| 4841-4841.7 | Hz |
| 4948-4948.7 | Hz |
| 4998-4998.7 | Hz |
| 5336-5336.7 | Hz |
| 5414-5414.7 | Hz |
| 5884-5884.7 | Hz |
| 6117-6117.7 | Hz |
| 6252-6252.7 | Hz |
| 6329-6329.7 | Hz |
| 6333-6333.7 | Hz |
| 6748-6748.7 | Hz |
| 6782-6782.7 | Hz |
| 7112-7112.7 | Hz |
| 7424-7424.7 | Hz |
| 7655-7655.7 | Hz |
| 7906-7906.7 | Hz |
| 7910-7910.7 | Hz |
| 7923-7923.7 | Hz |
| 8373-8373.7 | Hz |
| 8445-8445.7 | Hz |
| 8765-8765.7 | Hz |
| 8829-8829.7 | Hz |
| 8852-8852.7 | Hz |
| 8877-8877.7 | Hz |
| 9078-9078.7 | Hz |
| 9512-9512.7 | Hz |
| 9635-9635.7 | Hz |
| 9681-9681.7 | Hz |
| 9886-9886.7 | Hz |
| 10735-10735.7 | Hz |
| 10748-10748.7 | Hz |
| 10781-10781.7 | Hz |
| 10813.2-10813.8 | Hz |
| 11134-11134.7 | Hz |
| 11155-11155.7 | Hz |
| 11164-11164.7 | Hz |
| 11264-11264.7 | Hz |
| 11381-11381.7 | Hz |
| 11416-11416.7 | Hz |
| 11438-11438.7 | Hz |
| 11469-11469.7 | Hz |
| 11628-11628.7 | Hz |
| 12330-12330.7 | Hz |
| 12391-12391.7 | Hz |
| 12880-12880.7 | Hz |
| 13467-13467.7 | Hz |
| 13562-13562.6 | Hz |
| 13805-13805.7 | Hz |
| 13828-13828.7 | Hz |
| 13834-13834.7 | Hz |
| 13857.4-13858 | Hz |
| 13858-13858.7 | Hz |
| 13871-13871.7 | Hz |
| 14009-14009.7 | Hz |
| 14034-14034.7 | Hz |
| 14153-14153.7 | Hz |
| 14489-14489.7 | Hz |
| 15071-15071.7 | Hz |
| 15149-15149.7 | Hz |
| 15233-15233.7 | Hz |
| 15253-15253.7 | Hz |
| 15263-15263.7 | Hz |
| 15435-15435.7 | Hz |
| 15545-15545.7 | Hz |
| 16176-16176.7 | Hz |
| 16329-16329.7 | Hz |
| 16679-16679.7 | Hz |
| 16804-16804.7 | Hz |
| 16821-16821.7 | Hz |
| 17333-17333.7 | Hz |
| 17435-17435.7 | Hz |
| 17974-17974.7 | Hz |
| 18260-18260.7 | Hz |
| 18641-18641.7 | Hz |
| 18831-18831.7 | Hz |
| 18884-18884.7 | Hz |
| 19453-19453.7 | Hz |
| 20948-20948.7 | Hz |
| 21119-21119.7 | Hz |
| 21167-21167.7 | Hz |
| 21636-21636.7 | Hz |
| 22312-22312.7 | Hz |
| 22361-22361.7 | Hz |
| 22419-22419.7 | Hz |
| 22430-22430.7 | Hz |
| 22443-22443.7 | Hz |
| 22459-22459.7 | Hz |
| 22879-22879.7 | Hz |
| 23117-23117.7 | Hz |
| 23137-23137.7 | Hz |
| 23412-23412.7 | Hz |
| 23548-23548.7 | Hz |
| 23686-23686.7 | Hz |
| 26142-26142.7 | Hz |
| 26465-26465.7 | Hz |
| 27328-27328.6 | Hz |
| 28653-28653.7 | Hz |
| 28744-28744.7 | Hz |
| 31572-31572.7 | Hz |
| 31633-31633.7 | Hz |
| 41062-41062.7 | Hz |
| 41325-41325.7 | Hz |
| 41473-41473.7 | Hz |
| 48683-48683.7 | Hz |
| 48793-48793.7 | Hz |
| 87507-87507.7 | Hz |

In certain embodiments, the frequencies listed above may also be used in combination with one or more of the frequencies disclosed in the '365 patent. Specifically, treatment modalities for treatment of prostate cancer in accordance with this disclosure may include application of some or all of the above listed frequencies with one or more of the frequencies disclosed in Table 20 for prostate cancer. Alternatively and/or additionally, the frequencies disclosed in the '365 patent may be used for treatment of prostate cancer using the methods and systems described herein.

TABLE 20
Amplitude-modulated frequencies for treatment of prostate cancer.

| | |
|---|---|
| 331.3 | Hz |
| 331.358 | Hz |
| 403.218 | Hz |
| 430.439 | Hz |
| 436.231 | Hz |
| 461.233 | Hz |
| 522.2 | Hz |
| 522.213 | Hz |
| 618.4 | Hz |
| 618.407 | Hz |
| 618.8 | Hz |
| 656.295 | Hz |
| 657.394 | Hz |
| 657.397 | Hz |
| 657.4 | Hz |
| 657.483 | Hz |
| 659.033 | Hz |
| 694.4 | Hz |
| 694.689 | Hz |
| 694.7 | Hz |
| 741.4 | Hz |
| 741.421 | Hz |
| 749.221 | Hz |
| 752.9 | Hz |
| 752.933 | Hz |
| 776.194 | Hz |
| 785.219 | Hz |
| 786.332 | Hz |
| 793.331 | Hz |
| 809.205 | Hz |
| 819.322 | Hz |
| 840.133 | Hz |
| 844.8 | Hz |
| 844.822 | Hz |
| 847.332 | Hz |
| 929.1 | Hz |
| 1083.309 | Hz |
| 1102.635 | Hz |
| 1102.71 | Hz |
| 1240.336 | Hz |
| 1372.934 | Hz |
| 1444.288 | Hz |
| 1486.322 | Hz |
| 1563.332 | Hz |
| 1591.322 | Hz |
| 1670.699 | Hz |
| 1697.321 | Hz |
| 1708.195 | Hz |
| 1741.939 | Hz |
| 1743.521 | Hz |
| 2031.448 | Hz |
| 2050.282 | Hz |
| 2076.519 | Hz |
| 2156.332 | Hz |
| 2229.515 | Hz |
| 2243.121 | Hz |
| 2381.443 | Hz |
| 2440.489 | Hz |
| 2475.912 | Hz |
| 2477.919 | Hz |
| 2551.332 | Hz |
| 2579.435 | Hz |
| 2628.324 | Hz |
| 2669.328 | Hz |
| 2824.832 | Hz |
| 2887.829 | Hz |
| 2891.331 | Hz |
| 3081.523 | Hz |
| 3133.309 | Hz |
| 3249.529 | Hz |
| 3250.125 | Hz |
| 3251.815 | Hz |
| 3264.827 | Hz |
| 3278.329 | Hz |
| 3281.432 | Hz |
| 3348.783 | Hz |
| 3519.118 | Hz |
| 3539.962 | Hz |
| 3551.318 | Hz |
| 3556.439 | Hz |
| 3572.321 | Hz |
| 3615.223 | Hz |
| 3670.129 | Hz |
| 3681.341 | Hz |
| 3686.021 | Hz |
| 3753.382 | Hz |
| 3774.923 | Hz |
| 3867.692 | Hz |
| 3909.333 | Hz |
| 3916.321 | Hz |
| 4031.233 | Hz |
| 4031.933 | Hz |
| 4038.203 | Hz |
| 4047.233 | Hz |
| 4066.222 | Hz |
| 4081.743 | Hz |
| 4084.319 | Hz |
| 4139.322 | Hz |
| 4153.192 | Hz |
| 4223.795 | Hz |
| 4231.221 | Hz |
| 4241.321 | Hz |
| 4320.513 | Hz |
| 4329.152 | Hz |
| 4380.321 | Hz |
| 4417.312 | Hz |
| 4489.452 | Hz |
| 4549.808 | Hz |
| 4558.306 | Hz |
| 4579.324 | Hz |
| 4638.293 | Hz |
| 4740.322 | Hz |
| 4854.318 | Hz |
| 4882.322 | Hz |
| 4978.822 | Hz |
| 5237.152 | Hz |
| 5264.222 | Hz |
| 5289.195 | Hz |
| 5426.323 | Hz |
| 5431.542 | Hz |
| 5455.593 | Hz |
| 6168.131 | Hz |
| 6345.332 | Hz |
| 6347.433 | Hz |
| 6363.284 | Hz |
| 6418.331 | Hz |
| 6496.231 | Hz |
| 6538.295 | Hz |
| 6577.421 | Hz |
| 6590.328 | Hz |
| 6651.276 | Hz |
| 6706.431 | Hz |
| 6743.322 | Hz |
| 6783.282 | Hz |
| 6850.197 | Hz |
| 6855.286 | Hz |
| 6864.896 | Hz |
| 6871.943 | Hz |
| 6878.356 | Hz |
| 6898.489 | Hz |
| 6973.393 | Hz |
| 7118.332 | Hz |
| 7120.932 | Hz |
| 7143.231 | Hz |
| 7146.509 | Hz |
| 7192.505 | Hz |
| 7251.309 | Hz |
| 7251.322 | Hz |
| 7278.124 | Hz |
| 7278.933 | Hz |
| 7279.335 | Hz |
| 7299.119 | Hz |
| 7527.229 | Hz |
| 7589.925 | Hz |
| 7699.193 | Hz |
| 7832.331 | Hz |
| 7842.184 | Hz |
| 7852.393 | Hz |
| 7872.333 | Hz |
| 8023.32 | Hz |
| 8096.939 | Hz |
| 8245.801 | Hz |
| 8315.291 | Hz |
| 8357.305 | Hz |
| 8408.121 | Hz |
| 8432.209 | Hz |
| 8535.238 | Hz |
| 8552.431 | Hz |
| 8585.224 | Hz |
| 8923.361 | Hz |
| 8935.752 | Hz |
| 9015.253 | Hz |
| 9018.233 | Hz |
| 9068.231 | Hz |
| 9137.232 | Hz |
| 9156.321 | Hz |
| 9351.931 | Hz |
| 9393.946 | Hz |
| 9694.179 | Hz |
| 9984.405 | Hz |
| 10226.223 | Hz |
| 10390.232 | Hz |
| 10442.221 | Hz |
| 10449.343 | Hz |
| 10459.084 | Hz |
| 10514.768 | Hz |
| 10651.311 | Hz |
| 10689.339 | Hz |
| 10772.419 | Hz |
| 10818.452 | Hz |
| 10843.543 | Hz |
| 11118.322 | Hz |
| 11165.239 | Hz |
| 11985.353 | Hz |
| 12209.329 | Hz |
| 12308.321 | Hz |
| 12489.233 | Hz |
| 12583.339 | Hz |
| 13820.329 | Hz |
| 14013.123 | Hz |
| 14129.213 | Hz |
| 14171.434 | Hz |
| 14681.329 | Hz |
| 14759.131 | Hz |
| 14986.794 | Hz |
| 15930.249 | Hz |
| 16026.623 | Hz |
| 16888.912 | Hz |
| 17091.189 | Hz |
| 17880.954 | Hz |
| 18021.222 | Hz |
| 18053.233 | Hz |
| 18247.532 | Hz |
| 18282.211 | Hz |
| 18610.232 | Hz |
| 18629.328 | Hz |
| 19469.318 | Hz |
| 19766.218 | Hz |
| 20159.434 | Hz |
| 21643.232 | Hz |
| 23022.481 | Hz |
| 23035.132 | Hz |
| 26718.23 | Hz |
| 30583.383 | Hz |
| 30653.323 | Hz |
| 30843.222 | Hz |
| 36065.221 | Hz |
| 60317.352 | Hz |

One or more of the AM frequencies of Table 19 may be used in combination with one or more of the frequencies provided in Table 20. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of prostate cancer, which frequencies are selected from Table 19 and, optionally from Table 20, provided that at least one or more frequencies are selected from Table 19.

Treatment of Rhabdomyosarcoma

Rhabdomyosarcoma may be treated according to the methods disclosed herein by the application of rhabdomyo sarcoma specific amplitude-modulated frequencies. Rhabdomyo sarcoma specific frequencies are provided below in Table 21. A patient suffering from rhabdomyosarcoma may be treated with any number of the frequencies provided in Table 21, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed rhabdomyosarcoma specific frequencies may be used in the treatment of rhabdomyo sarcoma. It should be noted that while the table below provides certain frequencies for treatment of rhabdomyosarcoma, the disclosure is not so limiting, and other frequencies discovered to be effective in the treatment of rhabdomyosarcoma are within the scope of this disclosure. Furthermore, each of the frequencies listed below include values that are close to (i.e., within +/−0.1 Hz), but not exactly, the number or range.

TABLE 21
Amplitude-modulated frequencies discovered to
be effective for treatment of rhabdomyosarcoma.

| | |
|---|---|
| 266.3-267 | Hz |
| 304-304.7 | Hz |
| 331-331.7 | Hz |
| 410-410.7 | Hz |
| 420-420.7 | Hz |
| 429-429.7 | Hz |
| 434-434.7 | Hz |
| 461-461.7 | Hz |
| 537.4-538 | Hz |
| 560-560.7 | Hz |
| 572-572.7 | Hz |
| 605-605.7 | Hz |
| 611-611.7 | Hz |
| 618.3-619 | Hz |
| 626-626.7 | Hz |
| 717-717.7 | Hz |
| 776-776.7 | Hz |
| 779-779.7 | Hz |
| 810-810.7 | Hz |
| 814-814.7 | Hz |
| 826-826.7 | Hz |
| 836.4-837 | Hz |
| 843-843.7 | Hz |
| 843-843.7 | Hz |
| 1314-1314.7 | Hz |
| 1411-1411.7 | Hz |
| 1467-1467.7 | Hz |
| 1785-1785.7 | Hz |
| 1816-1816.7 | Hz |
| 1903-1903.7 | Hz |
| 2073.3-2074 | Hz |
| 2155-2155.7 | Hz |
| 2287-2287.7 | Hz |
| 2381-2381.7 | Hz |
| 2542-2542.7 | Hz |
| 2711-2711.6 | Hz |
| 2854-2854.6 | Hz |
| 2911-2911.7 | Hz |
| 3085.7-3086.3 | Hz |
| 3121-3121.7 | Hz |
| 3232-3232.7 | Hz |
| 3438-3438.7 | Hz |
| 3518-3518.7 | Hz |
| 3544-3544.7 | Hz |
| 3569-3569.7 | Hz |
| 3618-3618.7 | Hz |
| 3910-3910.7 | Hz |
| 3917-3917.7 | Hz |
| 4198-4198.7 | Hz |
| 4233.3-4234 | Hz |
| 4241-4241.7 | Hz |
| 4337-4337.7 | Hz |
| 4358-4358.7 | Hz |
| 4424-4424.7 | Hz |
| 4436-4436.7 | Hz |
| 4485-4485.7 | Hz |
| 4731-4731.7 | Hz |
| 4749-4749.7 | Hz |
| 4776-4776.7 | Hz |
| 5151-5151.7 | Hz |
| 5521-5521.7 | Hz |
| 5545.3-5546 | Hz |
| 5577.3-5578 | Hz |
| 5618-5618.7 | Hz |
| 5631-5631.7 | Hz |
| 5696-5696.7 | Hz |
| 6058-6058.7 | Hz |
| 6443-6443.7 | Hz |
| 6472-6472.6 | Hz |
| 6558-6558.7 | Hz |
| 6651-6651.7 | Hz |
| 6730-6730.7 | Hz |
| 7168.4-7169 | Hz |
| 7406-7406.7 | Hz |
| 7440-7440.7 | Hz |
| 7452.3-7453 | Hz |
| 7649-7649.7 | Hz |
| 7808-7808.7 | Hz |
| 8542-8542.7 | Hz |
| 8546-8546.7 | Hz |
| 8562-8562.7 | Hz |
| 8576-8576.7 | Hz |
| 8698-8698.7 | Hz |
| 8881-8881.7 | Hz |
| 9040-9040.7 | Hz |
| 9074-9074.7 | Hz |
| 9189-9189.6 | Hz |
| 9484.3-9485 | Hz |
| 9851-9851.7 | Hz |
| 9943.4-9944 | Hz |
| 10870-10870.7 | Hz |
| 10891-10891.7 | Hz |
| 12086-12086.7 | Hz |
| 12648-12648.7 | Hz |
| 15195-15195.7 | Hz |
| 17316-17316.7 | Hz |
| 18021-18021.7 | Hz |
| 18567-18567.7 | Hz |
| 19384-19384.7 | Hz |
| 20097-20097.7 | Hz |
| 20278.4-20279 | Hz |
| 21854-21854.7 | Hz |
| 21890-21890.7 | Hz |
| 24198-24198.7 | Hz |
| 24413-24413.7 | Hz |
| 24789-24789.7 | Hz |
| 26534-26534.7 | Hz |
| 27713-27713.7 | Hz |
| 28222-28222.7 | Hz |
| 40489-40489.7 | Hz |
| 40762-40762.7 | Hz |
| 41279-41279.7 | Hz |
| 42923-42923.7 | Hz |
| 43166-43166.7 | Hz |
| 43692-43692.7 | Hz |
| 48583-48583.7 | Hz |
| 49434-49434.7 | Hz |
| 66553-66553.7 | Hz |
| 79658-79658.7 | Hz |

Treatment of Colorectal Cancer

Colorectal cancer may be treated according to the methods disclosed herein by the application of colorectal cancer specific amplitude-modulated frequencies. Colorectal cancer specific frequencies are provided below in Table 22. A patient suffering from colorectal cancer may be treated with any number of the frequencies provided in Table 22, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed colorectal cancer specific frequencies may be used in the treatment of colorectal cancer. It should be noted that while the table below provides certain frequencies for treatment of colorectal cancer, the disclosure is not so limiting, and other frequencies discovered to be effective in the treatment of colorectal cancer are within the scope of this disclosure. Furthermore, each of the frequencies listed below include values that are close to (i.e., within +/−0.1 Hz), but not exactly, the number or range.

TABLE 22
Amplitude-modulated frequencies discovered to be
effective for treatment of colorectal cancer.

| | |
|---|---|
| 605-605.7 | Hz |
| 826-826.7 | Hz |
| 834-834.7 | Hz |
| 841.9-842.6 | Hz |
| 1808-1808.6 | Hz |
| 1928-1928.7 | Hz |
| 1989-1989.7 | Hz |
| 2439.7-2440.3 | Hz |
| 2714-2714.7 | Hz |
| 2747-2747.7 | Hz |
| 2759-2759.7 | Hz |
| 2807-2807.7 | Hz |
| 2826-2826.7 | Hz |
| 2981-2981.7 | Hz |
| 3209-3209.7 | Hz |
| 3213-3213.7 | Hz |
| 3244-3244.7 | Hz |
| 3261-3261.7 | Hz |
| 3548-3548.7 | Hz |
| 3634-3634.7 | Hz |
| 3646.4-3647 | Hz |
| 3915-3915.7 | Hz |
| 3922-3922.7 | Hz |
| 3940-3940.7 | Hz |
| 3946-3946.7 | Hz |
| 4135-4135.7 | Hz |
| 4155-4155.7 | Hz |
| 4224-4224.7 | Hz |
| 4241-4241.7 | Hz |
| 4283-4283.7 | Hz |
| 4323-4323.7 | Hz |
| 4438.7-4439.2 | Hz |
| 4447-4447.7 | Hz |
| 4708-4708.7 | Hz |
| 4713-4713.7 | Hz |
| 4718-4718.7 | Hz |
| 4718-4718.7 | Hz |
| 4722-4722.7 | Hz |
| 4776-4776.7 | Hz |
| 4850-4850.7 | Hz |
| 4874-4874.7 | Hz |
| 5205-5205.7 | Hz |
| 5209-5209.7 | Hz |
| 5209-5209.7 | Hz |
| 5221-5221.7 | Hz |
| 5232-5232.7 | Hz |
| 5265-5265.7 | Hz |
| 5427-5427.7 | Hz |
| 5484.4-5485 | Hz |
| 6074-6074.7 | Hz |
| 6112-6112.7 | Hz |
| 6159-6159.7 | Hz |
| 6361-6361.7 | Hz |
| 6438-6438.7 | Hz |
| 6468-6468.7 | Hz |
| 6537-6537.7 | Hz |
| 6648-6648.7 | Hz |
| 6869-6869.7 | Hz |
| 6878-6878.7 | Hz |
| 6914-6914.7 | Hz |
| 6974-6974.7 | Hz |
| 6982-6982.7 | Hz |
| 6986-6986.7 | Hz |
| 7109-7109.7 | Hz |
| 7117-7117.7 | Hz |
| 7137-7137.7 | Hz |
| 7149-7149.7 | Hz |
| 7191.7-7192.3 | Hz |
| 7531-7531.7 | Hz |
| 7560-7560.7 | Hz |
| 7879-7879.7 | Hz |
| 7936-7936.7 | Hz |
| 7970-7970.7 | Hz |
| 7994-7994.7 | Hz |
| 8038.4-8039 | Hz |
| 8413-8413.7 | Hz |
| 8438-8438.7 | Hz |
| 8467-8467.7 | Hz |
| 8771-8771.7 | Hz |
| 8809-8809.7 | Hz |
| 8889-8889.7 | Hz |
| 9071-9071.7 | Hz |
| 9084-9084.7 | Hz |
| 9731-9731.7 | Hz |
| 10566-10566.7 | Hz |
| 10831.7-10832.1 | Hz |
| 10845-10845.7 | Hz |
| 10859-10859.7 | Hz |
| 10906-10906.7 | Hz |
| 10947-10947.7 | Hz |
| 10974-10974.7 | Hz |
| 11110-11110.7 | Hz |
| 11225-11225.7 | Hz |
| 11234-11234.7 | Hz |
| 11255-11255.7 | Hz |
| 11387-11387.7 | Hz |
| 11530-11530.7 | Hz |
| 11576-11576.7 | Hz |
| 11621-11621.7 | Hz |
| 11630-11630.7 | Hz |
| 11638-11638.7 | Hz |
| 11681-11681.7 | Hz |
| 11911-11911.7 | Hz |
| 11934-11934.7 | Hz |
| 11949-11949.7 | Hz |
| 12182-12182.7 | Hz |
| 12373-12373.7 | Hz |
| 12575-12575.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 13418-13418.7 | Hz |
| 13475-13475.7 | Hz |
| 13611-13611.7 | Hz |
| 13615-13615.7 | Hz |
| 13670-13670.7 | Hz |
| 13717-13717.7 | Hz |
| 13736-13736.7 | Hz |
| 13742-13742.7 | Hz |
| 13773-13773.7 | Hz |
| 13775-13775.7 | Hz |
| 13978.3-13979 | Hz |
| 14182-14182.7 | Hz |
| 14417-14417.7 | Hz |
| 14422-14422.7 | Hz |
| 14441-14441.7 | Hz |
| 14443-14443.7 | Hz |
| 14570-14570.7 | Hz |
| 14621-14621.7 | Hz |
| 14630-14630.7 | Hz |
| 14671-14671.7 | Hz |
| 14674-14674.7 | Hz |
| 14863-14863.7 | Hz |
| 14877-14877.7 | Hz |
| 14911-14911.7 | Hz |
| 14957-14957.7 | Hz |
| 14982-14982.7 | Hz |
| 15109-15109.7 | Hz |
| 15133-15133.7 | Hz |
| 15154-15154.7 | Hz |
| 15230-15230.7 | Hz |
| 15250-15250.7 | Hz |
| 15278-15278.7 | Hz |
| 16274-16274.7 | Hz |
| 17106-17106.7 | Hz |
| 17123-17123.7 | Hz |
| 17472-17472.7 | Hz |
| 17515-17515.7 | Hz |
| 17578.4-17579 | Hz |
| 17808-17808.7 | Hz |
| 17825-17825.7 | Hz |
| 18157-18157.7 | Hz |
| 18171-18171.7 | Hz |
| 18294-18294.7 | Hz |
| 18372-18372.7 | Hz |
| 18375-18375.7 | Hz |
| 18396-18396.7 | Hz |
| 18533-18533.7 | Hz |
| 18635-18635.7 | Hz |
| 19342-19342.7 | Hz |
| 19437-19437.7 | Hz |
| 19447-19447.7 | Hz |
| 19829-19829.7 | Hz |
| 20173-20173.7 | Hz |
| 20499-20499.7 | Hz |
| 21389-21389.7 | Hz |
| 21490-21490.7 | Hz |
| 21663-21663.7 | Hz |
| 21833-21833.7 | Hz |
| 22167-22167.7 | Hz |
| 22423.4-22424 | Hz |
| 22559.3-22560 | Hz |
| 22743-22743.7 | Hz |
| 23246-23246.7 | Hz |
| 23249-23249.7 | Hz |
| 23259-23259.7 | Hz |
| 23344-23344.7 | Hz |
| 23624-23624.7 | Hz |
| 23667-23667.7 | Hz |
| 24142-24142.7 | Hz |
| 26113-26113.7 | Hz |
| 26403-26403.7 | Hz |
| 28212-28212.7 | Hz |
| 28272-28272.7 | Hz |
| 28537-28537.7 | Hz |
| 30075-30075.7 | Hz |
| 30373-30373.7 | Hz |
| 31847-31847.7 | Hz |
| 33546-33546.7 | Hz |
| 35324-35324.7 | Hz |
| 35543-35543.7 | Hz |
| 42544-42544.7 | Hz |
| 42692.3-42693 | Hz |
| 42812-42812.7 | Hz |
| 42813-42813.7 | Hz |
| 43162-43162.7 | Hz |
| 43325-43325.7 | Hz |
| 44652-44652.7 | Hz |
| 46340-46340.7 | Hz |
| 46746-46746.7 | Hz |
| 48653-48653.7 | Hz |
| 51946-51946.7 | Hz |
| 52752-52752.7 | Hz |
| 80715-80715.7 | Hz |
| 94494-94494.7 | Hz |

In certain embodiments, the frequencies listed above may also be used in combination with one or more of the frequencies disclosed in the '365 patent (reproduced below). Specifically, treatment modalities for treatment of colorectal cancer in accordance with this disclosure may include application of some or all of the above listed frequencies with one or more of the frequencies disclosed in Table 23 for colorectal cancer. Alternatively and/or additionally, the frequencies disclosed in the '365 patent may be used for treatment of colorectal cancer using the methods and systems described herein.

TABLE 23
Amplitude-modulated frequencies for treatment of colon cancer.

| | |
|---|---|
| 78.76 | Hz |
| 796.562 | Hz |
| 841.541 | Hz |
| 842.783 | Hz |
| 914.429 | Hz |
| 1162.117 | Hz |
| 1372.207 | Hz |
| 1372.934 | Hz |
| 1718.532 | Hz |
| 2243.169 | Hz |
| 2278.312 | Hz |
| 2286.5 | Hz |
| 2286.519 | Hz |
| 2334.178 | Hz |
| 2423.292 | Hz |
| 2454.423 | Hz |
| 2464.229 | Hz |
| 2598.853 | Hz |
| 2623.048 | Hz |
| 3131.123 | Hz |
| 3161.465 | Hz |
| 3175.313 | Hz |
| 3249.529 | Hz |
| 3363.229 | Hz |
| 3373.892 | Hz |
| 3390.925 | Hz |
| 3409.179 | Hz |
| 3432.274 | Hz |
| 3509.522 | Hz |
| 3531.422 | Hz |
| 3533.328 | Hz |
| 3766.296 | Hz |
| 4040.839 | Hz |
| 4081.022 | Hz |
| 4123.953 | Hz |
| 4146.274 | Hz |
| 4233.822 | Hz |
| 4282.332 | Hz |
| 4318.222 | Hz |
| 4344.082 | Hz |
| 4416.221 | Hz |
| 4481.242 | Hz |
| 4724.263 | Hz |
| 4751.319 | Hz |
| 4755.323 | Hz |
| 4788.485 | Hz |
| 5149.331 | Hz |
| 5217.402 | Hz |
| 5386.212 | Hz |
| 5407.192 | Hz |
| 5426.323 | Hz |
| 5496.434 | Hz |
| 5555.212 | Hz |
| 5572.032 | Hz |
| 5634.933 | Hz |
| 5724.231 | Hz |
| 5758.378 | Hz |
| 5787.342 | Hz |
| 5948.897 | Hz |
| 5967.448 | Hz |
| 5976.825 | Hz |
| 6182.322 | Hz |
| 6292.379 | Hz |
| 6324.493 | Hz |
| 6341.248 | Hz |
| 6471.322 | Hz |
| 6477.218 | Hz |
| 6558.342 | Hz |
| 6855.286 | Hz |
| 7129.843 | Hz |
| 7140.187 | Hz |
| 7162.422 | Hz |
| 7368.222 | Hz |
| 7645.859 | Hz |
| 7829.234 | Hz |
| 7866.229 | Hz |
| 7877.334 | Hz |
| 8013.314 | Hz |
| 8374.942 | Hz |
| 8384.228 | Hz |
| 8408.121 | Hz |
| 8534.111 | Hz |
| 8568.033 | Hz |
| 8573.122 | Hz |
| 9226.222 | Hz |
| 9351.9 | Hz |
| 9737.211 | Hz |
| 9744.193 | Hz |
| 9942.321 | Hz |
| 10301.371 | Hz |
| 10401.515 | Hz |
| 10872.693 | Hz |
| 11220.222 | Hz |
| 11283.378 | Hz |
| 12256.432 | Hz |
| 13749.858 | Hz |
| 15231.548 | Hz |
| 15248.324 | Hz |
| 58191.928 | Hz |
| 60317.352 | Hz |

One or more of the AM frequencies of Table 22 may be used in combination with one or more of the frequencies provided in Table 23. In preferred embodiments, 10 or more, 15 or more, or 20 or more, 25 or more, or 30 or more, 35 or more, or 40 or more, 45 or more, or 50 or more, 60 or more, 70 or more, 80 or more, 90 or more, or 100 or more frequencies are used in the methods for the treatment of colorectal cancer, which frequencies are selected from Table 22 and, optionally from Table 23, provided that at least one or more frequencies are selected from Table 22.

In certain embodiments, the frequencies disclosed in the '365 patent may be used for treatment of a diseased state using the methods and systems described herein.

Treatment of Ovarian Cancer

Ovarian cancer may be treated according to the methods disclosed herein by the application of ovarian cancer specific amplitude-modulated frequencies. Ovarian cancer specific frequencies are provided below in Table 24. A patient suffering from ovarian cancer may be treated with any number of the frequencies provided in Table 24, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more, 90 or more, or 100 or more of the listed ovarian cancer specific frequencies may be used in the treatment of ovarian cancer. It should be noted that while the table below provides certain frequencies for treatment of ovarian cancer, the disclosure is not so limiting, and other frequencies discovered to be effective in the treatment of ovarian cancer are within the scope of this disclosure.

TABLE 24
Amplitude-modulated frequencies for treatment of ovarian cancer.

| | |
|---|---|
| 78.76 | Hz |
| 181.821 | Hz |
| 367.211 | Hz |
| 403.218 | Hz |
| 410.245 | Hz |
| 414.817 | Hz |
| 436.332 | Hz |
| 447.942 | Hz |
| 481.191 | Hz |
| 489.292 | Hz |
| 537.914 | Hz |
| 559.292 | Hz |
| 608.321 | Hz |
| 618.407 | Hz |
| 621.321 | Hz |
| 655.435 | Hz |
| 657.394 | Hz |
| 657.397 | Hz |
| 657.483 | Hz |
| 664.211 | Hz |
| 694.689 | Hz |
| 708.787 | Hz |
| 708.8 | Hz |
| 708.821 | Hz |
| 708.822 | Hz |
| 734.921 | Hz |
| 749.221 | Hz |
| 764.232 | Hz |
| 778.295 | Hz |
| 779.403 | Hz |
| 806.021 | Hz |
| 806.389 | Hz |
| 809.313 | Hz |
| 824.327 | Hz |
| 825.145 | Hz |
| 835.129 | Hz |
| 839.521 | Hz |
| 841.208 | Hz |
| 843.312 | Hz |
| 925.309 | Hz |
| 956.984 | Hz |
| 958.929 | Hz |
| 985.313 | Hz |
| 1024.208 | Hz |
| 1102.635 | Hz |
| 1121.329 | Hz |
| 1159.738 | Hz |
| 1221.321 | Hz |
| 1372.207 | Hz |
| 1396.498 | Hz |
| 1502.181 | Hz |
| 1518.208 | Hz |
| 1552.123 | Hz |
| 1579.212 | Hz |
| 1624.802 | Hz |
| 1656.431 | Hz |
| 1670.699 | Hz |
| 1679.432 | Hz |
| 1696.403 | Hz |
| 1759.318 | Hz |
| 1762.938 | Hz |
| 1771.402 | Hz |
| 1775.313 | Hz |
| 1821.729 | Hz |
| 1990.482 | Hz |
| 2016.323 | Hz |
| 2031.448 | Hz |
| 2034.231 | Hz |
| 2050.282 | Hz |
| 2053.396 | Hz |
| 2082.234 | Hz |
| 2089.092 | Hz |
| 2221.323 | Hz |
| 2228.832 | Hz |
| 2229.515 | Hz |
| 2253.704 | Hz |
| 2254.329 | Hz |
| 2262.853 | Hz |
| 2278.312 | Hz |
| 2332.949 | Hz |
| 2348.233 | Hz |
| 2381.443 | Hz |
| 2413.193 | Hz |
| 2415.243 | Hz |
| 2425.222 | Hz |
| 2433.321 | Hz |
| 2439.253 | Hz |
| 2465.23 | Hz |
| 2477.919 | Hz |
| 2669.177 | Hz |
| 2715.232 | Hz |
| 2733.843 | Hz |
| 2771.211 | Hz |
| 2802.339 | Hz |
| 2812.321 | Hz |
| 2831.386 | Hz |
| 2835.332 | Hz |
| 2851.347 | Hz |
| 2856.253 | Hz |
| 2873.542 | Hz |
| 2877.192 | Hz |
| 2885.322 | Hz |
| 2887.385 | Hz |
| 2894.972 | Hz |
| 2973.771 | Hz |
| 3080.592 | Hz |
| 3157.483 | Hz |
| 3160.321 | Hz |
| 3161.465 | Hz |
| 3185.129 | Hz |
| 3223.232 | Hz |
| 3238.148 | Hz |
| 3240.111 | Hz |
| 3249.529 | Hz |
| 3254.122 | Hz |
| 3262.145 | Hz |
| 3264.241 | Hz |
| 3265.121 | Hz |
| 3282.235 | Hz |
| 3283.392 | Hz |
| 3296.431 | Hz |
| 3314.321 | Hz |
| 3361.671 | Hz |
| 3366.311 | Hz |
| 3459.408 | Hz |
| 3461.322 | Hz |
| 3523.215 | Hz |
| 3527.233 | Hz |
| 3542.213 | Hz |
| 3590.376 | Hz |
| 3629.232 | Hz |
| 3632.793 | Hz |
| 3636.289 | Hz |
| 3637.085 | Hz |
| 3669.513 | Hz |
| 3770.189 | Hz |
| 3858.916 | Hz |
| 3872.321 | Hz |
| 3919.232 | Hz |
| 3941.739 | Hz |
| 3957.185 | Hz |
| 3975.228 | Hz |
| 3975.383 | Hz |
| 4061.131 | Hz |
| 4072.322 | Hz |
| 4139.322 | Hz |
| 4169.451 | Hz |
| 4174.259 | Hz |
| 4241.321 | Hz |
| 4243.393 | Hz |
| 4261.228 | Hz |
| 4279.113 | Hz |
| 4309.335 | Hz |
| 4314.188 | Hz |
| 4318.222 | Hz |
| 4328.928 | Hz |
| 4340.833 | Hz |
| 4380.321 | Hz |
| 4394.134 | Hz |
| 4412.252 | Hz |
| 4424.236 | Hz |
| 4439.341 | Hz |
| 4442.161 | Hz |
| 4447.221 | Hz |
| 4458.339 | Hz |
| 4556.322 | Hz |
| 4566.009 | Hz |
| 4579.981 | Hz |
| 4682.643 | Hz |
| 4718.331 | Hz |
| 4749.302 | Hz |
| 4765.331 | Hz |
| 4779.194 | Hz |
| 4912.923 | Hz |
| 4917.202 | Hz |
| 5011.325 | Hz |
| 5149.331 | Hz |
| 5228.172 | Hz |
| 5237.132 | Hz |
| 5313.353 | Hz |
| 5745.218 | Hz |
| 5757.897 | Hz |
| 5762.386 | Hz |
| 5812.322 | Hz |
| 5869.321 | Hz |
| 5882.292 | Hz |
| 5921.249 | Hz |
| 5991.932 | Hz |
| 6069.458 | Hz |
| 6071.319 | Hz |
| 6083.214 | Hz |
| 6111.819 | Hz |
| 6161.782 | Hz |
| 6169.341 | Hz |
| 6275.232 | Hz |
| 6294.929 | Hz |
| 6350.333 | Hz |
| 6356.321 | Hz |
| 6406.891 | Hz |
| 6407.207 | Hz |
| 6450.787 | Hz |
| 6477.098 | Hz |
| 6477.929 | Hz |
| 6478.338 | Hz |
| 6504.983 | Hz |
| 6543.421 | Hz |
| 6552.24 | Hz |
| 6661.09 | Hz |
| 6663.955 | Hz |
| 6753.338 | Hz |
| 6789.211 | Hz |
| 6851.323 | Hz |
| 6855.286 | Hz |
| 6875.232 | Hz |
| 6882.949 | Hz |
| 7047.223 | Hz |
| 7206.403 | Hz |
| 7232.214 | Hz |
| 7257.489 | Hz |
| 7276.209 | Hz |
| 7279.335 | Hz |
| 7281.219 | Hz |
| 7285.223 | Hz |
| 7285.693 | Hz |
| 7289.192 | Hz |
| 7326.229 | Hz |
| 7399.223 | Hz |
| 7429.212 | Hz |
| 7460.932 | Hz |
| 7480.228 | Hz |
| 7488.742 | Hz |
| 7495.763 | Hz |
| 7539.432 | Hz |
| 7564.185 | Hz |
| 7650.028 | Hz |
| 7689.728 | Hz |
| 7780.294 | Hz |
| 8021.921 | Hz |
| 8038.961 | Hz |
| 8040.322 | Hz |
| 8044.233 | Hz |
| 8054.413 | Hz |
| 8095.313 | Hz |
| 8141.174 | Hz |
| 8143.491 | Hz |
| 8164.332 | Hz |
| 8261.121 | Hz |
| 8302.285 | Hz |
| 8309.752 | Hz |
| 8372.532 | Hz |
| 8408.121 | Hz |
| 8424.229 | Hz |
| 8428.313 | Hz |
| 8430.142 | Hz |
| 8435.451 | Hz |
| 8486.421 | Hz |
| 8492.797 | Hz |
| 8548.324 | Hz |
| 8554.361 | Hz |
| 8562.965 | Hz |
| 8578.193 | Hz |
| 8579.323 | Hz |
| 8579.333 | Hz |
| 8597.409 | Hz |
| 8642.181 | Hz |
| 8655.818 | Hz |
| 8758.341 | Hz |
| 8779.323 | Hz |
| 8792.231 | Hz |
| 8819.127 | Hz |
| 8831.132 | Hz |
| 8863.232 | Hz |
| 9028.031 | Hz |
| 9049.205 | Hz |
| 9173.264 | Hz |
| 9175.311 | Hz |
| 9184.338 | Hz |
| 9186.919 | Hz |
| 9393.946 | Hz |
| 9482.409 | Hz |
| 9658.296 | Hz |
| 9737.211 | Hz |
| 9746.232 | Hz |
| 9859.322 | Hz |
| 9922.231 | Hz |
| 10020.213 | Hz |
| 10032.684 | Hz |
| 10435.191 | Hz |
| 10446.028 | Hz |
| 10449.221 | Hz |
| 10457.329 | Hz |
| 10478.221 | Hz |
| 10498.339 | Hz |
| 10545.313 | Hz |
| 10639.345 | Hz |
| 10720.221 | Hz |
| 10743.118 | Hz |
| 10813.981 | Hz |
| 10832.421 | Hz |
| 10838.243 | Hz |
| 10862.429 | Hz |
| 10865.127 | Hz |
| 10917.229 | Hz |
| 10977.188 | Hz |
| 11120.209 | Hz |
| 11143.409 | Hz |
| 11177.289 | Hz |
| 11177.409 | Hz |
| 11321.491 | Hz |
| 11359.093 | Hz |
| 11540.212 | Hz |
| 11673.031 | Hz |
| 11731.295 | Hz |
| 11793.886 | Hz |
| 11895.229 | Hz |
| 12074.531 | Hz |
| 12216.212 | Hz |
| 12223.329 | Hz |
| 12243.132 | Hz |
| 12253.329 | Hz |
| 12260.933 | Hz |
| 12292.222 | Hz |
| 12357.353 | Hz |
| 12527.032 | Hz |
| 12668.194 | Hz |
| 12743.197 | Hz |
| 12755.333 | Hz |
| 12947.311 | Hz |
| 13477.293 | Hz |
| 13582.122 | Hz |
| 13636.082 | Hz |
| 13717.221 | Hz |
| 13756.503 | Hz |
| 13825.295 | Hz |
| 13829.195 | Hz |
| 14188.611 | Hz |
| 14410.949 | Hz |
| 14436.201 | Hz |
| 14528.429 | Hz |
| 14537.218 | Hz |
| 14563.821 | Hz |
| 14835.809 | Hz |
| 14947.184 | Hz |
| 14948.323 | Hz |
| 15429.139 | Hz |
| 15443.309 | Hz |
| 15450.183 | Hz |
| 16026.221 | Hz |
| 16062.401 | Hz |
| 16081.291 | Hz |
| 16144.343 | Hz |
| 16331.323 | Hz |
| 17316.328 | Hz |
| 17930.967 | Hz |
| 17932.432 | Hz |
| 17951.395 | Hz |
| 17970.122 | Hz |
| 18242.181 | Hz |
| 18254.323 | Hz |
| 18265.238 | Hz |
| 18337.222 | Hz |
| 18344.212 | Hz |
| 18378.321 | Hz |
| 18921.415 | Hz |
| 18926.951 | Hz |
| 18931.327 | Hz |
| 19124.197 | Hz |
| 19133.123 | Hz |
| 19321.231 | Hz |
| 19686.593 | Hz |
| 114508.332 | Hz |

Treatment of Kidney Cancer

Kidney cancer may be treated according to the methods disclosed herein by the application of kidney cancer specific amplitude-modulated frequencies. Kidney cancer specific frequencies are provided below in Table 25. A patient suffering from kidney cancer may be treated with any number of the frequencies provided in Table 25, however it is generally preferable to employ as many of the frequencies as is practical for the treatment of the patient. Accordingly, 10 or more, 15 or more, or 20 or more, 25 or more, or 30 or more, 35 or more of the listed kidney cancer specific frequencies may be used in the treatment of kidney cancer. It should be noted that while the table below provides certain frequencies for treatment of kidney cancer, the disclosure is not so limiting, and other frequencies discovered to be effective in the treatment of kidney cancer are within the scope of this disclosure.

TABLE 25
Amplitude-modulated frequencies for treatment of kidney cancer.

| | |
|---|---|
| 628.321 | Hz |
| 631.141 | Hz |
| 643.312 | Hz |
| 812.512 | Hz |
| 826.321 | Hz |
| 1240.336 | Hz |
| 1372.934 | Hz |
| 2082.241 | Hz |
| 2156.931 | Hz |
| 2254.329 | Hz |
| 2286.5 | Hz |
| 3555.209 | Hz |
| 3928.343 | Hz |
| 4329.152 | Hz |
| 4420.932 | Hz |
| 4819.228 | Hz |
| 4828.321 | Hz |
| 5314.322 | Hz |
| 6007.332 | Hz |
| 7054.279 | Hz |
| 7074.929 | Hz |
| 7254.343 | Hz |
| 8041.289 | Hz |
| 8727.224 | Hz |
| 8760.983 | Hz |
| 8831.132 | Hz |
| 8870.228 | Hz |
| 8923.1 | Hz |
| 10565.321 | Hz |
| 10586.229 | Hz |
| 11561.221 | Hz |
| 11846.212 | Hz |
| 12631.331 | Hz |
| 12693.272 | Hz |
| 14411.321 | Hz |
| 20178.941 | Hz |

Treatment of Bladder Cancer

Bladder cancer may be treated according to the methods disclosed herein by the application of bladder cancer specific amplitude-modulated frequencies. Bladder cancer specific frequencies are provided below in Table 26. A patient suffering from bladder cancer may be treated with any number of the frequencies provided in Table 26, however it is generally preferable to employ as many of the frequencies as is practical for the treatment of the patient. Accordingly, 10 or more, 15 or more, or 20 or more, 25 or more, or 30 or more of the listed bladder cancer specific frequencies may be used in the treatment of bladder cancer. It should be noted that while the table below provides certain frequencies for treatment of bladder cancer, the disclosure is not so limiting, and other frequencies discovered to be effective in the treatment of bladder cancer are within the scope of this disclosure.

TABLE 26
Amplitude-modulated frequencies for treatment of bladder cancer.

| | |
|---|---|
| 623.243 | Hz |
| 757.084 | Hz |
| 870.4 | Hz |
| 2454.423 | Hz |
| 2480.191 | Hz |
| 2581.101 | Hz |
| 2715.232 | Hz |
| 3042.012 | Hz |
| 3196.194 | Hz |
| 3265.323 | Hz |
| 3438.109 | Hz |
| 3692.319 | Hz |
| 3952.308 | Hz |
| 5230.227 | Hz |
| 6022.942 | Hz |
| 6061.711 | Hz |
| 6710.899 | Hz |
| 6721.912 | Hz |
| 7181.784 | Hz |
| 7212.826 | Hz |
| 7458.209 | Hz |
| 8235.21 | Hz |
| 8749.232 | Hz |
| 8767.189 | Hz |
| 9354.812 | Hz |
| 9611.339 | Hz |
| 12532.729 | Hz |
| 13467.209 | Hz |
| 13777.9 | Hz |
| 14015.241 | Hz |
| 18524.419 | Hz |

Treatment of Lung Cancer

Lung cancer may be treated according to the methods disclosed herein by the application of lung cancer specific amplitude-modulated frequencies. Lung cancer specific frequencies are provided below in Table 27. A patient suffering from lung cancer may be treated with any number of the frequencies provided in Table 27, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more, 60 or more, 70 or more, 80 or more of the listed lung cancer specific frequencies may be used in the treatment of lung cancer. It should be noted that while the table below provides certain frequencies for treatment of lung cancer, the disclosure is not so limiting, and other frequencies discovered to be effective in the treatment of lung cancer are within the scope of this disclosure.

TABLE 27
Amplitude-modulated frequencies for treatment of lung cancer.

| | |
|---|---|
| 304.148 | Hz |
| 694.7 | Hz |
| 694.727 | Hz |
| 708.8 | Hz |
| 708.841 | Hz |
| 1587.811 | Hz |
| 1759.318 | Hz |
| 1873.477 | Hz |
| 2253.704 | Hz |
| 2391.312 | Hz |
| 2454.232 | Hz |
| 2729.929 | Hz |
| 2741.261 | Hz |
| 2761.312 | Hz |
| 2784.491 | Hz |
| 2812.443 | Hz |
| 2855.218 | Hz |
| 2859.495 | Hz |
| 3128.822 | Hz |
| 3139.297 | Hz |
| 3193.212 | Hz |
| 3348.783 | Hz |
| 3360.971 | Hz |
| 3366.311 | Hz |
| 3373.892 | Hz |
| 3440.212 | Hz |
| 3461.322 | Hz |
| 3682.489 | Hz |
| 3727.231 | Hz |
| 3749.882 | Hz |
| 3769.942 | Hz |
| 4131.235 | Hz |
| 4158.393 | Hz |
| 4243.393 | Hz |
| 4347.733 | Hz |
| 4373.411 | Hz |
| 4378.321 | Hz |
| 4416.221 | Hz |
| 4481.242 | Hz |
| 4777.521 | Hz |
| 4798.422 | Hz |
| 4837.241 | Hz |
| 4959.842 | Hz |
| 5013.321 | Hz |
| 5047.523 | Hz |
| 5068.322 | Hz |
| 5371.922 | Hz |
| 5538.432 | Hz |
| 5548.879 | Hz |
| 5679.309 | Hz |
| 5734.143 | Hz |
| 5787.342 | Hz |
| 6445.309 | Hz |
| 6838.434 | Hz |
| 6870.955 | Hz |
| 6879.216 | Hz |
| 7079.411 | Hz |
| 7216.288 | Hz |
| 7376.089 | Hz |
| 7761.289 | Hz |
| 8082.173 | Hz |
| 8281.259 | Hz |
| 8352.189 | Hz |
| 8442.473 | Hz |
| 8773.916 | Hz |
| 8935.752 | Hz |
| 9121.223 | Hz |
| 9181.434 | Hz |
| 9317.913 | Hz |
| 9363.896 | Hz |
| 9736.919 | Hz |
| 9753.321 | Hz |
| 10424.908 | Hz |
| 10452.913 | Hz |
| 10824.609 | Hz |
| 11656.329 | Hz |
| 12748.919 | Hz |
| 15774.291 | Hz |
| 15798.333 | Hz |
| 16510.333 | Hz |

Treatment of Leiomyo Sarcoma

Leiomyo sarcoma may be treated according to the methods disclosed herein by the application of leiomyosarcoma specific amplitude-modulated frequencies. Leiomyo sarcoma specific frequencies are provided below in Table 28. A patient suffering from leiomyosarcoma may be treated with any number of the frequencies provided in Table 28, however it is generally preferable to employ as many of the frequencies as is practical for the treatment of the patient. Accordingly, 10 or more, 15 or more, or 20 or more, 25 or more, or 30 or more, 35 or more of the listed leiomyosarcoma specific frequencies may be used in the treatment of leiomyosarcoma. It should be noted that while the table below provides certain frequencies for treatment of leiomyosarcoma, the disclosure is not so limiting, and other frequencies discovered to be effective in the treatment of leiomyo sarcoma are within the scope of this disclosure.

TABLE 28
Amplitude-modulated frequencies
for treatment of leiomyosarcoma.

| | |
|---|---|
| 836.923 | Hz |
| 843.181 | Hz |
| 1411.241 | Hz |
| 2073.721 | Hz |
| 2381.443 | Hz |
| 2711.019 | Hz |
| 2911.329 | Hz |
| 3232.185 | Hz |
| 3518.321 | Hz |
| 3544.209 | Hz |
| 3569.219 | Hz |
| 4233.822 | Hz |
| 4241.321 | Hz |
| 4266.591 | Hz |
| 4337.322 | Hz |
| 4424.112 | Hz |
| 4436.111 | Hz |
| 4485.22 | Hz |
| 5545.521 | Hz |
| 5577.841 | Hz |
| 5631.422 | Hz |
| 5696.184 | Hz |
| 6472.098 | Hz |
| 6558.342 | Hz |
| 6651.276 | Hz |
| 7168.892 | Hz |
| 7406.309 | Hz |
| 7452.528 | Hz |
| 7649.209 | Hz |
| 7808.352 | Hz |
| 9040.313 | Hz |
| 9074.294 | Hz |
| 9189.092 | Hz |
| 9484.512 | Hz |
| 9943.972 | Hz |
| 12086.394 | Hz |

Treatment of Leukemia and Chronic Lymphoid Cancer

Leukemia and chronic lymphoid cancer may be treated according to the methods disclosed herein by the application of leukemia and chronic lymphoid cancer specific amplitude-modulated frequencies. Leukemia and chronic lymphoid cancer specific frequencies are provided below in Table 29. A patient suffering from leukemia and chronic lymphoid cancer may be treated with any number of the frequencies provided in Table 29, however it is generally preferable to employ as many of the frequencies as is practical for the treatment of the patient. Accordingly, 10 or more, 15 or more of the listed leukemia and chronic lymphoid cancer specific frequencies may be used in the treatment of leukemia and chronic lymphoid cancer. It should be noted that while the table below provides certain frequencies for treatment of leukemia and chronic lymphoid cancer, the disclosure is not so limiting, and other frequencies discovered to be effective in the treatment of leukemia and chronic lymphoid cancer are within the scope of this disclosure.

TABLE 29
Amplitude-modulated frequencies for treatment
of leukemia and chronic lymphoid cancer.

| | |
|---|---|
| 814.413 | Hz |
| 825.145 | Hz |
| 2415.243 | Hz |
| 2436.316 | Hz |
| 2874.432 | Hz |
| 2891.029 | Hz |
| 3361.671 | Hz |
| 5245.452 | Hz |
| 5557.333 | Hz |
| 6850.197 | Hz |
| 6919.322 | Hz |
| 7587.224 | Hz |
| 7629.318 | Hz |
| 8172.405 | Hz |
| 8272.338 | Hz |
| 8438.453 | Hz |
| 12950.331 | Hz |

Treatment of Myeloma

Myeloma may be treated according to the methods disclosed herein by the application of myeloma specific amplitude-modulated frequencies. Myeloma specific frequencies are provided below in Table 30. A patient suffering from myeloma may be treated with any number of the frequencies provided in Table 30, however it is generally preferable to employ as many of the frequencies as is practical for the treatment of the patient. Accordingly, 10 or more, 15 or more, or 20 or more of the listed myeloma specific frequencies may be used in the treatment of myeloma. It should be noted that while the table below provides certain frequencies for treatment of myeloma, the disclosure is not so limiting, and other frequencies discovered to be effective in the treatment of myeloma are within the scope of this disclosure.

TABLE 30
Amplitude-modulated frequencies for treatment of myeloma.

| | |
|---|---|
| 656.339 | Hz |
| 765.196 | Hz |
| 2336.238 | Hz |
| 2372.122 | Hz |
| 2381.443 | Hz |
| 2425.394 | Hz |
| 2741.261 | Hz |
| 2883.618 | Hz |
| 2919.273 | Hz |
| 3265.323 | Hz |
| 3564.455 | Hz |
| 3580.25 | Hz |
| 3584.291 | Hz |
| 3674.292 | Hz |
| 5249.331 | Hz |
| 7967.311 | Hz |
| 7973.125 | Hz |
| 8049.952 | Hz |
| 8283.329 | Hz |
| 10351.323 | Hz |

Treatment of Lymphoma

Lymphoma may be treated according to the methods disclosed herein by the application of lymphoma specific amplitude-modulated frequencies. Lymphoma specific frequencies are provided below in Table 31. A patient suffering from lymphoma may be treated with any number of the frequencies provided in Table 31, however it is generally preferable to employ as many of the frequencies as is practical for the treatment of the patient. Accordingly, 10 or more, 15 or more of the listed lymphoma specific frequencies may be used in the treatment of lymphoma. It should be noted that while the table below provides certain frequencies for treatment of lymphoma, the disclosure is not so limiting, and other frequencies discovered to be effective in the treatment of lymphoma are within the scope of this disclosure.

TABLE 31
Amplitude-modulated frequencies for treatment of lymphoma.

| | |
|---|---|
| 752.5 | Hz |
| 976.3 | Hz |
| 1558.223 | Hz |
| 2310.912 | Hz |
| 2477.919 | Hz |
| 2560.843 | Hz |
| 3348.783 | Hz |
| 3371.216 | Hz |
| 3605.432 | Hz |
| 3623.198 | Hz |
| 3838.281 | Hz |
| 3838.48 | Hz |
| 5102 | Hz |
| 5696.932 | Hz |
| 5724.231 | Hz |
| 6358.194 | Hz |
| 7472.211 | Hz |
| 8062.121 | Hz |
| 8222.222 | Hz |

Treatment of Brain Cancer

Brain cancer may be treated according to the methods disclosed herein by the application of brain cancer specific amplitude-modulated frequencies. Brain cancer specific frequencies are provided below in Table 32. A patient suffering from brain cancer may be treated with any number of the frequencies provided in Table 32, however it is generally preferable to employ as many of the frequencies as is practical for the treatment of the patient. Accordingly, 10 or more, 15 or more, or 20 or more, 25 or more, or 30 or more, 35 or more, or 40 or more, 45 or more, or 50 or more of the listed brain cancer specific frequencies may be used in the treatment of brain cancer. It should be noted that while the table below provides certain frequencies for treatment of brain cancer, the disclosure is not so limiting, and other frequencies discovered to be effective in the treatment of brain cancer are within the scope of this disclosure.

TABLE 32
Amplitude-modulated frequencies for treatment of brain cancer.

| | |
|---|---|
| 1372.934 | Hz |
| 2318.182 | Hz |
| 2381.443 | Hz |
| 2425.394 | Hz |
| 2442.423 | Hz |
| 2478.973 | Hz |
| 2654.513 | Hz |
| 2661.324 | Hz |
| 2686.105 | Hz |
| 2690.179 | Hz |
| 3249.332 | Hz |
| 3277.509 | Hz |
| 3335.279 | Hz |
| 3348.783 | Hz |
| 3436.211 | Hz |
| 3916.321 | Hz |
| 4031.933 | Hz |
| 4086.091 | Hz |
| 4241.321 | Hz |
| 4318.222 | Hz |
| 4334.33 | Hz |
| 4358.333 | Hz |
| 4393.419 | Hz |
| 4454.194 | Hz |
| 4515.789 | Hz |
| 4619.324 | Hz |
| 4723.937 | Hz |
| 4853.286 | Hz |
| 5289.231 | Hz |
| 5378.099 | Hz |
| 5426.323 | Hz |
| 5640.981 | Hz |
| 6316.211 | Hz |
| 6459.203 | Hz |
| 6474.332 | Hz |
| 6626.572 | Hz |
| 6855.286 | Hz |
| 6915.886 | Hz |
| 6943.386 | Hz |
| 7151.264 | Hz |
| 7182.922 | Hz |
| 7194.897 | Hz |
| 7323.209 | Hz |
| 7390.343 | Hz |
| 7796.221 | Hz |
| 7961.122 | Hz |
| 8128.942 | Hz |
| 8245.109 | Hz |
| 8272.281 | Hz |
| 8358.154 | Hz |
| 8408.121 | Hz |
| 9138.82 | Hz |
| 10719.318 | Hz |
| 11556.241 | Hz |
| 12828.633 | Hz |
| 14515.962 | Hz |
| 14586.765 | Hz. |

The following are synopses relate to exemplary uses of the electronic devices of the present invention and the cancer specific frequencies for the treatment of patients:

### Example A

A 79-year-old man was diagnosed with hepatitis A and B negative hepatocellular carcinoma. He underwent left hepatectomy the following month, which revealed the presence of a poorly differentiated hepatocellular carcinoma. The tumors largest diameter was 10 cm and was staged as pT3NxMx. After five months from diagnosis, the patient had evidence of disease progression with four new lesions identified within the right lobe of the liver and new mediastinal adenopathy, and chemoembolization with doxorubicin and lipiodol was performed over the next month. A follow up MRI performed the following month showed progression of disease. Treatment with sorafenib was initiated when the patient's performance status was KPS 80%, ECOG 1 with a Child Pugh of A6. Simultaneous compassionate treatment with the TheraBionic device emitting 206 hepatocellular carcinoma (HCC) specific frequencies was administered 3 hours a day. The patient had a complete response by marker as the level of alpha-fetoprotein (AFP) decreased from 92,620·0 international units/ml (IU/ml) prior to treatment initiation to 4.18 IU/ml, measured a year from the original diagnosis and 4 months after commencing treatment with the HCC specific frequencies.

Nexavar was discontinued in the beginning of the third year following the initial diagnosis because of intolerable side effects. One month after discontinuation of Nexavar, there was evidence of early disease progression with a doubling of the AFP level, and near doubling of the levels of liver enzymes ASAP, ALAT, and gamma GT. The patient was re-examined for tumor-specific frequencies using the method described above. The analysis revealed pulse pressure changes for additional HCC specific frequencies. Fifty HCC frequencies were added to the 206 HCC frequencies, i.e., the patient began receiving 256 frequencies with each treatment. A new MRI of the abdomen obtained in seven months later revealed the emergence of new tumor nodules. The patient was re-examined on the following month using the method described above. The analysis revealed pulse pressure changes for 12 additional HCC specific frequencies, bringing the total number to 268 HCC specific frequencies. The same approach was used whenever there was either clear progression by changes in AFP levels and/or significant enlargement of existing tumor masses and/or emergence of new tumor masses. Addition of new HCC specific frequencies led to significant decrease or stabilization of tumor masses as well as decreases in AFP levels. In the last year of treatment, the patient received a total of 422 HCC specific frequencies.

The patient received treatment with the TheraBionic device for 67 months, when he became unable to receive treatment on a regular basis because of a hip fracture secondary to a fall, generalized weakness, and worsening renal failure. He passed away in six months later. In summary, addition of HCC specific frequencies led to repeated objective clinical responses assessed both radiologically and by tumor marker, which led to an exceptionally long survival (more than 6 years) in a patient with rapidly progressive advanced hepatocellular carcinoma.

### Example B

87-year-old man with a long-standing history of type II diabetes was diagnosed with advanced unresectable multifocal hepatocellular carcinoma. He declined treatment with Nexavar and requested compassionate use treatment with the TheraBionic device. He began receiving treatment with 313 HCC specific frequencies two months after diagnosis. The patient had stable disease for 13 months when he had evidence of disease progression by RECIST criteria. He began receiving treatment with 355 HCC specific frequencies within the following month. A follow-up MRI of the abdomen obtained after two months revealed the presence of necrosis within 2 lesions and stable disease in the others. Hence, the addition of 42 HCC specific frequencies led to a radiological response. A follow-up MRI of the abdomen obtained two months later showed stable disease by RECIST criteria. A follow-up MRI of the liver obtained after three months showed progression of disease by RECIST criteria with the appearance of new liver lesions. Two weeks later, the patient was examined with the method described above and changes in pulse pressure were observed for 49 additional HCC frequencies. Hence, the patient began receiving treatment with 404 frequencies as of that month. A repeat MRI of the abdomen obtained three months later showed stable disease. The patient continued to do well over the next two months when he had gastrointestinal bleeding due to esophageal varices. He declined treatment for esophageal varices and passed away later that month. Hence, treatment of this patient demonstrates the clinical efficacy of additional HCC frequencies, which led to both objective response and stable disease.

Background

A portable and programmable device as described herein that is capable of delivering low levels of amplitude-modulated electromagnetic fields has been developed. The device emits a 27 MHz radiofrequency signal, amplitude-modulated at cancer-specific frequencies ranging from 0.1 to 150 kHz with high precision. The device is connected to a spoon-like coupler, which is placed in the patient's mouth during treatment.

### Example 1: Treatment of Breast Cancer

Breast cancer may be treated according to the methods disclosed herein by the application of breast cancer specific amplitude-modulated frequencies. Breast cancer specific frequencies are provided below in Table 33.

TABLE 33
Amplitude-modulated frequencies discovered to
be effective for treatment of breast cancer.

| | |
|---|---|
| 374-374.7 | Hz |
| 419.8-420.4 | Hz |
| 420.4-420.8 | Hz |
| 429-429.7 | Hz |
| 1336-1336.7 | Hz |
| 1369-1369.7 | Hz |
| 1728-1728.7 | Hz |
| 1771.8-1772.3 | Hz |
| 1772.3-1772.8 | Hz |
| 2811-2811.7 | Hz |
| 2824-2824.7 | Hz |
| 2840-2840.7 | Hz |
| 2858.8-2859.35 | Hz |
| 2860-2860.7 | Hz |
| 2878-2878.7 | Hz |
| 2890-2890.7 | Hz |
| 3209-3209.7 | Hz |
| 3322-3322.7 | Hz |
| 3360-3360.7 | Hz |
| 3663-3663.7 | Hz |
| 3839-3839.7 | Hz |
| 3842.4-3843 | Hz |
| 3854-3854.7 | Hz |
| 3936-3936.7 | Hz |
| 4323-4323.7 | Hz |
| 4329-4329.7 | Hz |
| 4416-4416.7 | Hz |
| 4430-4430.7 | Hz |
| 4477.4-4478 | Hz |
| 4480.9-4481.35 | Hz |
| 4714-4714.7 | Hz |
| 4805.3-4806 | Hz |
| 4813-4813.7 | Hz |
| 4819-4819.7 | Hz |
| 4822-4822.7 | Hz |
| 4823-4823.7 | Hz |
| 4833-4833.7 | Hz |
| 4890-4890.7 | Hz |
| 5154-5154.7 | Hz |
| 5243-5243.7 | Hz |
| 5407.3-5408 | Hz |
| 5716-5716.7 | Hz |
| 5976-5976.7 | Hz |
| 6024-6024.7 | Hz |
| 6070-6070.7 | Hz |
| 6094-6094.7 | Hz |
| 6414-6414.7 | Hz |
| 6717-6717.7 | Hz |
| 6751-6751.7 | Hz |
| 6968.4-6969 | Hz |
| 7013-7013.7 | Hz |
| 7042-7042.7 | Hz |
| 7080-7080.7 | Hz |
| 7145.4-7146 | Hz |
| 7151-7151.7 | Hz |
| 7158-7158.7 | Hz |
| 7225-7225.7 | Hz |
| 8332-8332.7 | Hz |
| 8824-8824.7 | Hz |
| 8837-8837.7 | Hz |
| 9935-9935.7 | Hz |
| 9961-9961.7 | Hz |
| 10013-10013.7 | Hz |
| 10064-10064.7 | Hz |
| 10339.8-10340.4 | Hz |
| 10384-10384.7 | Hz |
| 10745-10745.7 | Hz |
| 10788-10788.7 | Hz |
| 10834-10834.7 | Hz |
| 10854-10854.7 | Hz |
| 10873-10873.7 | Hz |
| 10894-10894.7 | Hz |
| 11163-11163.7 | Hz |
| 11414-11414.7 | Hz |
| 11421-11421.7 | Hz |
| 11429-11429.7 | Hz |
| 11719-11719.7 | Hz |
| 11745-11745.7 | Hz |
| 11931-11931.7 | Hz |
| 11974-11974.7 | Hz |
| 11993-11993.7 | Hz |
| 12118-12118.7 | Hz |
| 12137-12137.7 | Hz |
| 12144-12144.7 | Hz |
| 12318-12318.7 | Hz |
| 12342-12342.7 | Hz |
| 12364-12364.7 | Hz |
| 12557-12557.7 | Hz |
| 12588-12588.7 | Hz |
| 13329-13329.7 | Hz |
| 13377-13377.7 | Hz |
| 13386-13386.7 | Hz |
| 13644-13644.7 | Hz |
| 13661-13661.7 | Hz |
| 13713-13713.7 | Hz |
| 14164-14164.7 | Hz |
| 14308-14308.7 | Hz |
| 14333.4-14333.9 | Hz |
| 14432-14432.7 | Hz |
| 14524-14524.7 | Hz |
| 14589-14589.7 | Hz |
| 14729-14729.7 | Hz |
| 14813-14813.7 | Hz |
| 14835-14835.7 | Hz |
| 14858-14858.7 | Hz |
| 14885-14885.7 | Hz |
| 15035-15035.6 | Hz |
| 15067-15067.7 | Hz |
| 15126-15126.7 | Hz |
| 15195-15195.7 | Hz |
| 15269-15269.7 | Hz |
| 15432-15432.7 | Hz |
| 15432-15432.7 | Hz |
| 15563-15563.7 | Hz |
| 15621-15621.7 | Hz |
| 15624-15624.7 | Hz |
| 15642-15642.7 | Hz |
| 15647-15647.7 | Hz |
| 15669-15669.7 | Hz |
| 15698-15698.7 | Hz |
| 15823-15823.7 | Hz |
| 16347-16347.7 | Hz |
| 17832-17832.7 | Hz |
| 18221-18221.7 | Hz |
| 18260-18260.7 | Hz |
| 19433-19433.7 | Hz |
| 19446-19446.7 | Hz |
| 19489.3-19490 | Hz |
| 19782-19782.7 | Hz |
| 19827-19827.7 | Hz |
| 21291.4-21292 | Hz |
| 21887-21887.7 | Hz |
| 22020-22020.7 | Hz |
| 22419-22419.7 | Hz |
| 22422-22422.7 | Hz |
| 22425-22425.7 | Hz |
| 22444-22444.7 | Hz |
| 22491-22491.7 | Hz |
| 22840.3-22841 | Hz |
| 22859-22859.7 | Hz |
| 22973-22973.7 | Hz |
| 22983-22983.7 | Hz |
| 23258-23258.7 | Hz |
| 23652-23652.7 | Hz |
| 24174-24174.7 | Hz |
| 24811-24811.7 | Hz |
| 26954-26954.7 | Hz |
| 27114-27114.7 | Hz |
| 30463.3-30464 | Hz |
| 34557.3-34558 | Hz |
| 34854-34854.7 | Hz |
| 38144-38144.7 | Hz |
| 38682-38682.7 | Hz |
| 41561-41561.7 | Hz |
| 41623-41623.7 | Hz |
| 41735-41735.7 | Hz |
| 42713-42713.7 | Hz |
| 44575.3-44576 | Hz |
| 46662-46662.7 | Hz |
| 48173-48173.7 | Hz |
| 50292-50292.7 | Hz |

### Example 2: Treatment of Neuroendocrine Tumors

Neuroendocrine tumors may be treated according to the methods disclosed herein by the application of neuroendocrine tumor specific amplitude-modulated frequencies. Neuroendocrine tumors specific frequencies are provided below in Table 34.

TABLE 34
Amplitude-modulated frequencies discovered to be
effective for treatment of neuroendocrine tumors.

| | |
|---|---|
| 843-843.7 | Hz |
| 1656-1656.7 | Hz |
| 2423-2423.7 | Hz |
| 2480-2480.7 | Hz |
| 2707-2707.7 | Hz |
| 2890-2890.7 | Hz |
| 3080.3-3081 | Hz |
| 3661-3661.7 | Hz |
| 3678-3678.7 | Hz |
| 3976.4-3977 | Hz |
| 4048-4048.7 | Hz |
| 4055-4055.7 | Hz |
| 4368.3-4369 | Hz |
| 4459-4459.7 | Hz |
| 4621-4621.7 | Hz |
| 4837-4837.7 | Hz |
| 4866-4866.7 | Hz |
| 5165.4-5166 | Hz |
| 5265-5265.7 | Hz |
| 5270-5270.7 | Hz |
| 6436-6436.7 | Hz |
| 7037-7037.7 | Hz |
| 8008-8008.7 | Hz |
| 8024-8024.7 | Hz |
| 8097-8097.7 | Hz |
| 8427-8427.7 | Hz |
| 8478.3-8479 | Hz |
| 8480-8480.7 | Hz |
| 8492-8492.7 | Hz |
| 8853-8853.7 | Hz |
| 8877-8877.7 | Hz |
| 9122-9122.7 | Hz |
| 9131-9131.7 | Hz |
| 9174-9174.7 | Hz |
| 10073-10073.7 | Hz |
| 10848.7-10849.3 | Hz |
| 11267-11267.7 | Hz |
| 11336-11336.7 | Hz |
| 11524-11524.7 | Hz |
| 11551.3-11552 | Hz |
| 11927.4-11928 | Hz |
| 12309-12309.7 | Hz |
| 13617-13617.7 | Hz |
| 13674-13674.7 | Hz |
| 13774-13774.7 | Hz |
| 14350-14350.7 | Hz |
| 14456-14456.7 | Hz |
| 14479-14479.7 | Hz |
| 14540-14540.7 | Hz |
| 14553-14553.7 | Hz |
| 14756-14756.7 | Hz |
| 14922-14922.7 | Hz |
| 14961-14961.7 | Hz |
| 15226-15226.7 | Hz |
| 15817-15817.7 | Hz |
| 15825-15825.7 | Hz |
| 15835-15835.7 | Hz |
| 15836-15836.7 | Hz |
| 15897-15897.7 | Hz |
| 16071-16071.7 | Hz |
| 17417-17417.7 | Hz |
| 17418-17418.7 | Hz |
| 17473-17473.7 | Hz |
| 17474-17474.7 | Hz |
| 17621-17621.7 | Hz |
| 17655-17655.7 | Hz |
| 18655-18655.7 | Hz |
| 18672-18672.7 | Hz |
| 18678-18678.7 | Hz |
| 19393-19393.7 | Hz |
| 19426-19426.7 | Hz |
| 19817-19817.7 | Hz |
| 19819-19819.7 | Hz |
| 19861-19861.7 | Hz |
| 19867-19867.7 | Hz |
| 20818-20818.7 | Hz |
| 21708-21708.7 | Hz |
| 21720-21720.7 | Hz |
| 21865-21865.7 | Hz |
| 21876-21876.7 | Hz |
| 22119-22119.7 | Hz |
| 22454-22454.7 | Hz |
| 24073-24073.7 | Hz |
| 30304-30304.7 | Hz |
| 31685-31685.7 | Hz |
| 43633-43633.7 | Hz |
| 44773-44773.7 | Hz |
| 46662-46662.7 | Hz |
| 46793-46793.7 | Hz |
| 48103-48103.7 | Hz |
| 50243-50243.7 | Hz. |

### Example 3: Treatment of Non-Hodgkin Lymphoma

Non-Hodgkin lymphoma tumors may be treated according to the methods disclosed herein by the application of non-Hodgkin lymphoma specific amplitude-modulated frequencies. Non-Hodgkin lymphoma tumors specific frequencies are provided below in Table 35.

TABLE 35
Amplitude-modulated frequencies discovered to be
effective for treatment of non-Hodgkin lymphoma.

| | |
|---|---|
| 3222-3222.7 | Hz |
| 3228-3228.7 | Hz |
| 3272-3272.7 | Hz |
| 3618-3618.7 | Hz |
| 4523-4523.7 | Hz |
| 4556-4556.7 | Hz |
| 4584-4584.7 | Hz |
| 5116-5116.7 | Hz |
| 5142-5142.7 | Hz |
| 6413-6413.7 | Hz |
| 6419.3-6420 | Hz |
| 6441-6441.7 | Hz |
| 6474-6474.7 | Hz |
| 6911.3-6912 | Hz |
| 6912-6912.7 | Hz |
| 6943-6943.7 | Hz |
| 7551-7551.7 | Hz |
| 7811-7811.7 | Hz |
| 7857-7857.7 | Hz |
| 7865-7865.7 | Hz |
| 8125-8125.7 | Hz |
| 9106-9106.7 | Hz |
| 9161-9161.7 | Hz |
| 9179-9179.7 | Hz |
| 9230-9230.7 | Hz |
| 9236-9236.7 | Hz |
| 9247-9247.7 | Hz |
| 11730.6-11731.1 | Hz |
| 11785-11785.7 | Hz |
| 13222-13222.7 | Hz |
| 13751-13751.7 | Hz |
| 13780-13780.7 | Hz |
| 15006-15006.7 | Hz |
| 15033-15033.7 | Hz |
| 15056-15056.7 | Hz |
| 15729-15729.7 | Hz |
| 15773-15773.7 | Hz |
| 17229-17229.7 | Hz |
| 17246-17246.7 | Hz |
| 17268-17268.7 | Hz |
| 20256-20256.7 | Hz |
| 22444-22444.7 | Hz |
| 30666-30666.7 | Hz |
| 30713-30713.7 | Hz |
| 30843-30843.7 | Hz |
| 38216-38216.7 | Hz |

### Example 4: Treatment of Adenocarcinoma of Pancreas

Adenocarcinoma of pancreas may be treated according to the methods disclosed herein by the application of adenocarcinoma of pancreas specific amplitude-modulated frequencies. Adenocarcinoma of pancreas specific frequencies are provided below in Table 36.

TABLE 36
Amplitude-modulated frequencies discovered to be effective
for treatment of adenocarcinoma of the pancreas.

| | |
|---|---|
| 403-403.7 | Hz |
| 441-441.7 | Hz |
| 527-527.7 | Hz |
| 624.32-624.8 | Hz |
| 633-633.7 | Hz |
| 727-727.7 | Hz |
| 759-759.7 | Hz |
| 785-785.7 | Hz |
| 811-811.7 | Hz |
| 829-829.7 | Hz |
| 841-841.7 | Hz |
| 962-962.7 | Hz |
| 964.5-965 | Hz |
| 972-972.7 | Hz |
| 974.4-975 | Hz |
| 977-977.7 | Hz |
| 1179.2-1179.8 | Hz |
| 1250.3-1251 | Hz |
| 1314-1314.7 | Hz |
| 1352-1352.7 | Hz |
| 1622-1622.7 | Hz |
| 1652-1652.7 | Hz |
| 1656-1656.7 | Hz |
| 1670.3-1671 | Hz |
| 1914-1914.7 | Hz |
| 2346-2346.7 | Hz |
| 2362-2362.7 | Hz |
| 2380-2380.7 | Hz |
| 2469.2-2469.7 | Hz |
| 2627-2627.7 | Hz |
| 2706-2706.7 | Hz |
| 2720.3-2721 | Hz |
| 2726-2726.7 | Hz |
| 2754-2754.7 | Hz |
| 2778-2778.7 | Hz |
| 2842-2842.7 | Hz |
| 2864-2864.7 | Hz |
| 2940-2940.7 | Hz |
| 2988-2988.7 | Hz |
| 3234-3234.7 | Hz |
| 3280.6-3281.3 | Hz |
| 3366-3366.7 | Hz |
| 3428-3428.7 | Hz |
| 3446-3446.7 | Hz |
| 3466-3466.7 | Hz |
| 3523-3523.7 | Hz |
| 3605-3605.7 | Hz |
| 3622.5-3623 | Hz |
| 3627-3627.7 | Hz |
| 3631-3631.7 | Hz |
| 3656-3656.7 | Hz |
| 3663-3663.7 | Hz |
| 3667-3667.7 | Hz |
| 3687-3687.7 | Hz |
| 4152-4152.7 | Hz |
| 4233-4233.7 | Hz |
| 4249-4249.7 | Hz |
| 4281-4281.7 | Hz |
| 4368.3-4369 | Hz |
| 4417-4417.7 | Hz |
| 4425-4425.7 | Hz |
| 4440.4-4441 | Hz |
| 4465-4465.7 | Hz |
| 4480.9-4481.35 | Hz |
| 4580.11-4580.7 | Hz |
| 4621-4621.7 | Hz |
| 4637-4637.7 | Hz |
| 4657-4657.6 | Hz |
| 4725-4725.7 | Hz |
| 4733-4733.7 | Hz |
| 4749-4749.7 | Hz |
| 4841-4841.7 | Hz |
| 4924-4924.7 | Hz |
| 4946-4946.7 | Hz |
| 5063-5063.7 | Hz |
| 5089-5089.7 | Hz |
| 5378.2-5378.7 | Hz |
| 5424-5424.7 | Hz |
| 5438-5438.7 | Hz |
| 6112-6112.7 | Hz |
| 6159-6159.7 | Hz |
| 6161.3-6162 | Hz |
| 6167-6167.7 | Hz |
| 6179-6179.7 | Hz |
| 6220.3-6221 | Hz |
| 6346-6346.7 | Hz |
| 6380.4-6381 | Hz |
| 6808-6808.7 | Hz |
| 6825-6825.7 | Hz |
| 6847-6847.7 | Hz |
| 6850-6850.7 | Hz |
| 6878-6878.7 | Hz |
| 7036-7036.7 | Hz |
| 7037-7037.7 | Hz |
| 7118-7118.7 | Hz |
| 7160-7160.7 | Hz |
| 7432-7432.7 | Hz |
| 7541-7541.7 | Hz |
| 7676-7676.7 | Hz |
| 7936-7936.7 | Hz |
| 8024-8024.7 | Hz |
| 8032-8032.7 | Hz |
| 8043-8043.7 | Hz |
| 8447-8447.7 | Hz |
| 8734-8734.7 | Hz |
| 8756-8756.7 | Hz |
| 8757-8757.7 | Hz |
| 8809-8809.7 | Hz |
| 8823-8823.7 | Hz |
| 8841-8841.7 | Hz |
| 8845-8845.7 | Hz |
| 8846-8846.7 | Hz |
| 8872-8872.7 | Hz |
| 9133-9133.7 | Hz |
| 9155.7-9156.2 | Hz |
| 9520-9520.7 | Hz |
| 9859-9859.7 | Hz |
| 10342-10342.6 | Hz |
| 10352-10352.7 | Hz |
| 10378-10378.7 | Hz |
| 10436-10436.7 | Hz |
| 10566.4-10567 | Hz |
| 10633-10633.7 | Hz |
| 10641-10641.7 | Hz |
| 10671-10671.7 | Hz |
| 10708-10708.7 | Hz |
| 10859-10859.7 | Hz |
| 10876-10876.7 | Hz |
| 11174-11174.7 | Hz |
| 11220-11220.7 | Hz |
| 11247-11247.7 | Hz |
| 11264-11264.7 | Hz |
| 11284-11284.7 | Hz |
| 11630-11630.7 | Hz |
| 11994-11994.7 | Hz |
| 12120.2-12120.7 | Hz |
| 12135-12135.7 | Hz |
| 12152-12152.7 | Hz |
| 12184-12184.7 | Hz |
| 12711-12711.7 | Hz |
| 13375-13375.7 | Hz |
| 13571-13571.7 | Hz |
| 13636.2-13636.7 | Hz |
| 13738.3-13739 | Hz |
| 13947-13947.7 | Hz |
| 14116-14116.7 | Hz |
| 14279-14279.7 | Hz |
| 14376-14376.7 | Hz |
| 14462-14462.7 | Hz |
| 14519-14519.7 | Hz |
| 14558-14558.7 | Hz |
| 14729-14729.7 | Hz |
| 14940.4-14941 | Hz |
| 14957-14957.7 | Hz |
| 15179-15179.7 | Hz |
| 15225-15225.6 | Hz |
| 15536-15536.7 | Hz |
| 15576-15576.7 | Hz |
| 15597-15597.7 | Hz |
| 15663-15663.7 | Hz |
| 15678-15678.7 | Hz |
| 15711-15711.7 | Hz |
| 15724-15724.7 | Hz |
| 15740-15740.7 | Hz |
| 15749-15749.7 | Hz |
| 15824-15824.7 | Hz |
| 15867-15867.7 | Hz |
| 16143-16143.7 | Hz |
| 17214-17214.7 | Hz |
| 17253-17253.7 | Hz |
| 17271-17271.7 | Hz |
| 17283-17283.7 | Hz |
| 17739-17739.7 | Hz |
| 17944-17944.7 | Hz |
| 17962-17962.7 | Hz |
| 18279-18279.7 | Hz |
| 18678.7-18679.3 | Hz |
| 18921-18921.7 | Hz |
| 18922-18922.7 | Hz |
| 19059-19059.7 | Hz |
| 19464-19464.7 | Hz |
| 19686-19686.4 | Hz |
| 19724-19724.7 | Hz |
| 20123-20123.7 | Hz |
| 20128-20128.7 | Hz |
| 20144-20144.7 | Hz |
| 20149.3-20150 | Hz |
| 20219-20219.7 | Hz |
| 20254-20254.7 | Hz |
| 21546-21546.7 | Hz |
| 21616-21616.7 | Hz |
| 21664-21664.7 | Hz |
| 21840-21840.7 | Hz |
| 21876-21876.7 | Hz |
| 21894-21894.7 | Hz |
| 22355-22355.7 | Hz |
| 22430-22430.7 | Hz |
| 22597-22597.7 | Hz |
| 22742-22742.7 | Hz |
| 22822-22822.7 | Hz |
| 22846-22846.7 | Hz |
| 22867-22867.7 | Hz |
| 23237-23237.7 | Hz |
| 23284-23284.7 | Hz |
| 24228-24228.7 | Hz |
| 24275-24275.7 | Hz |
| 24508-24508.7 | Hz |
| 24609.3-24610 | Hz |
| 24841-24841.7 | Hz |
| 27478-27478.7 | Hz |
| 32143-32143.7 | Hz |
| 32493-32493.7 | Hz |
| 33546-33546.7 | Hz |
| 40133-40133.7 | Hz |
| 40595-40595.7 | Hz |
| 44913-44913.7 | Hz |
| 45474-45474.7 | Hz |
| 46672.3-46673 | Hz |
| 48204-48204.7 | Hz |
| 48314-48314.7 | Hz |
| 50072-50072.7 | Hz |
| 50254-50254.7 | Hz |
| 52812-52812.7 | Hz |
| 79867-79867.7 | Hz |
| 80993.3-80994 | Hz |
| 91877-91877.7 | Hz |
| 95742-95742.7 | Hz |

### Example 5: Treatment of Head and Neck Cancer

Head and neck cancer may be treated according to the methods disclosed herein by the application of head and neck cancer specific amplitude-modulated frequencies. Head and neck cancer specific frequencies are provided below in Table 37.

TABLE 37
Amplitude-modulated frequencies discovered to be
effective for treatment of head and neck cancer.

| | |
|---|---|
| 1858-1858.7 | Hz |
| 2893-2893.7 | Hz |
| 5152-5152.7 | Hz |
| 6897-6897.7 | Hz |
| 7080-7080.7 | Hz |
| 7136-7136.7 | Hz |
| 7274-7274.7 | Hz |
| 8755-8755.7 | Hz |
| 9573-9573.7 | Hz |
| 9656-9656.7 | Hz |
| 10439-10439.7 | Hz |
| 10484-10484.7 | Hz |
| 10645-10645.7 | Hz |
| 10673-10673.7 | Hz |
| 11559-11559.7 | Hz |
| 14052-14052.7 | Hz |
| 17379-17379.7 | Hz |
| 19269-19269.7 | Hz |

### Example 6: Treatment of Gastric Cancer

Gastric cancer may be treated according to the methods disclosed herein by the application of gastric cancer specific amplitude-modulated frequencies. Gastric cancer specific frequencies are provided below in Table 38.

TABLE 38
Amplitude-modulated frequencies discovered to
be effective for treatment of gastric cancer.

| | |
|---|---|
| 374-374.7 | Hz |
| 560-560.7 | Hz |
| 628-628.7 | Hz |
| 642.4-643 | Hz |
| 708.3-709 | Hz |
| 749-749.7 | Hz |
| 806-806.6 | Hz |
| 825-825.7 | Hz |
| 843-843.7 | Hz |
| 914-914.7 | Hz |
| 959-959.7 | Hz |
| 969-969.7 | Hz |
| 1157-1157.7 | Hz |
| 1436-1436.7 | Hz |
| 1656-1656.7 | Hz |
| 2379.3-2380 | Hz |
| 2417-2417.7 | Hz |
| 2436-2436.7 | Hz |
| 2455-2455.7 | Hz |
| 2478-2478.7 | Hz |
| 2747-2747.7 | Hz |
| 2762-2762.7 | Hz |
| 2774-2774.7 | Hz |
| 2856.4-2857 | Hz |
| 2891-2891.7 | Hz |
| 2981-2981.7 | Hz |
| 3104.3-3105 | Hz |
| 3133-3133.7 | Hz |
| 3522-3522.7 | Hz |
| 3537-3537.7 | Hz |
| 3549-3549.7 | Hz |
| 3556-3556.7 | Hz |
| 3595-3595.7 | Hz |
| 3927-3927.7 | Hz |
| 4154-4154.7 | Hz |
| 4548-4548.7 | Hz |
| 4723-4723.7 | Hz |
| 5286-5286.7 | Hz |
| 5494.4-5495 | Hz |
| 5548.3-5549 | Hz |
| 5742-5742.7 | Hz |
| 6010-6010.7 | Hz |
| 6064-6064.7 | Hz |
| 6329-6329.7 | Hz |
| 6444-6444.7 | Hz |
| 6469-6469.7 | Hz |
| 6488-6488.7 | Hz |
| 7222.4-7223 | Hz |
| 7646-7646.7 | Hz |
| 7748-7748.7 | Hz |
| 7855-7855.7 | Hz |
| 7927-7927.7 | Hz |
| 7951-7951.7 | Hz |
| 7970-7970.7 | Hz |
| 7979-7979.7 | Hz |
| 8039-8039.7 | Hz |
| 8055.4-8056 | Hz |
| 8926-8926.7 | Hz |
| 9109-9109.7 | Hz |
| 9123-9123.7 | Hz |
| 9136-9136.7 | Hz |
| 9146-9146.7 | Hz |
| 9157-9157.7 | Hz |
| 9194-9194.7 | Hz |
| 10137-10137.7 | Hz |
| 10345-10345.7 | Hz |
| 10727-10727.7 | Hz |
| 10802-10802.7 | Hz |
| 11533-11533.7 | Hz |
| 12165-12165.7 | Hz |
| 13241-13241.7 | Hz |
| 13307-13307.7 | Hz |
| 13330.7-13331.2 | Hz |
| 13408-13408.7 | Hz |
| 13434-13434.7 | Hz |
| 13714-13714.7 | Hz |
| 13746-13746.7 | Hz |
| 14016.4-14017 | Hz |
| 14160-14160.7 | Hz |
| 14483-14483.7 | Hz |
| 14511-14511.7 | Hz |
| 14670.4-14671 | Hz |
| 16309-16309.7 | Hz |
| 17418-17418.7 | Hz |
| 17455-17455.7 | Hz |
| 17485-17485.7 | Hz |
| 17581-17581.7 | Hz |
| 19125-19125.7 | Hz |
| 21430-21430.7 | Hz |
| 21714-21714.7 | Hz |
| 23946-23946.7 | Hz |
| 39586-39586.7 | Hz |
| 40734-40734.7 | Hz |
| 41172-41172.7 | Hz |
| 42146-42146.7 | Hz |
| 46143-46143.7 | Hz |

### Example 7: Treatment of Glioblastoma

Glioblastoma may be treated according to the methods disclosed herein by the application of glioblastoma specific amplitude-modulated frequencies. Glioblastoma specific frequencies are provided below in Table 39.

TABLE 39
Amplitude-modulated frequencies discovered to
be effective for treatment of glioblastoma.

| | |
|---|---|
| 262-262.7 | Hz |
| 295-295.6 | Hz |
| 415-415.7 | Hz |
| 418-418.7 | Hz |
| 423-423.7 | Hz |
| 434-434.7 | Hz |
| 440.4-441 | Hz |
| 476-476.7 | Hz |
| 556-556.7 | Hz |
| 608-608.7 | Hz |
| 612-612.7 | Hz |
| 627-627.7 | Hz |
| 656-656.7 | Hz |
| 694-694.7 | Hz |
| 708.3-709 | Hz |
| 741-741.7 | Hz |
| 752.4-753 | Hz |
| 757-757.6 | Hz |
| 759-759.7 | Hz |
| 776-776.7 | Hz |
| 796.3-797 | Hz |
| 836-836.7 | Hz |
| 941-941.7 | Hz |
| 1027-1027.7 | Hz |
| 1045-1045.7 | Hz |
| 1195-1195.7 | Hz |
| 1868-1868.7 | Hz |
| 1897-1897.7 | Hz |
| 2044-2044.7 | Hz |
| 2439-2439.7 | Hz |
| 2612-2612.7 | Hz |
| 2632-2632.7 | Hz |
| 2653.3-2654 | Hz |
| 2686-2686.7 | Hz |
| 2690-2690.7 | Hz |
| 2697-2697.7 | Hz |
| 2738-2738.7 | Hz |
| 3157-3157.7 | Hz |
| 3218-3218.7 | Hz |
| 3227.3-3228 | Hz |
| 3249-3249.7 | Hz |
| 3277.3-3278 | Hz |
| 3281.6-3282.1 | Hz |
| 3436-3436.7 | Hz |
| 3633-3633.7 | Hz |
| 3656-3656.7 | Hz |
| 3962-3962.7 | Hz |
| 4148-4148.7 | Hz |
| 4241-4241.7 | Hz |
| 4410-4410.7 | Hz |
| 4416-4416.7 | Hz |
| 4454-4454.7 | Hz |
| 4619-4619.7 | Hz |
| 4632-4632.7 | Hz |
| 4853-4853.7 | Hz |
| 5139-5139.7 | Hz |
| 5156-5156.7 | Hz |
| 5996-5996.7 | Hz |
| 6292-6292.7 | Hz |
| 6297-6297.7 | Hz |
| 6459-6459.7 | Hz |
| 6474-6474.7 | Hz |
| 6492.3-6493 | Hz |
| 6626.3-6627 | Hz |
| 6727.4-6728 | Hz |
| 6809-6809.6 | Hz |
| 6841-6841.7 | Hz |
| 6915.3-6916 | Hz |
| 6943-6943.7 | Hz |
| 7110-7110.7 | Hz |
| 7182.4-7183 | Hz |
| 7194.3-7195 | Hz |
| 7268-7268.7 | Hz |
| 7323-7323.7 | Hz |
| 7480-7480.7 | Hz |
| 7663-7663.7 | Hz |
| 7796-7796.7 | Hz |
| 7936-7936.7 | Hz |
| 8070-8070.7 | Hz |
| 8128.4-8129 | Hz |
| 8145-8145.7 | Hz |
| 8158-8158.7 | Hz |
| 8314-8314.7 | Hz |
| 8355-8355.7 | Hz |
| 8484-8484.7 | Hz |
| 9138.3-9139 | Hz |
| 9155.7-9156.2 | Hz |
| 9921-9921.7 | Hz |
| 10427-10427.7 | Hz |
| 10468-10468.7 | Hz |
| 10478-10478.7 | Hz |
| 10719-10719.7 | Hz |
| 10802-10802.7 | Hz |
| 10809-10809.7 | Hz |
| 10851-10851.7 | Hz |
| 11556-11556.7 | Hz |
| 11913-11913.7 | Hz |
| 12828.3-12829 | Hz |
| 13686.4-13687 | Hz |
| 14249-14249.7 | Hz |
| 14515.4-14516 | Hz |
| 14586.3-14587 | Hz |
| 14796-14796.7 | Hz |
| 15067-15067.7 | Hz |
| 15112-15112.7 | Hz |
| 17095-17095.7 | Hz |
| 17167-17167.7 | Hz |
| 18090-18090.7 | Hz |
| 18670.3-18671 | Hz |
| 18895-18895.7 | Hz |
| 19026-19026.7 | Hz |
| 20237-20237.7 | Hz |
| 20254-20254.7 | Hz |
| 20286-20286.7 | Hz |
| 24196-24196.7 | Hz |
| 25199-25199.7 | Hz |
| 26073-26073.7 | Hz |
| 26163-26163.7 | Hz |
| 26164-26164.7 | Hz |
| 30759-30759.7 | Hz |
| 32591.3-32592 | Hz |
| 36343-36343.7 | Hz |
| 41883-41883.7 | Hz |
| 44517-44517.7 | Hz |
| 44531-44531.7 | Hz |
| 45490-45490.7 | Hz |
| 47297-47297.7 | Hz |
| 49434-49434.7 | Hz |
| 50229-50229.7 | Hz |
| 69481-69481.7 | Hz |
| 78477.4-78478 | Hz |
| 80993.3-80994 | Hz |
| 81999-81999.7 | Hz |
| 85810-85810.7 | Hz |
| 89748-89748.7 | Hz |
| 92182-92182.7 | Hz |
| 95245-95245.7 | Hz |

### Example 8: Treatment of Squamous Cell Carcinoma of the Anal Canal

Squamous cell carcinoma of the anal canal may be treated according to the methods disclosed herein by the application of squamous cell carcinoma of the anal canal specific amplitude-modulated frequencies. Squamous cell carcinoma of the anal canal specific frequencies are provided below in Table 40.

TABLE 40
Amplitude-modulated frequencies discovered to be effective
for treatment of squamous cell carcinoma of the anal canal.

| | |
|---|---|
| 295-295.6 | Hz |
| 304-304.7 | Hz |
| 436-436.7 | Hz |
| 581-581.7 | Hz |
| 679-679.7 | Hz |
| 694.3-695 | Hz |
| 708.3-709 | Hz |
| 796.3-797 | Hz |
| 1759-1759.7 | Hz |
| 1858-1858.7 | Hz |
| 1873-1873.7 | Hz |
| 2442-2442.7 | Hz |
| 2522-2522.7 | Hz |
| 2648-2648.7 | Hz |
| 2893-2893.7 | Hz |
| 2975-2975.7 | Hz |
| 2981-2981.7 | Hz |
| 3238-3238.7 | Hz |
| 3249.3-3250 | Hz |
| 3250.3-3250.8 | Hz |
| 3461-3461.7 | Hz |
| 3531-3531.7 | Hz |
| 3535-3535.7 | Hz |
| 3681-3681.7 | Hz |
| 4279-4279.7 | Hz |
| 4440.4-4441 | Hz |
| 4718-4718.7 | Hz |
| 4876-4876.7 | Hz |
| 5034-5034.7 | Hz |
| 5068-5068.7 | Hz |
| 5151-5151.7 | Hz |
| 5249-5249.7 | Hz |
| 5286-5286.7 | Hz |
| 5869-5869.7 | Hz |
| 5881-5881.7 | Hz |
| 6298.4-6299 | Hz |
| 6851-6851.7 | Hz |
| 6879-6879.7 | Hz |
| 6882.4-6883 | Hz |
| 6897-6897.7 | Hz |
| 6980.3-6981 | Hz |
| 7136-7136.7 | Hz |
| 7274-7274.7 | Hz |
| 7673-7673.7 | Hz |
| 7930-7930.7 | Hz |
| 7981-7981.7 | Hz |
| 8328-8328.7 | Hz |
| 8432-8432.7 | Hz |
| 8755-8755.7 | Hz |
| 8949-8949.7 | Hz |
| 9656-9656.7 | Hz |
| 10439-10439.7 | Hz |
| 10484-10484.7 | Hz |
| 11145-11145.7 | Hz |
| 11159-11159.7 | Hz |
| 11253-11253.7 | Hz |
| 11260-11260.7 | Hz |
| 11277.3-11278 | Hz |
| 11559-11559.7 | Hz |
| 11699-11699.7 | Hz |
| 11905-11905.7 | Hz |
| 12139-12139.7 | Hz |
| 13819-13819.7 | Hz |
| 13847-13847.7 | Hz |
| 13903-13903.7 | Hz |
| 17379-17379.7 | Hz |
| 18618-18618.7 | Hz |
| 18742-18742.7 | Hz |
| 21445-21445.7 | Hz |
| 21467-21467.7 | Hz |
| 22763-22763.7 | Hz |
| 33536-33536.7 | Hz |
| 37527-37527.7 | Hz |
| 37546-37546.7 | Hz |
| 53079-53079.7 | Hz |
| 58094-58094.7 | Hz |
| 59288-59288.7 | Hz |
| 59997-59997.7 | Hz |
| 88281-88281.7 | Hz |

### Example 9: Treatment of Hepatocellular Carcinoma

Hepatocellular carcinoma may be treated according to the methods disclosed herein by the application of hepatocellular carcinoma specific amplitude-modulated frequencies. Hepatocellular carcinoma specific frequencies are provided below in Table 41.

TABLE 41
Amplitude-modulated frequencies discovered to be effective
for treatment of hepatocellular carcinoma.

| | |
|---|---|
| 439-439.7 | Hz |
| 594-594.7 | Hz |
| 737-737.7 | Hz |
| 817-817.7 | Hz |
| 966-966.7 | Hz |
| 996-996.7 | Hz |
| 1785-1785.7 | Hz |
| 1823-1823.7 | Hz |
| 1881-1881.7 | Hz |
| 1963-1963.7 | Hz |
| 1973-1973.7 | Hz |
| 2189-2189.7 | Hz |
| 2321-2321.7 | Hz |
| 2372.3-2372.8 | Hz |
| 2459-2459.7 | Hz |
| 2647-2647.7 | Hz |
| 2825-2825.7 | Hz |
| 2836-2836.7 | Hz |
| 2860-2860.7 | Hz |
| 2971-2971.6 | Hz |
| 3171-3171.7 | Hz |
| 3188-3188.7 | Hz |
| 3749-3749.7 | Hz |
| 3762-3762.7 | Hz |
| 3823-3823.7 | Hz |
| 4007-4007.7 | Hz |
| 4013-4013.7 | Hz |
| 4454-4454.7 | Hz |
| 4997-4997.7 | Hz |
| 5151-5151.7 | Hz |
| 5239-5239.7 | Hz |
| 5322-5322.7 | Hz |
| 6317-6317.7 | Hz |
| 6330-6330.7 | Hz |
| 6334-6334.7 | Hz |
| 6432-6432.7 | Hz |
| 6840-6840.7 | Hz |
| 7098-7098.7 | Hz |
| 7169-7169.7 | Hz |
| 7239-7239.7 | Hz |
| 7338-7338.7 | Hz |
| 7378-7378.7 | Hz |
| 7481-7481.7 | Hz |
| 7510-7510.7 | Hz |
| 7719-7719.7 | Hz |
| 7748-7748.7 | Hz |
| 7787-7787.7 | Hz |
| 8388-8388.7 | Hz |
| 8412-8412.7 | Hz |
| 8522-8522.7 | Hz |
| 8845-8845.7 | Hz |
| 8935-8935.6 | Hz |
| 8949-8949.7 | Hz |
| 9273-9273.7 | Hz |
| 9588-9588.7 | Hz |
| 9695-9695.7 | Hz |
| 10387-10387.7 | Hz |
| 10641-10641.7 | Hz |
| 10731-10731.7 | Hz |
| 10740-10740.7 | Hz |
| 10762-10762.7 | Hz |
| 10768-10768.7 | Hz |
| 10776-10776.7 | Hz |
| 10805-10805.7 | Hz |
| 10831-10831.7 | Hz |
| 10889-10889.7 | Hz |
| 11209-11209.7 | Hz |
| 11243-11243.7 | Hz |
| 11247-11247.7 | Hz |
| 11275-11275.7 | Hz |
| 11295-11295.7 | Hz |
| 11569-11569.7 | Hz |
| 11590-11590.7 | Hz |
| 11946-11946.7 | Hz |
| 12498-12498.7 | Hz |
| 12572-12572.7 | Hz |
| 12649-12649.7 | Hz |
| 13215-13215.7 | Hz |
| 13252-13252.7 | Hz |
| 13735-13735.7 | Hz |
| 13856-13856.7 | Hz |
| 14114-14114.7 | Hz |
| 14170.7-14171.3 | Hz |
| 14532-14532.7 | Hz |
| 14574-14574.7 | Hz |
| 15521-15521.7 | Hz |
| 15528-15528.7 | Hz |
| 15546.3-15547 | Hz |
| 15589-15589.7 | Hz |
| 15982-15982.7 | Hz |
| 16381-16381.7 | Hz |
| 17556-17556.7 | Hz |
| 17749-17749.7 | Hz |
| 17978-17978.7 | Hz |
| 18030-18030.7 | Hz |
| 18246-18246.7 | Hz |
| 18567-18567.7 | Hz |
| 18686-18686.7 | Hz |
| 18842-18842.7 | Hz |
| 18897-18897.7 | Hz |
| 19510-19510.7 | Hz |
| 20324-20324.7 | Hz |
| 20825-20825.7 | Hz |
| 20908-20908.7 | Hz |
| 21616-21616.7 | Hz |
| 21778-21778.7 | Hz |
| 21876-21876.7 | Hz |
| 22353-22353.7 | Hz |
| 22459-22459.7 | Hz |
| 22535-22535.7 | Hz |
| 22709-22709.7 | Hz |
| 23509-23509.7 | Hz |
| 23526-23526.7 | Hz |
| 23560-23560.7 | Hz |
| 23589-23589.7 | Hz |
| 23957-23957.7 | Hz |
| 24014-24014.7 | Hz |
| 24117-24117.7 | Hz |
| 24869-24869.7 | Hz |
| 25449-25449.7 | Hz |
| 25454-25454.7 | Hz |
| 26722-26722.7 | Hz |
| 26861-26861.7 | Hz |
| 30942-30942.7 | Hz |
| 31713-31713.7 | Hz |
| 32866-32866.7 | Hz |
| 32870-32870.7 | Hz |
| 33765-33765.7 | Hz |
| 34405-34405.7 | Hz |
| 34562-34562.7 | Hz |
| 34984-34984.6 | Hz |
| 36410-36410.7 | Hz |
| 38483-38483.7 | Hz |
| 38659-38659.7 | Hz |
| 39035-39035.7 | Hz |
| 39223-39223.7 | Hz |
| 39510-39510.7 | Hz |
| 39729-39729.7 | Hz |
| 40693-40693.7 | Hz |
| 41244-41244.7 | Hz |
| 41574-41574.7 | Hz |
| 42913-42913.7 | Hz |
| 42923-42923.7 | Hz |
| 43243-43243.7 | Hz |
| 43413-43413.7 | Hz |
| 43542-43542.7 | Hz |
| 43603-43603.7 | Hz |
| 44742-44742.7 | Hz |
| 47693.4-47694 | Hz |
| 51592-51592.7 | Hz |
| 53947-53947.7 | Hz |
| 55072-55072.7 | Hz |
| 56093-56093.7 | Hz |
| 59089-59089.7 | Hz |
| 66969-66969.7 | Hz |
| 67683-67683.7 | Hz |
| 76537-76537.7 | Hz |
| 78229-78229.7 | Hz |
| 82845-82845.7 | Hz |
| 83612-83612.7 | Hz |
| 84086-84086.7 | Hz |
| 91245-91245.7 | Hz |
| 92361.3-92362 | Hz |
| 93332-93332.7 | Hz |
| 93530-93530.7 | Hz |
| 94968.3-94969 | Hz |
| 99281-99281.7 | Hz |
| 99846-99846.7 | Hz |

### Example 10: Treatment of Cholangiocarcinoma

Cholangiocarcinoma may be treated according to the methods disclosed herein by the application of cholangiocarcinoma specific amplitude-modulated frequencies. Cholangiocarcinoma specific frequencies are provided below in Table 42.

TABLE 42
Amplitude-modulated frequencies discovered to be
effective for treatment of cholangiocarcinoma

| | |
|---|---|
| 334-334.7 | Hz |
| 380-380.7 | Hz |
| 410-410.7 | Hz |
| 427-427.6 | Hz |
| 434-434.7 | Hz |
| 476-476.7 | Hz |
| 527-527.7 | Hz |
| 560-560.7 | Hz |
| 624-624.7 | Hz |
| 642.4-643 | Hz |
| 657-657.7 | Hz |
| 677.4-678 | Hz |
| 728-728.7 | Hz |
| 806-806.6 | Hz |
| 809-809.7 | Hz |
| 845-845.7 | Hz |
| 920-920.7 | Hz |
| 961-961.7 | Hz |
| 963.8-964.25 | Hz |
| 964.3-964.8 | Hz |
| 974.4-975 | Hz |
| 1156.3-1157 | Hz |
| 1157-1157.7 | Hz |
| 1755-1755.7 | Hz |
| 1873-1873.7 | Hz |
| 1967-1967.7 | Hz |
| 2017.4-2018 | Hz |
| 2021-2021.7 | Hz |
| 2023-2023.7 | Hz |
| 2035-2035.7 | Hz |
| 2053-2053.7 | Hz |
| 2065-2065.7 | Hz |
| 2083-2083.7 | Hz |
| 2094-2094.7 | Hz |
| 2111-2111.7 | Hz |
| 2128-2128.7 | Hz |
| 2278-2278.7 | Hz |
| 2284-2284.7 | Hz |
| 2308-2308.7 | Hz |
| 2324-2324.7 | Hz |
| 2338-2338.7 | Hz |
| 2362-2362.7 | Hz |
| 2381-2381.7 | Hz |
| 2419-2419.7 | Hz |
| 2425-2425.7 | Hz |
| 2430-2430.7 | Hz |
| 2431-2431.6 | Hz |
| 2471-2471.7 | Hz |
| 2480-2480.7 | Hz |
| 2522-2522.7 | Hz |
| 2636-2636.7 | Hz |
| 2669-2669.7 | Hz |
| 2679-2679.7 | Hz |
| 2685-2685.6 | Hz |
| 2716-2716.6 | Hz |
| 2743.4-2744 | Hz |
| 2835-2835.7 | Hz |
| 2854-2854.7 | Hz |
| 2865-2865.7 | Hz |
| 2873.3-2874 | Hz |
| 2886-2886.7 | Hz |
| 3009-3009.7 | Hz |
| 3020-3020.7 | Hz |
| 3042-3042.6 | Hz |
| 3076.3-3077 | Hz |
| 3104.3-3105 | Hz |
| 3135-3135.7 | Hz |
| 3143-3143.7 | Hz |
| 3160.4-3161 | Hz |
| 3167-3167.7 | Hz |
| 3206-3206.7 | Hz |
| 3255-3255.7 | Hz |
| 3267-3267.7 | Hz |
| 3269-3269.7 | Hz |
| 3330.4-3331 | Hz |
| 3516-3516.7 | Hz |
| 3531-3531.7 | Hz |
| 3549-3549.7 | Hz |
| 3564-3564.7 | Hz |
| 3572-3572.7 | Hz |
| 3576-3576.7 | Hz |
| 3619-3619.7 | Hz |
| 3630-3630.7 | Hz |
| 3637.7-3638.3 | Hz |
| 3644-3644.7 | Hz |
| 3696-3696.7 | Hz |
| 3749.3-3750 | Hz |
| 3947-3947.7 | Hz |
| 3954-3954.7 | Hz |
| 4013.4-4014 | Hz |
| 4079.4-4080 | Hz |
| 4123.4-4124 | Hz |
| 4281-4281.7 | Hz |
| 4289-4289.7 | Hz |
| 4312.4-4313 | Hz |
| 4418-4418.7 | Hz |
| 4426-4426.7 | Hz |
| 4441-4441.7 | Hz |
| 4685-4685.6 | Hz |
| 4707-4707.7 | Hz |
| 4715-4715.7 | Hz |
| 4732-4732.7 | Hz |
| 4732-4732.7 | Hz |
| 4748-4748.7 | Hz |
| 4767-4767.7 | Hz |
| 4812-4812.7 | Hz |
| 4821-4821.7 | Hz |
| 4873-4873.7 | Hz |
| 4874-4874.7 | Hz |
| 4876-4876.7 | Hz |
| 4941-4941.7 | Hz |
| 5209.4-5210 | Hz |
| 5229-5229.7 | Hz |
| 5243-5243.7 | Hz |
| 5247-5247.7 | Hz |
| 5262-5262.7 | Hz |
| 5271-5271.7 | Hz |
| 5387.3-5388 | Hz |
| 5573-5573.7 | Hz |
| 5671-5671.7 | Hz |
| 5679-5679.7 | Hz |
| 5966-5966.7 | Hz |
| 5976.3-5977 | Hz |
| 6064-6064.7 | Hz |
| 6086-6086.7 | Hz |
| 6180-6180.7 | Hz |
| 6479.4-6480 | Hz |
| 6513-6513.7 | Hz |
| 6711-6711.7 | Hz |
| 6779-6779.7 | Hz |
| 6915.3-6916 | Hz |
| 7019-7019.7 | Hz |
| 7043-7043.7 | Hz |
| 7130-7130.7 | Hz |
| 7213-7213.7 | Hz |
| 7228.3-7229 | Hz |
| 7234-7234.7 | Hz |
| 7246-7246.7 | Hz |
| 7268-7268.7 | Hz |
| 7367-7367.7 | Hz |
| 7482-7482.7 | Hz |
| 7510.4-7511 | Hz |
| 7680.3-7681 | Hz |
| 7862-7862.7 | Hz |
| 7935-7935.7 | Hz |
| 8012.3-8013 | Hz |
| 8013-8013.7 | Hz |
| 8026-8026.7 | Hz |
| 8028-8028.7 | Hz |
| 8028-8028.7 | Hz |
| 8055.4-8056 | Hz |
| 8072-8072.7 | Hz |
| 8074-8074.7 | Hz |
| 8079-8079.7 | Hz |
| 8141-8141.7 | Hz |
| 8336-8336.7 | Hz |
| 8394.3-8395 | Hz |
| 8421-8421.7 | Hz |
| 8432-8432.7 | Hz |
| 8452-8452.7 | Hz |
| 8492-8492.7 | Hz |
| 8509.4-8510 | Hz |
| 8542-8542.7 | Hz |
| 8779-8779.7 | Hz |
| 8789.3-8790 | Hz |
| 8818-8818.7 | Hz |
| 8852-8852.7 | Hz |
| 8993-8993.7 | Hz |
| 9023-9023.7 | Hz |
| 9112-9112.7 | Hz |
| 9332-9332.7 | Hz |
| 9753-9753.7 | Hz |
| 9846-9846.7 | Hz |
| 10140-10140.7 | Hz |
| 10317-10317.7 | Hz |
| 10362-10362.7 | Hz |
| 10488-10488.7 | Hz |
| 10661-10661.7 | Hz |
| 10665.4-10666 | Hz |
| 10690-10690.7 | Hz |
| 10866-10866.7 | Hz |
| 11081-11081.7 | Hz |
| 11109.3-11110 | Hz |
| 11219-11219.7 | Hz |
| 11582-11582.7 | Hz |
| 11619-11619.7 | Hz |
| 11637-11637.7 | Hz |
| 11802.3-11803 | Hz |
| 11809-11809.7 | Hz |
| 11831-11831.7 | Hz |
| 11872-11872.7 | Hz |
| 11904.3-11905 | Hz |
| 12024.3-12025 | Hz |
| 12228-12228.7 | Hz |
| 12247-12247.7 | Hz |
| 12260.4-12261 | Hz |
| 12274-12274.7 | Hz |
| 12682-12682.7 | Hz |
| 12752-12752.7 | Hz |
| 12783-12783.7 | Hz |
| 12785-12785.7 | Hz |
| 13127-13127.7 | Hz |
| 13457-13457.7 | Hz |
| 13609-13609.7 | Hz |
| 13734-13734.7 | Hz |
| 13747-13747.7 | Hz |
| 13755-13755.7 | Hz |
| 13760-13760.7 | Hz |
| 13761-13761.7 | Hz |
| 13767-13767.7 | Hz |
| 13981-13981.7 | Hz |
| 14005-14005.7 | Hz |
| 14027-14027.7 | Hz |
| 14070-14070.7 | Hz |
| 14078-14078.7 | Hz |
| 14356-14356.7 | Hz |
| 14531-14531.7 | Hz |
| 14545-14545.7 | Hz |
| 14564-14564.7 | Hz |
| 14828-14828.7 | Hz |
| 14867-14867.7 | Hz |
| 14913.4-14914 | Hz |
| 14940-14940.7 | Hz |
| 15173-15173.7 | Hz |
| 15224-15224.7 | Hz |
| 15244-15244.7 | Hz |
| 15432-15432.7 | Hz |
| 15460-15460.7 | Hz |
| 15515-15515.7 | Hz |
| 15539-15539.7 | Hz |
| 15550.4-15551 | Hz |
| 15857-15857.7 | Hz |
| 16082-16082.7 | Hz |
| 16515-16515.7 | Hz |
| 17636-17636.7 | Hz |
| 17673-17673.7 | Hz |
| 18087-18087.7 | Hz |
| 18222-18222.7 | Hz |
| 18521-18521.7 | Hz |
| 18585-18585.7 | Hz |
| 19347-19347.7 | Hz |
| 19889-19889.7 | Hz |
| 21466-21466.7 | Hz |
| 21567-21567.7 | Hz |
| 21844-21844.7 | Hz |
| 21870-21870.7 | Hz |
| 21894-21894.7 | Hz |
| 22073-22073.7 | Hz |
| 22119-22119.7 | Hz |
| 22411-22411.7 | Hz |
| 22437-22437.7 | Hz |
| 22479-22479.7 | Hz |
| 22582-22582.7 | Hz |
| 22730-22730.6 | Hz |
| 22832-22832.7 | Hz |
| 22863-22863.7 | Hz |
| 23237-23237.7 | Hz |
| 23261-23261.7 | Hz |
| 23444-23444.7 | Hz |
| 26212-26212.7 | Hz |
| 27291-27291.7 | Hz |
| 31051-31051.7 | Hz |
| 35296-35296.7 | Hz |
| 37680-37680.7 | Hz |
| 39053-39053.7 | Hz |
| 39183-39183.7 | Hz |
| 39423-39423.7 | Hz |
| 40111-40111.7 | Hz |
| 41557-41557.7 | Hz |
| 42293-42293.7 | Hz |
| 43172-43172.7 | Hz |
| 43379-43379.7 | Hz |
| 44855-44855.7 | Hz |
| 45163-45163.7 | Hz |
| 45362-45362.7 | Hz |
| 46666-46666.7 | Hz |
| 46673-46673.7 | Hz |
| 46689-46689.6 | Hz |
| 51008-51008.7 | Hz |
| 52063-52063.7 | Hz |
| 52786-52786.7 | Hz |
| 52912-52912.7 | Hz |
| 55723-55723.7 | Hz |
| 61287-61287.7 | Hz |
| 61789-61789.7 | Hz |
| 71635-71635.7 | Hz |
| 86521-86521.7 | Hz |
| 87798.3-87799 | Hz |
| 88898-88898.7 | Hz |
| 91271-91271.7 | Hz |
| 92787-92787.7 | Hz |

### Example 11: Treatment of Mesothelioma

Mesothelioma may be treated according to the methods disclosed herein by the application of mesothelioma specific amplitude-modulated frequencies. Mesothelioma specific frequencies are provided below in Table 43.

TABLE 43
Amplitude-modulated frequencies discovered to
be effective for treatment of mesothelioma.

| | |
|---|---|
| 262-262.7 | Hz |
| 412-412.7 | Hz |
| 418-418.7 | Hz |
| 429-429.7 | Hz |
| 441-441.7 | Hz |
| 442-442.7 | Hz |
| 465-465.7 | Hz |
| 485-485.7 | Hz |
| 517-517.7 | Hz |
| 556-556.7 | Hz |
| 581-581.7 | Hz |
| 612-612.7 | Hz |
| 626-626.7 | Hz |
| 638-638.7 | Hz |
| 664-664.7 | Hz |
| 694.3-695 | Hz |
| 708.3-709 | Hz |
| 734.4-735 | Hz |
| 761-761.7 | Hz |
| 764-764.7 | Hz |
| 776-776.7 | Hz |
| 806-806.6 | Hz |
| 831-831.7 | Hz |
| 883-883.7 | Hz |
| 1191-1191.7 | Hz |
| 1591-1591.7 | Hz |
| 2475.4-2476 | Hz |
| 2581-2581.7 | Hz |
| 2749-2749.7 | Hz |
| 2988-2988.7 | Hz |
| 3042-3042.6 | Hz |
| 3131-3131.7 | Hz |
| 3193-3193.7 | Hz |
| 3240-3240.7 | Hz |
| 3335-3335.7 | Hz |
| 3371-3371.7 | Hz |
| 3518-3518.7 | Hz |
| 3523-3523.7 | Hz |
| 3530-3530.7 | Hz |
| 3556-3556.7 | Hz |
| 3621-3621.7 | Hz |
| 3656-3656.7 | Hz |
| 3674-3674.7 | Hz |
| 3681.7-3682.3 | Hz |
| 3817-3817.7 | Hz |
| 3833-3833.7 | Hz |
| 3877-3877.7 | Hz |
| 3948-3948.7 | Hz |
| 3958-3958.6 | Hz |
| 4044-4044.7 | Hz |
| 4190-4190.7 | Hz |
| 4261-4261.7 | Hz |
| 4330-4330.7 | Hz |
| 5496-5496.7 | Hz |
| 5640.4-5641 | Hz |
| 6416.3-6417 | Hz |
| 6441-6441.7 | Hz |
| 6717-6717.7 | Hz |
| 6758-6758.7 | Hz |
| 6783-6783.7 | Hz |
| 7110-7110.7 | Hz |
| 7140-7140.7 | Hz |
| 7154-7154.7 | Hz |
| 7156-7156.7 | Hz |
| 7253-7253.7 | Hz |
| 7283-7283.7 | Hz |
| 7546-7546.7 | Hz |
| 7725-7725.7 | Hz |
| 8386-8386.7 | Hz |
| 8562.4-8563 | Hz |
| 8810-8810.7 | Hz |
| 8827.4-8828 | Hz |
| 8843-8843.7 | Hz |
| 8859.3-8860 | Hz |
| 8863-8863.7 | Hz |
| 8877-8877.7 | Hz |
| 8881-8881.7 | Hz |
| 9075-9075.7 | Hz |
| 9929-9929.7 | Hz |
| 9947-9947.7 | Hz |
| 9970-9970.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11083-11083.7 | Hz |
| 11124-11124.7 | Hz |
| 11540-11540.7 | Hz |
| 11831-11831.7 | Hz |
| 11929-11929.7 | Hz |
| 11956-11956.7 | Hz |
| 12267-12267.7 | Hz |
| 12929-12929.7 | Hz |
| 13674-13674.7 | Hz |
| 13742-13742.7 | Hz |
| 14285-14285.7 | Hz |
| 14947-14947.7 | Hz |
| 15531-15531.7 | Hz |
| 17737-17737.7 | Hz |
| 17757-17757.7 | Hz |
| 18121-18121.7 | Hz |
| 18126-18126.7 | Hz |
| 18140-18140.7 | Hz |
| 18921-18921.7 | Hz |
| 19406-19406.7 | Hz |
| 19476-19476.7 | Hz |
| 19577-19577.7 | Hz |
| 20634-20634.7 | Hz |
| 21372-21372.7 | Hz |
| 22411-22411.7 | Hz |
| 22519-22519.7 | Hz |
| 22538-22538.7 | Hz |
| 22548-22548.7 | Hz |
| 23111-23111.7 | Hz |
| 23921-23921.7 | Hz |
| 24112-24112.7 | Hz |
| 24146-24146.7 | Hz |
| 39153-39153.7 | Hz |

### Example 12: Treatment of Thyroid Cancer

Thyroid cancer may be treated according to the methods disclosed herein by the application of thyroid cancer specific amplitude-modulated frequencies. Thyroid cancer specific frequencies are provided below in Table 44.

TABLE 44
Amplitude-modulated frequencies discovered to
be effective for treatment of thyroid cancer.

| | |
|---|---|
| 834-834.7 | Hz |
| 836-836.7 | Hz |
| 845-845.7 | Hz |
| 1213-1213.7 | Hz |
| 1220-1220.7 | Hz |
| 1252-1252.7 | Hz |
| 1815-1815.7 | Hz |
| 1966-1966.7 | Hz |
| 2050-2050.7 | Hz |
| 2313-2313.7 | Hz |
| 2318-2318.7 | Hz |
| 2320-2320.7 | Hz |
| 2328-2328.7 | Hz |
| 2336-2336.7 | Hz |
| 2359-2359.7 | Hz |
| 2373-2373.7 | Hz |
| 2380-2380.7 | Hz |
| 2425-2425.7 | Hz |
| 2429-2429.7 | Hz |
| 2436-2436.7 | Hz |
| 2513-2513.7 | Hz |
| 2629-2629.7 | Hz |
| 2653.7-2654.4 | Hz |
| 2830-2830.7 | Hz |
| 2834-2834.7 | Hz |
| 3030-3030.7 | Hz |
| 3056-3056.7 | Hz |
| 3609-3609.7 | Hz |
| 3646-3646.7 | Hz |
| 3674-3674.7 | Hz |
| 3694-3694.7 | Hz |
| 3908.7-3909.22 | Hz |
| 3981-3981.7 | Hz |
| 4027-4027.7 | Hz |
| 4386.4-4387 | Hz |
| 4425.7-4426.22 | Hz |
| 4792-4792.7 | Hz |
| 4844-4844.7 | Hz |
| 4870-4870.7 | Hz |
| 5259-5259.7 | Hz |
| 5510-5510.7 | Hz |
| 5533-5533.7 | Hz |
| 5714-5714.7 | Hz |
| 5741-5741.7 | Hz |
| 5742-5742.7 | Hz |
| 5752-5752.7 | Hz |
| 5766-5766.7 | Hz |
| 6392-6392.7 | Hz |
| 6423-6423.7 | Hz |
| 6781-6781.7 | Hz |
| 6832-6832.7 | Hz |
| 6864-6864.7 | Hz |
| 7404.4-7405 | Hz |
| 7526-7526.6 | Hz |
| 7646-7646.7 | Hz |
| 7652-7652.7 | Hz |
| 7913-7913.7 | Hz |
| 8217.4-8218 | Hz |
| 8251-8251.7 | Hz |
| 8347-8347.7 | Hz |
| 8731-8731.7 | Hz |
| 8740-8740.7 | Hz |
| 8749-8749.7 | Hz |
| 8814-8814.7 | Hz |
| 9169-9169.7 | Hz |
| 9506-9506.7 | Hz |
| 10159-10159.7 | Hz |
| 10395-10395.7 | Hz |
| 10541-10541.7 | Hz |
| 10830-10830.7 | Hz |
| 10844-10844.7 | Hz |
| 10878-10878.7 | Hz |
| 10993-10993.7 | Hz |
| 11117-11117.7 | Hz |
| 11779-11779.7 | Hz |
| 11867-11867.7 | Hz |
| 12302-12302.7 | Hz |
| 12356-12356.7 | Hz |
| 13117-13117.7 | Hz |
| 13373-13373.7 | Hz |
| 13679-13679.7 | Hz |
| 13694-13694.7 | Hz |
| 14111-14111.7 | Hz |
| 14116-14116.7 | Hz |
| 14156-14156.7 | Hz |
| 14174-14174.7 | Hz |
| 14281-14281.7 | Hz |
| 14523-14523.7 | Hz |
| 14585-14585.7 | Hz |
| 14690-14690.7 | Hz |
| 14716-14716.7 | Hz |
| 14912-14912.7 | Hz |
| 14974-14974.7 | Hz |
| 15223-15223.7 | Hz |
| 15236-15236.7 | Hz |
| 15329-15329.7 | Hz |
| 15615-15615.7 | Hz |
| 15867-15867.7 | Hz |
| 15919-15919.7 | Hz |
| 15942-15942.7 | Hz |
| 16096-16096.7 | Hz |
| 18040-18040.7 | Hz |
| 18182-18182.7 | Hz |
| 18211-18211.7 | Hz |
| 18246-18246.7 | Hz |
| 18274-18274.7 | Hz |
| 18411-18411.7 | Hz |
| 18454-18454.7 | Hz |
| 18462-18462.7 | Hz |
| 18596-18596.7 | Hz |
| 19365-19365.7 | Hz |
| 19419-19419.7 | Hz |
| 19715-19715.7 | Hz |
| 19744-19744.7 | Hz |
| 19823-19823.7 | Hz |
| 20229-20229.7 | Hz |
| 20284-20284.7 | Hz |
| 20513-20513.7 | Hz |
| 20747-20747.7 | Hz |
| 21963-21963.7 | Hz |
| 22338-22338.7 | Hz |
| 22344-22344.7 | Hz |
| 22350-22350.7 | Hz |
| 22422-22422.7 | Hz |
| 22484-22484.7 | Hz |
| 22932-22932.7 | Hz |
| 23012-23012.7 | Hz |
| 23044-23044.7 | Hz |
| 23216-23216.7 | Hz |
| 23659-23659.7 | Hz |
| 23730-23730.7 | Hz |
| 24060-24060.7 | Hz |
| 24085-24085.7 | Hz |
| 26044-26044.7 | Hz |
| 26343-26343.7 | Hz |
| 27214-27214.7 | Hz |
| 28361-28361.7 | Hz |
| 33366-33366.7 | Hz |
| 33414-33414.7 | Hz |
| 33553-33553.7 | Hz |
| 34614-34614.7 | Hz |
| 35826-35826.7 | Hz |
| 40133-40133.7 | Hz |
| 40394-40394.7 | Hz |
| 40896-40896.7 | Hz |
| 40899-40899.7 | Hz |
| 41424-41424.7 | Hz |
| 41534-41534.7 | Hz |
| 41859-41859.7 | Hz |
| 41861-41861.7 | Hz |
| 42402-42402.7 | Hz |
| 43851-43851.7 | Hz |
| 44162-44162.7 | Hz |
| 44692-44692.7 | Hz |
| 45393-45393.7 | Hz |
| 45526-45526.7 | Hz |
| 46282-46282.7 | Hz |
| 46285-46285.7 | Hz |
| 47036-47036.7 | Hz |
| 48314-48314.7 | Hz |
| 48535-48535.7 | Hz |
| 52097-52097.7 | Hz |
| 54293-54293.7 | Hz |
| 54297-54297.7 | Hz |
| 55592-55592.7 | Hz |
| 57927-57927.7 | Hz |
| 59907-59907.7 | Hz |
| 65586-65586.7 | Hz |
| 67697-67697.6 | Hz |
| 71204-71204.7 | Hz |
| 71447-71447.7 | Hz |
| 74017-74017.7 | Hz |
| 76227-76227.7 | Hz |
| 76659-76659.7 | Hz |
| 82024-82024.7 | Hz |

### Example 13: Treatment of Prostate Cancer

Prostate cancer may be treated according to the methods disclosed herein by the application of prostate cancer specific amplitude-modulated frequencies. Prostate cancer specific frequencies are provided below in Table 45.

TABLE 45
Amplitude-modulated frequencies discovered to
be effective for treatment of prostate cancer.

| | |
|---|---|
| 408-408.7 | Hz |
| 429.7-430.3 | Hz |
| 561-561.7 | Hz |
| 568-568.7 | Hz |
| 572-572.7 | Hz |
| 631-631.7 | Hz |
| 709-709.7 | Hz |
| 717-717.7 | Hz |
| 733-733.7 | Hz |
| 810-810.7 | Hz |
| 817-817.7 | Hz |
| 830-830.7 | Hz |
| 1668-1668.7 | Hz |
| 1808-1808.6 | Hz |
| 2483-2483.7 | Hz |
| 2518-2518.7 | Hz |
| 2824-2824.7 | Hz |
| 3207-3207.7 | Hz |
| 3245-3245.7 | Hz |
| 3251-3251.7 | Hz |
| 3266.3-3267 | Hz |
| 3279.3-3280 | Hz |
| 3529-3529.7 | Hz |
| 3562-3562.7 | Hz |
| 3571-3571.7 | Hz |
| 3627-3627.7 | Hz |
| 3661-3661.7 | Hz |
| 3731-3731.7 | Hz |
| 3743-3743.7 | Hz |
| 3756-3756.7 | Hz |
| 3787-3787.7 | Hz |
| 3962-3962.7 | Hz |
| 4152-4152.7 | Hz |
| 4409-4409.7 | Hz |
| 4476-4476.7 | Hz |
| 4806-4806.7 | Hz |
| 4809-4809.7 | Hz |
| 4823-4823.7 | Hz |
| 4841-4841.7 | Hz |
| 4948-4948.7 | Hz |
| 4998-4998.7 | Hz |
| 5336-5336.7 | Hz |
| 5414-5414.7 | Hz |
| 5884-5884.7 | Hz |
| 6117-6117.7 | Hz |
| 6252-6252.7 | Hz |
| 6329-6329.7 | Hz |
| 6333-6333.7 | Hz |
| 6748-6748.7 | Hz |
| 6782-6782.7 | Hz |
| 7112-7112.7 | Hz |
| 7424-7424.7 | Hz |
| 7655-7655.7 | Hz |
| 7906-7906.7 | Hz |
| 7910-7910.7 | Hz |
| 7923-7923.7 | Hz |
| 8373-8373.7 | Hz |
| 8445-8445.7 | Hz |
| 8765-8765.7 | Hz |
| 8829-8829.7 | Hz |
| 8852-8852.7 | Hz |
| 8877-8877.7 | Hz |
| 9078-9078.7 | Hz |
| 9512-9512.7 | Hz |
| 9635-9635.7 | Hz |
| 9681-9681.7 | Hz |
| 9886-9886.7 | Hz |
| 10735-10735.7 | Hz |
| 10748-10748.7 | Hz |
| 10781-10781.7 | Hz |
| 10813.2-10813.8 | Hz |
| 11134-11134.7 | Hz |
| 11155-11155.7 | Hz |
| 11164-11164.7 | Hz |
| 11264-11264.7 | Hz |
| 11381-11381.7 | Hz |
| 11416-11416.7 | Hz |
| 11438-11438.7 | Hz |
| 11469-11469.7 | Hz |
| 11628-11628.7 | Hz |
| 12330-12330.7 | Hz |
| 12391-12391.7 | Hz |
| 12880-12880.7 | Hz |
| 13467-13467.7 | Hz |
| 13562-13562.6 | Hz |
| 13805-13805.7 | Hz |
| 13828-13828.7 | Hz |
| 13834-13834.7 | Hz |
| 13857.4-13858 | Hz |
| 13858-13858.7 | Hz |
| 13871-13871.7 | Hz |
| 14009-14009.7 | Hz |
| 14034-14034.7 | Hz |
| 14153-14153.7 | Hz |
| 14489-14489.7 | Hz |
| 15071-15071.7 | Hz |
| 15149-15149.7 | Hz |
| 15233-15233.7 | Hz |
| 15253-15253.7 | Hz |
| 15263-15263.7 | Hz |
| 15435-15435.7 | Hz |
| 15545-15545.7 | Hz |
| 16176-16176.7 | Hz |
| 16329-16329.7 | Hz |
| 16679-16679.7 | Hz |
| 16804-16804.7 | Hz |
| 16821-16821.7 | Hz |
| 17333-17333.7 | Hz |
| 17435-17435.7 | Hz |
| 17974-17974.7 | Hz |
| 18260-18260.7 | Hz |
| 18641-18641.7 | Hz |
| 18831-18831.7 | Hz |
| 18884-18884.7 | Hz |
| 19453-19453.7 | Hz |
| 20948-20948.7 | Hz |
| 21119-21119.7 | Hz |
| 21167-21167.7 | Hz |
| 21636-21636.7 | Hz |
| 22312-22312.7 | Hz |
| 22361-22361.7 | Hz |
| 22419-22419.7 | Hz |
| 22430-22430.7 | Hz |
| 22443-22443.7 | Hz |
| 22459-22459.7 | Hz |
| 22879-22879.7 | Hz |
| 23117-23117.7 | Hz |
| 23137-23137.7 | Hz |
| 23412-23412.7 | Hz |
| 23548-23548.7 | Hz |
| 23686-23686.7 | Hz |
| 26142-26142.7 | Hz |
| 26465-26465.7 | Hz |
| 27328-27328.6 | Hz |
| 28653-28653.7 | Hz |
| 28744-28744.7 | Hz |
| 31572-31572.7 | Hz |
| 31633-31633.7 | Hz |
| 41062-41062.7 | Hz |
| 41325-41325.7 | Hz |
| 41473-41473.7 | Hz |
| 48683-48683.7 | Hz |
| 48793-48793.7 | Hz |
| 87507-87507.7 | Hz |

### Example 14: Treatment of Rhabdomyo Sarcoma

Rhabdomyo sarcoma may be treated according to the methods disclosed herein by the application of rhabdomyo sarcoma specific amplitude-modulated frequencies. Rhabdomyosarcoma specific frequencies are provided below in Table 46.

TABLE 46
Amplitude-modulated frequencies discovered to
be effective for treatment of rhabdomyosarcoma.

| | |
|---|---|
| 266.3-267 | Hz |
| 304-304.7 | Hz |
| 331-331.7 | Hz |
| 410-410.7 | Hz |
| 420-420.7 | Hz |
| 429-429.7 | Hz |
| 434-434.7 | Hz |
| 461-461.7 | Hz |
| 537.4-538 | Hz |
| 560-560.7 | Hz |
| 572-572.7 | Hz |
| 605-605.7 | Hz |
| 611-611.7 | Hz |
| 618.3-619 | Hz |
| 626-626.7 | Hz |
| 717-717.7 | Hz |
| 776-776.7 | Hz |
| 779-779.7 | Hz |
| 810-810.7 | Hz |
| 814-814.7 | Hz |
| 826-826.7 | Hz |
| 836.4-837 | Hz |
| 843-843.7 | Hz |
| 843-843.7 | Hz |
| 1314-1314.7 | Hz |
| 1411-1411.7 | Hz |
| 1467-1467.7 | Hz |
| 1785-1785.7 | Hz |
| 1816-1816.7 | Hz |
| 1903-1903.7 | Hz |
| 2073.3-2074 | Hz |
| 2155-2155.7 | Hz |
| 2287-2287.7 | Hz |
| 2381-2381.7 | Hz |
| 2542-2542.7 | Hz |
| 2711-2711.6 | Hz |
| 2854-2854.6 | Hz |
| 2911-2911.7 | Hz |
| 3085.7-3086.3 | Hz |
| 3121-3121.7 | Hz |
| 3232-3232.7 | Hz |
| 3438-3438.7 | Hz |
| 3518-3518.7 | Hz |
| 3544-3544.7 | Hz |
| 3569-3569.7 | Hz |
| 3618-3618.7 | Hz |
| 3910-3910.7 | Hz |
| 3917-3917.7 | Hz |
| 4198-4198.7 | Hz |
| 4233.3-4234 | Hz |
| 4241-4241.7 | Hz |
| 4337-4337.7 | Hz |
| 4358-4358.7 | Hz |
| 4424-4424.7 | Hz |
| 4436-4436.7 | Hz |
| 4485-4485.7 | Hz |
| 4731-4731.7 | Hz |
| 4749-4749.7 | Hz |
| 4776-4776.7 | Hz |
| 5151-5151.7 | Hz |
| 5521-5521.7 | Hz |
| 5545.3-5546 | Hz |
| 5577.3-5578 | Hz |
| 5618-5618.7 | Hz |
| 5631-5631.7 | Hz |
| 5696-5696.7 | Hz |
| 6058-6058.7 | Hz |
| 6443-6443.7 | Hz |
| 6472-6472.6 | Hz |
| 6558-6558.7 | Hz |
| 6651-6651.7 | Hz |
| 6730-6730.7 | Hz |
| 7168.4-7169 | Hz |
| 7406-7406.7 | Hz |
| 7440-7440.7 | Hz |
| 7452.3-7453 | Hz |
| 7649-7649.7 | Hz |
| 7808-7808.7 | Hz |
| 8542-8542.7 | Hz |
| 8546-8546.7 | Hz |
| 8562-8562.7 | Hz |
| 8576-8576.7 | Hz |
| 8698-8698.7 | Hz |
| 8881-8881.7 | Hz |
| 9040-9040.7 | Hz |
| 9074-9074.7 | Hz |
| 9189-9189.6 | Hz |
| 9484.3-9485 | Hz |
| 9851-9851.7 | Hz |
| 9943.4-9944 | Hz |
| 10870-10870.7 | Hz |
| 10891-10891.7 | Hz |
| 12086-12086.7 | Hz |
| 12648-12648.7 | Hz |
| 15195-15195.7 | Hz |
| 17316-17316.7 | Hz |
| 18021-18021.7 | Hz |
| 18567-18567.7 | Hz |
| 19384-19384.7 | Hz |
| 20097-20097.7 | Hz |
| 20278.4-20279 | Hz |
| 21854-21854.7 | Hz |
| 21890-21890.7 | Hz |
| 24198-24198.7 | Hz |
| 24413-24413.7 | Hz |
| 24789-24789.7 | Hz |
| 26534-26534.7 | Hz |
| 27713-27713.7 | Hz |
| 28222-28222.7 | Hz |
| 40489-40489.7 | Hz |
| 40762-40762.7 | Hz |
| 41279-41279.7 | Hz |
| 42923-42923.7 | Hz |
| 43166-43166.7 | Hz |
| 43692-43692.7 | Hz |
| 48583-48583.7 | Hz |
| 49434-49434.7 | Hz |
| 66553-66553.7 | Hz |
| 79658-79658.7 | Hz |

### Example 15: Treatment of Colorectal Cancer

Colorectal cancer may be treated according to the methods disclosed herein by the application of colorectal cancer specific amplitude-modulated frequencies. Colorectal cancer specific frequencies are provided below in Table 47.

TABLE 47
Amplitude-modulated frequencies discovered to be
effective for treatment of colorectal cancer.

| | |
|---|---|
| 605-605.7 | Hz |
| 826-826.7 | Hz |
| 834-834.7 | Hz |
| 841.9-842.6 | Hz |
| 1808-1808.6 | Hz |
| 1928-1928.7 | Hz |
| 1989-1989.7 | Hz |
| 2439.7-2440.3 | Hz |
| 2714-2714.7 | Hz |
| 2747-2747.7 | Hz |
| 2759-2759.7 | Hz |
| 2807-2807.7 | Hz |
| 2826-2826.7 | Hz |
| 2981-2981.7 | Hz |
| 3209-3209.7 | Hz |
| 3213-3213.7 | Hz |
| 3244-3244.7 | Hz |
| 3261-3261.7 | Hz |
| 3548-3548.7 | Hz |
| 3634-3634.7 | Hz |
| 3646.4-3647 | Hz |
| 3915-3915.7 | Hz |
| 3922-3922.7 | Hz |
| 3940-3940.7 | Hz |
| 3946-3946.7 | Hz |
| 4135-4135.7 | Hz |
| 4155-4155.7 | Hz |
| 4224-4224.7 | Hz |
| 4241-4241.7 | Hz |
| 4283-4283.7 | Hz |
| 4323-4323.7 | Hz |
| 4438.7-4439.2 | Hz |
| 4447-4447.7 | Hz |
| 4708-4708.7 | Hz |
| 4713-4713.7 | Hz |
| 4718-4718.7 | Hz |
| 4718-4718.7 | Hz |
| 4722-4722.7 | Hz |
| 4776-4776.7 | Hz |
| 4850-4850.7 | Hz |
| 4874-4874.7 | Hz |
| 5205-5205.7 | Hz |
| 5209-5209.7 | Hz |
| 5209-5209.7 | Hz |
| 5221-5221.7 | Hz |
| 5232-5232.7 | Hz |
| 5265-5265.7 | Hz |
| 5427-5427.7 | Hz |
| 5484.4-5485 | Hz |
| 6074-6074.7 | Hz |
| 6112-6112.7 | Hz |
| 6159-6159.7 | Hz |
| 6361-6361.7 | Hz |
| 6438-6438.7 | Hz |
| 6468-6468.7 | Hz |
| 6537-6537.7 | Hz |
| 6648-6648.7 | Hz |
| 6869-6869.7 | Hz |
| 6878-6878.7 | Hz |
| 6914-6914.7 | Hz |
| 6974-6974.7 | Hz |
| 6982-6982.7 | Hz |
| 6986-6986.7 | Hz |
| 7109-7109.7 | Hz |
| 7117-7117.7 | Hz |
| 7137-7137.7 | Hz |
| 7149-7149.7 | Hz |
| 7191.7-7192.3 | Hz |
| 7531-7531.7 | Hz |
| 7560-7560.7 | Hz |
| 7879-7879.7 | Hz |
| 7936-7936.7 | Hz |
| 7970-7970.7 | Hz |
| 7994-7994.7 | Hz |
| 8038.4-8039 | Hz |
| 8413-8413.7 | Hz |
| 8438-8438.7 | Hz |
| 8467-8467.7 | Hz |
| 8771-8771.7 | Hz |
| 8809-8809.7 | Hz |
| 8889-8889.7 | Hz |
| 9071-9071.7 | Hz |
| 9084-9084.7 | Hz |
| 9731-9731.7 | Hz |
| 10566-10566.7 | Hz |
| 10831.7-10832.1 | Hz |
| 10845-10845.7 | Hz |
| 10859-10859.7 | Hz |
| 10906-10906.7 | Hz |
| 10947-10947.7 | Hz |
| 10974-10974.7 | Hz |
| 11110-11110.7 | Hz |
| 11225-11225.7 | Hz |
| 11234-11234.7 | Hz |
| 11255-11255.7 | Hz |
| 11387-11387.7 | Hz |
| 11530-11530.7 | Hz |
| 11576-11576.7 | Hz |
| 11621-11621.7 | Hz |
| 11630-11630.7 | Hz |
| 11638-11638.7 | Hz |
| 11681-11681.7 | Hz |
| 11911-11911.7 | Hz |
| 11934-11934.7 | Hz |
| 11949-11949.7 | Hz |
| 12182-12182.7 | Hz |
| 12373-12373.7 | Hz |
| 12575-12575.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 13418-13418.7 | Hz |
| 13475-13475.7 | Hz |
| 13611-13611.7 | Hz |
| 13615-13615.7 | Hz |
| 13670-13670.7 | Hz |
| 13717-13717.7 | Hz |
| 13736-13736.7 | Hz |
| 13742-13742.7 | Hz |
| 13773-13773.7 | Hz |
| 13775-13775.7 | Hz |
| 13978.3-13979 | Hz |
| 14182-14182.7 | Hz |
| 14417-14417.7 | Hz |
| 14422-14422.7 | Hz |
| 14441-14441.7 | Hz |
| 14443-14443.7 | Hz |
| 14570-14570.7 | Hz |
| 14621-14621.7 | Hz |
| 14630-14630.7 | Hz |
| 14671-14671.7 | Hz |
| 14674-14674.7 | Hz |
| 14863-14863.7 | Hz |
| 14877-14877.7 | Hz |
| 14911-14911.7 | Hz |
| 14957-14957.7 | Hz |
| 14982-14982.7 | Hz |
| 15109-15109.7 | Hz |
| 15133-15133.7 | Hz |
| 15154-15154.7 | Hz |
| 15230-15230.7 | Hz |
| 15250-15250.7 | Hz |
| 15278-15278.7 | Hz |
| 16274-16274.7 | Hz |
| 17106-17106.7 | Hz |
| 17123-17123.7 | Hz |
| 17472-17472.7 | Hz |
| 17515-17515.7 | Hz |
| 17578.4-17579 | Hz |
| 17808-17808.7 | Hz |
| 17825-17825.7 | Hz |
| 18157-18157.7 | Hz |
| 18171-18171.7 | Hz |
| 18294-18294.7 | Hz |
| 18372-18372.7 | Hz |
| 18375-18375.7 | Hz |
| 18396-18396.7 | Hz |
| 18533-18533.7 | Hz |
| 18635-18635.7 | Hz |
| 19342-19342.7 | Hz |
| 19437-19437.7 | Hz |
| 19447-19447.7 | Hz |
| 19829-19829.7 | Hz |
| 20173-20173.7 | Hz |
| 20499-20499.7 | Hz |
| 21389-21389.7 | Hz |
| 21490-21490.7 | Hz |
| 21663-21663.7 | Hz |
| 21833-21833.7 | Hz |
| 22167-22167.7 | Hz |
| 22423.4-22424 | Hz |
| 22559.3-22560 | Hz |
| 22743-22743.7 | Hz |
| 23246-23246.7 | Hz |
| 23249-23249.7 | Hz |
| 23259-23259.7 | Hz |
| 23344-23344.7 | Hz |
| 23624-23624.7 | Hz |
| 23667-23667.7 | Hz |
| 24142-24142.7 | Hz |
| 26113-26113.7 | Hz |
| 26403-26403.7 | Hz |
| 28212-28212.7 | Hz |
| 28272-28272.7 | Hz |
| 28537-28537.7 | Hz |
| 30075-30075.7 | Hz |
| 30373-30373.7 | Hz |
| 31847-31847.7 | Hz |
| 33546-33546.7 | Hz |
| 35324-35324.7 | Hz |
| 35543-35543.7 | Hz |
| 42544-42544.7 | Hz |
| 42692.3-42693 | Hz |
| 42812-42812.7 | Hz |
| 42813-42813.7 | Hz |
| 43162-43162.7 | Hz |
| 43325-43325.7 | Hz |
| 44652-44652.7 | Hz |
| 46340-46340.7 | Hz |
| 46746-46746.7 | Hz |
| 48653-48653.7 | Hz |
| 51946-51946.7 | Hz |
| 52752-52752.7 | Hz |
| 80715-80715.7 | Hz |
| 94494-94494.7 | Hz |

Conclusions:

The treatment of cancer according to the method and devices described herein is a safe and promising novel treatment modality for multiple types of cancer. Following extended trials, it has been determined that application to subjects of the frequencies provided herein enhances the efficacy of treatment and yields therapeutic effects in patients whose tumors have become resistant to therapy. It is accordingly preferred that most (i.e., over 50%) or all of the determined listed frequencies be applied to the subject. The mechanism of including additional frequencies is attributed to either or both of inter-active synergism between applied frequencies or between cells which have been influenced by the treatment and additive effects of the additional frequencies.

Of further note is the fact that different patients suffering from the same type of tumor cell growth practically exhibit the above-mentioned physiological responses at the same well-defined AM frequencies. Furthermore, AM frequencies which differ only very slightly (less than 0.001% at higher frequencies) from the frequencies listed, in general elicit reduced or no physiological response by subjects exposed to excitation at such very slightly different frequency. In view of these determinations, the electronic system of the present invention may be adapted to screen a subject for physiological responses over a broad range of frequencies to determine the presence or absence tumor cells and, if positive, then to note at which defined frequencies physiological responses are elicited. These frequencies will in general match with the defined frequencies listed in one of the Examples above or such further examples as may be developed and hence the nature of the tumor will be known. The electronic system of the invention is therefore a valuable diagnostic tool for diagnosing the presence or absence and identities of types of tumor cell growths or cancers. Furthermore, the electronic system of the invention is of value for predicting whether a patient will benefit from the application of a given series of modulation frequencies. The system therefore possesses a capability of predicting responses to treatment, thereby enhancing the possibility to select optimal modes of treatment.

The sequence of well-defined frequencies is preferably applied sequentially for determined periods of time, e.g., 3 seconds for each frequency, but several frequencies may also be applied simultaneously or may be applied in any order or in a random order. This means that a cycle of application involving 180 frequencies would take nearly 10 minutes time. Advantageous effects may, however, also arise from applying individual well-defined frequencies for differing time periods, e.g., some for 3 seconds, some for 6 seconds, etc.

Therapeutic dosages to be applied to a subject suffering from the presence of tumor cell growth or cancer are determined by the time of application of the low energy electromagnetic emissions to the subject and will depend on the nature of the cancer and the overall condition of the subject. In general, however, greatest experience has been gained in treating terminally ill subjects expected to survive no longer than about three months and who have agreed to discontinue alternative forms of cancer treatments such as chemo-therapy or radioactive treatment. In these severe cases, convenient times of treatment are recommended, e.g., 3 times 1 hour daily treatment. However, with the use of alternative forms of application, i.e., other than by means of a mouth probe, continuous application is possible and may be desirable.

While the invention has been described with specific embodiments, other alternatives, modifications and variations will be apparent to those skilled in the art. Accordingly, it will be intended to include all such alternatives, modifications, and variations within the spirit and scope of the appended claims.

1. A method of treating a subject suffering from cancer, the method comprising:
exposing the subject to low energy high frequency radiation, wherein the low energy high frequency radiation comprises one or more amplitude-modulated output signals,
wherein the one or more amplitude-modulated output signal(s):
have a carrier frequency from about 1 KHz to 5000 MHz; and
have amplitude modulation frequencies from about 0.1 Hz to about 150,000 Hz, and wherein the amplitude modulation frequencies are selected to be cancer-specific frequencies;
wherein the subject is treated with one or more cancer-specific amplitude modulation frequencies selected from Table A:
TABLE A

| | |
|---|---|
| 262-262.7 | Hz |
| 266.3-267 | Hz |
| 295-295.6 | Hz |
| 334-334.7 | Hz |
| 374-374.7 | Hz |
| 408-408.7 | Hz |
| 415-415.7 | Hz |
| 418-418.7 | Hz |
| 419.8-420.4 | Hz |
| 420-420.7 | Hz |
| 420.4-420.8 | Hz |
| 429-429.7 | Hz |
| 429.7-430.3 | Hz |
| 439-439.7 | Hz |
| 441-441.7 | Hz |
| 442-442.7 | Hz |
| 465-465.7 | Hz |
| 485-485.7 | Hz |
| 527-527.7 | Hz |
| 556-556.7 | Hz |
| 561-561.7 | Hz |
| 568-568.7 | Hz |
| 572-572.7 | Hz |
| 581-581.7 | Hz |
| 594-594.7 | Hz |
| 605-605.7 | Hz |
| 611-611.7 | Hz |
| 612-612.7 | Hz |
| 624.32-624.8 | Hz |
| 626-626.7 | Hz |
| 627-627.7 | Hz |
| 631-631.7 | Hz |
| 633-633.7 | Hz |
| 638-638.7 | Hz |
| 677.4-678 | Hz |
| 679-679.7 | Hz |
| 709-709.7 | Hz |
| 717-717.7 | Hz |
| 727-727.7 | Hz |
| 733-733.7 | Hz |
| 737-737.7 | Hz |
| 759-759.7 | Hz |
| 761-761.7 | Hz |
| 810-810.7 | Hz |
| 811-811.7 | Hz |
| 817-817.7 | Hz |
| 826-826.7 | Hz |
| 829-829.7 | Hz |
| 830-830.7 | Hz |
| 834-834.7 | Hz |
| 836-836.7 | Hz |
| 883-883.7 | Hz |
| 959-959.7 | Hz |
| 961-961.7 | Hz |
| 963.8-964.25 | Hz |
| 964.5-965 | Hz |
| 966-966.7 | Hz |
| 969-969.7 | Hz |
| 972-972.7 | Hz |
| 974.4-975 | Hz |
| 977-977.7 | Hz |
| 996-996.7 | Hz |
| 1027-1027.7 | Hz |
| 1045-1045.7 | Hz |
| 1157-1157.7 | Hz |
| 1179.2-1179.8 | Hz |
| 1195-1195.7 | Hz |
| 1213-1213.7 | Hz |
| 1220-1220.7 | Hz |
| 1252-1252.7 | Hz |
| 1314-1314.7 | Hz |
| 1336-1336.7 | Hz |
| 1352-1352.7 | Hz |
| 1369-1369.7 | Hz |
| 1436-1436.7 | Hz |
| 1467-1467.7 | Hz |
| 1622-1622.7 | Hz |
| 1652-1652.7 | Hz |
| 1668-1668.7 | Hz |
| 1728-1728.7 | Hz |
| 1759-1759.7 | Hz |
| 1771.7-1772.3 | Hz |
| 1772.3-1772.8 | Hz |
| 1785-1785.7 | Hz |
| 1808-1808.6 | Hz |
| 1815-1815.7 | Hz |
| 1823-1823.7 | Hz |
| 1858-1858.7 | Hz |
| 1868-1868.7 | Hz |
| 1881-1881.7 | Hz |
| 1897-1897.7 | Hz |
| 1903-1903.7 | Hz |
| 1914-1914.7 | Hz |
| 1928-1928.7 | Hz |
| 1963-1963.7 | Hz |
| 1966-1966.7 | Hz |
| 1967-1967.7 | Hz |
| 1973-1973.7 | Hz |
| 1989-1989.7 | Hz |
| 2021-2021.7 | Hz |
| 2023-2023.7 | Hz |
| 2035-2035.7 | Hz |
| 2044-2044.7 | Hz |
| 2065-2065.7 | Hz |
| 2094-2094.7 | Hz |
| 2111-2111.7 | Hz |
| 2128-2128.7 | Hz |
| 2189-2189.7 | Hz |
| 2284-2284.7 | Hz |
| 2287-2287.7 | Hz |
| 2313-2313.7 | Hz |
| 2321-2321.7 | Hz |
| 2328-2328.7 | Hz |
| 2346-2346.7 | Hz |
| 2359-2359.7 | Hz |
| 2372.3-2372.8 | Hz |
| 2373-2373.7 | Hz |
| 2380-2380.7 | Hz |
| 2429-2429.7 | Hz |
| 2439.7-2440.3 | Hz |
| 2455-2455.7 | Hz |
| 2459-2459.7 | Hz |
| 2469.2-2469.7 | Hz |
| 2483-2483.7 | Hz |
| 2513-2513.7 | Hz |
| 2518-2518.7 | Hz |
| 2612-2612.7 | Hz |
| 2627-2627.7 | Hz |
| 2629-2629.7 | Hz |
| 2632-2632.7 | Hz |
| 2636-2636.7 | Hz |
| 2647-2647.7 | Hz |
| 2648-2648.7 | Hz |
| 2653.3-2654 | Hz |
| 2653.7-2654.4 | Hz |
| 2679-2679.7 | Hz |
| 2697-2697.7 | Hz |
| 2706-2706.7 | Hz |
| 2707-2707.7 | Hz |
| 2714-2714.7 | Hz |
| 2720.3-2721 | Hz |
| 2726-2726.7 | Hz |
| 2738-2738.7 | Hz |
| 2747-2747.7 | Hz |
| 2754-2754.7 | Hz |
| 2759-2759.7 | Hz |
| 2762-2762.7 | Hz |
| 2774-2774.7 | Hz |
| 2778-2778.7 | Hz |
| 2807-2807.7 | Hz |
| 2811-2811.7 | Hz |
| 2824-2824.7 | Hz |
| 2825-2825.7 | Hz |
| 2826-2826.7 | Hz |
| 2830-2830.7 | Hz |
| 2834-2834.7 | Hz |
| 2836-2836.7 | Hz |
| 2840-2840.7 | Hz |
| 2842-2842.7 | Hz |
| 2854-2854.6 | Hz |
| 2854-2854.7 | Hz |
| 2858.8-2859.35 | Hz |
| 2860-2860.7 | Hz |
| 2864-2864.7 | Hz |
| 2865-2865.7 | Hz |
| 2878-2878.7 | Hz |
| 2890-2890.7 | Hz |
| 2893-2893.7 | Hz |
| 2940-2940.7 | Hz |
| 2971-2971.6 | Hz |
| 2975-2975.7 | Hz |
| 2981-2981.7 | Hz |
| 3030-3030.7 | Hz |
| 3056-3056.7 | Hz |
| 3085.7-3086.3 | Hz |
| 3121-3121.7 | Hz |
| 3133-3133.7 | Hz |
| 3135-3135.7 | Hz |
| 3143-3143.7 | Hz |
| 3171-3171.7 | Hz |
| 3188-3188.7 | Hz |
| 3207-3207.7 | Hz |
| 3209-3209.7 | Hz |
| 3213-3213.7 | Hz |
| 3218-3218.7 | Hz |
| 3222-3222.7 | Hz |
| 3227.3-3228 | Hz |
| 3228-3228.7 | Hz |
| 3234-3234.7 | Hz |
| 3244-3244.7 | Hz |
| 3245-3245.7 | Hz |
| 3250.3-3250.8 | Hz |
| 3251-3251.7 | Hz |
| 3261-3261.7 | Hz |
| 3267-3267.7 | Hz |
| 3269-3269.7 | Hz |
| 3272-3272.7 | Hz |
| 3279.3-3280 | Hz |
| 3280.6-3281.3 | Hz |
| 3281.6-3282.1 | Hz |
| 3322-3322.7 | Hz |
| 3360-3360.7 | Hz |
| 3371-3371.7 | Hz |
| 3428-3428.7 | Hz |
| 3446-3446.7 | Hz |
| 3466-3466.7 | Hz |
| 3522-3522.7 | Hz |
| 3529-3529.7 | Hz |
| 3537-3537.7 | Hz |
| 3548-3548.7 | Hz |
| 3556-3556.7 | Hz |
| 3562-3562.7 | Hz |
| 3571-3571.7 | Hz |
| 3595-3595.7 | Hz |
| 3609-3609.7 | Hz |
| 3618-3618.7 | Hz |
| 3621-3621.7 | Hz |
| 3622.5-3623 | Hz |
| 3627-3627.7 | Hz |
| 3630-3630.7 | Hz |
| 3631-3631.7 | Hz |
| 3633-3633.7 | Hz |
| 3634-3634.7 | Hz |
| 3637.7-3638.3 | Hz |
| 3644-3644.7 | Hz |
| 3646-3646.7 | Hz |
| 3646.4-3647 | Hz |
| 3656-3656.7 | Hz |
| 3661-3661.7 | Hz |
| 3663-3663.7 | Hz |
| 3667-3667.7 | Hz |
| 3678-3678.7 | Hz |
| 3681.7-3682.3 | Hz |
| 3687-3687.7 | Hz |
| 3694-3694.7 | Hz |
| 3731-3731.7 | Hz |
| 3743-3743.7 | Hz |
| 3749.3-3750 | Hz |
| 3756-3756.7 | Hz |
| 3762-3762.7 | Hz |
| 3787-3787.7 | Hz |
| 3817-3817.7 | Hz |
| 3823-3823.7 | Hz |
| 3833-3833.7 | Hz |
| 3839-3839.7 | Hz |
| 3842.4-3843 | Hz |
| 3854-3854.7 | Hz |
| 3877-3877.7 | Hz |
| 3908.7-3909.22 | Hz |
| 3910-3910.7 | Hz |
| 3915-3915.7 | Hz |
| 3922-3922.7 | Hz |
| 3936-3936.7 | Hz |
| 3940-3940.7 | Hz |
| 3946-3946.7 | Hz |
| 3947-3947.7 | Hz |
| 3948-3948.7 | Hz |
| 3954-3954.7 | Hz |
| 3958-3958.6 | Hz |
| 3962-3962.7 | Hz |
| 3981-3981.7 | Hz |
| 4007-4007.7 | Hz |
| 4013-4013.7 | Hz |
| 4027-4027.7 | Hz |
| 4048-4048.7 | Hz |
| 4055-4055.7 | Hz |
| 4135-4135.7 | Hz |
| 4148-4148.7 | Hz |
| 4152-4152.7 | Hz |
| 4155-4155.7 | Hz |
| 4190-4190.7 | Hz |
| 4198-4198.7 | Hz |
| 4224-4224.7 | Hz |
| 4233-4233.7 | Hz |
| 4249-4249.7 | Hz |
| 4281-4281.7 | Hz |
| 4283-4283.7 | Hz |
| 4323-4323.7 | Hz |
| 4368.3-4369 | Hz |
| 4386.4-4387 | Hz |
| 4409-4409.7 | Hz |
| 4410-4410.7 | Hz |
| 4418-4418.7 | Hz |
| 4425-4425.7 | Hz |
| 4425.7-4426.22 | Hz |
| 4430-4430.7 | Hz |
| 4438.7-4439.2 | Hz |
| 4441-4441.7 | Hz |
| 4459-4459.7 | Hz |
| 4465-4465.7 | Hz |
| 4476-4476.7 | Hz |
| 4477.4-4478 | Hz |
| 4523-4523.7 | Hz |
| 4548-4548.7 | Hz |
| 4580.11-4580.7 | Hz |
| 4584-4584.7 | Hz |
| 4621-4621.7 | Hz |
| 4632-4632.7 | Hz |
| 4637-4637.7 | Hz |
| 4657-4657.6 | Hz |
| 4707-4707.7 | Hz |
| 4708-4708.7 | Hz |
| 4713-4713.7 | Hz |
| 4714-4714.7 | Hz |
| 4722-4722.7 | Hz |
| 4723-4723.7 | Hz |
| 4725-4725.7 | Hz |
| 4731-4731.7 | Hz |
| 4733-4733.7 | Hz |
| 4748-4748.7 | Hz |
| 4776-4776.7 | Hz |
| 4792-4792.7 | Hz |
| 4805.3-4806 | Hz |
| 4806-4806.7 | Hz |
| 4809-4809.7 | Hz |
| 4812-4812.7 | Hz |
| 4813-4813.7 | Hz |
| 4821-4821.7 | Hz |
| 4822-4822.7 | Hz |
| 4823-4823.7 | Hz |
| 4833-4833.7 | Hz |
| 4841-4841.7 | Hz |
| 4844-4844.7 | Hz |
| 4850-4850.7 | Hz |
| 4866-4866.7 | Hz |
| 4870-4870.7 | Hz |
| 4874-4874.7 | Hz |
| 4890-4890.7 | Hz |
| 4924-4924.7 | Hz |
| 4941-4941.7 | Hz |
| 4946-4946.7 | Hz |
| 4948-4948.7 | Hz |
| 4997-4997.7 | Hz |
| 4998-4998.7 | Hz |
| 5034-5034.7 | Hz |
| 5063-5063.7 | Hz |
| 5089-5089.7 | Hz |
| 5116-5116.7 | Hz |
| 5139-5139.7 | Hz |
| 5142-5142.7 | Hz |
| 5152-5152.7 | Hz |
| 5154-5154.7 | Hz |
| 5156-5156.7 | Hz |
| 5165.4-5166 | Hz |
| 5205-5205.7 | Hz |
| 5209-5209.7 | Hz |
| 5209.4-5210 | Hz |
| 5221-5221.7 | Hz |
| 5229-5229.7 | Hz |
| 5232-5232.7 | Hz |
| 5239-5239.7 | Hz |
| 5243-5243.7 | Hz |
| 5259-5259.7 | Hz |
| 5265-5265.7 | Hz |
| 5270-5270.7 | Hz |
| 5286-5286.7 | Hz |
| 5322-5322.7 | Hz |
| 5336-5336.7 | Hz |
| 5378.2-5378.7 | Hz |
| 5407.3-5408 | Hz |
| 5414-5414.7 | Hz |
| 5424-5424.7 | Hz |
| 5427-5427.7 | Hz |
| 5438-5438.7 | Hz |
| 5484.4-5485 | Hz |
| 5510-5510.7 | Hz |
| 5533-5533.7 | Hz |
| 5618-5618.7 | Hz |
| 5640.4-5641 | Hz |
| 5671-5671.7 | Hz |
| 5714-5714.7 | Hz |
| 5716-5716.7 | Hz |
| 5741-5741.7 | Hz |
| 5742-5742.7 | Hz |
| 5752-5752.7 | Hz |
| 5766-5766.7 | Hz |
| 5881-5881.7 | Hz |
| 5884-5884.7 | Hz |
| 5976-5976.7 | Hz |
| 5996-5996.7 | Hz |
| 6010-6010.7 | Hz |
| 6024-6024.7 | Hz |
| 6058-6058.7 | Hz |
| 6070-6070.7 | Hz |
| 6074-6074.7 | Hz |
| 6094-6094.7 | Hz |
| 6112-6112.7 | Hz |
| 6117-6117.7 | Hz |
| 6159-6159.7 | Hz |
| 6167-6167.7 | Hz |
| 6179-6179.7 | Hz |
| 6220.3-6221 | Hz |
| 6252-6252.7 | Hz |
| 6297-6297.7 | Hz |
| 6298.4-6299 | Hz |
| 6317-6317.7 | Hz |
| 6330-6330.7 | Hz |
| 6333-6333.7 | Hz |
| 6334-6334.7 | Hz |
| 6346-6346.7 | Hz |
| 6380.4-6381 | Hz |
| 6392-6392.7 | Hz |
| 6413-6413.7 | Hz |
| 6414-6414.7 | Hz |
| 6416.3-6417 | Hz |
| 6419.3-6420 | Hz |
| 6423-6423.7 | Hz |
| 6432-6432.7 | Hz |
| 6436-6436.7 | Hz |
| 6438-6438.7 | Hz |
| 6441-6441.7 | Hz |
| 6443-6443.7 | Hz |
| 6444-6444.7 | Hz |
| 6468-6468.7 | Hz |
| 6469-6469.7 | Hz |
| 6479.4-6480 | Hz |
| 6488-6488.7 | Hz |
| 6492.3-6493 | Hz |
| 6513-6513.7 | Hz |
| 6537-6537.7 | Hz |
| 6648-6648.7 | Hz |
| 6717-6717.7 | Hz |
| 6727.4-6728 | Hz |
| 6730-6730.7 | Hz |
| 6748-6748.7 | Hz |
| 6751-6751.7 | Hz |
| 6781-6781.7 | Hz |
| 6782-6782.7 | Hz |
| 6808-6808.7 | Hz |
| 6809-6809.6 | Hz |
| 6825-6825.7 | Hz |
| 6832-6832.7 | Hz |
| 6840-6840.7 | Hz |
| 6841-6841.7 | Hz |
| 6847-6847.7 | Hz |
| 6864-6864.7 | Hz |
| 6869-6869.7 | Hz |
| 6897-6897.7 | Hz |
| 6911.3-6912 | Hz |
| 6912-6912.7 | Hz |
| 6914-6914.7 | Hz |
| 6968.4-6969 | Hz |
| 6974-6974.7 | Hz |
| 6982-6982.7 | Hz |
| 6986-6986.7 | Hz |
| 7013-7013.7 | Hz |
| 7037-7037.7 | Hz |
| 7042-7042.7 | Hz |
| 7080-7080.7 | Hz |
| 7098-7098.7 | Hz |
| 7109-7109.7 | Hz |
| 7110-7110.7 | Hz |
| 7112-7112.7 | Hz |
| 7117-7117.7 | Hz |
| 7136-7136.7 | Hz |
| 7137-7137.7 | Hz |
| 7145.4-7146 | Hz |
| 7149-7149.7 | Hz |
| 7151-7151.7 | Hz |
| 7158-7158.7 | Hz |
| 7160-7160.7 | Hz |
| 7169-7169.7 | Hz |
| 7191.7-7192.3 | Hz |
| 7222.4-7223 | Hz |
| 7225-7225.7 | Hz |
| 7234-7234.7 | Hz |
| 7239-7239.7 | Hz |
| 7246-7246.7 | Hz |
| 7253-7253.7 | Hz |
| 7268-7268.7 | Hz |
| 7274-7274.7 | Hz |
| 7283-7283.7 | Hz |
| 7338-7338.7 | Hz |
| 7367-7367.7 | Hz |
| 7378-7378.7 | Hz |
| 7404.4-7405 | Hz |
| 7424-7424.7 | Hz |
| 7440-7440.7 | Hz |
| 7481-7481.7 | Hz |
| 7510-7510.7 | Hz |
| 7526-7526.6 | Hz |
| 7531-7531.7 | Hz |
| 7546-7546.7 | Hz |
| 7551-7551.7 | Hz |
| 7560-7560.7 | Hz |
| 7646-7646.7 | Hz |
| 7652-7652.7 | Hz |
| 7655-7655.7 | Hz |
| 7673-7673.7 | Hz |
| 7676-7676.7 | Hz |
| 7719-7719.7 | Hz |
| 7748-7748.7 | Hz |
| 7787-7787.7 | Hz |
| 7808-7808.7 | Hz |
| 7811-7811.7 | Hz |
| 7855-7855.7 | Hz |
| 7857-7857.7 | Hz |
| 7865-7865.7 | Hz |
| 7879-7879.7 | Hz |
| 7906-7906.7 | Hz |
| 7910-7910.7 | Hz |
| 7913-7913.7 | Hz |
| 7923-7923.7 | Hz |
| 7927-7927.7 | Hz |
| 7930-7930.7 | Hz |
| 7936-7936.7 | Hz |
| 7951-7951.7 | Hz |
| 7970-7970.7 | Hz |
| 7981-7981.7 | Hz |
| 7994-7994.7 | Hz |
| 8012.3-8013 | Hz |
| 8013-8013.7 | Hz |
| 8024-8024.7 | Hz |
| 8026-8026.7 | Hz |
| 8032-8032.7 | Hz |
| 8039-8039.7 | Hz |
| 8043-8043.7 | Hz |
| 8074-8074.7 | Hz |
| 8079-8079.7 | Hz |
| 8097-8097.7 | Hz |
| 8125-8125.7 | Hz |
| 8145-8145.7 | Hz |
| 8158-8158.7 | Hz |
| 8217.4-8218 | Hz |
| 8251-8251.7 | Hz |
| 8314-8314.7 | Hz |
| 8332-8332.7 | Hz |
| 8347-8347.7 | Hz |
| 8355-8355.7 | Hz |
| 8373-8373.7 | Hz |
| 8386-8386.7 | Hz |
| 8388-8388.7 | Hz |
| 8412-8412.7 | Hz |
| 8413-8413.7 | Hz |
| 8421-8421.7 | Hz |
| 8427-8427.7 | Hz |
| 8445-8445.7 | Hz |
| 8447-8447.7 | Hz |
| 8467-8467.7 | Hz |
| 8478.3-8479 | Hz |
| 8480-8480.7 | Hz |
| 8484-8484.7 | Hz |
| 8509.4-8510 | Hz |
| 8522-8522.7 | Hz |
| 8546-8546.7 | Hz |
| 8562-8562.7 | Hz |
| 8576-8576.7 | Hz |
| 8698-8698.7 | Hz |
| 8731-8731.7 | Hz |
| 8734-8734.7 | Hz |
| 8740-8740.7 | Hz |
| 8755-8755.7 | Hz |
| 8756-8756.7 | Hz |
| 8757-8757.7 | Hz |
| 8765-8765.7 | Hz |
| 8771-8771.7 | Hz |
| 8789.3-8790 | Hz |
| 8809-8809.7 | Hz |
| 8810-8810.7 | Hz |
| 8814-8814.7 | Hz |
| 8823-8823.7 | Hz |
| 8824-8824.7 | Hz |
| 8827.4-8828 | Hz |
| 8829-8829.7 | Hz |
| 8837-8837.7 | Hz |
| 8841-8841.7 | Hz |
| 8843-8843.7 | Hz |
| 8845-8845.7 | Hz |
| 8846-8846.7 | Hz |
| 8853-8853.7 | Hz |
| 8859.3-8860 | Hz |
| 8872-8872.7 | Hz |
| 8877-8877.7 | Hz |
| 8881-8881.7 | Hz |
| 8889-8889.7 | Hz |
| 8926-8926.7 | Hz |
| 8935-8935.6 | Hz |
| 8949-8949.7 | Hz |
| 9023-9023.7 | Hz |
| 9071-9071.7 | Hz |
| 9075-9075.7 | Hz |
| 9078-9078.7 | Hz |
| 9084-9084.7 | Hz |
| 9106-9106.7 | Hz |
| 9109-9109.7 | Hz |
| 9112-9112.7 | Hz |
| 9122-9122.7 | Hz |
| 9123-9123.7 | Hz |
| 9133-9133.7 | Hz |
| 9136-9136.7 | Hz |
| 9146-9146.7 | Hz |
| 9155.7-9156.2 | Hz |
| 9157-9157.7 | Hz |
| 9161-9161.7 | Hz |
| 9169-9169.7 | Hz |
| 9174-9174.7 | Hz |
| 9179-9179.7 | Hz |
| 9194-9194.7 | Hz |
| 9230-9230.7 | Hz |
| 9236-9236.7 | Hz |
| 9247-9247.7 | Hz |
| 9273-9273.7 | Hz |
| 9506-9506.7 | Hz |
| 9512-9512.7 | Hz |
| 9573-9573.7 | Hz |
| 9588-9588.7 | Hz |
| 9635-9635.7 | Hz |
| 9656-9656.7 | Hz |
| 9681-9681.7 | Hz |
| 9695-9695.7 | Hz |
| 9731-9731.7 | Hz |
| 9846-9846.7 | Hz |
| 9851-9851.7 | Hz |
| 9886-9886.7 | Hz |
| 9921-9921.7 | Hz |
| 9929-9929.7 | Hz |
| 9935-9935.7 | Hz |
| 9947-9947.7 | Hz |
| 9961-9961.7 | Hz |
| 9970-9970.7 | Hz |
| 10013-10013.7 | Hz |
| 10064-10064.7 | Hz |
| 10073-10073.7 | Hz |
| 10137-10137.7 | Hz |
| 10140-10140.7 | Hz |
| 10159-10159.7 | Hz |
| 10339.8-10340.4 | Hz |
| 10342-10342.6 | Hz |
| 10345-10345.7 | Hz |
| 10352-10352.7 | Hz |
| 10362-10362.7 | Hz |
| 10378-10378.7 | Hz |
| 10384-10384.7 | Hz |
| 10387-10387.7 | Hz |
| 10395-10395.7 | Hz |
| 10427-10427.7 | Hz |
| 10436-10436.7 | Hz |
| 10439-10439.7 | Hz |
| 10484-10484.7 | Hz |
| 10488-10488.7 | Hz |
| 10541-10541.7 | Hz |
| 10566-10566.7 | Hz |
| 10566.4-10567 | Hz |
| 10633-10633.7 | Hz |
| 10641-10641.7 | Hz |
| 10645-10645.7 | Hz |
| 10661-10661.7 | Hz |
| 10665.4-10666 | Hz |
| 10671-10671.7 | Hz |
| 10673-10673.7 | Hz |
| 10690-10690.7 | Hz |
| 10708-10708.7 | Hz |
| 10727-10727.7 | Hz |
| 10731-10731.7 | Hz |
| 10735-10735.7 | Hz |
| 10740-10740.7 | Hz |
| 10745-10745.7 | Hz |
| 10748-10748.7 | Hz |
| 10762-10762.7 | Hz |
| 10768-10768.7 | Hz |
| 10776-10776.7 | Hz |
| 10781-10781.7 | Hz |
| 10788-10788.7 | Hz |
| 10802-10802.7 | Hz |
| 10805-10805.7 | Hz |
| 10809-10809.7 | Hz |
| 10813.2-10813.8 | Hz |
| 10830-10830.7 | Hz |
| 10831-10831.7 | Hz |
| 10831.7-10832.1 | Hz |
| 10834-10834.7 | Hz |
| 10844-10844.7 | Hz |
| 10845-10845.7 | Hz |
| 10848.7-10849.3 | Hz |
| 10851-10851.7 | Hz |
| 10854-10854.7 | Hz |
| 10859-10859.7 | Hz |
| 10870-10870.7 | Hz |
| 10873-10873.7 | Hz |
| 10876-10876.7 | Hz |
| 10878-10878.7 | Hz |
| 10889-10889.7 | Hz |
| 10891-10891.7 | Hz |
| 10894-10894.7 | Hz |
| 10906-10906.7 | Hz |
| 10947-10947.7 | Hz |
| 10974-10974.7 | Hz |
| 10993-10993.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11081-11081.7 | Hz |
| 11083-11083.7 | Hz |
| 11109.3-11110 | Hz |
| 11110-11110.7 | Hz |
| 11117-11117.7 | Hz |
| 11124-11124.7 | Hz |
| 11134-11134.7 | Hz |
| 11155-11155.7 | Hz |
| 11159-11159.7 | Hz |
| 11163-11163.7 | Hz |
| 11164-11164.7 | Hz |
| 11174-11174.7 | Hz |
| 11209-11209.7 | Hz |
| 11219-11219.7 | Hz |
| 11225-11225.7 | Hz |
| 11234-11234.7 | Hz |
| 11243-11243.7 | Hz |
| 11247-11247.7 | Hz |
| 11253-11253.7 | Hz |
| 11255-11255.7 | Hz |
| 11260-11260.7 | Hz |
| 11264-11264.7 | Hz |
| 11267-11267.7 | Hz |
| 11275-11275.7 | Hz |
| 11277.3-11278 | Hz |
| 11284-11284.7 | Hz |
| 11295-11295.7 | Hz |
| 11336-11336.7 | Hz |
| 11381-11381.7 | Hz |
| 11387-11387.7 | Hz |
| 11414-11414.7 | Hz |
| 11416-11416.7 | Hz |
| 11429-11429.7 | Hz |
| 11438-11438.7 | Hz |
| 11469-11469.7 | Hz |
| 11524-11524.7 | Hz |
| 11530-11530.7 | Hz |
| 11533-11533.7 | Hz |
| 11551.3-11552 | Hz |
| 11556-11556.7 | Hz |
| 11569-11569.7 | Hz |
| 11576-11576.7 | Hz |
| 11582-11582.7 | Hz |
| 11590-11590.7 | Hz |
| 11619-11619.7 | Hz |
| 11621-11621.7 | Hz |
| 11628-11628.7 | Hz |
| 11630-11630.7 | Hz |
| 11637-11637.7 | Hz |
| 11638-11638.7 | Hz |
| 11681-11681.7 | Hz |
| 11699-11699.7 | Hz |
| 11719-11719.7 | Hz |
| 11730.6-11731.1 | Hz |
| 11745-11745.7 | Hz |
| 11779-11779.7 | Hz |
| 11785-11785.7 | Hz |
| 11809-11809.7 | Hz |
| 11831-11831.7 | Hz |
| 11867-11867.7 | Hz |
| 11872-11872.7 | Hz |
| 11905-11905.7 | Hz |
| 11911-11911.7 | Hz |
| 11913-11913.7 | Hz |
| 11929-11929.7 | Hz |
| 11931-11931.7 | Hz |
| 11934-11934.7 | Hz |
| 11946-11946.7 | Hz |
| 11949-11949.7 | Hz |
| 11956-11956.7 | Hz |
| 11974-11974.7 | Hz |
| 11993-11993.7 | Hz |
| 11994-11994.7 | Hz |
| 12118-12118.7 | Hz |
| 12120.2-12120.7 | Hz |
| 12135-12135.7 | Hz |
| 12137-12137.7 | Hz |
| 12144-12144.7 | Hz |
| 12152-12152.7 | Hz |
| 12165-12165.7 | Hz |
| 12182-12182.7 | Hz |
| 12184-12184.7 | Hz |
| 12302-12302.7 | Hz |
| 12309-12309.7 | Hz |
| 12318-12318.7 | Hz |
| 12330-12330.7 | Hz |
| 12342-12342.7 | Hz |
| 12356-12356.7 | Hz |
| 12364-12364.7 | Hz |
| 12373-12373.7 | Hz |
| 12391-12391.7 | Hz |
| 12498-12498.7 | Hz |
| 12557-12557.7 | Hz |
| 12572-12572.7 | Hz |
| 12575-12575.7 | Hz |
| 12588-12588.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 12649-12649.7 | Hz |
| 12682-12682.7 | Hz |
| 12711-12711.7 | Hz |
| 12752-12752.7 | Hz |
| 12783-12783.7 | Hz |
| 12880-12880.7 | Hz |
| 12929-12929.7 | Hz |
| 13117-13117.7 | Hz |
| 13127-13127.7 | Hz |
| 13215-13215.7 | Hz |
| 13222-13222.7 | Hz |
| 13241-13241.7 | Hz |
| 13252-13252.7 | Hz |
| 13307-13307.7 | Hz |
| 13329-13329.7 | Hz |
| 13330.7-13331.2 | Hz |
| 13373-13373.7 | Hz |
| 13375-13375.7 | Hz |
| 13377-13377.7 | Hz |
| 13386-13386.7 | Hz |
| 13408-13408.7 | Hz |
| 13418-13418.7 | Hz |
| 13434-13434.7 | Hz |
| 13475-13475.7 | Hz |
| 13562-13562.6 | Hz |
| 13571-13571.7 | Hz |
| 13609-13609.7 | Hz |
| 13611-13611.7 | Hz |
| 13615-13615.7 | Hz |
| 13617-13617.7 | Hz |
| 13636.2-13636.7 | Hz |
| 13644-13644.7 | Hz |
| 13661-13661.7 | Hz |
| 13670-13670.7 | Hz |
| 13679-13679.7 | Hz |
| 13686.4-13687 | Hz |
| 13694-13694.7 | Hz |
| 13713-13713.7 | Hz |
| 13714-13714.7 | Hz |
| 13734-13734.7 | Hz |
| 13736-13736.7 | Hz |
| 13738.3-13739 | Hz |
| 13742-13742.7 | Hz |
| 13746-13746.7 | Hz |
| 13747-13747.7 | Hz |
| 13751-13751.7 | Hz |
| 13755-13755.7 | Hz |
| 13760-13760.7 | Hz |
| 13761-13761.7 | Hz |
| 13767-13767.7 | Hz |
| 13773-13773.7 | Hz |
| 13774-13774.7 | Hz |
| 13775-13775.7 | Hz |
| 13780-13780.7 | Hz |
| 13805-13805.7 | Hz |
| 13819-13819.7 | Hz |
| 13828-13828.7 | Hz |
| 13834-13834.7 | Hz |
| 13847-13847.7 | Hz |
| 13856-13856.7 | Hz |
| 13857.4-13858 | Hz |
| 13858-13858.7 | Hz |
| 13871-13871.7 | Hz |
| 13903-13903.7 | Hz |
| 13947-13947.7 | Hz |
| 13978.3-13979 | Hz |
| 13981-13981.7 | Hz |
| 14005-14005.7 | Hz |
| 14009-14009.7 | Hz |
| 14016.4-14017 | Hz |
| 14027-14027.7 | Hz |
| 14034-14034.7 | Hz |
| 14052-14052.7 | Hz |
| 14070-14070.7 | Hz |
| 14078-14078.7 | Hz |
| 14111-14111.7 | Hz |
| 14114-14114.7 | Hz |
| 14116-14116.7 | Hz |
| 14153-14153.7 | Hz |
| 14156-14156.7 | Hz |
| 14160-14160.7 | Hz |
| 14164-14164.7 | Hz |
| 14170.7-14171.3 | Hz |
| 14174-14174.7 | Hz |
| 14182-14182.7 | Hz |
| 14249-14249.7 | Hz |
| 14279-14279.7 | Hz |
| 14281-14281.7 | Hz |
| 14285-14285.7 | Hz |
| 14308-14308.7 | Hz |
| 14333.4-14333.9 | Hz |
| 14350-14350.7 | Hz |
| 14356-14356.7 | Hz |
| 14376-14376.7 | Hz |
| 14417-14417.7 | Hz |
| 14422-14422.7 | Hz |
| 14432-14432.7 | Hz |
| 14441-14441.7 | Hz |
| 14443-14443.7 | Hz |
| 14456-14456.7 | Hz |
| 14462-14462.7 | Hz |
| 14479-14479.7 | Hz |
| 14483-14483.7 | Hz |
| 14489-14489.7 | Hz |
| 14511-14511.7 | Hz |
| 14523-14523.7 | Hz |
| 14524-14524.7 | Hz |
| 14531-14531.7 | Hz |
| 14532-14532.7 | Hz |
| 14540-14540.7 | Hz |
| 14545-14545.7 | Hz |
| 14553-14553.7 | Hz |
| 14558-14558.7 | Hz |
| 14564-14564.7 | Hz |
| 14570-14570.7 | Hz |
| 14574-14574.7 | Hz |
| 14585-14585.7 | Hz |
| 14589-14589.7 | Hz |
| 14621-14621.7 | Hz |
| 14630-14630.7 | Hz |
| 14670.4-14671 | Hz |
| 14671-14671.7 | Hz |
| 14674-14674.7 | Hz |
| 14690-14690.7 | Hz |
| 14716-14716.7 | Hz |
| 14729-14729.7 | Hz |
| 14756-14756.7 | Hz |
| 14796-14796.7 | Hz |
| 14813-14813.7 | Hz |
| 14835-14835.7 | Hz |
| 14858-14858.7 | Hz |
| 14863-14863.7 | Hz |
| 14867-14867.7 | Hz |
| 14877-14877.7 | Hz |
| 14885-14885.7 | Hz |
| 14911-14911.7 | Hz |
| 14912-14912.7 | Hz |
| 14913.4-14914 | Hz |
| 14922-14922.7 | Hz |
| 14940-14940.7 | Hz |
| 14940.4-14941 | Hz |
| 14957-14957.7 | Hz |
| 14961-14961.7 | Hz |
| 14974-14974.7 | Hz |
| 14982-14982.7 | Hz |
| 15006-15006.7 | Hz |
| 15033-15033.7 | Hz |
| 15035-15035.6 | Hz |
| 15056-15056.7 | Hz |
| 15067-15067.7 | Hz |
| 15071-15071.7 | Hz |
| 15109-15109.7 | Hz |
| 15112-15112.7 | Hz |
| 15126-15126.7 | Hz |
| 15133-15133.7 | Hz |
| 15154-15154.7 | Hz |
| 15173-15173.7 | Hz |
| 15179-15179.7 | Hz |
| 15195-15195.7 | Hz |
| 15223-15223.7 | Hz |
| 15224-15224.7 | Hz |
| 15225-15225.6 | Hz |
| 15226-15226.7 | Hz |
| 15230-15230.7 | Hz |
| 15233-15233.7 | Hz |
| 15236-15236.7 | Hz |
| 15244-15244.7 | Hz |
| 15250-15250.7 | Hz |
| 15253-15253.7 | Hz |
| 15263-15263.7 | Hz |
| 15269-15269.7 | Hz |
| 15278-15278.7 | Hz |
| 15329-15329.7 | Hz |
| 15432-15432.7 | Hz |
| 15435-15435.7 | Hz |
| 15460-15460.7 | Hz |
| 15515-15515.7 | Hz |
| 15521-15521.7 | Hz |
| 15528-15528.7 | Hz |
| 15531-15531.7 | Hz |
| 15536-15536.7 | Hz |
| 15539-15539.7 | Hz |
| 15545-15545.7 | Hz |
| 15546.3-15547 | Hz |
| 15550.4-15551 | Hz |
| 15563-15563.7 | Hz |
| 15576-15576.7 | Hz |
| 15589-15589.7 | Hz |
| 15615-15615.7 | Hz |
| 15621-15621.7 | Hz |
| 15624-15624.7 | Hz |
| 15642-15642.7 | Hz |
| 15647-15647.7 | Hz |
| 15663-15663.7 | Hz |
| 15669-15669.7 | Hz |
| 15678-15678.7 | Hz |
| 15698-15698.7 | Hz |
| 15711-15711.7 | Hz |
| 15724-15724.7 | Hz |
| 15729-15729.7 | Hz |
| 15740-15740.7 | Hz |
| 15749-15749.7 | Hz |
| 15773-15773.7 | Hz |
| 15817-15817.7 | Hz |
| 15823-15823.7 | Hz |
| 15824-15824.7 | Hz |
| 15825-15825.7 | Hz |
| 15835-15835.7 | Hz |
| 15836-15836.7 | Hz |
| 15857-15857.7 | Hz |
| 15867-15867.7 | Hz |
| 15897-15897.7 | Hz |
| 15919-15919.7 | Hz |
| 15942-15942.7 | Hz |
| 15982-15982.7 | Hz |
| 16071-16071.7 | Hz |
| 16082-16082.7 | Hz |
| 16096-16096.7 | Hz |
| 16143-16143.7 | Hz |
| 16176-16176.7 | Hz |
| 16274-16274.7 | Hz |
| 16309-16309.7 | Hz |
| 16329-16329.7 | Hz |
| 16347-16347.7 | Hz |
| 16381-16381.7 | Hz |
| 16515-16515.7 | Hz |
| 16679-16679.7 | Hz |
| 16804-16804.7 | Hz |
| 16821-16821.7 | Hz |
| 17095-17095.7 | Hz |
| 17106-17106.7 | Hz |
| 17123-17123.7 | Hz |
| 17167-17167.7 | Hz |
| 17214-17214.7 | Hz |
| 17229-17229.7 | Hz |
| 17246-17246.7 | Hz |
| 17268-17268.7 | Hz |
| 17271-17271.7 | Hz |
| 17283-17283.7 | Hz |
| 17333-17333.7 | Hz |
| 17379-17379.7 | Hz |
| 17417-17417.7 | Hz |
| 17418-17418.7 | Hz |
| 17435-17435.7 | Hz |
| 17455-17455.7 | Hz |
| 17472-17472.7 | Hz |
| 17473-17473.7 | Hz |
| 17474-17474 7 | Hz |
| 17485-17485.7 | Hz |
| 17515-17515.7 | Hz |
| 17556-17556.7 | Hz |
| 17578.4-17579 | Hz |
| 17581-17581.7 | Hz |
| 17621-17621.7 | Hz |
| 17636-17636.7 | Hz |
| 17655-17655.7 | Hz |
| 17673-17673.7 | Hz |
| 17737-17737.7 | Hz |
| 17739-17739.7 | Hz |
| 17749-17749.7 | Hz |
| 17757-17757.7 | Hz |
| 17808-17808.7 | Hz |
| 17825-17825.7 | Hz |
| 17832-17832.7 | Hz |
| 17944-17944.7 | Hz |
| 17962-17962.7 | Hz |
| 17974-17974.7 | Hz |
| 17978-17978.7 | Hz |
| 18021-18021.7 | Hz |
| 18040-18040.7 | Hz |
| 18087-18087.7 | Hz |
| 18090-18090.7 | Hz |
| 18126-18126.7 | Hz |
| 18140-18140.7 | Hz |
| 18157-18157.7 | Hz |
| 18171-18171.7 | Hz |
| 18182-18182.7 | Hz |
| 18211-18211.7 | Hz |
| 18221-18221.7 | Hz |
| 18222-18222.7 | Hz |
| 18246-18246.7 | Hz |
| 18260-18260.7 | Hz |
| 18274-18274.7 | Hz |
| 18279-18279.7 | Hz |
| 18294-18294.7 | Hz |
| 18372-18372.7 | Hz |
| 18375-18375.7 | Hz |
| 18396-18396.7 | Hz |
| 18411-18411.7 | Hz |
| 18454-18454.7 | Hz |
| 18462-18462.7 | Hz |
| 18521-18521.7 | Hz |
| 18533-18533.7 | Hz |
| 18567-18567.7 | Hz |
| 18585-18585.7 | Hz |
| 18596-18596.7 | Hz |
| 18618-18618.7 | Hz |
| 18635-18635.7 | Hz |
| 18641-18641.7 | Hz |
| 18655-18655.7 | Hz |
| 18670.3-18671 | Hz |
| 18672-18672.7 | Hz |
| 18678-18678.7 | Hz |
| 18678.7-18679.3 | Hz |
| 18686-18686.7 | Hz |
| 18742-18742.7 | Hz |
| 18831-18831.7 | Hz |
| 18842-18842.7 | Hz |
| 18884-18884.7 | Hz |
| 18895-18895.7 | Hz |
| 18897-18897.7 | Hz |
| 18922-18922.7 | Hz |
| 19026-19026.7 | Hz |
| 19059-19059.7 | Hz |
| 19125-19125.7 | Hz |
| 19269-19269.7 | Hz |
| 19342-19342.7 | Hz |
| 19365-19365.7 | Hz |
| 19384-19384.7 | Hz |
| 19393-19393.7 | Hz |
| 19419-19419.7 | Hz |
| 19426-19426.7 | Hz |
| 19433-19433.7 | Hz |
| 19437-19437.7 | Hz |
| 19446-19446.7 | Hz |
| 19447-19447.7 | Hz |
| 19453-19453.7 | Hz |
| 19464-19464.7 | Hz |
| 19476-19476.7 | Hz |
| 19489.3-19490 | Hz |
| 19510-19510.7 | Hz |
| 19577-19577.7 | Hz |
| 19686-19686.4 | Hz |
| 19715-19715.7 | Hz |
| 19724-19724.7 | Hz |
| 19744-19744.7 | Hz |
| 19782-19782.7 | Hz |
| 19817-19817.7 | Hz |
| 19819-19819.7 | Hz |
| 19823-19823.7 | Hz |
| 19827-19827.7 | Hz |
| 19829-19829.7 | Hz |
| 19861-19861.7 | Hz |
| 19867-19867.7 | Hz |
| 19889-19889.7 | Hz |
| 20097-20097.7 | Hz |
| 20123-20123.7 | Hz |
| 20128-20128.7 | Hz |
| 20144-20144.7 | Hz |
| 20149.3-20150 | Hz |
| 20173-20173.7 | Hz |
| 20219-20219.7 | Hz |
| 20229-20229.7 | Hz |
| 20237-20237.7 | Hz |
| 20254-20254.7 | Hz |
| 20256-20256.7 | Hz |
| 20278.4-20279 | Hz |
| 20284-20284.7 | Hz |
| 20286-20286.7 | Hz |
| 20324-20324.7 | Hz |
| 20499-20499.7 | Hz |
| 20513-20513.7 | Hz |
| 20634-20634.7 | Hz |
| 20747-20747.7 | Hz |
| 20818-20818.7 | Hz |
| 20825-20825.7 | Hz |
| 20908-20908.7 | Hz |
| 20948-20948.7 | Hz |
| 21119-21119.7 | Hz |
| 21167-21167.7 | Hz |
| 21291.4-21292 | Hz |
| 21372-21372.7 | Hz |
| 21389-21389.7 | Hz |
| 21430-21430.7 | Hz |
| 21445-21445.7 | Hz |
| 21466-21466.7 | Hz |
| 21467-21467.7 | Hz |
| 21490-21490.7 | Hz |
| 21546-21546.7 | Hz |
| 21567-21567.7 | Hz |
| 21616-21616.7 | Hz |
| 21636-21636.7 | Hz |
| 21663-21663.7 | Hz |
| 21664-21664.7 | Hz |
| 21708-21708.7 | Hz |
| 21714-21714.7 | Hz |
| 21720-21720.7 | Hz |
| 21778-21778.7 | Hz |
| 21833-21833.7 | Hz |
| 21840-21840.7 | Hz |
| 21844-21844.7 | Hz |
| 21854-21854.7 | Hz |
| 21865-21865.7 | Hz |
| 21870-21870.7 | Hz |
| 21876-21876.7 | Hz |
| 21887-21887.7 | Hz |
| 21890-21890.7 | Hz |
| 21894-21894.7 | Hz |
| 21963-21963.7 | Hz |
| 22020-22020.7 | Hz |
| 22073-22073.7 | Hz |
| 22119-22119.7 | Hz |
| 22167-22167.7 | Hz |
| 22312-22312.7 | Hz |
| 22338-22338.7 | Hz |
| 22344-22344.7 | Hz |
| 22350-22350.7 | Hz |
| 22353-22353.7 | Hz |
| 22355-22355.7 | Hz |
| 22361-22361.7 | Hz |
| 22411-22411.7 | Hz |
| 22419-22419.7 | Hz |
| 22422-22422.7 | Hz |
| 22423.4-22424 | Hz |
| 22425-22425.7 | Hz |
| 22430-22430.7 | Hz |
| 22437-22437.7 | Hz |
| 22443-22443.7 | Hz |
| 22444-22444.7 | Hz |
| 22454-22454.7 | Hz |
| 22459-22459.7 | Hz |
| 22484-22484.7 | Hz |
| 22491-22491.7 | Hz |
| 22519-22519.7 | Hz |
| 22535-22535.7 | Hz |
| 22538-22538.7 | Hz |
| 22548-22548.7 | Hz |
| 22559.3-22560 | Hz |
| 22582-22582.7 | Hz |
| 22597-22597.7 | Hz |
| 22709-22709.7 | Hz |
| 22730-22730.6 | Hz |
| 22742-22742.7 | Hz |
| 22743-22743.7 | Hz |
| 22763-22763.7 | Hz |
| 22822-22822.7 | Hz |
| 22832-22832.7 | Hz |
| 22840.3-22841 | Hz |
| 22846-22846.7 | Hz |
| 22859-22859.7 | Hz |
| 22863-22863.7 | Hz |
| 22867-22867.7 | Hz |
| 22879-22879.7 | Hz |
| 22932-22932.7 | Hz |
| 22973-22973.7 | Hz |
| 22983-22983.7 | Hz |
| 23012-23012.7 | Hz |
| 23044-23044.7 | Hz |
| 23111-23111.7 | Hz |
| 23117-23117.7 | Hz |
| 23137-23137.7 | Hz |
| 23216-23216.7 | Hz |
| 23237-23237.7 | Hz |
| 23246-23246.7 | Hz |
| 23249-23249.7 | Hz |
| 23258-23258.7 | Hz |
| 23259-23259.7 | Hz |
| 23261-23261.7 | Hz |
| 23284-23284.7 | Hz |
| 23344-23344.7 | Hz |
| 23412-23412.7 | Hz |
| 23444-23444.7 | Hz |
| 23509-23509.7 | Hz |
| 23526-23526.7 | Hz |
| 23548-23548.7 | Hz |
| 23560-23560.7 | Hz |
| 23589-23589.7 | Hz |
| 23624-23624.7 | Hz |
| 23652-23652.7 | Hz |
| 23659-23659.7 | Hz |
| 23667-23667.7 | Hz |
| 23686-23686.7 | Hz |
| 23730-23730.7 | Hz |
| 23921-23921.7 | Hz |
| 23946-23946.7 | Hz |
| 23957-23957.7 | Hz |
| 24014-24014.7 | Hz |
| 24060-24060.7 | Hz |
| 24073-24073.7 | Hz |
| 24085-24085.7 | Hz |
| 24112-24112.7 | Hz |
| 24117-24117.7 | Hz |
| 24142-24142.7 | Hz |
| 24146-24146.7 | Hz |
| 24174-24174.7 | Hz |
| 24196-24196.7 | Hz |
| 24198-24198.7 | Hz |
| 24228-24228.7 | Hz |
| 24275-24275.7 | Hz |
| 24413-24413.7 | Hz |
| 24508-24508.7 | Hz |
| 24609.3-24610 | Hz |
| 24789-24789.7 | Hz |
| 24811-24811.7 | Hz |
| 24841-24841.7 | Hz |
| 24869-24869.7 | Hz |
| 25199-25199.7 | Hz |
| 25449-25449.7 | Hz |
| 25454-25454.7 | Hz |
| 26044-26044.7 | Hz |
| 26073-26073.7 | Hz |
| 26113-26113.7 | Hz |
| 26142-26142.7 | Hz |
| 26163-26163.7 | Hz |
| 26164-26164.7 | Hz |
| 26212-26212.7 | Hz |
| 26343-26343.7 | Hz |
| 26403-26403.7 | Hz |
| 26465-26465.7 | Hz |
| 26534-26534.7 | Hz |
| 26722-26722.7 | Hz |
| 26861-26861.7 | Hz |
| 26954-26954.7 | Hz |
| 27114-27114.7 | Hz |
| 27214-27214.7 | Hz |
| 27291-27291.7 | Hz |
| 27328-27328.6 | Hz |
| 27478-27478.7 | Hz; |
| 27713-27713.7 | Hz |
| 28212-28212.7 | Hz |
| 28222-28222.7 | Hz |
| 28272-28272.7 | Hz |
| 28361-28361.7 | Hz |
| 28537-28537.7 | Hz |
| 28653-28653.7 | Hz |
| 28744-28744.7 | Hz |
| 30075-30075.7 | Hz |
| 30304-30304.7 | Hz |
| 30373-30373.7 | Hz |
| 30463.3-30464 | Hz |
| 30666-30666.7 | Hz |
| 30713-30713.7 | Hz |
| 30759-30759.7 | Hz |
| 30942-30942.7 | Hz |
| 31051-31051.7 | Hz |
| 31572-31572.7 | Hz |
| 31633-31633.7 | Hz |
| 31685-31685.7 | Hz |
| 31713-31713.7 | Hz |
| 31847-31847.7 | Hz |
| 32143-32143.7 | Hz |
| 32493-32493.7 | Hz |
| 32591.3-32592 | Hz |
| 32866-32866.7 | Hz |
| 32870-32870.7 | Hz |
| 33366-33366.7 | Hz |
| 33414-33414.7 | Hz |
| 33536-33536.7 | Hz |
| 33546-33546.7 | Hz |
| 33553-33553.7 | Hz |
| 33765-33765.7 | Hz |
| 34405-34405.7 | Hz |
| 34557.3-34558 | Hz |
| 34562-34562.7 | Hz |
| 34614-34614.7 | Hz |
| 34854-34854.7 | Hz |
| 34984-34984.6 | Hz |
| 35296-35296.7 | Hz |
| 35324-35324.7 | Hz |
| 35543-35543.7 | Hz |
| 35826-35826.7 | Hz |
| 36343-36343.7 | Hz |
| 36410-36410.7 | Hz |
| 37527-37527.7 | Hz |
| 37546-37546.7 | Hz |
| 37680-37680.7 | Hz |
| 38144-38144.7 | Hz |
| 38216-38216.7 | Hz |
| 38483-38483.7 | Hz |
| 38659-38659.7 | Hz |
| 38682-38682.7 | Hz |
| 39035-39035.7 | Hz |
| 39053-39053.7 | Hz |
| 39153-39153.7 | Hz |
| 39183-39183.7 | Hz |
| 39223-39223.7 | Hz |
| 39423-39423.7 | Hz |
| 39510-39510.7 | Hz |
| 39586-39586.7 | Hz |
| 39729-39729.7 | Hz |
| 40111-40111.7 | Hz |
| 40133-40133.7 | Hz |
| 40394-40394.7 | Hz |
| 40489-40489.7 | Hz |
| 40595-40595.7 | Hz |
| 40693-40693.7 | Hz |
| 40734-40734.7 | Hz |
| 40762-40762.7 | Hz |
| 40896-40896.7 | Hz |
| 40899-40899.7 | Hz |
| 41062-41062.7 | Hz |
| 41172-41172.7 | Hz |
| 41244-41244.7 | Hz |
| 41279-41279.7 | Hz |
| 41325-41325.7 | Hz |
| 41424-41424.7 | Hz |
| 41473-41473.7 | Hz |
| 41534-41534.7 | Hz |
| 41557-41557.7 | Hz |
| 41561-41561.7 | Hz |
| 41574-41574.7 | Hz |
| 41623-41623.7 | Hz |
| 41735-41735.7 | Hz |
| 41859-41859.7 | Hz |
| 41861-41861.7 | Hz |
| 41883-41883.7 | Hz |
| 42146-42146.7 | Hz |
| 42293-42293.7 | Hz |
| 42402-42402.7 | Hz |
| 42544-42544.7 | Hz |
| 42692.3-42693 | Hz |
| 42713-42713.7 | Hz |
| 42812-42812.7 | Hz |
| 42813-42813.7 | Hz |
| 42913-42913.7 | Hz |
| 42923-42923.7 | Hz |
| 43162-43162.7 | Hz |
| 43166-43166.7 | Hz |
| 43172-43172.7 | Hz |
| 43243-43243.7 | Hz |
| 43325-43325.7 | Hz |
| 43379-43379.7 | Hz |
| 43413-43413.7 | Hz |
| 43542-43542.7 | Hz |
| 43603-43603.7 | Hz |
| 43633-43633.7 | Hz |
| 43692-43692.7 | Hz |
| 43851-43851.7 | Hz |
| 44162-44162.7 | Hz |
| 44517-44517.7 | Hz |
| 44531-44531.7 | Hz |
| 44575.3-44576 | Hz |
| 44652-44652.7 | Hz |
| 44692-44692.7 | Hz |
| 44742-44742.7 | Hz |
| 44773-44773.7 | Hz |
| 44855-44855.7 | Hz |
| 44913-44913.7 | Hz |
| 45163-45163.7 | Hz |
| 45362-45362.7 | Hz |
| 45393-45393.7 | Hz |
| 45474-45474.7 | Hz |
| 45490-45490.7 | Hz |
| 45526-45526.7 | Hz |
| 46112-46112.7 | Hz |
| 46143-46143.7 | Hz |
| 46282-46282.7 | Hz |
| 46285-46285.7 | Hz |
| 46340-46340.7 | Hz |
| 46662-46662.7 | Hz |
| 46666-46666.7 | Hz |
| 46672.3-46673 | Hz |
| 46673-46673.7 | Hz |
| 46689-46689.6 | Hz |
| 46746-46746.7 | Hz |
| 46793-46793.7 | Hz |
| 47036-47036.7 | Hz |
| 47297-47297.7 | Hz |
| 47693.4-47694 | Hz |
| 48103-48103.7 | Hz |
| 48173-48173.7 | Hz |
| 48204-48204.7 | Hz |
| 48314-48314.7 | Hz |
| 48535-48535.7 | Hz |
| 48583-48583.7 | Hz |
| 48653-48653.7 | Hz |
| 48683-48683.7 | Hz |
| 48793-48793.7 | Hz |
| 49434-49434.7 | Hz |
| 50072-50072.7 | Hz |
| 50229-50229.7 | Hz |
| 50243-50243.7 | Hz |
| 50254-50254.7 | Hz |
| 50292-50292.7 | Hz |
| 51008-51008.7 | Hz |
| 51592-51592.7 | Hz |
| 51946-51946.7 | Hz |
| 52063-52063.7 | Hz |
| 52097-52097.7 | Hz |
| 52752-52752.7 | Hz |
| 52786-52786.7 | Hz |
| 52812-52812.7 | Hz |
| 52912-52912.7 | Hz |
| 53079-53079.7 | Hz |
| 53947-53947.7 | Hz |
| 54293-54293.7 | Hz |
| 54297-54297.7 | Hz |
| 55072-55072.7 | Hz |
| 55592-55592.7 | Hz |
| 55723-55723.7 | Hz |
| 56093-56093.7 | Hz |
| 57927-57927.7 | Hz |
| 58094-58094.7 | Hz |
| 59089-59089.7 | Hz |
| 59288-59288.7 | Hz |
| 59907-59907.7 | Hz |
| 59997-59997.7 | Hz |
| 61287-61287.7 | Hz |
| 61789-61789.7 | Hz |
| 65586-65586.7 | Hz |
| 66553-66553.7 | Hz |
| 66969-66969.7 | Hz |
| 67683-67683.7 | Hz |
| 67697-67697.6 | Hz |
| 69481-69481.7 | Hz |
| 71204-71204.7 | Hz |
| 71447-71447.7 | Hz |
| 71635-71635.7 | Hz |
| 74017-74017.7 | Hz |
| 76227-76227.7 | Hz |
| 76537-76537.7 | Hz |
| 76659-76659.7 | Hz |
| 78229-78229.7 | Hz |
| 78477.4-78478 | Hz |
| 79658-79658.7 | Hz |
| 79867-79867.7 | Hz |
| 80715-80715.7 | Hz |
| 80993.3-80994 | Hz |
| 81999-81999.7 | Hz |
| 82024-82024.7 | Hz |
| 82845-82845.7 | Hz |
| 83612-83612.7 | Hz |
| 84086-84086.7 | Hz |
| 85810-85810.7 | Hz |
| 86521-86521.7 | Hz |
| 87507-87507.7 | Hz |
| 87798.3-87799 | Hz |
| 88281-88281.7 | Hz |
| 88898-88898.7 | Hz |
| 89748-89748.7 | Hz |
| 91245-91245.7 | Hz |
| 91271-91271.7 | Hz |
| 91877-91877.7 | Hz |
| 92182-92182.7 | Hz |
| 92361.3-92362 | Hz |
| 92787-92787.7 | Hz |
| 93332-93332.7 | Hz |
| 93530-93530.7 | Hz |
| 94494-94494.7 | Hz |
| 94968.3-94969 | Hz |
| 95245-95245.7 | Hz |
| 95742-95742.7 | Hz |
| 99281-99281.7 | Hz |
| 99846-99846.7 | Hz |

2. The method of claim 1, wherein 10 or more cancer-specific amplitude modulation frequencies are selected from Table A, and optionally from any of Tables 2, 4, 7, 13, 16, 18, 20 and 23-32, provided that at least one or more frequencies are selected from Table A.

3. The method of claim 1, the cancer is selected from the group consisting of: breast cancer, neuroendocrine tumors, non-Hodgkin lymphoma, adenocarcinoma, head and neck cancer, gastric cancer, glioblastoma, squamous cell carcinoma, hepatocellular carcinoma, cholangiocarcinoma, mesothelioma, thyroid cancer, prostate cancer, rhabdomyosarcoma, lung cancer, kidney cancer, ovarian cancer, bladder cancer, leiomyosarcoma, myeloma, lymphoma, leukemia, chronic lymphoid cancer, brain cancer, and colorectal cancer.

4. The method according to claim 1, wherein the carrier frequency is from about 0.1-1000 MHz.

5. The method according to claim 1, wherein the modulation frequency is from 100 Hz to 99,000 Hz.

6. The method according to claim 1, wherein the frequency of each of the one or more modulated output signals is controlled to within an accuracy of 1 part per 10,000 relative to a reference amplitude modulation frequency.

7. The method according to claim 1, wherein each of the one or more modulated output signals is maintained at a stability during emission of at least 10⁻⁵.

8. The method according to claim 1, wherein specific absorption rate (SAR) of the low energy high frequency radiation absorbed by the patient is from about 1 microWatt per kilogram of tissue to about 50 Watts per kilogram of tissue.

9. The method according to claim 1, wherein the low energy high frequency radiation is applied to the subject undergoing treatment via an electrically conductive probe.

10. The method of claim 9, wherein the electrically conductive probe is configured for contact with a mucosa of the subject or with the skin of the subject.

11. The method according to claim 1, wherein the one or more modulated output signals are generated either sequentially or simultaneously.

12. The method according to claim 1, wherein the amplitude modulation frequency has been determined or predetermined by a bio-feedback process involving measurements of one or more physiological responses by subjects pre-diagnosed with the cancer type upon exposure of the pre-diagnosed subjects to the amplitude modulation frequency.

13. The method according to claim 1, wherein the low energy high frequency radiation comprises 40 or more modulated output signals.

14. The method according to claim 3, wherein the cancer is breast cancer and wherein the subject is treated with 10 or more frequencies comprising:

| | |
|---|---|
| 374-374.7 | Hz |
| 419.8-420.4 | Hz |
| 420.4-420.8 | Hz |
| 429-429.7 | Hz |
| 1336-1336.7 | Hz |
| 1369-1369.7 | Hz |
| 1728-1728.7 | Hz |
| 1771.8-1772.3 | Hz |
| 1772.3-1772.8 | Hz |
| 2811-2811.7 | Hz |
| 2824-2824.7 | Hz |
| 2840-2840.7 | Hz |
| 2858.8-2859.35 | Hz |
| 2860-2860.7 | Hz |
| 2878-2878.7 | Hz |
| 2890-2890.7 | Hz |
| 3209-3209.7 | Hz |
| 3322-3322.7 | Hz |
| 3360-3360.7 | Hz |
| 3663-3663.7 | Hz |
| 3839-3839.7 | Hz |
| 3842.4-3843 | Hz |
| 3854-3854.7 | Hz |
| 3936-3936.7 | Hz |
| 4323-4323.7 | Hz |
| 4329-4329.7 | Hz |
| 4416-4416.7 | Hz |
| 4430-4430.7 | Hz |
| 4477.4-4478 | Hz |
| 4480.9-4481.35 | Hz |
| 4714-4714.7 | Hz |
| 4805.3-4806 | Hz |
| 4813-4813.7 | Hz |
| 4819-4819.7 | Hz |
| 4822-4822.7 | Hz |
| 4823-4823.7 | Hz |
| 4833-4833.7 | Hz |
| 4890-4890.7 | Hz |
| 5154-5154.7 | Hz |
| 5243-5243.7 | Hz |
| 5407.3-5408 | Hz |
| 5716-5716.7 | Hz |
| 5976-5976.7 | Hz |
| 6024-6024.7 | Hz |
| 6070-6070.7 | Hz |
| 6094-6094.7 | Hz |
| 6414-6414.7 | Hz |
| 6717-6717.7 | Hz |
| 6751-6751.7 | Hz |
| 6968.4-6969 | Hz |
| 7013-7013.7 | Hz |
| 7042-7042.7 | Hz |
| 7080-7080.7 | Hz |
| 7145.4-7146 | Hz |
| 7151-7151.7 | Hz |
| 7158-7158.7 | Hz |
| 7225-7225.7 | Hz |
| 8332-8332.7 | Hz |
| 8824-8824.7 | Hz |
| 8837-8837.7 | Hz |
| 9935-9935.7 | Hz |
| 9961-9961.7 | Hz |
| 10013-10013.7 | Hz |
| 10064-10064.7 | Hz |
| 10339.8-10340.4 | Hz |
| 10384-10384.7 | Hz |
| 10745-10745.7 | Hz |
| 10788-10788.7 | Hz |
| 10834-10834.7 | Hz |
| 10854-10854.7 | Hz |
| 10873-10873.7 | Hz |
| 10894-10894.7 | Hz |
| 11163-11163.7 | Hz |
| 11414-11414.7 | Hz |
| 11421-11421.7 | Hz |
| 11429-11429.7 | Hz |
| 11719-11719.7 | Hz |
| 11745-11745.7 | Hz |
| 11931-11931.7 | Hz |
| 11974-11974.7 | Hz |
| 11993-11993.7 | Hz |
| 12118-12118.7 | Hz |
| 12137-12137.7 | Hz |
| 12144-12144.7 | Hz |
| 12318-12318.7 | Hz |
| 12342-12342.7 | Hz |
| 12364-12364.7 | Hz |
| 12557-12557.7 | Hz |
| 12588-12588.7 | Hz |
| 13329-13329.7 | Hz |
| 13377-13377.7 | Hz |
| 13386-13386.7 | Hz |
| 13644-13644.7 | Hz |
| 13661-13661.7 | Hz |
| 13713-13713.7 | Hz |
| 14164-14164.7 | Hz |
| 14308-14308.7 | Hz |
| 14333.4-14333.9 | Hz |
| 14432-14432.7 | Hz |
| 14524-14524.7 | Hz |
| 14589-14589.7 | Hz |
| 14729-14729.7 | Hz |
| 14813-14813.7 | Hz |
| 14835-14835.7 | Hz |
| 14858-14858.7 | Hz |
| 14885-14885.7 | Hz |
| 15035-15035.6 | Hz |
| 15067-15067.7 | Hz |
| 15126-15126.7 | Hz |
| 15195-15195.7 | Hz |
| 15269-15269.7 | Hz |
| 15432-15432.7 | Hz |
| 15432-15432.7 | Hz |
| 15563-15563.7 | Hz |
| 15621-15621.7 | Hz |
| 15624-15624.7 | Hz |
| 15642-15642.7 | Hz |
| 15647-15647.7 | Hz |
| 15669-15669.7 | Hz |
| 15698-15698.7 | Hz |
| 15823-15823.7 | Hz |
| 16347-16347.7 | Hz |
| 17832-17832.7 | Hz |
| 18221-18221.7 | Hz |
| 18260-18260.7 | Hz |
| 19433-19433.7 | Hz |
| 19446-19446.7 | Hz |
| 19489.3-19490 | Hz |
| 19782-19782.7 | Hz |
| 19827-19827.7 | Hz |
| 21291.4-21292 | Hz |
| 21887-21887.7 | Hz |
| 22020-22020.7 | Hz |
| 22419-22419.7 | Hz |
| 22422-22422.7 | Hz |
| 22425-22425.7 | Hz |
| 22444-22444.7 | Hz |
| 22491-22491.7 | Hz |
| 22840.3-22841 | Hz |
| 22859-22859.7 | Hz |
| 22973-22973.7 | Hz |
| 22983-22983.7 | Hz |
| 23258-23258.7 | Hz |
| 23652-23652.7 | Hz |
| 24174-24174.7 | Hz |
| 24811-24811.7 | Hz |
| 26954-26954.7 | Hz |
| 27114-27114.7 | Hz |
| 30463.3-30464 | Hz |
| 34557.3-34558 | Hz |
| 34854-34854.7 | Hz |
| 38144-38144.7 | Hz |
| 38682-38682.7 | Hz |
| 41561-41561.7 | Hz |
| 41623-41623.7 | Hz |
| 41735-41735.7 | Hz |
| 42713-42713.7 | Hz |
| 44575.3-44576 | Hz |
| 46662-46662.7 | Hz |
| 48173-48173.7 | Hz |
| 50292-50292.7 | Hz |

15. The method according to claim 3, wherein the cancer is neuroendocrine cancer and wherein the subject is treated with 10 or more frequencies comprising:

| | |
|---|---|
| 843-843.7 | Hz |
| 1656-1656.7 | Hz |
| 2423-2423.7 | Hz |
| 2480-2480.7 | Hz |
| 2707-2707.7 | Hz |
| 2890-2890.7 | Hz |
| 3080.3-3081 | Hz |
| 3661-3661.7 | Hz |
| 3678-3678.7 | Hz |
| 3976.4-3977 | Hz |
| 4048-4048.7 | Hz |
| 4055-4055.7 | Hz |
| 4368.3-4369 | Hz |
| 4459-4459.7 | Hz |
| 4621-4621.7 | Hz |
| 4837-4837.7 | Hz |
| 4866-4866.7 | Hz |
| 5165.4-5166 | Hz |
| 5265-5265.7 | Hz |
| 5270-5270.7 | Hz |
| 6436-6436.7 | Hz |
| 7037-7037.7 | Hz |
| 8008-8008.7 | Hz |
| 8024-8024.7 | Hz |
| 8097-8097.7 | Hz |
| 8427-8427.7 | Hz |
| 8478.3-8479 | Hz |
| 8480-8480.7 | Hz |
| 8492-8492.7 | Hz |
| 8853-8853.7 | Hz |
| 8877-8877.7 | Hz |
| 9122-9122.7 | Hz |
| 9131-9131.7 | Hz |
| 9174-9174.7 | Hz |
| 10073-10073.7 | Hz |
| 10848.7-10849.3 | Hz |
| 11267-11267.7 | Hz |
| 11336-11336.7 | Hz |
| 11524-11524.7 | Hz |
| 11551.3-11552 | Hz |
| 11927.4-11928 | Hz |
| 12309-12309.7 | Hz |
| 13617-13617.7 | Hz |
| 13674-13674.7 | Hz |
| 13774-13774.7 | Hz |
| 14350-14350.7 | Hz |
| 14456-14456.7 | Hz |
| 14479-14479.7 | Hz |
| 14540-14540.7 | Hz |
| 14553-14553.7 | Hz |
| 14756-14756.7 | Hz |
| 14922-14922.7 | Hz |
| 14961-14961.7 | Hz |
| 15226-15226.7 | Hz |
| 15817-15817.7 | Hz |
| 15825-15825.7 | Hz |
| 15835-15835.7 | Hz |
| 15836-15836.7 | Hz |
| 15897-15897.7 | Hz |
| 16071-16071.1 | Hz |
| 17417-17417.7 | Hz |
| 17418-17418.7 | Hz |
| 17473-17473.7 | Hz |
| 17474-17474.7 | Hz |
| 17621-17621.7 | Hz |
| 17655-17655.7 | Hz |
| 18655-18655.7 | Hz |
| 18672-18672.7 | Hz |
| 18678-18678.7 | Hz |
| 19393-19393.7 | Hz |
| 19426-19426.7 | Hz |
| 19817-19817.7 | Hz |
| 19819-19819.7 | Hz |
| 19861-19861.7 | Hz |
| 19867-19867.7 | Hz |
| 20818-20818.7 | Hz |
| 21708-21708.7 | Hz |
| 21720-21720.7 | Hz |
| 21865-21865.7 | Hz |
| 21876-21876.7 | Hz |
| 22119-22119.7 | Hz |
| 22454-22454.7 | Hz |
| 24073-24073.7 | Hz |
| 30304-30304.7 | Hz |
| 31685-31685.7 | Hz |
| 43633-43633.7 | Hz |
| 44773-44773.7 | Hz |
| 46662-46662.7 | Hz |
| 46793-46793.7 | Hz |
| 48103-48103.7 | Hz |
| 50243-50243.7 | Hz. |

16. The method according to claim 3, wherein the cancer is non-Hodgkin lymphoma and wherein the subject is treated with 10 or more frequencies comprising:

| | |
|---|---|
| 3222-3222.7 | Hz |
| 3228-3228.7 | Hz |
| 3272-3272.7 | Hz |
| 3618-3618.7 | Hz |
| 4523-4523.7 | Hz |
| 4556-4556.7 | Hz |
| 4584-4584.7 | Hz |
| 5116-5116.7 | Hz |
| 5142-5142.7 | Hz |
| 6413-6413.7 | Hz |
| 6419.3-6420 | Hz |
| 6441-6441.7 | Hz |
| 6474-6474.7 | Hz |
| 6911.3-6912 | Hz |
| 6912-6912.7 | Hz |
| 6943-6943.7 | Hz |
| 7551-7551.7 | Hz |
| 7811-7811.7 | Hz |
| 7857-7857.7 | Hz |
| 7865-7865.7 | Hz |
| 8125-8125.7 | Hz |
| 9106-9106.7 | Hz |
| 9161-9161.7 | Hz |
| 9179-9179.7 | Hz |
| 9230-9230.7 | Hz |
| 9236-9236.7 | Hz |
| 9247-9247.7 | Hz |
| 11730.6-11731.1 | Hz |
| 11785-11785.7 | Hz |
| 13222-13222.7 | Hz |
| 13751-13751.7 | Hz |
| 13780-13780.7 | Hz |
| 15006-15006.7 | Hz |
| 15033-15033.7 | Hz |
| 15056-15056.7 | Hz |
| 15729-15729.7 | Hz |
| 15773-15773.7 | Hz |
| 17229-17229.7 | Hz |
| 17246-17246.7 | Hz |
| 17268-17268.7 | Hz |
| 20256-20256.7 | Hz |
| 22444-22444.7 | Hz |
| 30666-30666.7 | Hz |
| 30713-30713.7 | Hz |
| 30843-30843.7 | Hz |
| 38216-38216.7 | Hz. |

17. The method according to claim 3, wherein the cancer is adenocarcinoma of the pancreas and wherein the subject is treated with 10 or more frequencies comprising:

| | |
|---|---|
| 403-403.7 | Hz |
| 441-441.7 | Hz |
| 527-527.7 | Hz |
| 624.32-624.8 | Hz |
| 633-633.7 | Hz |
| 727-727.7 | Hz |
| 759-759.7 | Hz |
| 785-785.7 | Hz |
| 811-811.7 | Hz |
| 829-829.7 | Hz |
| 841-841.7 | Hz |
| 962-962.7 | Hz |
| 964.5-965 | Hz |
| 972-972.7 | Hz |
| 974.4-975 | Hz |
| 977-977.7 | Hz |
| 1179.2-1179.8 | Hz |
| 1250.3-1251 | Hz |
| 1314-1314.7 | Hz |
| 1352-1352.7 | Hz |
| 1622-1622.7 | Hz |
| 1652-1652.7 | Hz |
| 1656-1656.7 | Hz |
| 1670.3-1671 | Hz |
| 1914-1914.7 | Hz |
| 2346-2346.7 | Hz |
| 2362-2362.7 | Hz |
| 2380-2380.7 | Hz |
| 2469.2-2469.7 | Hz |
| 2627-2627.7 | Hz |
| 2706-2706.7 | Hz |
| 2720.3-2721 | Hz |
| 2726-2726.7 | Hz |
| 2754-2754.7 | Hz |
| 2778-2778.7 | Hz |
| 2842-2842.7 | Hz |
| 2864-2864.7 | Hz |
| 2940-2940.7 | Hz |
| 2988-2988.7 | Hz |
| 3234-3234.7 | Hz |
| 3280.6-3281.3 | Hz |
| 3366-3366.7 | Hz |
| 3428-3428.7 | Hz |
| 3446-3446.7 | Hz |
| 3466-3466.7 | Hz |
| 3523-3523.7 | Hz |
| 3605-3605.7 | Hz |
| 3622.5-3623 | Hz |
| 3627-3627.7 | Hz |
| 3631-3631.7 | Hz |
| 3656-3656.7 | Hz |
| 3663-3663.7 | Hz |
| 3667-3667.7 | Hz |
| 3687-3687.7 | Hz |
| 4152-4152.7 | Hz |
| 4233-4233.7 | Hz |
| 4249-4249.7 | Hz |
| 4281-4281.7 | Hz |
| 4368.3-4369 | Hz |
| 4417-4417.7 | Hz |
| 4425-4425.7 | Hz |
| 4440.4-4441 | Hz |
| 4465-4465.7 | Hz |
| 4480.9-4481.35 | Hz |
| 4580.11-4580.7 | Hz |
| 4621-4621.7 | Hz |
| 4637-4637.7 | Hz |
| 4657-4657.6 | Hz |
| 4725-4725.7 | Hz |
| 4733-4733.7 | Hz |
| 4749-4749.7 | Hz |
| 4841-4841.7 | Hz |
| 4924-4924.7 | Hz |
| 4946-4946.7 | Hz |
| 5063-5063.7 | Hz |
| 5089-5089.7 | Hz |
| 5378.2-5378.7 | Hz |
| 5424-5424.7 | Hz |
| 5438-5438.7 | Hz |
| 6112-6112.7 | Hz |
| 6159-6159.7 | Hz |
| 6161.3-6162 | Hz |
| 6167-6167.7 | Hz |
| 6179-6179.7 | Hz |
| 6220.3-6221 | Hz |
| 6346-6346.7 | Hz |
| 6380.4-6381 | Hz |
| 6808-6808.7 | Hz |
| 6825-6825.7 | Hz |
| 6847-6847.7 | Hz |
| 6850-6850.7 | Hz |
| 6878-6878.7 | Hz |
| 7036-7036.7 | Hz |
| 7037-7037.7 | Hz |
| 7118-7118.7 | Hz |
| 7160-7160.7 | Hz |
| 7432-7432.7 | Hz |
| 7541-7541.7 | Hz |
| 7676-7676.7 | Hz |
| 7936-7936.7 | Hz |
| 8024-8024.7 | Hz |
| 8032-8032.7 | Hz |
| 8043-8043.7 | Hz |
| 8447-8447.7 | Hz |
| 8734-8734.7 | Hz |
| 8756-8756.7 | Hz |
| 8757-8757.7 | Hz |
| 8809-8809.7 | Hz |
| 8823-8823.7 | Hz |
| 8841-8841.7 | Hz |
| 8845-8845.7 | Hz |
| 8846-8846.7 | Hz |
| 8872-8872.7 | Hz |
| 9133-9133.7 | Hz |
| 9155.7-9156.2 | Hz |
| 9520-9520.7 | Hz |
| 9859-9859.7 | Hz |
| 10342-10342.6 | Hz |
| 10352-10352.7 | Hz |
| 10378-10378.7 | Hz |
| 10436-10436.7 | Hz |
| 10566.4-10567 | Hz |
| 10633-10633.7 | Hz |
| 10641-10641.7 | Hz |
| 10671-10671.7 | Hz |
| 10708-10708.7 | Hz |
| 10859-10859.7 | Hz |
| 10876-10876.7 | Hz |
| 11174-11174.7 | Hz |
| 11220-11220.7 | Hz |
| 11247-11247.7 | Hz |
| 11264-11264.7 | Hz |
| 11284-11284.7 | Hz |
| 11630-11630.7 | Hz |
| 11994-11994.7 | Hz |
| 12120.2-12120.7 | Hz |
| 12135-12135.7 | Hz |
| 12152-12152.7 | Hz |
| 12184-12184.7 | Hz |
| 12711-12711.7 | Hz |
| 13375-13375.7 | Hz |
| 13571-13571.7 | Hz |
| 13636.2-13636.7 | Hz |
| 13738.3-13739 | Hz |
| 13947-13947.7 | Hz |
| 14116-14116.7 | Hz |
| 14279-14279.7 | Hz |
| 14376-14376.7 | Hz |
| 14462-14462.7 | Hz |
| 14519-14519.7 | Hz |
| 14558-14558.7 | Hz |
| 14729-14729.7 | Hz |
| 14940.4-14941 | Hz |
| 14957-14957.7 | Hz |
| 15179-15179.7 | Hz |
| 15225-15225.6 | Hz |
| 15536-15536.7 | Hz |
| 15576-15576.7 | Hz |
| 15597-15597.7 | Hz |
| 15663-15663.7 | Hz |
| 15678-15678.7 | Hz |
| 15711-15711.7 | Hz |
| 15724-15724.7 | Hz |
| 15740-15740.7 | Hz |
| 15749-15749.7 | Hz |
| 15824-15824.7 | Hz |
| 15867-15867.7 | Hz |
| 16143-16143.7 | Hz |
| 17214-17214.7 | Hz |
| 17253-17253.7 | Hz |
| 17271-17271.7 | Hz |
| 17283-17283.7 | Hz |
| 17739-17739.7 | Hz |
| 17944-17944.7 | Hz |
| 17962-17962.7 | Hz |
| 18279-18279.7 | Hz |
| 18678.7-18679.3 | Hz |
| 18921-18921.7 | Hz |
| 18922-18922.7 | Hz |
| 19059-19059.7 | Hz |
| 19464-19464.7 | Hz |
| 19686-19686.4 | Hz |
| 19724-19724.7 | Hz |
| 20123-20123.7 | Hz |
| 20128-20128.7 | Hz |
| 20144-20144.7 | Hz |
| 20149.3-20150 | Hz |
| 20219-20219.7 | Hz |
| 20254-20254.7 | Hz |
| 21546-21546.7 | Hz |
| 21616-21616.7 | Hz |
| 21664-21664.7 | Hz |
| 21840-21840.7 | Hz |
| 21876-21876.7 | Hz |
| 21894-21894.7 | Hz |
| 22355-22355.7 | Hz |
| 22430-22430.7 | Hz |
| 22597-22597.7 | Hz |
| 22742-22742.7 | Hz |
| 22822-22822.7 | Hz |
| 22846-22846.7 | Hz |
| 22867-22867.7 | Hz |
| 23237-23237.7 | Hz |
| 23284-23284.7 | Hz |
| 24228-24228.7 | Hz |
| 24275-24275.7 | Hz |
| 24508-24508.7 | Hz |
| 24609.3-24610 | Hz |
| 24841-24841.7 | Hz |
| 27478-27478.7 | Hz |
| 32143-32143.7 | Hz |
| 32493-32493.7 | Hz |
| 33546-33546.7 | Hz |
| 40133-40133.7 | Hz |
| 40595-40595.7 | Hz |
| 44913-44913.7 | Hz |
| 45474-45474.7 | Hz |
| 46672.3-46673 | Hz |
| 48204-48204.7 | Hz |
| 48314-48314.7 | Hz |
| 50072-50072.7 | Hz |
| 50254-50254.7 | Hz |
| 52812-52812.7 | Hz |
| 79867-79867.7 | Hz |
| 80993.3-80994 | Hz |
| 91877-91877.7 | Hz |
| 95742-95742.7 | Hz. |

18. The method according to claim 3, wherein the cancer is head and neck cancer and wherein the subject is treated with 10 or more frequencies comprising:

| | |
|---|---|
| 1858-1858.7 | Hz |
| 2893-2893.7 | Hz |
| 5152-5152.7 | Hz |
| 6897-6897.7 | Hz |
| 7080-7080.7 | Hz |
| 7136-7136.7 | Hz |
| 7274-7274.7 | Hz |
| 8755-8755.7 | Hz |
| 9573-9573.7 | Hz |
| 9656-9656.7 | Hz |
| 10439-10439.7 | Hz |
| 10484-10484.7 | Hz |
| 10645-10645.7 | Hz |
| 10673-10673.7 | Hz |
| 11559-11559.7 | Hz |
| 14052-14052.7 | Hz |
| 17379-17379.7 | Hz |
| 19269-19269.7 | Hz |

19. The method according to claim 3, wherein the cancer is gastric cancer and wherein the subject is treated with 10 or more frequencies comprising:

| | |
|---|---|
| 374-374.7 | Hz |
| 560-560.7 | Hz |
| 628-628.7 | Hz |
| 642.4-643 | Hz |
| 708.3-709 | Hz |
| 749-749.7 | Hz |
| 806-806.6 | Hz |
| 825-825.7 | Hz |
| 843-843.7 | Hz |
| 914-914.7 | Hz |
| 959-959.7 | Hz |
| 969-969.7 | Hz |
| 1157-1157.7 | Hz |
| 1436-1436.7 | Hz |
| 1656-1656.7 | Hz |
| 2379.3-2380 | Hz |
| 2417-2417.7 | Hz |
| 2436-2436.7 | Hz |
| 2455-2455.7 | Hz |
| 2478-2478.7 | Hz |
| 2747-2747.7 | Hz |
| 2762-2762.7 | Hz |
| 2774-2774.7 | Hz |
| 2856.4-2857 | Hz |
| 2891-2891.7 | Hz |
| 2981-2981.7 | Hz |
| 3104.3-3105 | Hz |
| 3133-3133.7 | Hz |
| 3522-3522.7 | Hz |
| 3537-3537.7 | Hz |
| 3549-3549.7 | Hz |
| 3556-3556.7 | Hz |
| 3595-3595.7 | Hz |
| 3927-3927.7 | Hz |
| 4154-4154.7 | Hz |
| 4548-4548.7 | Hz |
| 4723-4723.7 | Hz |
| 5286-5286.7 | Hz |
| 5494.4-5495 | Hz |
| 5548.3-5549 | Hz |
| 5742-5742.7 | Hz |
| 6010-6010.7 | Hz |
| 6064-6064.7 | Hz |
| 6329-6329.7 | Hz |
| 6444-6444.7 | Hz |
| 6469-6469.7 | Hz |
| 6488-6488.7 | Hz |
| 7222.4-7223 | Hz |
| 7646-7646.7 | Hz |
| 7748-7748.7 | Hz |
| 7855-7855.7 | Hz |
| 7927-7927.7 | Hz |
| 7951-7951.7 | Hz |
| 7970-7970.7 | Hz |
| 7979-7979.7 | Hz |
| 8039-8039.7 | Hz |
| 8055.4-8056 | Hz |
| 8926-8926.7 | Hz |
| 9109-9109.7 | Hz |
| 9123-9123.7 | Hz |
| 9136-9136.7 | Hz |
| 9146-9146.7 | Hz |
| 9157-9157.7 | Hz |
| 9194-9194.7 | Hz |
| 10137-10137.7 | Hz |
| 10345-10345.7 | Hz |
| 10727-10727.7 | Hz |
| 10802-10802.7 | Hz |
| 11533-11533.7 | Hz |
| 12165-12165.7 | Hz |
| 13241-13241.7 | Hz |
| 13307-13307.7 | Hz |
| 13330.7-13331.2 | Hz |
| 13408-13408.7 | Hz |
| 13434-13434.7 | Hz |
| 13714-13714.7 | Hz |
| 13746-13746.7 | Hz |
| 14016.4-14017 | Hz |
| 14160-14160.7 | Hz |
| 14483-14483.7 | Hz |
| 14511-14511.7 | Hz |
| 14670.4-14671 | Hz |
| 16309-16309.7 | Hz |
| 17418-17418.7 | Hz |
| 17455-17455.7 | Hz |
| 17485-17485.7 | Hz |
| 17581-17581.7 | Hz |
| 19125-19125.7 | Hz |
| 21430-21430.7 | Hz |
| 21714-21714.7 | Hz |
| 23946-23946.7 | Hz |
| 39586-39586.7 | Hz |
| 40734-40734.7 | Hz |
| 41172-41172.7 | Hz |
| 42146-42146.7 | Hz |
| 46143-46143.7 | Hz. |

20. The method according to claim 3, wherein the cancer is glioblastoma and wherein the subject is treated with 10 or more frequencies comprising:

| | |
|---|---|
| 262-262.7 | Hz |
| 295-295.6 | Hz |
| 415-415.7 | Hz |
| 418-418.7 | Hz |
| 423-423.7 | Hz |
| 434-434.7 | Hz |
| 440.4-441 | Hz |
| 476-476.7 | Hz |
| 556-556.7 | Hz |
| 608-608.7 | Hz |
| 612-612.7 | Hz |
| 627-627.7 | Hz |
| 656-656.7 | Hz |
| 694-694.7 | Hz |
| 708.3-709 | Hz |
| 741-741.7 | Hz |
| 752.4-753 | Hz |
| 757-757.6 | Hz |
| 759-759.7 | Hz |
| 776-776.7 | Hz |
| 796.3-797 | Hz |
| 836-836.7 | Hz |
| 941-941.7 | Hz |
| 1027-1027.7 | Hz |
| 1045-1045.7 | Hz |
| 1195-1195.7 | Hz |
| 1868-1868.7 | Hz |
| 1897-1897.7 | Hz |
| 2044-2044.7 | Hz |
| 2439-2439.7 | Hz |
| 2612-2612.7 | Hz |
| 2632-2632.7 | Hz |
| 2653.3-2654 | Hz |
| 2686-2686.7 | Hz |
| 2690-2690.7 | Hz |
| 2697-2697.7 | Hz |
| 2738-2738.7 | Hz |
| 3157-3157.7 | Hz |
| 3218-3218.7 | Hz |
| 3227.3-3228 | Hz |
| 3249-3249.7 | Hz |
| 3277.3-3278 | Hz |
| 3281.6-3282.1 | Hz |
| 3436-3436.7 | Hz |
| 3633-3633.7 | Hz |
| 3656-3656.7 | Hz |
| 3962-3962.7 | Hz |
| 4148-4148.7 | Hz |
| 4241-4241.7 | Hz |
| 4410-4410.7 | Hz |
| 4416-4416.7 | Hz |
| 4454-4454.7 | Hz |
| 4619-4619.7 | Hz |
| 4632-4632.7 | Hz |
| 4853-4853.7 | Hz |
| 5139-5139.7 | Hz |
| 5156-5156.7 | Hz |
| 5996-5996.7 | Hz |
| 6292-6292.7 | Hz |
| 6297-6297.7 | Hz |
| 6459-6459.7 | Hz |
| 6474-6474.7 | Hz |
| 6492.3-6493 | Hz |
| 6626.3-6627 | Hz |
| 6727.4-6728 | Hz |
| 6809-6809.6 | Hz |
| 6841-6841.7 | Hz |
| 6915.3-6916 | Hz |
| 6943-6943.7 | Hz |
| 7110-7110.7 | Hz |
| 7182.4-7183 | Hz |
| 7194.3-7195 | Hz |
| 7268-7268.7 | Hz |
| 7323-7323.7 | Hz |
| 7480-7480.7 | Hz |
| 7663-7663.7 | Hz |
| 7796-7796.7 | Hz |
| 7936-7936.7 | Hz |
| 8070-8070.7 | Hz |
| 8128.4-8129 | Hz |
| 8145-8145.7 | Hz |
| 8158-8158.7 | Hz |
| 8314-8314.7 | Hz |
| 8355-8355.7 | Hz |
| 8484-8484.7 | Hz |
| 9138.3-9139 | Hz |
| 9155.7-9156.2 | Hz |
| 9921-9921.7 | Hz |
| 10427-10427.7 | Hz |
| 10468-10468.7 | Hz |
| 10478-10478.7 | Hz |
| 10719-10719.7 | Hz |
| 10802-10802.7 | Hz |
| 10809-10809.7 | Hz |
| 10851-10851.7 | Hz |
| 11556-11556.7 | Hz |
| 11913-11913.7 | Hz |
| 12828.3-12829 | Hz |
| 13686.4-13687 | Hz |
| 14249-14249.7 | Hz |
| 14515.4-14516 | Hz |
| 14586.3-14587 | Hz |
| 14796-14796.7 | Hz |
| 15067-15067.7 | Hz |
| 15112-15112.7 | Hz |
| 17095-17095.7 | Hz |
| 17167-17167.7 | Hz |
| 18090-18090.7 | Hz |
| 18670.3-18671 | Hz |
| 18895-18895.7 | Hz |
| 19026-19026.7 | Hz |
| 20237-20237.7 | Hz |
| 20254-20254.7 | Hz |
| 20286-20286.7 | Hz |
| 24196-24196.7 | Hz |
| 25199-25199.7 | Hz |
| 26073-26073.7 | Hz |
| 26163-26163.7 | Hz |
| 26164-26164.7 | Hz |
| 30759-30759.7 | Hz |
| 32591.3-32592 | Hz |
| 36343-36343.7 | Hz |
| 41883-41883.7 | Hz |
| 44517-44517.7 | Hz |
| 44531-44531.7 | Hz |
| 45490-45490.7 | Hz |
| 47297-47297.7 | Hz |
| 49434-49434.7 | Hz |
| 50229-50229.7 | Hz |
| 69481-69481.7 | Hz |
| 78477.4-78478 | Hz |
| 80993.3-80994 | Hz |
| 81999-81999.7 | Hz |
| 85810-85810.7 | Hz |
| 89748-89748.7 | Hz |
| 92182-92182.7 | Hz |
| 95245-95245.7 | Hz. |

21. The method according to claim 3, wherein the cancer is squamous cell carcinoma of the anal canal and wherein the subject is treated with 10 or more frequencies comprising:

| | |
|---|---|
| 295-295.6 | Hz |
| 304-304.7 | Hz |
| 436-436.7 | Hz |
| 581-581.7 | Hz |
| 679-679.7 | Hz |
| 694.3-695 | Hz |
| 708.3-709 | Hz |
| 796.3-797 | Hz |
| 1759-1759.7 | Hz |
| 1858-1858.7 | Hz |
| 1873-1873.7 | Hz |
| 2442-2442.7 | Hz |
| 2522-2522.7 | Hz |
| 2648-2648.7 | Hz |
| 2893-2893.7 | Hz |
| 2975-2975.7 | Hz |
| 2981-2981.7 | Hz |
| 3238-3238.7 | Hz |
| 3249.3-3250 | Hz |
| 3250.3-3250.8 | Hz |
| 3461-3461.7 | Hz |
| 3531-3531.7 | Hz |
| 3535-3535.7 | Hz |
| 3681-3681.7 | Hz |
| 4279-4279.7 | Hz |
| 4440.4-4441 | Hz |
| 4718-4718.7 | Hz |
| 4876-4876.7 | Hz |
| 5034-5034.7 | Hz |
| 5068-5068.7 | Hz |
| 5151-5151.7 | Hz |
| 5249-5249.7 | Hz |
| 5286-5286.7 | Hz |
| 5869-5869.7 | Hz |
| 5881-5881.7 | Hz |
| 6298.4-6299 | Hz |
| 6851-6851.7 | Hz |
| 6879-6879.7 | Hz |
| 6882.4-6883 | Hz |
| 6897-6897.7 | Hz |
| 6980.3-6981 | Hz |
| 7136-7136.7 | Hz |
| 7274-7274.7 | Hz |
| 7673-7673.7 | Hz |
| 7930-7930.7 | Hz |
| 7981-7981.7 | Hz |
| 8328-8328.7 | Hz |
| 8432-8432.7 | Hz |
| 8755-8755.7 | Hz |
| 8949-8949.7 | Hz |
| 9656-9656.7 | Hz |
| 10439-10439.7 | Hz |
| 10484-10484.7 | Hz |
| 11145-11145.7 | Hz |
| 11159-11159.7 | Hz |
| 11253-11253.7 | Hz |
| 11260-11260.7 | Hz |
| 11277.3-11278 | Hz |
| 11559-11559.7 | Hz |
| 11699-11699.7 | Hz |
| 11905-11905.7 | Hz |
| 12139-12139.7 | Hz |
| 13819-13819.7 | Hz |
| 13847-13847.7 | Hz |
| 13903-13903.7 | Hz |
| 17379-17379.7 | Hz |
| 18618-18618.7 | Hz |
| 18742-18742.7 | Hz |
| 21445-21445.7 | Hz |
| 21467-21467.7 | Hz |
| 22763-22763.7 | Hz |
| 33536-33536.7 | Hz |
| 37527-37527.7 | Hz |
| 37546-37546.7 | Hz |
| 53079-53079.7 | Hz |
| 58094-58094.7 | Hz |
| 59288-59288.7 | Hz |
| 59997-59997.7 | Hz |
| 88281-88281.7 | Hz. |

22. The method according to claim 3, wherein the cancer is hepatocellular carcinoma and wherein the subject is treated with 10 or more frequencies comprising:

| | |
|---|---|
| 439-439.7 | Hz |
| 594-594.7 | Hz |
| 737-737.7 | Hz |
| 817-817.7 | Hz |
| 966-966.7 | Hz |
| 996-996.7 | Hz |
| 1785-1785.7 | Hz |
| 1823-1823.7 | Hz |
| 1881-1881.7 | Hz |
| 1963-1963.7 | Hz |
| 1973-1973.7 | Hz |
| 2189-2189.7 | Hz |
| 2321-2321.7 | Hz |
| 2372.3-2372.8 | Hz |
| 2459-2459.7 | Hz |
| 2647-2647.7 | Hz |
| 2825-2825.7 | Hz |
| 2836-2836.7 | Hz |
| 2860-2860.7 | Hz |
| 2971-2971.6 | Hz |
| 3171-3171.7 | Hz |
| 3188-3188.7 | Hz |
| 3749-3749.7 | Hz |
| 3762-3762.7 | Hz |
| 3823-3823.7 | Hz |
| 4007-4007.7 | Hz |
| 4013-4013.7 | Hz |
| 4454-4454.7 | Hz |
| 4997-4997.7 | Hz |
| 5151-5151.7 | Hz |
| 5239-5239.7 | Hz |
| 5322-5322.7 | Hz |
| 6317-6317.7 | Hz |
| 6330-6330.7 | Hz |
| 6334-6334.7 | Hz |
| 6432-6432.7 | Hz |
| 6840-6840.7 | Hz |
| 7098-7098.7 | Hz |
| 7169-7169.7 | Hz |
| 7239-7239.7 | Hz |
| 7338-7338.7 | Hz |
| 7378-7378.7 | Hz |
| 7481-7481.7 | Hz |
| 7510-7510.7 | Hz |
| 7719-7719.7 | Hz |
| 7748-7748.7 | Hz |
| 7787-7787.7 | Hz |
| 8388-8388.7 | Hz |
| 8412-8412.7 | Hz |
| 8522-8522.7 | Hz |
| 8845-8845.7 | Hz |
| 8935-8935.6 | Hz |
| 8949-8949.7 | Hz |
| 9273-9273.7 | Hz |
| 9588-9588.7 | Hz |
| 9695-9695.7 | Hz |
| 10387-10387.7 | Hz |
| 10641-10641.7 | Hz |
| 10731-10731.7 | Hz |
| 10740-10740.7 | Hz |
| 10762-10762.7 | Hz |
| 10768-10768.7 | Hz |
| 10776-10776.7 | Hz |
| 10805-10805.7 | Hz |
| 10831-10831.7 | Hz |
| 10889-10889.7 | Hz |
| 11209-11209.7 | Hz |
| 11243-11243.7 | Hz |
| 11247-11247.7 | Hz |
| 11275-11275.7 | Hz |
| 11295-11295.7 | Hz |
| 11569-11569.7 | Hz |
| 11590-11590.7 | Hz |
| 11946-11946.7 | Hz |
| 12498-12498.7 | Hz |
| 12572-12572.7 | Hz |
| 12649-12649.7 | Hz |
| 13215-13215.7 | Hz |
| 13252-13252.7 | Hz |
| 13735-13735.7 | Hz |
| 13856-13856.7 | Hz |
| 14114-14114.7 | Hz |
| 14170.7-14171.3 | Hz |
| 14532-14532.7 | Hz |
| 14574-14574.7 | Hz |
| 15521-15521.7 | Hz |
| 15528-15528.7 | Hz |
| 15546.3-15547 | Hz |
| 15589-15589.7 | Hz |
| 15982-15982.7 | Hz |
| 16381-16381.7 | Hz |
| 17556-17556.7 | Hz |
| 17749-17749.7 | Hz |
| 17978-17978.7 | Hz |
| 18030-18030.7 | Hz |
| 18246-18246.7 | Hz |
| 18567-18567.7 | Hz |
| 18686-18686.7 | Hz |
| 18842-18842.7 | Hz |
| 18897-18897.7 | Hz |
| 19510-19510.7 | Hz |
| 20324-20324.7 | Hz |
| 20825-20825.7 | Hz |
| 20908-20908.7 | Hz |
| 21616-21616.7 | Hz |
| 21778-21778.7 | Hz |
| 21876-21876.7 | Hz |
| 22353-22353.7 | Hz |
| 22459-22459.7 | Hz |
| 22535-22535.7 | Hz |
| 22709-22709.7 | Hz |
| 23509-23509.7 | Hz |
| 23526-23526.7 | Hz |
| 23560-23560.7 | Hz |
| 23589-23589.7 | Hz |
| 23957-23957.7 | Hz |
| 24014-24014.7 | Hz |
| 24117-24117.7 | Hz |
| 24869-24869.7 | Hz |
| 25449-25449.7 | Hz |
| 25454-25454.7 | Hz |
| 26722-26722.7 | Hz |
| 26861-26861.7 | Hz |
| 30942-30942.7 | Hz |
| 31713-31713.7 | Hz |
| 32866-32866.7 | Hz |
| 32870-32870.7 | Hz |
| 33765-33765.7 | Hz |
| 34405-34405.7 | Hz |
| 34562-34562.7 | Hz |
| 34984-34984.6 | Hz |
| 36410-36410.7 | Hz |
| 38483-38483.7 | Hz |
| 38659-38659.7 | Hz |
| 39035-39035.7 | Hz |
| 39223-39223.7 | Hz |
| 39510-39510.7 | Hz |
| 39729-39729.7 | Hz |
| 40693-40693.7 | Hz |
| 41244-41244.7 | Hz |
| 41574-41574.7 | Hz |
| 42913-42913.7 | Hz |
| 42923-42923.7 | Hz |
| 43243-43243.7 | Hz |
| 43413-43413.7 | Hz |
| 43542-43542.7 | Hz |
| 43603-43603.7 | Hz |
| 44742-44742.7 | Hz |
| 47693.4-47694 | Hz |
| 51592-51592.7 | Hz |
| 53947-53947.7 | Hz |
| 55072-55072.7 | Hz |
| 56093-56093.7 | Hz |
| 59089-59089.7 | Hz |
| 66969-66969.7 | Hz |
| 67683-67683.7 | Hz |
| 76537-76537.7 | Hz |
| 78229-78229.7 | Hz |
| 82845-82845.7 | Hz |
| 83612-83612.7 | Hz |
| 84086-84086.7 | Hz |
| 91245-91245.7 | Hz |
| 92361.3-92362 | Hz |
| 93332-93332.7 | Hz |
| 93530-93530.7 | Hz |
| 94968.3-94969 | Hz |
| 99281-99281.7 | Hz |
| 99846-99846.7 | Hz |

23. The method according to claim 3, wherein the cancer is cholangiocarcinoma and wherein the subject is treated with 10 or more frequencies comprising:

| | |
|---|---|
| 334-334.7 | Hz |
| 380-380.7 | Hz |
| 410-410.7 | Hz |
| 427-427.6 | Hz |
| 434-434.7 | Hz |
| 476-476.7 | Hz |
| 527-527.7 | Hz |
| 560-560.7 | Hz |
| 624-624.7 | Hz |
| 642.4-643 | Hz |
| 657-657.7 | Hz |
| 677.4-678 | Hz |
| 728-728.7 | Hz |
| 806-806.6 | Hz |
| 809-809.7 | Hz |
| 845-845.7 | Hz |
| 920-920.7 | Hz |
| 961-961.7 | Hz |
| 963.8-964.25 | Hz |
| 964.3-964.8 | Hz |
| 974.4-975 | Hz |
| 1156.3-1157 | Hz |
| 1157-1157.7 | Hz |
| 1755-1755.7 | Hz |
| 1873-1873.7 | Hz |
| 1967-1967.7 | Hz |
| 2017.4-2018 | Hz |
| 2021-2021.7 | Hz |
| 2023-2023.7 | Hz |
| 2035-2035.7 | Hz |
| 2053-2053.7 | Hz |
| 2065-2065.7 | Hz |
| 2083-2083.7 | Hz |
| 2094-2094.7 | Hz |
| 2111-2111.7 | Hz |
| 2128-2128.7 | Hz |
| 2278-2278.7 | Hz |
| 2284-2284.7 | Hz |
| 2308-2308.7 | Hz |
| 2324-2324.7 | Hz |
| 2338-2338.7 | Hz |
| 2362-2362.7 | Hz |
| 2381-2381.7 | Hz |
| 2419-2419.7 | Hz |
| 2425-2425.7 | Hz |
| 2430-2430.7 | Hz |
| 2431-2431.6 | Hz |
| 2471-2471.7 | Hz |
| 2480-2480.7 | Hz |
| 2522-2522.7 | Hz |
| 2636-2636.7 | Hz |
| 2669-2669.7 | Hz |
| 2679-2679.7 | Hz |
| 2685-2685.6 | Hz |
| 2716-2716.6 | Hz |
| 2743.4-2744 | Hz |
| 2835-2835.7 | Hz |
| 2854-2854.7 | Hz |
| 2865-2865.7 | Hz |
| 2873.3-2874 | Hz |
| 2886-2886.7 | Hz |
| 3009-3009.7 | Hz |
| 3020-3020.7 | Hz |
| 3042-3042.6 | Hz |
| 3076.3-3077 | Hz |
| 3104.3-3105 | Hz |
| 3135-3135.7 | Hz |
| 3143-3143.7 | Hz |
| 3160.4-3161 | Hz |
| 3167-3167.7 | Hz |
| 3206-3206.7 | Hz |
| 3255-3255.7 | Hz |
| 3267-3267.7 | Hz |
| 3269-3269.7 | Hz |
| 3330.4-3331 | Hz |
| 3516-3516.7 | Hz |
| 3531-3531.7 | Hz |
| 3549-3549.7 | Hz |
| 3564-3564.7 | Hz |
| 3572-3572.7 | Hz |
| 3576-3576.7 | Hz |
| 3619-3619.7 | Hz |
| 3630-3630.7 | Hz |
| 3637.7-3638.3 | Hz |
| 3644-3644.7 | Hz |
| 3696-3696.7 | Hz |
| 3749.3-3750 | Hz |
| 3947-3947.7 | Hz |
| 3954-3954.7 | Hz |
| 4013.4-4014 | Hz |
| 4079.4-4080 | Hz |
| 4123.4-4124 | Hz |
| 4281-4281.7 | Hz |
| 4289-4289.7 | Hz |
| 4312.4-4313 | Hz |
| 4418-4418.7 | Hz |
| 4426-4426.7 | Hz |
| 4441-4441.7 | Hz |
| 4685-4685.6 | Hz |
| 4707-4707.7 | Hz |
| 4715-4715.7 | Hz |
| 4732-4732.7 | Hz |
| 4732-4732.7 | Hz |
| 4748-4748.7 | Hz |
| 4767-4767.7 | Hz |
| 4812-4812.7 | Hz |
| 4821-4821.7 | Hz |
| 4873-4873.7 | Hz |
| 4874-4874.7 | Hz |
| 4876-4876.7 | Hz |
| 4941-4941.7 | Hz |
| 5209.4-5210 | Hz |
| 5229-5229.7 | Hz |
| 5243-5243.7 | Hz |
| 5247-5247.7 | Hz |
| 5262-5262.7 | Hz |
| 5271-5271.7 | Hz |
| 5387.3-5388 | Hz |
| 5573-5573.7 | Hz |
| 5671-5671.7 | Hz |
| 5679-5679.7 | Hz |
| 5966-5966.7 | Hz |
| 5976.3-5977 | Hz |
| 6064-6064.7 | Hz |
| 6086-6086.7 | Hz |
| 6180-6180.7 | Hz |
| 6479.4-6480 | Hz |
| 6513-6513.7 | Hz |
| 6711-6711.7 | Hz |
| 6779-6779.7 | Hz |
| 6915.3-6916 | Hz |
| 7019-7019.7 | Hz |
| 7043-7043.7 | Hz |
| 7130-7130.7 | Hz |
| 7213-7213.7 | Hz |
| 7228.3-7229 | Hz |
| 7234-7234.7 | Hz |
| 7246-7246.7 | Hz |
| 7268-7268.7 | Hz |
| 7367-7367.7 | Hz |
| 7482-7482.7 | Hz |
| 7510.4-7511 | Hz |
| 7680.3-7681 | Hz |
| 7862-7862.7 | Hz |
| 7935-7935.7 | Hz |
| 8012.3-8013 | Hz |
| 8013-8013.7 | Hz |
| 8026-8026.7 | Hz |
| 8028-8028.7 | Hz |
| 8028-8028.7 | Hz |
| 8055.4-8056 | Hz |
| 8072-8072.7 | Hz |
| 8074-8074.7 | Hz |
| 8079-8079.7 | Hz |
| 8141-8141.7 | Hz |
| 8336-8336.7 | Hz |
| 8394.3-8395 | Hz |
| 8421-8421.7 | Hz |
| 8432-8432.7 | Hz |
| 8452-8452.7 | Hz |
| 8492-8492.7 | Hz |
| 8509.4-8510 | Hz |
| 8542-8542.7 | Hz |
| 8779-8779.7 | Hz |
| 8789.3-8790 | Hz |
| 8818-8818.7 | Hz |
| 8852-8852.7 | Hz |
| 8993-8993.7 | Hz |
| 9023-9023.7 | Hz |
| 9112-9112.7 | Hz |
| 9332-9332.7 | Hz |
| 9753-9753.7 | Hz |
| 9846-9846.7 | Hz |
| 10140-10140.7 | Hz |
| 10317-10317.7 | Hz |
| 10362-10362.7 | Hz |
| 10488-10488.7 | Hz |
| 10661-10661.7 | Hz |
| 10665.4-10666 | Hz |
| 10690-10690.7 | Hz |
| 10866-10866.7 | Hz |
| 11081-11081.7 | Hz |
| 11109.3-11110 | Hz |
| 11219-11219.7 | Hz |
| 11582-11582.7 | Hz |
| 11619-11619.7 | Hz |
| 11637-11637.7 | Hz |
| 11802.3-11803 | Hz |
| 11809-11809.7 | Hz |
| 11831-11831.7 | Hz |
| 11872-11872.7 | Hz |
| 11904.3-11905 | Hz |
| 12024.3-12025 | Hz |
| 12228-12228.7 | Hz |
| 12247-12247.7 | Hz |
| 12260.4-12261 | Hz |
| 12274-12274.7 | Hz |
| 12682-12682.7 | Hz |
| 12752-12752.7 | Hz |
| 12783-12783.7 | Hz |
| 12785-12785.7 | Hz |
| 13127-13127.7 | Hz |
| 13457-13457.7 | Hz |
| 13609-13609.7 | Hz |
| 13734-13734.7 | Hz |
| 13747-13747.7 | Hz |
| 13755-13755.7 | Hz |
| 13760-13760.7 | Hz |
| 13761-13761.7 | Hz |
| 13767-13767.7 | Hz |
| 13981-13981.7 | Hz |
| 14005-14005.7 | Hz |
| 14027-14027.7 | Hz |
| 14070-14070.7 | Hz |
| 14078-14078.7 | Hz |
| 14356-14356.7 | Hz |
| 14531-14531.7 | Hz |
| 14545-14545.7 | Hz |
| 14564-14564.7 | Hz |
| 14828-14828.7 | Hz |
| 14867-14867.7 | Hz |
| 14913.4-14914 | Hz |
| 14940-14940.7 | Hz |
| 15173-15173.7 | Hz |
| 15224-15224.7 | Hz |
| 15244-15244.7 | Hz |
| 15432-15432.7 | Hz |
| 15460-15460.7 | Hz |
| 15515-15515.7 | Hz |
| 15539-15539.7 | Hz |
| 15550.4-15551 | Hz |
| 15857-15857.7 | Hz |
| 16082-16082.7 | Hz |
| 16515-16515.7 | Hz |
| 17636-17636.7 | Hz |
| 17673-17673.7 | Hz |
| 18087-18087.7 | Hz |
| 18222-18222.7 | Hz |
| 18521-18521.7 | Hz |
| 18585-18585.7 | Hz |
| 19347-19347.7 | Hz |
| 19889-19889.7 | Hz |
| 21466-21466.7 | Hz |
| 21567-21567.7 | Hz |
| 21844-21844.7 | Hz |
| 21870-21870.7 | Hz |
| 21894-21894.7 | Hz |
| 22073-22073.7 | Hz |
| 22119-22119.7 | Hz |
| 22411-22411.7 | Hz |
| 22437-22437.7 | Hz |
| 22479-22479.7 | Hz |
| 22582-22582.7 | Hz |
| 22730-22730.6 | Hz |
| 22832-22832.7 | Hz |
| 22863-22863.7 | Hz |
| 23237-23237.7 | Hz |
| 23261-23261.7 | Hz |
| 23444-23444.7 | Hz |
| 26212-26212.7 | Hz |
| 27291-27291.7 | Hz |
| 31051-31051.7 | Hz |
| 35296-35296.7 | Hz |
| 37680-37680.7 | Hz |
| 39053-39053.7 | Hz |
| 39183-39183.7 | Hz |
| 39423-39423.7 | Hz |
| 40111-40111.7 | Hz |
| 41557-41557.7 | Hz |
| 42293-42293.7 | Hz |
| 43172-43172.7 | Hz |
| 43379-43379.7 | Hz |
| 44855-44855.7 | Hz |
| 45163-45163.7 | Hz |
| 45362-45362.7 | Hz |
| 46666-46666.7 | Hz |
| 46673-46673.7 | Hz |
| 46689-46689.6 | Hz |
| 51008-51008.7 | Hz |
| 52063-52063.7 | Hz |
| 52786-52786.7 | Hz |
| 52912-52912.7 | Hz |
| 55723-55723.7 | Hz |
| 61287-61287.7 | Hz |
| 61789-61789.7 | Hz |
| 71635-71635.7 | Hz |
| 86521-86521.7 | Hz |
| 87798.3-87799 | Hz |
| 88898-88898.7 | Hz |
| 91271-91271.7 | Hz |
| 92787-92787.7 | Hz. |

24. The method according to claim 3, wherein the cancer is mesothelioma and wherein the subject is treated with 10 or more frequencies comprising:

| | |
|---|---|
| 262-262.7 | Hz |
| 412-412.7 | Hz |
| 418-418.7 | Hz |
| 429-429.7 | Hz |
| 441-441.7 | Hz |
| 442-442.7 | Hz |
| 465-465.7 | Hz |
| 485-485.7 | Hz |
| 517-517.7 | Hz |
| 556-556.7 | Hz |
| 581-581.7 | Hz |
| 612-612.7 | Hz |
| 626-626.7 | Hz |
| 638-638.7 | Hz |
| 664-664.7 | Hz |
| 694.3-695 | Hz |
| 708.3-709 | Hz |
| 734.4-735 | Hz |
| 761-761.7 | Hz |
| 764-764.7 | Hz |
| 776-776.7 | Hz |
| 806-806.6 | Hz |
| 831-831.7 | Hz |
| 883-883.7 | Hz |
| 1191-1191.7 | Hz |
| 1591-1591.7 | Hz |
| 2475.4-2476 | Hz |
| 2581-2581.7 | Hz |
| 2749-2749.7 | Hz |
| 2988-2988.7 | Hz |
| 3042-3042.6 | Hz |
| 3131-3131.7 | Hz |
| 3193-3193.7 | Hz |
| 3240-3240.7 | Hz |
| 3335-3335.7 | Hz |
| 3371-3371.7 | Hz |
| 3518-3518.7 | Hz |
| 3523-3523.7 | Hz |
| 3530-3530.7 | Hz |
| 3556-3556.7 | Hz |
| 3621-3621.7 | Hz |
| 3656-3656.7 | Hz |
| 3674-3674.7 | Hz |
| 3681.7-3682.3 | Hz |
| 3817-3817.7 | Hz |
| 3833-3833.7 | Hz |
| 3877-3877.7 | Hz |
| 3948-3948.7 | Hz |
| 3958-3958.6 | Hz |
| 4044-4044.7 | Hz |
| 4190-4190.7 | Hz |
| 4261-4261.7 | Hz |
| 4330-4330.7 | Hz |
| 5496-5496.7 | Hz |
| 5640.4-5641 | Hz |
| 6416.3-6417 | Hz |
| 6441-6441.7 | Hz |
| 6717-6717.7 | Hz |
| 6758-6758.7 | Hz |
| 6783-6783.7 | Hz |
| 7110-7110.7 | Hz |
| 7140-7140.7 | Hz |
| 7154-7154.7 | Hz |
| 7156-7156.7 | Hz |
| 7253-7253.7 | Hz |
| 7283-7283.7 | Hz |
| 7546-7546.7 | Hz |
| 7725-7725.7 | Hz |
| 8386-8386.7 | Hz |
| 8562.4-8563 | Hz |
| 8810-8810.7 | Hz |
| 8827.4-8828 | Hz |
| 8843-8843.7 | Hz |
| 8859.3-8860 | Hz |
| 8863-8863.7 | Hz |
| 8877-8877.7 | Hz |
| 8881-8881.7 | Hz |
| 9075-9075.7 | Hz |
| 9929-9929.7 | Hz |
| 9947-9947.7 | Hz |
| 9970-9970.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11083-11083.7 | Hz |
| 11124-11124.7 | Hz |
| 11540-11540.7 | Hz |
| 11831-11831.7 | Hz |
| 11929-11929.7 | Hz |
| 11956-11956.7 | Hz |
| 12267-12267.7 | Hz |
| 12929-12929.7 | Hz |
| 13674-13674.7 | Hz |
| 13742-13742.7 | Hz |
| 14285-14285.7 | Hz |
| 14947-14947.7 | Hz |
| 15531-15531.7 | Hz |
| 17737-17737.7 | Hz |
| 17757-17757.7 | Hz |
| 18121-18121.7 | Hz |
| 18126-18126.7 | Hz |
| 18140-18140.7 | Hz |
| 18921-18921.7 | Hz |
| 19406-19406.7 | Hz |
| 19476-19476.7 | Hz |
| 19577-19577.7 | Hz |
| 20634-20634.7 | Hz |
| 21372-21372.7 | Hz |
| 22411-22411.7 | Hz |
| 22519-22519.7 | Hz |
| 22538-22538.7 | Hz |
| 22548-22548.7 | Hz |
| 23111-23111.7 | Hz |
| 23921-23921.7 | Hz |
| 24112-24112.7 | Hz |
| 24146-24146.7 | Hz |
| 39153-39153.7 | Hz. |

25. The method according to claim 3, wherein the cancer is thyroid cancer and wherein the subject is treated with 10 or more frequencies comprising:

| | |
|---|---|
| 834-834.7 | Hz |
| 836-836.7 | Hz |
| 845-845.7 | Hz |
| 1213-1213.7 | Hz |
| 1220-1220.7 | Hz |
| 1252-1252.7 | Hz |
| 1815-1815.7 | Hz |
| 1966-1966.7 | Hz |
| 2050-2050.7 | Hz |
| 2313-2313.7 | Hz |
| 2318-2318.7 | Hz |
| 2320-2320.7 | Hz |
| 2328-2328.7 | Hz |
| 2336-2336.7 | Hz |
| 2359-2359.7 | Hz |
| 2373-2373.7 | Hz |
| 2380-2380.7 | Hz |
| 2425-2425.7 | Hz |
| 2429-2429.7 | Hz |
| 2436-2436.7 | Hz |
| 2513-2513.7 | Hz |
| 2629-2629.7 | Hz |
| 2653.7-2654.4 | Hz |
| 2830-2830.7 | Hz |
| 2834-2834.7 | Hz |
| 3030-3030.7 | Hz |
| 3056-3056.7 | Hz |
| 3609-3609.7 | Hz |
| 3646-3646.7 | Hz |
| 3674-3674.7 | Hz |
| 3694-3694.7 | Hz |
| 3908.7-3909.22 | Hz |
| 3981-3981.7 | Hz |
| 4027-4027.7 | Hz |
| 4386.4-4387 | Hz |
| 4425.7-4426.22 | Hz |
| 4792-4792.7 | Hz |
| 4844-4844.7 | Hz |
| 4870-4870.7 | Hz |
| 5259-5259.7 | Hz |
| 5510-5510.7 | Hz |
| 5533-5533.7 | Hz |
| 5714-5714.7 | Hz |
| 5741-5741.7 | Hz |
| 5742-5742.7 | Hz |
| 5752-5752.7 | Hz |
| 5766-5766.7 | Hz |
| 6392-6392.7 | Hz |
| 6423-6423.7 | Hz |
| 6781-6781.7 | Hz |
| 6832-6832.7 | Hz |
| 6864-6864.7 | Hz |
| 7404.4-7405 | Hz |
| 7526-7526.6 | Hz |
| 7646-7646.7 | Hz |
| 7652-7652.7 | Hz |
| 7913-7913.7 | Hz |
| 8217.4-8218 | Hz |
| 8251-8251.7 | Hz |
| 8347-8347.7 | Hz |
| 8731-8731.7 | Hz |
| 8740-8740.7 | Hz |
| 8749-8749.7 | Hz |
| 8814-8814.7 | Hz |
| 9169-9169.7 | Hz |
| 9506-9506.7 | Hz |
| 10159-10159.7 | Hz |
| 10395-10395.7 | Hz |
| 10541-10541.7 | Hz |
| 10830-10830.7 | Hz |
| 10844-10844.7 | Hz |
| 10878-10878.7 | Hz |
| 10993-10993.7 | Hz |
| 11117-11117.7 | Hz |
| 11779-11779.7 | Hz |
| 11867-11867.7 | Hz |
| 12302-12302.7 | Hz |
| 12356-12356.7 | Hz |
| 13117-13117.7 | Hz |
| 13373-13373.7 | Hz |
| 13679-13679.7 | Hz |
| 13694-13694.7 | Hz |
| 14111-14111.7 | Hz |
| 14116-14116.7 | Hz |
| 14156-14156.7 | Hz |
| 14174-14174.7 | Hz |
| 14281-14281.7 | Hz |
| 14523-14523.7 | Hz |
| 14585-14585.7 | Hz |
| 14690-14690.7 | Hz |
| 14716-14716.7 | Hz |
| 14912-14912.7 | Hz |
| 14974-14974.7 | Hz |
| 15223-15223.7 | Hz |
| 15236-15236.7 | Hz |
| 15329-15329.7 | Hz |
| 15615-15615.7 | Hz |
| 15867-15867.7 | Hz |
| 15919-15919.7 | Hz |
| 15942-15942.7 | Hz |
| 16096-16096.7 | Hz |
| 18040-18040.7 | Hz |
| 18182-18182.7 | Hz |
| 18211-18211.7 | Hz |
| 18246-18246.7 | Hz |
| 18274-18274.7 | Hz |
| 18411-18411.7 | Hz |
| 18454-18454.7 | Hz |
| 18462-18462.7 | Hz |
| 18596-18596.7 | Hz |
| 19365-19365.7 | Hz |
| 19419-19419.7 | Hz |
| 19715-19715.7 | Hz |
| 19744-19744.7 | Hz |
| 19823-19823.7 | Hz |
| 20229-20229.7 | Hz |
| 20284-20284.7 | Hz |
| 20513-20513.7 | Hz |
| 20747-20747.7 | Hz |
| 21963-21963.7 | Hz |
| 22338-22338.7 | Hz |
| 22344-22344.7 | Hz |
| 22350-22350.7 | Hz |
| 22422-22422.7 | Hz |
| 22484-22484.7 | Hz |
| 22932-22932.7 | Hz |
| 23012-23012.7 | Hz |
| 23044-23044.7 | Hz |
| 23216-23216.7 | Hz |
| 23659-23659.7 | Hz |
| 23730-23730.7 | Hz |
| 24060-24060.7 | Hz |
| 24085-24085.7 | Hz |
| 26044-26044.7 | Hz |
| 26343-26343.7 | Hz |
| 27214-27214.7 | Hz |
| 28361-28361.7 | Hz |
| 33366-33366.7 | Hz |
| 33414-33414.7 | Hz |
| 33553-33553.7 | Hz |
| 34614-34614.7 | Hz |
| 35826-35826.7 | Hz |
| 40133-40133.7 | Hz |
| 40394-40394.7 | Hz |
| 40896-40896.7 | Hz |
| 40899-40899.7 | Hz |
| 41424-41424.7 | Hz |
| 41534-41534.7 | Hz |
| 41859-41859.7 | Hz |
| 41861-41861.7 | Hz |
| 42402-42402.7 | Hz |
| 43851-43851.7 | Hz |
| 44162-44162.7 | Hz |
| 44692-44692.7 | Hz |
| 45393-45393.7 | Hz |
| 45526-45526.7 | Hz |
| 46282-46282.7 | Hz |
| 46285-46285.7 | Hz |
| 47036-47036.7 | Hz |
| 48314-48314.7 | Hz |
| 48535-48535.7 | Hz |
| 52097-52097.7 | Hz |
| 54293-54293.7 | Hz |
| 54297-54297.7 | Hz |
| 55592-55592.7 | Hz |
| 57927-57927.7 | Hz |
| 59907-59907.7 | Hz |
| 65586-65586.7 | Hz |
| 67697-67697.6 | Hz |
| 71204-71204.7 | Hz |
| 71447-71447.7 | Hz |
| 74017-74017.7 | Hz |
| 76227-76227.7 | Hz |
| 76659-76659.7 | Hz |
| 82024-82024.7 | Hz |

26. The method according to claim 3, wherein the cancer is prostate cancer and wherein the subject is treated with 10 or more frequencies comprising:

| | |
|---|---|
| 408-408.7 | Hz |
| 429.7-430.3 | Hz |
| 561-561.7 | Hz |
| 568-568.7 | Hz |
| 572-572.7 | Hz |
| 631-631.7 | Hz |
| 709-709.7 | Hz |
| 717-717.7 | Hz |
| 733-733.7 | Hz |
| 810-810.7 | Hz |
| 817-817.7 | Hz |
| 830-830.7 | Hz |
| 1668-1668.7 | Hz |
| 1808-1808.6 | Hz |
| 2483-2483.7 | Hz |
| 2518-2518.7 | Hz |
| 2824-2824.7 | Hz |
| 3207-3207.7 | Hz |
| 3245-3245.7 | Hz |
| 3251-3251.7 | Hz |
| 3266.3-3267 | Hz |
| 3279.3-3280 | Hz |
| 3529-3529.7 | Hz |
| 3562-3562.7 | Hz |
| 3571-3571.7 | Hz |
| 3627-3627.7 | Hz |
| 3661-3661.7 | Hz |
| 3731-3731.7 | Hz |
| 3743-3743.7 | Hz |
| 3756-3756.7 | Hz |
| 3787-3787.7 | Hz |
| 3962-3962.7 | Hz |
| 4152-4152.7 | Hz |
| 4409-4409.7 | Hz |
| 4476-4476.7 | Hz |
| 4806-4806.7 | Hz |
| 4809-4809.7 | Hz |
| 4823-4823.7 | Hz |
| 4841-4841.7 | Hz |
| 4948-4948.7 | Hz |
| 4998-4998.7 | Hz |
| 5336-5336.7 | Hz |
| 5414-5414.7 | Hz |
| 5884-5884.7 | Hz |
| 6117-6117.7 | Hz |
| 6252-6252.7 | Hz |
| 6329-6329.7 | Hz |
| 6333-6333.7 | Hz |
| 6748-6748.7 | Hz |
| 6782-6782.7 | Hz |
| 7112-7112.7 | Hz |
| 7424-7424.7 | Hz |
| 7655-7655.7 | Hz |
| 7906-7906.7 | Hz |
| 7910-7910.7 | Hz |
| 7923-7923.7 | Hz |
| 8373-8373.7 | Hz |
| 8445-8445.7 | Hz |
| 8765-8765.7 | Hz |
| 8829-8829.7 | Hz |
| 8852-8852.7 | Hz |
| 8877-8877.7 | Hz |
| 9078-9078.7 | Hz |
| 9512-9512.7 | Hz |
| 9635-9635.7 | Hz |
| 9681-9681.7 | Hz |
| 9886-9886.7 | Hz |
| 10735-10735.7 | Hz |
| 10748-10748.7 | Hz |
| 10781-10781.7 | Hz |
| 10813.2-10813.8 | Hz |
| 11134-11134.7 | Hz |
| 11155-11155.7 | Hz |
| 11164-11164.7 | Hz |
| 11264-11264.7 | Hz |
| 11381-11381.7 | Hz |
| 11416-11416.7 | Hz |
| 11438-11438.7 | Hz |
| 11469-11469.7 | Hz |
| 11628-11628.7 | Hz |
| 12330-12330.7 | Hz |
| 12391-12391.7 | Hz |
| 12880-12880.7 | Hz |
| 13467-13467.7 | Hz |
| 13562-13562.6 | Hz |
| 13805-13805.7 | Hz |
| 13828-13828.7 | Hz |
| 13834-13834.7 | Hz |
| 13857.4-13858 | Hz |
| 13858-13858.7 | Hz |
| 13871-13871.7 | Hz |
| 14009-14009.7 | Hz |
| 14034-14034.7 | Hz |
| 14153-14153.7 | Hz |
| 14489-14489.7 | Hz |
| 15071-15071.7 | Hz |
| 15149-15149.7 | Hz |
| 15233-15233.7 | Hz |
| 15253-15253.7 | Hz |
| 15263-15263.7 | Hz |
| 15435-15435.7 | Hz |
| 15545-15545.7 | Hz |
| 16176-16176.7 | Hz |
| 16329-16329.7 | Hz |
| 16679-16679.7 | Hz |
| 16804-16804.7 | Hz |
| 16821-16821.7 | Hz |
| 17333-17333.7 | Hz |
| 17435-17435.7 | Hz |
| 17974-17974.7 | Hz |
| 18260-18260.7 | Hz |
| 18641-18641.7 | Hz |
| 18831-18831.7 | Hz |
| 18884-18884.7 | Hz |
| 19453-19453.7 | Hz |
| 20948-20948.7 | Hz |
| 21119-21119.7 | Hz |
| 21167-21167.7 | Hz |
| 21636-21636.7 | Hz |
| 22312-22312.7 | Hz |
| 22361-22361.7 | Hz |
| 22419-22419.7 | Hz |
| 22430-22430.7 | Hz |
| 22443-22443.7 | Hz |
| 22459-22459.7 | Hz |
| 22879-22879.7 | Hz |
| 23117-23117.7 | Hz |
| 23137-23137.7 | Hz |
| 23412-23412.7 | Hz |
| 23548-23548.7 | Hz |
| 23686-23686.7 | Hz |
| 26142-26142.7 | Hz |
| 26465-26465.7 | Hz |
| 27328-27328.6 | Hz |
| 28653-28653.7 | Hz |
| 28744-28744.7 | Hz |
| 31572-31572.7 | Hz |
| 31633-31633.7 | Hz |
| 41062-41062.7 | Hz |
| 41325-41325.7 | Hz |
| 41473-41473.7 | Hz |
| 48683-48683.7 | Hz |
| 48793-48793.7 | Hz |
| 87507-87507.7 | Hz. |

27. The method according to claim 3, wherein the cancer is rhabdomyosarcoma and wherein the subject is treated with 10 or more frequencies comprising:

| | |
|---|---|
| 266.3-267 | Hz |
| 304-304.7 | Hz |
| 331-331.7 | Hz |
| 410-410.7 | Hz |
| 420-420.7 | Hz |
| 429-429.7 | Hz |
| 434-434.7 | Hz |
| 461-461.7 | Hz |
| 537.4-538 | Hz |
| 560-560.7 | Hz |
| 572-572.7 | Hz |
| 605-605.7 | Hz |
| 611-611.7 | Hz |
| 618.3-619 | Hz |
| 626-626.7 | Hz |
| 717-717.7 | Hz |
| 776-776.7 | Hz |
| 779-779.7 | Hz |
| 810-810.7 | Hz |
| 814-814.7 | Hz |
| 826-826.7 | Hz |
| 836.4-837 | Hz |
| 843-843.7 | Hz |
| 843-843.7 | Hz |
| 1314-1314.7 | Hz |
| 1411-1411.7 | Hz |
| 1467-1467.7 | Hz |
| 1785-1785.7 | Hz |
| 1816-1816.7 | Hz |
| 1903-1903.7 | Hz |
| 2073.3-2074 | Hz |
| 2155-2155.7 | Hz |
| 2287-2287.7 | Hz |
| 2381-2381.7 | Hz |
| 2542-2542.7 | Hz |
| 2711-2711.6 | Hz |
| 2854-2854.6 | Hz |
| 2911-2911.7 | Hz |
| 3085.7-3086.3 | Hz |
| 3121-3121.7 | Hz |
| 3232-3232.7 | Hz |
| 3438-3438.7 | Hz |
| 3518-3518.7 | Hz |
| 3544-3544.7 | Hz |
| 3569-3569.7 | Hz |
| 3618-3618.7 | Hz |
| 3910-3910.7 | Hz |
| 3917-3917.7 | Hz |
| 4198-4198.7 | Hz |
| 4233.3-4234 | Hz |
| 4241-4241.7 | Hz |
| 4337-4337.7 | Hz |
| 4358-4358.7 | Hz |
| 4424-4424.7 | Hz |
| 4436-4436.7 | Hz |
| 4485-4485.7 | Hz |
| 4731-4731.7 | Hz |
| 4749-4749.7 | Hz |
| 4776-4776.7 | Hz |
| 5151-5151.7 | Hz |
| 5521-5521.7 | Hz |
| 5545.3-5546 | Hz |
| 5577.3-5578 | Hz |
| 5618-5618.7 | Hz |
| 5631-5631.7 | Hz |
| 5696-5696.7 | Hz |
| 6058-6058.7 | Hz |
| 6443-6443.7 | Hz |
| 6472-6472.6 | Hz |
| 6558-6558.7 | Hz |
| 6651-6651.7 | Hz |
| 6730-6730.7 | Hz |
| 7168.4-7169 | Hz |
| 7406-7406.7 | Hz |
| 7440-7440.7 | Hz |
| 7452.3-7453 | Hz |
| 7649-7649.7 | Hz |
| 7808-7808.7 | Hz |
| 8542-8542.7 | Hz |
| 8546-8546.7 | Hz |
| 8562-8562.7 | Hz |
| 8576-8576.7 | Hz |
| 8698-8698.7 | Hz |
| 8881-8881.7 | Hz |
| 9040-9040.7 | Hz |
| 9074-9074.7 | Hz |
| 9189-9189.6 | Hz |
| 9484.3-9485 | Hz |
| 9851-9851.7 | Hz |
| 9943.4-9944 | Hz |
| 10870-10870.7 | Hz |
| 10891-10891.7 | Hz |
| 12086-12086.7 | Hz |
| 12648-12648.7 | Hz |
| 15195-15195.7 | Hz |
| 17316-17316.7 | Hz |
| 18021-18021.7 | Hz |
| 18567-18567.7 | Hz |
| 19384-19384.7 | Hz |
| 20097-20097.7 | Hz |
| 20278.4-20279 | Hz |
| 21854-21854.7 | Hz |
| 21890-21890.7 | Hz |
| 24198-24198.7 | Hz |
| 24413-24413.7 | Hz |
| 24789-24789.7 | Hz |
| 26534-26534.7 | Hz |
| 27713-27713.7 | Hz |
| 28222-28222.7 | Hz |
| 40489-40489.7 | Hz |
| 40762-40762.7 | Hz |
| 41279-41279.7 | Hz |
| 42923-42923.7 | Hz |
| 43166-43166.7 | Hz |
| 43692-43692.7 | Hz |
| 48583-48583.7 | Hz |
| 49434-49434.7 | Hz |
| 66553-66553.7 | Hz |
| 79658-79658.7 | Hz. |

28. The method according to claim 3, wherein the cancer is colorectal cancer and wherein the subject is treated with 10 or more frequencies comprising:

| | |
|---|---|
| 605-605.7 | Hz |
| 826-826.7 | Hz |
| 834-834.7 | Hz |
| 841.9-842.6 | Hz |
| 1808-1808.6 | Hz |
| 1928-1928.7 | Hz |
| 1989-1989.7 | Hz |
| 2439.7-2440.3 | Hz |
| 2714-2714.7 | Hz |
| 2747-2747.7 | Hz |
| 2759-2759.7 | Hz |
| 2807-2807.7 | Hz |
| 2826-2826.7 | Hz |
| 2981-2981.7 | Hz |
| 3209-3209.7 | Hz |
| 3213-3213.7 | Hz |
| 3244-3244.7 | Hz |
| 3261-3261.7 | Hz |
| 3548-3548.7 | Hz |
| 3634-3634.7 | Hz |
| 3646.4-3647 | Hz |
| 3915-3915.7 | Hz |
| 3922-3922.7 | Hz |
| 3940-3940.7 | Hz |
| 3946-3946.7 | Hz |
| 4135-4135.7 | Hz |
| 4155-4155.7 | Hz |
| 4224-4224.7 | Hz |
| 4241-4241.7 | Hz |
| 4283-4283.7 | Hz |
| 4323-4323.7 | Hz |
| 4438.7-4439.2 | Hz |
| 4447-4447.7 | Hz |
| 4708-4708.7 | Hz |
| 4713-4713.7 | Hz |
| 4718-4718.7 | Hz |
| 4718-4718.7 | Hz |
| 4722-4722.7 | Hz |
| 4776-4776.7 | Hz |
| 4850-4850.7 | Hz |
| 4874-4874.7 | Hz |
| 5205-5205.7 | Hz |
| 5209-5209.7 | Hz |
| 5209-5209.7 | Hz |
| 5221-5221.7 | Hz |
| 5232-5232.7 | Hz |
| 5265-5265.7 | Hz |
| 5427-5427.7 | Hz |
| 5484.4-5485 | Hz |
| 6074-6074.7 | Hz |
| 6112-6112.7 | Hz |
| 6159-6159.7 | Hz |
| 6361-6361.7 | Hz |
| 6438-6438.7 | Hz |
| 6468-6468.7 | Hz |
| 6537-6537.7 | Hz |
| 6648-6648.7 | Hz |
| 6869-6869.7 | Hz |
| 6878-6878.7 | Hz |
| 6914-6914.7 | Hz |
| 6974-6974.7 | Hz |
| 6982-6982.7 | Hz |
| 6986-6986.7 | Hz |
| 7109-7109.7 | Hz |
| 7117-7117.7 | Hz |
| 7137-7137.7 | Hz |
| 7149-7149.7 | Hz |
| 7191.7-7192.3 | Hz |
| 7531-7531.7 | Hz |
| 7560-7560.7 | Hz |
| 7879-7879.7 | Hz |
| 7936-7936.7 | Hz |
| 7970-7970.7 | Hz |
| 7994-7994.7 | Hz |
| 8038.4-8039 | Hz |
| 8413-8413.7 | Hz |
| 8438-8438.7 | Hz |
| 8467-8467.7 | Hz |
| 8771-8771.7 | Hz |
| 8809-8809.7 | Hz |
| 8889-8889.7 | Hz |
| 9071-9071.7 | Hz |
| 9084-9084.7 | Hz |
| 9731-9731.7 | Hz |
| 10566-10566.7 | Hz |
| 10831.7-10832.1 | Hz |
| 10845-10845.7 | Hz |
| 10859-10859.7 | Hz |
| 10906-10906.7 | Hz |
| 10947-10947.7 | Hz |
| 10974-10974.7 | Hz |
| 11110-11110.7 | Hz |
| 11225-11225.7 | Hz |
| 11234-11234.7 | Hz |
| 11255-11255.7 | Hz |
| 11387-11387.7 | Hz |
| 11530-11530.7 | Hz |
| 11576-11576.7 | Hz |
| 11621-11621.7 | Hz |
| 11630-11630.7 | Hz |
| 11638-11638.7 | Hz |
| 11681-11681.7 | Hz |
| 11911-11911.7 | Hz |
| 11934-11934.7 | Hz |
| 11949-11949.7 | Hz |
| 12182-12182.7 | Hz |
| 12373-12373.7 | Hz |
| 12575-12575.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 13418-13418.7 | Hz |
| 13475-13475.7 | Hz |
| 13611-13611.7 | Hz |
| 13615-13615.7 | Hz |
| 13670-13670.7 | Hz |
| 13717-13717.7 | Hz |
| 13736-13736.7 | Hz |
| 13742-13742.7 | Hz |
| 13773-13773.7 | Hz |
| 13775-13775.7 | Hz |
| 13978.3-13979 | Hz |
| 14182-14182.7 | Hz |
| 14417-14417.7 | Hz |
| 14422-14422.7 | Hz |
| 14441-14441.7 | Hz |
| 14443-14443.7 | Hz |
| 14570-14570.7 | Hz |
| 14621-14621.7 | Hz |
| 14630-14630.7 | Hz |
| 14671-14671.7 | Hz |
| 14674-14674.7 | Hz |
| 14863-14863.7 | Hz |
| 14877-14877.7 | Hz |
| 14911-14911.7 | Hz |
| 14957-14957.7 | Hz |
| 14982-14982.7 | Hz |
| 15109-15109.7 | Hz |
| 15133-15133.7 | Hz |
| 15154-15154.7 | Hz |
| 15230-15230.7 | Hz |
| 15250-15250.7 | Hz |
| 15278-15278.7 | Hz |
| 16274-16274.7 | Hz |
| 17106-17106.7 | Hz |
| 17123-17123.7 | Hz |
| 17472-17472.7 | Hz |
| 17515-17515.7 | Hz |
| 17578.4-17579 | Hz |
| 17808-17808.7 | Hz |
| 17825-17825.7 | Hz |
| 18157-18157.7 | Hz |
| 18171-18171.7 | Hz |
| 18294-18294.7 | Hz |
| 18372-18372.7 | Hz |
| 18375-18375.7 | Hz |
| 18396-18396.7 | Hz |
| 18533-18533.7 | Hz |
| 18635-18635.7 | Hz |
| 19342-19342.7 | Hz |
| 19437-19437.7 | Hz |
| 19447-19447.7 | Hz |
| 19829-19829.7 | Hz |
| 20173-20173.7 | Hz |
| 20499-20499.7 | Hz |
| 21389-21389.7 | Hz |
| 21490-21490.7 | Hz |
| 21663-21663.7 | Hz |
| 21833-21833.7 | Hz |
| 22167-22167.7 | Hz |
| 22423.4-22424 | Hz |
| 22559.3-22560 | Hz |
| 22743-22743.7 | Hz |
| 23246-23246.7 | Hz |
| 23249-23249.7 | Hz |
| 23259-23259.7 | Hz |
| 23344-23344.7 | Hz |
| 23624-23624.7 | Hz |
| 23667-23667.7 | Hz |
| 24142-24142.7 | Hz |
| 26113-26113.7 | Hz |
| 26403-26403.7 | Hz |
| 28212-28212.7 | Hz |
| 28272-28272.7 | Hz |
| 28537-28537.7 | Hz |
| 30075-30075.7 | Hz |
| 30373-30373.7 | Hz |
| 31847-31847.7 | Hz |
| 33546-33546.7 | Hz |
| 35324-35324.7 | Hz |
| 35543-35543.7 | Hz |
| 42544-42544.7 | Hz |
| 42692.3-42693 | Hz |
| 42812-42812.7 | Hz |
| 42813-42813.7 | Hz |
| 43162-43162.7 | Hz |
| 43325-43325.7 | Hz |
| 44652-44652.7 | Hz |
| 46340-46340.7 | Hz |
| 46746-46746.7 | Hz |
| 48653-48653.7 | Hz |
| 51946-51946.7 | Hz |
| 52752-52752.7 | Hz |
| 80715-80715.7 | Hz |
| 94494-94494.7 | Hz. |

29. An apparatus for treating a subject suffering from cancer, the apparatus comprising:
a conductive applicator configured to apply to the subject low energy high frequency radiation, wherein the low energy high frequency radiation comprises one or more amplitude-modulated output signals; and
a frequency synthesizer coupled to conductive applicator and configured to generate the one or more amplitude-modulated output signals by generating:
a carrier frequency signal having a carrier frequency from about 1 KHz to 5000 MHz; and
amplitude modulation frequency signals having amplitude modulation frequencies from about 0.1 Hz to about 150,000 Hz, wherein the amplitude modulation frequencies are selected to be cancer-specific frequencies;
wherein the amplitude modulation frequency signals comprise 10 or more cancer-specific amplitude modulation frequencies selected from Table A:
TABLE A

| | |
|---|---|
| 262-262.7 | Hz |
| 266.3-267 | Hz |
| 295-295.6 | Hz |
| 334-334.7 | Hz |
| 374-374.7 | Hz |
| 408-408.7 | Hz |
| 415-415.7 | Hz |
| 418-418.7 | Hz |
| 419.8-420.4 | Hz |
| 420-420.7 | Hz |
| 420.4-420.8 | Hz |
| 429-429.7 | Hz |
| 429.7-430.3 | Hz |
| 439-439.7 | Hz |
| 441-441 7 | Hz |
| 442-442.7 | Hz |
| 465-465.7 | Hz |
| 485-485.7 | Hz |
| 527-527.7 | Hz |
| 556-556.7 | Hz |
| 561-561.7 | Hz |
| 568-568.7 | Hz |
| 572-572.7 | Hz |
| 581-581.7 | Hz |
| 594-594.7 | Hz |
| 605-605.7 | Hz |
| 611-611.7 | Hz |
| 612-612.7 | Hz |
| 624.32-624.8 | Hz |
| 626-626.7 | Hz |
| 627-627.7 | Hz |
| 631-631.7 | Hz |
| 633-633.7 | Hz |
| 638-638.7 | Hz |
| 677.4-678 | Hz |
| 679-679.7 | Hz |
| 709-709.7 | Hz |
| 717-717.7 | Hz |
| 727-727.7 | Hz |
| 733-733.7 | Hz |
| 737-737.7 | Hz |
| 759-759.7 | Hz |
| 761-761.7 | Hz |
| 810-810.7 | Hz |
| 811-811.7 | Hz |
| 817-817.7 | Hz |
| 826-826.7 | Hz |
| 829-829.7 | Hz |
| 830-830.7 | Hz |
| 834-834.7 | Hz |
| 836-836.7 | Hz |
| 883-883.7 | Hz |
| 959-959.7 | Hz |
| 961-961.7 | Hz |
| 963.8-964.25 | Hz |
| 964.5-965 | Hz |
| 966-966.7 | Hz |
| 969-969.7 | Hz |
| 972-972.7 | Hz |
| 974.4-975 | Hz |
| 977-977.7 | Hz |
| 996-996.7 | Hz |
| 1027-1027.7 | Hz |
| 1045-1045.7 | Hz |
| 1157-1157.7 | Hz |
| 1179.2-1179.8 | Hz |
| 1195-1195.7 | Hz |
| 1213-1213.7 | Hz |
| 1220-1220.7 | Hz |
| 1252-1252.7 | Hz |
| 1314-1314.7 | Hz |
| 1336-1336.7 | Hz |
| 1352-1352.7 | Hz |
| 1369-1369.7 | Hz |
| 1436-1436.7 | Hz |
| 1467-1467.7 | Hz |
| 1622-1622.7 | Hz |
| 1652-1652.7 | Hz |
| 1668-1668.7 | Hz |
| 1728-1728.7 | Hz |
| 1759-1759.7 | Hz |
| 1771.7-1772.3 | Hz |
| 1772.3-1772.8 | Hz |
| 1785-1785.7 | Hz |
| 1808-1808.6 | Hz |
| 1815-1815.7 | Hz |
| 1823-1823.7 | Hz |
| 1858-1858.7 | Hz |
| 1868-1868.7 | Hz |
| 1881-1881.7 | Hz |
| 1897-1897.7 | Hz |
| 1903-1903.7 | Hz |
| 1914-1914.7 | Hz |
| 1928-1928.7 | Hz |
| 1963-1963.7 | Hz |
| 1966-1966.7 | Hz |
| 1967-1967.7 | Hz |
| 1973-1973.7 | Hz |
| 1989-1989.7 | Hz |
| 2021-2021.7 | Hz |
| 2023-2023.7 | Hz |
| 2035-2035.7 | Hz |
| 2044-2044.7 | Hz |
| 2065-2065.7 | Hz |
| 2094-2094.7 | Hz |
| 2111-2111.7 | Hz |
| 2128-2128.7 | Hz |
| 2189-2189.7 | Hz |
| 2284-2284.7 | Hz |
| 2287-2287.7 | Hz |
| 2313-2313.7 | Hz |
| 2321-2321.7 | Hz |
| 2328-2328.7 | Hz |
| 2346-2346.7 | Hz |
| 2359-2359.7 | Hz |
| 2372.3-2372.8 | Hz |
| 2373-2373.7 | Hz |
| 2380-2380.7 | Hz |
| 2429-2429.7 | Hz |
| 2439.7-2440.3 | Hz |
| 2455-2455.7 | Hz |
| 2459-2459.7 | Hz |
| 2469.2-2469.7 | Hz |
| 2483-2483.7 | Hz |
| 2513-2513.7 | Hz |
| 2518-2518.7 | Hz |
| 2612-2612.7 | Hz |
| 2627-2627.7 | Hz |
| 2629-2629.7 | Hz |
| 2632-2632.7 | Hz |
| 2636-2636.7 | Hz |
| 2647-2647.7 | Hz |
| 2648-2648.7 | Hz |
| 2653.3-2654 | Hz |
| 2653.7-2654.4 | Hz |
| 2679-2679.7 | Hz |
| 2697-2697.7 | Hz |
| 2706-2706.7 | Hz |
| 2707-2707.7 | Hz |
| 2714-2714.7 | Hz |
| 2720.3-2721 | Hz |
| 2726-2726.7 | Hz |
| 2738-2738.7 | Hz |
| 2747-2747.7 | Hz |
| 2754-2754.7 | Hz |
| 2759-2759.7 | Hz |
| 2762-2762.7 | Hz |
| 2774-2774.7 | Hz |
| 2778-2778.7 | Hz |
| 2807-2807.7 | Hz |
| 2811-2811.7 | Hz |
| 2824-2824.7 | Hz |
| 2825-2825.7 | Hz |
| 2826-2826.7 | Hz |
| 2830-2830.7 | Hz |
| 2834-2834.7 | Hz |
| 2836-2836.7 | Hz |
| 2840-2840.7 | Hz |
| 2842-2842.7 | Hz |
| 2854-2854.6 | Hz |
| 2854-2854.7 | Hz |
| 2858.8-2859.35 | Hz |
| 2860-2860.7 | Hz |
| 2864-2864.7 | Hz |
| 2865-2865.7 | Hz |
| 2878-2878.7 | Hz |
| 2890-2890.7 | Hz |
| 2893-2893.7 | Hz |
| 2940-2940.7 | Hz |
| 2971-2971.6 | Hz |
| 2975-2975.7 | Hz |
| 2981-2981.7 | Hz |
| 3030-3030.7 | Hz |
| 3056-3056.7 | Hz |
| 3085.7-3086.3 | Hz |
| 3121-3121.7 | Hz |
| 3133-3133.7 | Hz |
| 3135-3135.7 | Hz |
| 3143-3143.7 | Hz |
| 3171-3171.7 | Hz |
| 3188-3188.7 | Hz |
| 3207-3207.7 | Hz |
| 3209-3209.7 | Hz |
| 3213-3213.7 | Hz |
| 3218-3218.7 | Hz |
| 3222-3222.7 | Hz |
| 3227.3-3228 | Hz |
| 3228-3228.7 | Hz |
| 3234-3234.7 | Hz |
| 3244-3244.7 | Hz |
| 3245-3245.7 | Hz |
| 3250.3-3250.8 | Hz |
| 3251-3251.7 | Hz |
| 3261-3261.7 | Hz |
| 3267-3267.7 | Hz |
| 3269-3269.7 | Hz |
| 3272-3272.7 | Hz |
| 3279.3-3280 | Hz |
| 3280.6-3281.3 | Hz |
| 3281.6-3282.1 | Hz |
| 3322-3322.7 | Hz |
| 3360-3360.7 | Hz |
| 3371-3371.7 | Hz |
| 3428-3428.7 | Hz |
| 3446-3446.7 | Hz |
| 3466-3466.7 | Hz |
| 3522-3522.7 | Hz |
| 3529-3529.7 | Hz |
| 3537-3537.7 | Hz |
| 3548-3548.7 | Hz |
| 3556-3556.7 | Hz |
| 3562-3562.7 | Hz |
| 3571-3571.7 | Hz |
| 3595-3595.7 | Hz |
| 3609-3609.7 | Hz |
| 3618-3618.7 | Hz |
| 3621-3621.7 | Hz |
| 3622.5-3623 | Hz |
| 3627-3627.7 | Hz |
| 3630-3630.7 | Hz |
| 3631-3631.7 | Hz |
| 3633-3633.7 | Hz |
| 3634-3634.7 | Hz |
| 3637.7-3638.3 | Hz |
| 3644-3644.7 | Hz |
| 3646-3646.7 | Hz |
| 3646.4-3647 | Hz |
| 3656-3656.7 | Hz |
| 3661-3661.7 | Hz |
| 3663-3663.7 | Hz |
| 3667-3667.7 | Hz |
| 3678-3678.7 | Hz |
| 3681.7-3682.3 | Hz |
| 3687-3687.7 | Hz |
| 3694-3694.7 | Hz |
| 3731-3731.7 | Hz |
| 3743-3743.7 | Hz |
| 3749.3-3750 | Hz |
| 3756-3756.7 | Hz |
| 3762-3762.7 | Hz |
| 3787-3787.7 | Hz |
| 3817-3817.7 | Hz |
| 3823-3823.7 | Hz |
| 3833-3833.7 | Hz |
| 3839-3839.7 | Hz |
| 3842.4-3843 | Hz |
| 3854-3854.7 | Hz |
| 3877-3877.7 | Hz |
| 3908.7-3909.22 | Hz |
| 3910-3910.7 | Hz |
| 3915-3915.7 | Hz |
| 3922-3922.7 | Hz |
| 3936-3936.7 | Hz |
| 3940-3940.7 | Hz |
| 3946-3946.7 | Hz |
| 3947-3947.7 | Hz |
| 3948-3948.7 | Hz |
| 3954-3954.7 | Hz |
| 3958-3958.6 | Hz |
| 3962-3962.7 | Hz |
| 3981-3981.7 | Hz |
| 4007-4007.7 | Hz |
| 4013-4013.7 | Hz |
| 4027-4027.7 | Hz |
| 4048-4048.7 | Hz |
| 4055-4055.7 | Hz |
| 4135-4135.7 | Hz |
| 4148-4148.7 | Hz |
| 4152-4152.7 | Hz |
| 4155-4155.7 | Hz |
| 4190-4190.7 | Hz |
| 4198-4198.7 | Hz |
| 4224-4224.7 | Hz |
| 4233-4233.7 | Hz |
| 4249-4249.7 | Hz |
| 4281-4281.7 | Hz |
| 4283-4283.7 | Hz |
| 4323-4323.7 | Hz |
| 4368.3-4369 | Hz |
| 4386.4-4387 | Hz |
| 4409-4409.7 | Hz |
| 4410-4410.7 | Hz |
| 4418-4418.7 | Hz |
| 4425-4425.7 | Hz |
| 4425.7-4426.22 | Hz |
| 4430-4430.7 | Hz |
| 4438.7-4439.2 | Hz |
| 4441-4441.7 | Hz |
| 4459-4459.7 | Hz |
| 4465-4465.7 | Hz |
| 4476-4476.7 | Hz |
| 4477.4-4478 | Hz |
| 4523-4523.7 | Hz |
| 4548-4548.7 | Hz |
| 4580.11-4580.7 | Hz |
| 4584-4584.7 | Hz |
| 4621-4621.7 | Hz |
| 4632-4632.7 | Hz |
| 4637-4637.7 | Hz |
| 4657-4657.6 | Hz |
| 4707-4707.7 | Hz |
| 4708-4708.7 | Hz |
| 4713-4713.7 | Hz |
| 4714-4714.7 | Hz |
| 4722-4722.7 | Hz |
| 4723-4723.7 | Hz |
| 4725-4725.7 | Hz |
| 4731-4731.7 | Hz |
| 4733-4733.7 | Hz |
| 4748-4748.7 | Hz |
| 4776-4776.7 | Hz |
| 4792-4792.7 | Hz |
| 4805.3-4806 | Hz |
| 4806-4806.7 | Hz |
| 4809-4809.7 | Hz |
| 4812-4812.7 | Hz |
| 4813-4813.7 | Hz |
| 4821-4821.7 | Hz |
| 4822-4822.7 | Hz |
| 4823-4823.7 | Hz |
| 4833-4833.7 | Hz |
| 4841-4841.7 | Hz |
| 4844-4844.7 | Hz |
| 4850-4850.7 | Hz |
| 4866-4866.7 | Hz |
| 4870-4870.7 | Hz |
| 4874-4874.7 | Hz |
| 4890-4890.7 | Hz |
| 4924-4924.7 | Hz |
| 4941-4941.7 | Hz |
| 4946-4946.7 | Hz |
| 4948-4948.7 | Hz |
| 4997-4997.7 | Hz |
| 4998-4998.7 | Hz |
| 5034-5034.7 | Hz |
| 5063-5063.7 | Hz |
| 5089-5089.7 | Hz |
| 5116-5116.7 | Hz |
| 5139-5139.7 | Hz |
| 5142-5142.7 | Hz |
| 5152-5152.7 | Hz |
| 5154-5154.7 | Hz |
| 5156-5156.7 | Hz |
| 5165.4-5166 | Hz |
| 5205-5205.7 | Hz |
| 5209-5209.7 | Hz |
| 5209.4-5210 | Hz |
| 5221-5221.7 | Hz |
| 5229-5229.7 | Hz |
| 5232-5232.7 | Hz |
| 5239-5239.7 | Hz |
| 5243-5243.7 | Hz |
| 5259-5259.7 | Hz |
| 5265-5265.7 | Hz |
| 5270-5270.7 | Hz |
| 5286-5286.7 | Hz |
| 5322-5322.7 | Hz |
| 5336-5336.7 | Hz |
| 5378.2-5378.7 | Hz |
| 5407.3-5408 | Hz |
| 5414-5414.7 | Hz |
| 5424-5424.7 | Hz |
| 5427-5427.7 | Hz |
| 5438-5438.7 | Hz |
| 5484.4-5485 | Hz |
| 5510-5510.7 | Hz |
| 5533-5533.7 | Hz |
| 5618-5618.7 | Hz |
| 5640.4-5641 | Hz |
| 5671-5671.7 | Hz |
| 5714-5714.7 | Hz |
| 5716-5716.7 | Hz |
| 5741-5741.7 | Hz |
| 5742-5742.7 | Hz |
| 5752-5752.7 | Hz |
| 5766-5766.7 | Hz |
| 5881-5881.7 | Hz |
| 5884-5884.7 | Hz |
| 5976-5976.7 | Hz |
| 5996-5996.7 | Hz |
| 6010-6010.7 | Hz |
| 6024-6024.7 | Hz |
| 6058-6058.7 | Hz |
| 6070-6070.7 | Hz |
| 6074-6074.7 | Hz |
| 6094-6094.7 | Hz |
| 6112-6112.7 | Hz |
| 6117-6117.7 | Hz |
| 6159-6159.7 | Hz |
| 6167-6167.7 | Hz |
| 6179-6179.7 | Hz |
| 6220.3-6221 | Hz |
| 6252-6252.7 | Hz |
| 6297-6297.7 | Hz |
| 6298.4-6299 | Hz |
| 6317-6317.7 | Hz |
| 6330-6330.7 | Hz |
| 6333-6333.7 | Hz |
| 6334-6334.7 | Hz |
| 6346-6346.7 | Hz |
| 6380.4-6381 | Hz |
| 6392-6392.7 | Hz |
| 6413-6413.7 | Hz |
| 6414-6414.7 | Hz |
| 6416.3-6417 | Hz |
| 6419.3-6420 | Hz |
| 6423-6423.7 | Hz |
| 6432-6432.7 | Hz |
| 6436-6436.7 | Hz |
| 6438-6438.7 | Hz |
| 6441-6441.7 | Hz |
| 6443-6443.7 | Hz |
| 6444-6444.7 | Hz |
| 6468-6468.7 | Hz |
| 6469-6469.7 | Hz |
| 6479.4-6480 | Hz |
| 6488-6488.7 | Hz |
| 6492.3-6493 | Hz |
| 6513-6513.7 | Hz |
| 6537-6537.7 | Hz |
| 6648-6648.7 | Hz |
| 6717-6717.7 | Hz |
| 6727.4-6728 | Hz |
| 6730-6730.7 | Hz |
| 6748-6748.7 | Hz |
| 6751-6751.7 | Hz |
| 6781-6781.7 | Hz |
| 6782-6782.7 | Hz |
| 6808-6808.7 | Hz |
| 6809-6809.6 | Hz |
| 6825-6825.7 | Hz |
| 6832-6832.7 | Hz |
| 6840-6840.7 | Hz |
| 6841-6841.7 | Hz |
| 6847-6847.7 | Hz |
| 6864-6864.7 | Hz |
| 6869-6869.7 | Hz |
| 6897-6897.7 | Hz |
| 6911.3-6912 | Hz |
| 6912-6912.7 | Hz |
| 6914-6914.7 | Hz |
| 6968.4-6969 | Hz |
| 6974-6974.7 | Hz |
| 6982-6982.7 | Hz |
| 6986-6986.7 | Hz |
| 7013-7013.7 | Hz |
| 7037-7037.7 | Hz |
| 7042-7042.7 | Hz |
| 7080-7080.7 | Hz |
| 7098-7098.7 | Hz |
| 7109-7109.7 | Hz |
| 7110-7110.7 | Hz |
| 7112-7112.7 | Hz |
| 7117-7117.7 | Hz |
| 7136-7136.7 | Hz |
| 7137-7137.7 | Hz |
| 7145.4-7146 | Hz |
| 7149-7149.7 | Hz |
| 7151-7151.7 | Hz |
| 7158-7158.7 | Hz |
| 7160-7160.7 | Hz |
| 7169-7169.7 | Hz |
| 7191.7-7192.3 | Hz |
| 7222.4-7223 | Hz |
| 7225-7225.7 | Hz |
| 7234-7234.7 | Hz |
| 7239-7239.7 | Hz |
| 7246-7246.7 | Hz |
| 7253-7253.7 | Hz |
| 7268-7268.7 | Hz |
| 7274-7274.7 | Hz |
| 7283-7283.7 | Hz |
| 7338-7338.7 | Hz |
| 7367-7367.7 | Hz |
| 7378-7378.7 | Hz |
| 7404.4-7405 | Hz |
| 7424-7424.7 | Hz |
| 7440-7440.7 | Hz |
| 7481-7481.7 | Hz |
| 7510-7510.7 | Hz |
| 7526-7526.6 | Hz |
| 7531-7531.7 | Hz |
| 7546-7546.7 | Hz |
| 7551-7551.7 | Hz |
| 7560-7560.7 | Hz |
| 7646-7646.7 | Hz |
| 7652-7652.7 | Hz |
| 7655-7655.7 | Hz |
| 7673-7673.7 | Hz |
| 7676-7676.7 | Hz |
| 7719-7719.7 | Hz |
| 7748-7748.7 | Hz |
| 7787-7787.7 | Hz |
| 7808-7808.7 | Hz |
| 7811-7811.7 | Hz |
| 7855-7855.7 | Hz |
| 7857-7857.7 | Hz |
| 7865-7865.7 | Hz |
| 7879-7879.7 | Hz |
| 7906-7906.7 | Hz |
| 7910-7910.7 | Hz |
| 7913-7913.7 | Hz |
| 7923-7923.7 | Hz |
| 7927-7927.7 | Hz |
| 7930-7930.7 | Hz |
| 7936-7936.7 | Hz |
| 7951-7951.7 | Hz |
| 7970-7970.7 | Hz |
| 7981-7981.7 | Hz |
| 7994-7994.7 | Hz |
| 8012.3-8013 | Hz |
| 8013-8013.7 | Hz |
| 8024-8024.7 | Hz |
| 8026-8026.7 | Hz |
| 8032-8032.7 | Hz |
| 8039-8039.7 | Hz |
| 8043-8043.7 | Hz |
| 8074-8074.7 | Hz |
| 8079-8079.7 | Hz |
| 8097-8097.7 | Hz |
| 8125-8125.7 | Hz |
| 8145-8145.7 | Hz |
| 8158-8158.7 | Hz |
| 8217.4-8218 | Hz |
| 8251-8251.7 | Hz |
| 8314-8314.7 | Hz |
| 8332-8332.7 | Hz |
| 8347-8347.7 | Hz |
| 8355-8355.7 | Hz |
| 8373-8373.7 | Hz |
| 8386-8386.7 | Hz |
| 8388-8388.7 | Hz |
| 8412-8412.7 | Hz |
| 8413-8413.7 | Hz |
| 8421-8421.7 | Hz |
| 8427-8427.7 | Hz |
| 8445-8445.7 | Hz |
| 8447-8447.7 | Hz |
| 8467-8467.7 | Hz |
| 8478.3-8479 | Hz |
| 8480-8480.7 | Hz |
| 8484-8484.7 | Hz |
| 8509.4-8510 | Hz |
| 8522-8522.7 | Hz |
| 8546-8546.7 | Hz |
| 8562-8562.7 | Hz |
| 8576-8576.7 | Hz |
| 8698-8698.7 | Hz |
| 8731-8731.7 | Hz |
| 8734-8734.7 | Hz |
| 8740-8740.7 | Hz |
| 8755-8755.7 | Hz |
| 8756-8756.7 | Hz |
| 8757-8757.7 | Hz |
| 8765-8765.7 | Hz |
| 8771-8771.7 | Hz |
| 8789.3-8790 | Hz |
| 8809-8809.7 | Hz |
| 8810-8810.7 | Hz |
| 8814-8814.7 | Hz |
| 8823-8823.7 | Hz |
| 8824-8824.7 | Hz |
| 8827.4-8828 | Hz |
| 8829-8829.7 | Hz |
| 8837-8837.7 | Hz |
| 8841-8841.7 | Hz |
| 8843-8843.7 | Hz |
| 8845-8845.7 | Hz |
| 8846-8846.7 | Hz |
| 8853-8853.7 | Hz |
| 8859.3-8860 | Hz |
| 8872-8872.7 | Hz |
| 8877-8877.7 | Hz |
| 8881-8881.7 | Hz |
| 8889-8889.7 | Hz |
| 8926-8926.7 | Hz |
| 8935-8935.6 | Hz |
| 8949-8949.7 | Hz |
| 9023-9023.7 | Hz |
| 9071-9071.7 | Hz |
| 9075-9075.7 | Hz |
| 9078-9078.7 | Hz |
| 9084-9084.7 | Hz |
| 9106-9106.7 | Hz |
| 9109-9109.7 | Hz |
| 9112-9112.7 | Hz |
| 9122-9122.7 | Hz |
| 9123-9123.7 | Hz |
| 9133-9133.7 | Hz |
| 9136-9136.7 | Hz |
| 9146-9146.7 | Hz |
| 9155.7-9156.2 | Hz |
| 9157-9157.7 | Hz |
| 9161-9161.7 | Hz |
| 9169-9169.7 | Hz |
| 9174-9174.7 | Hz |
| 9179-9179.7 | Hz |
| 9194-9194.7 | Hz |
| 9230-9230.7 | Hz |
| 9236-9236.7 | Hz |
| 9247-9247.7 | Hz |
| 9273-9273.7 | Hz |
| 9506-9506.7 | Hz |
| 9512-9512.7 | Hz |
| 9573-9573.7 | Hz |
| 9588-9588.7 | Hz |
| 9635-9635.7 | Hz |
| 9656-9656.7 | Hz |
| 9681-9681.7 | Hz |
| 9695-9695.7 | Hz |
| 9731-9731.7 | Hz |
| 9846-9846.7 | Hz |
| 9851-9851.7 | Hz |
| 9886-9886.7 | Hz |
| 9921-9921.7 | Hz |
| 9929-9929.7 | Hz |
| 9935-9935.7 | Hz |
| 9947-9947.7 | Hz |
| 9961-9961.7 | Hz |
| 9970-9970.7 | Hz |
| 10013-10013.7 | Hz |
| 10064-10064.7 | Hz |
| 10073-10073.7 | Hz |
| 10137-10137.7 | Hz |
| 10140-10140.7 | Hz |
| 10159-10159.7 | Hz |
| 10339.8-10340.4 | Hz |
| 10342-10342.6 | Hz |
| 10345-10345.7 | Hz |
| 10352-10352.7 | Hz |
| 10362-10362.7 | Hz |
| 10378-10378.7 | Hz |
| 10384-10384.7 | Hz |
| 10387-10387.7 | Hz |
| 10395-10395.7 | Hz |
| 10427-10427.7 | Hz |
| 10436-10436.7 | Hz |
| 10439-10439.7 | Hz |
| 10484-10484.7 | Hz |
| 10488-10488.7 | Hz |
| 10541-10541.7 | Hz |
| 10566-10566.7 | Hz |
| 10566.4-10567 | Hz |
| 10633-10633.7 | Hz |
| 10641-10641.7 | Hz |
| 10645-10645.7 | Hz |
| 10661-10661.7 | Hz |
| 10665.4-10666 | Hz |
| 10671-10671.7 | Hz |
| 10673-10673.7 | Hz |
| 10690-10690.7 | Hz |
| 10708-10708.7 | Hz |
| 10727-10727.7 | Hz |
| 10731-10731.7 | Hz |
| 10735-10735.7 | Hz |
| 10740-10740.7 | Hz |
| 10745-10745.7 | Hz |
| 10748-10748.7 | Hz |
| 10762-10762.7 | Hz |
| 10768-10768.7 | Hz |
| 10776-10776.7 | Hz |
| 10781-10781.7 | Hz |
| 10788-10788.7 | Hz |
| 10802-10802.7 | Hz |
| 10805-10805.7 | Hz |
| 10809-10809.7 | Hz |
| 10813.2-10813.8 | Hz |
| 10830-10830.7 | Hz |
| 10831-10831.7 | Hz |
| 10831.7-10832.1 | Hz |
| 10834-10834.7 | Hz |
| 10844-10844.7 | Hz |
| 10845-10845.7 | Hz |
| 10848.7-10849.3 | Hz |
| 10851-10851.7 | Hz |
| 10854-10854.7 | Hz |
| 10859-10859.7 | Hz |
| 10870-10870.7 | Hz |
| 10873-10873.7 | Hz |
| 10876-10876.7 | Hz |
| 10878-10878.7 | Hz |
| 10889-10889.7 | Hz |
| 10891-10891.7 | Hz |
| 10894-10894.7 | Hz |
| 10906-10906.7 | Hz |
| 10947-10947.7 | Hz |
| 10974-10974.7 | Hz |
| 10993-10993.7 | Hz |
| 11023-11023.7 | Hz |
| 11031-11031.7 | Hz |
| 11042-11042.7 | Hz |
| 11046-11046.7 | Hz |
| 11074-11074.7 | Hz |
| 11081-11081.7 | Hz |
| 11083-11083.7 | Hz |
| 11109.3-11110 | Hz |
| 11110-11110.7 | Hz |
| 11117-11117.7 | Hz |
| 11124-11124.7 | Hz |
| 11134-11134.7 | Hz |
| 11155-11155.7 | Hz |
| 11159-11159.7 | Hz |
| 11163-11163.7 | Hz |
| 11164-11164.7 | Hz |
| 11174-11174.7 | Hz |
| 11209-11209.7 | Hz |
| 11219-11219.7 | Hz |
| 11225-11225.7 | Hz |
| 11234-11234.7 | Hz |
| 11243-11243.7 | Hz |
| 11247-11247.7 | Hz |
| 11253-11253.7 | Hz |
| 11255-11255.7 | Hz |
| 11260-11260.7 | Hz |
| 11264-11264.7 | Hz |
| 11267-11267.7 | Hz |
| 11275-11275.7 | Hz |
| 11277.3-11278 | Hz |
| 11284-11284.7 | Hz |
| 11295-11295.7 | Hz |
| 11336-11336.7 | Hz |
| 11381-11381.7 | Hz |
| 11387-11387.7 | Hz |
| 11414-11414.7 | Hz |
| 11416-11416.7 | Hz |
| 11429-11429.7 | Hz |
| 11438-11438.7 | Hz |
| 11469-11469.7 | Hz |
| 11524-11524.7 | Hz |
| 11530-11530.7 | Hz |
| 11533-11533.7 | Hz |
| 11551.3-11552 | Hz |
| 11556-11556.7 | Hz |
| 11569-11569.7 | Hz |
| 11576-11576.7 | Hz |
| 11582-11582.7 | Hz |
| 11590-11590.7 | Hz |
| 11619-11619.7 | Hz |
| 11621-11621.7 | Hz |
| 11628-11628.7 | Hz |
| 11630-11630.7 | Hz |
| 11637-11637.7 | Hz |
| 11638-11638.7 | Hz |
| 11681-11681.7 | Hz |
| 11699-11699.7 | Hz |
| 11719-11719.7 | Hz |
| 11730.6-11731.1 | Hz |
| 11745-11745.7 | Hz |
| 11779-11779.7 | Hz |
| 11785-11785.7 | Hz |
| 11809-11809.7 | Hz |
| 11831-11831.7 | Hz |
| 11867-11867.7 | Hz |
| 11872-11872.7 | Hz |
| 11905-11905.7 | Hz |
| 11911-11911.7 | Hz |
| 11913-11913.7 | Hz |
| 11929-11929.7 | Hz |
| 11931-11931.7 | Hz |
| 11934-11934.7 | Hz |
| 11946-11946.7 | Hz |
| 11949-11949.7 | Hz |
| 11956-11956.7 | Hz |
| 11974-11974.7 | Hz |
| 11993-11993.7 | Hz |
| 11994-11994.7 | Hz |
| 12118-12118.7 | Hz |
| 12120.2-12120.7 | Hz |
| 12135-12135.7 | Hz |
| 12137-12137.7 | Hz |
| 12144-12144.7 | Hz |
| 12152-12152.7 | Hz |
| 12165-12165.7 | Hz |
| 12182-12182.7 | Hz |
| 12184-12184.7 | Hz |
| 12302-12302.7 | Hz |
| 12309-12309.7 | Hz |
| 12318-12318.7 | Hz |
| 12330-12330.7 | Hz |
| 12342-12342.7 | Hz |
| 12356-12356.7 | Hz |
| 12364-12364.7 | Hz |
| 12373-12373.7 | Hz |
| 12391-12391.7 | Hz |
| 12498-12498.7 | Hz |
| 12557-12557.7 | Hz |
| 12572-12572.7 | Hz |
| 12575-12575.7 | Hz |
| 12588-12588.7 | Hz |
| 12589-12589.7 | Hz |
| 12617-12617.7 | Hz |
| 12629-12629.7 | Hz |
| 12637-12637.7 | Hz |
| 12645-12645.7 | Hz |
| 12649-12649.7 | Hz |
| 12682-12682.7 | Hz |
| 12711-12711.7 | Hz |
| 12752-12752.7 | Hz |
| 12783-12783.7 | Hz |
| 12880-12880.7 | Hz |
| 12929-12929.7 | Hz |
| 13117-13117.7 | Hz |
| 13127-13127.7 | Hz |
| 13215-13215.7 | Hz |
| 13222-13222.7 | Hz |
| 13241-13241.7 | Hz |
| 13252-13252.7 | Hz |
| 13307-13307.7 | Hz |
| 13329-13329.7 | Hz |
| 13330.7-13331.2 | Hz |
| 13373-13373.7 | Hz |
| 13375-13375.7 | Hz |
| 13377-13377.7 | Hz |
| 13386-13386.7 | Hz |
| 13408-13408.7 | Hz |
| 13418-13418.7 | Hz |
| 13434-13434.7 | Hz |
| 13475-13475.7 | Hz |
| 13562-13562.6 | Hz |
| 13571-13571.7 | Hz |
| 13609-13609.7 | Hz |
| 13611-13611.7 | Hz |
| 13615-13615.7 | Hz |
| 13617-13617.7 | Hz |
| 13636.2-13636.7 | Hz |
| 13644-13644.7 | Hz |
| 13661-13661.7 | Hz |
| 13670-13670.7 | Hz |
| 13679-13679.7 | Hz |
| 13686.4-13687 | Hz |
| 13694-13694.7 | Hz |
| 13713-13713.7 | Hz |
| 13714-13714.7 | Hz |
| 13734-13734.7 | Hz |
| 13736-13736.7 | Hz |
| 13738.3-13739 | Hz |
| 13742-13742.7 | Hz |
| 13746-13746.7 | Hz |
| 13747-13747.7 | Hz |
| 13751-13751.7 | Hz |
| 13755-13755.7 | Hz |
| 13760-13760.7 | Hz |
| 13761-13761.7 | Hz |
| 13767-13767.7 | Hz |
| 13773-13773.7 | Hz |
| 13774-13774.7 | Hz |
| 13775-13775.7 | Hz |
| 13780-13780.7 | Hz |
| 13805-13805.7 | Hz |
| 13819-13819.7 | Hz |
| 13828-13828.7 | Hz |
| 13834-13834.7 | Hz |
| 13847-13847.7 | Hz |
| 13856-13856.7 | Hz |
| 13857.4-13858 | Hz |
| 13858-13858.7 | Hz |
| 13871-13871.7 | Hz |
| 13903-13903.7 | Hz |
| 13947-13947.7 | Hz |
| 13978.3-13979 | Hz |
| 13981-13981.7 | Hz |
| 14005-14005.7 | Hz |
| 14009-14009.7 | Hz |
| 14016.4-14017 | Hz |
| 14027-14027.7 | Hz |
| 14034-14034.7 | Hz |
| 14052-14052.7 | Hz |
| 14070-14070.7 | Hz |
| 14078-14078.7 | Hz |
| 14111-14111.7 | Hz |
| 14114-14114.7 | Hz |
| 14116-14116.7 | Hz |
| 14153-14153.7 | Hz |
| 14156-14156.7 | Hz |
| 14160-14160.7 | Hz |
| 14164-14164.7 | Hz |
| 14170.7-14171.3 | Hz |
| 14174-14174.7 | Hz |
| 14182-14182.7 | Hz |
| 14249-14249.7 | Hz |
| 14279-14279.7 | Hz |
| 14281-14281.7 | Hz |
| 14285-14285.7 | Hz |
| 14308-14308.7 | Hz |
| 14333.4-14333.9 | Hz |
| 14350-14350.7 | Hz |
| 14356-14356.7 | Hz |
| 14376-14376.7 | Hz |
| 14417-14417.7 | Hz |
| 14422-14422.7 | Hz |
| 14432-14432.7 | Hz |
| 14441-14441.7 | Hz |
| 14443-14443.7 | Hz |
| 14456-14456.7 | Hz |
| 14462-14462.7 | Hz |
| 14479-14479.7 | Hz |
| 14483-14483.7 | Hz |
| 14489-14489.7 | Hz |
| 14511-14511.7 | Hz |
| 14523-14523.7 | Hz |
| 14524-14524.7 | Hz |
| 14531-14531.7 | Hz |
| 14532-14532.7 | Hz |
| 14540-14540.7 | Hz |
| 14545-14545.7 | Hz |
| 14553-14553.7 | Hz |
| 14558-14558.7 | Hz |
| 14564-14564.7 | Hz |
| 14570-14570.7 | Hz |
| 14574-14574.7 | Hz |
| 14585-14585.7 | Hz |
| 14589-14589.7 | Hz |
| 14621-14621.7 | Hz |
| 14630-14630.7 | Hz |
| 14670.4-14671 | Hz |
| 14671-14671.7 | Hz |
| 14674-14674.7 | Hz |
| 14690-14690.7 | Hz |
| 14716-14716.7 | Hz |
| 14729-14729.7 | Hz |
| 14756-14756.7 | Hz |
| 14796-14796.7 | Hz |
| 14813-14813.7 | Hz |
| 14835-14835.7 | Hz |
| 14858-14858.7 | Hz |
| 14863-14863.7 | Hz |
| 14867-14867.7 | Hz |
| 14877-14877.7 | Hz |
| 14885-14885.7 | Hz |
| 14911-14911.7 | Hz |
| 14912-14912.7 | Hz |
| 14913.4-14914 | Hz |
| 14922-14922.7 | Hz |
| 14940-14940.7 | Hz |
| 14940.4-14941 | Hz |
| 14957-14957.7 | Hz |
| 14961-14961.7 | Hz |
| 14974-14974.7 | Hz |
| 14982-14982.7 | Hz |
| 15006-15006.7 | Hz |
| 15033-15033.7 | Hz |
| 15035-15035.6 | Hz |
| 15056-15056.7 | Hz |
| 15067-15067.7 | Hz |
| 15071-15071.7 | Hz |
| 15109-15109.7 | Hz |
| 15112-15112.7 | Hz |
| 15126-15126.7 | Hz |
| 15133-15133.7 | Hz |
| 15154-15154.7 | Hz |
| 15173-15173.7 | Hz |
| 15179-15179.7 | Hz |
| 15195-15195.7 | Hz |
| 15223-15223.7 | Hz |
| 15224-15224.7 | Hz |
| 15225-15225.6 | Hz |
| 15226-15226.7 | Hz |
| 15230-15230.7 | Hz |
| 15233-15233.7 | Hz |
| 15236-15236.7 | Hz |
| 15244-15244.7 | Hz |
| 15250-15250.7 | Hz |
| 15253-15253.7 | Hz |
| 15263-15263.7 | Hz |
| 15269-15269.7 | Hz |
| 15278-15278.7 | Hz |
| 15329-15329.7 | Hz |
| 15432-15432.7 | Hz |
| 15435-15435.7 | Hz |
| 15460-15460.7 | Hz |
| 15515-15515.7 | Hz |
| 15521-15521.7 | Hz |
| 15528-15528.7 | Hz |
| 15531-15531.7 | Hz |
| 15536-15536.7 | Hz |
| 15539-15539.7 | Hz |
| 15545-15545.7 | Hz |
| 15546.3-15547 | Hz |
| 15550.4-15551 | Hz |
| 15563-15563.7 | Hz |
| 15576-15576.7 | Hz |
| 15589-15589.7 | Hz |
| 15615-15615.7 | Hz |
| 15621-15621.7 | Hz |
| 15624-15624.7 | Hz |
| 15642-15642.7 | Hz |
| 15647-15647.7 | Hz |
| 15663-15663.7 | Hz |
| 15669-15669.7 | Hz |
| 15678-15678.7 | Hz |
| 15698-15698.7 | Hz |
| 15711-15711.7 | Hz |
| 15724-15724.7 | Hz |
| 15729-15729.7 | Hz |
| 15740-15740.7 | Hz |
| 15749-15749.7 | Hz |
| 15773-15773.7 | Hz |
| 15817-15817.7 | Hz |
| 15823-15823.7 | Hz |
| 15824-15824.7 | Hz |
| 15825-15825.7 | Hz |
| 15835-15835.7 | Hz |
| 15836-15836.7 | Hz |
| 15857-15857.7 | Hz |
| 15867-15867.7 | Hz |
| 15897-15897.7 | Hz |
| 15919-15919.7 | Hz |
| 15942-15942.7 | Hz |
| 15982-15982.7 | Hz |
| 16071-16071.7 | Hz |
| 16082-16082.7 | Hz |
| 16096-16096.7 | Hz |
| 16143-16143.7 | Hz |
| 16176-16176.7 | Hz |
| 16274-16274.7 | Hz |
| 16309-16309.7 | Hz |
| 16329-16329.7 | Hz |
| 16347-16347.7 | Hz |
| 16381-16381.7 | Hz |
| 16515-16515.7 | Hz |
| 16679-16679.7 | Hz |
| 16804-16804.7 | Hz |
| 16821-16821.7 | Hz |
| 17095-17095.7 | Hz |
| 17106-17106.7 | Hz |
| 17123-17123.7 | Hz |
| 17167-17167.7 | Hz |
| 17214-17214.7 | Hz |
| 17229-17229.7 | Hz |
| 17246-17246.7 | Hz |
| 17268-17268.7 | Hz |
| 17271-17271.7 | Hz |
| 17283-17283.7 | Hz |
| 17333-17333.7 | Hz |
| 17379-17379.7 | Hz |
| 17417-17417.7 | Hz |
| 17418-17418.7 | Hz |
| 17435-17435.7 | Hz |
| 17455-17455.7 | Hz |
| 17472-17472.7 | Hz |
| 17473-17473.7 | Hz |
| 17474-17474.7 | Hz |
| 17485-17485.7 | Hz |
| 17515-17515.7 | Hz |
| 17556-17556.7 | Hz |
| 17578.4-17579 | Hz |
| 17581-17581.7 | Hz |
| 17621-17621.7 | Hz |
| 17636-17636.7 | Hz |
| 17655-17655.7 | Hz |
| 17673-17673.7 | Hz |
| 17737-17737.7 | Hz |
| 17739-17739.7 | Hz |
| 17749-17749.7 | Hz |
| 17757-17757.7 | Hz |
| 17808-17808.7 | Hz |
| 17825-17825.7 | Hz |
| 17832-17832.7 | Hz |
| 17944-17944.7 | Hz |
| 17962-17962.7 | Hz |
| 17974-17974.7 | Hz |
| 17978-17978.7 | Hz |
| 18021-18021.7 | Hz |
| 18040-18040.7 | Hz |
| 18087-18087.7 | Hz |
| 18090-18090.7 | Hz |
| 18126-18126.7 | Hz |
| 18140-18140.7 | Hz |
| 18157-18157.7 | Hz |
| 18171-18171.7 | Hz |
| 18182-18182.7 | Hz |
| 18211-18211.7 | Hz |
| 18221-18221.7 | Hz |
| 18222-18222.7 | Hz |
| 18246-18246.7 | Hz |
| 18260-18260.7 | Hz |
| 18274-18274.7 | Hz |
| 18279-18279.7 | Hz |
| 18294-18294.7 | Hz |
| 18372-18372.7 | Hz |
| 18375-18375.7 | Hz |
| 18396-18396.7 | Hz |
| 18411-18411.7 | Hz |
| 18454-18454.7 | Hz |
| 18462-18462.7 | Hz |
| 18521-18521.7 | Hz |
| 18533-18533.7 | Hz |
| 18567-18567.7 | Hz |
| 18585-18585.7 | Hz |
| 18596-18596.7 | Hz |
| 18618-18618.7 | Hz |
| 18635-18635.7 | Hz |
| 18641-18641.7 | Hz |
| 18655-18655.7 | Hz |
| 18670.3-18671 | Hz |
| 18672-18672.7 | Hz |
| 18678-18678.7 | Hz |
| 18678.7-18679.3 | Hz |
| 18686-18686.7 | Hz |
| 18742-18742.7 | Hz |
| 18831-18831.7 | Hz |
| 18842-18842.7 | Hz |
| 18884-18884.7 | Hz |
| 18895-18895.7 | Hz |
| 18897-18897.7 | Hz |
| 18922-18922.7 | Hz |
| 19026-19026.7 | Hz |
| 19059-19059.7 | Hz |
| 19125-19125.7 | Hz |
| 19269-19269.7 | Hz |
| 19342-19342.7 | Hz |
| 19365-19365.7 | Hz |
| 19384-19384.7 | Hz |
| 19393-19393.7 | Hz |
| 19419-19419.7 | Hz |
| 19426-19426.7 | Hz |
| 19433-19433.7 | Hz |
| 19437-19437.7 | Hz |
| 19446-19446.7 | Hz |
| 19447-19447.7 | Hz |
| 19453-19453.7 | Hz |
| 19464-19464.7 | Hz |
| 19476-19476.7 | Hz |
| 19489.3-19490 | Hz |
| 19510-19510.7 | Hz |
| 19577-19577.7 | Hz |
| 19686-19686.4 | Hz |
| 19715-19715.7 | Hz |
| 19724-19724.7 | Hz |
| 19744-19744.7 | Hz |
| 19782-19782.7 | Hz |
| 19817-19817.7 | Hz |
| 19819-19819.7 | Hz |
| 19823-19823.7 | Hz |
| 19827-19827.7 | Hz |
| 19829-19829.7 | Hz |
| 19861-19861.7 | Hz |
| 19867-19867.7 | Hz |
| 19889-19889.7 | Hz |
| 20097-20097.7 | Hz |
| 20123-20123.7 | Hz |
| 20128-20128.7 | Hz |
| 20144-20144.7 | Hz |
| 20149.3-20150 | Hz |
| 20173-20173.7 | Hz |
| 20219-20219.7 | Hz |
| 20229-20229.7 | Hz |
| 20237-20237.7 | Hz |
| 20254-20254.7 | Hz |
| 20256-20256.7 | Hz |
| 20278.4-20279 | Hz |
| 20284-20284.7 | Hz |
| 20286-20286.7 | Hz |
| 20324-20324.7 | Hz |
| 20499-20499.7 | Hz |
| 20513-20513.7 | Hz |
| 20634-20634.7 | Hz |
| 20747-20747.7 | Hz |
| 20818-20818.7 | Hz |
| 20825-20825.7 | Hz |
| 20908-20908.7 | Hz |
| 20948-20948.7 | Hz |
| 21119-21119.7 | Hz |
| 21167-21167.7 | Hz |
| 21291.4-21292 | Hz |
| 21372-21372.7 | Hz |
| 21389-21389.7 | Hz |
| 21430-21430.7 | Hz |
| 21445-21445.7 | Hz |
| 21466-21466.7 | Hz |
| 21467-21467.7 | Hz |
| 21490-21490.7 | Hz |
| 21546-21546.7 | Hz |
| 21567-21567.7 | Hz |
| 21616-21616.7 | Hz |
| 21636-21636.7 | Hz |
| 21663-21663.7 | Hz |
| 21664-21664.7 | Hz |
| 21708-21708.7 | Hz |
| 21714-21714.7 | Hz |
| 21720-21720.7 | Hz |
| 21778-21778.7 | Hz |
| 21833-21833.7 | Hz |
| 21840-21840.7 | Hz |
| 21844-21844.7 | Hz |
| 21854-21854.7 | Hz |
| 21865-21865.7 | Hz |
| 21870-21870.7 | Hz |
| 21876-21876.7 | Hz |
| 21887-21887.7 | Hz |
| 21890-21890.7 | Hz |
| 21894-21894.7 | Hz |
| 21963-21963.7 | Hz |
| 22020-22020.7 | Hz |
| 22073-22073.7 | Hz |
| 22119-22119.7 | Hz |
| 22167-22167.7 | Hz |
| 22312-22312.7 | Hz |
| 22338-22338.7 | Hz |
| 22344-22344.7 | Hz |
| 22350-22350.7 | Hz |
| 22353-22353.7 | Hz |
| 22355-22355.7 | Hz |
| 22361-22361.7 | Hz |
| 22411-22411.7 | Hz |
| 22419-22419.7 | Hz |
| 22422-22422.7 | Hz |
| 22423.4-22424 | Hz |
| 22425-22425.7 | Hz |
| 22430-22430.7 | Hz |
| 22437-22437.7 | Hz |
| 22443-22443.7 | Hz |
| 22444-22444.7 | Hz |
| 22454-22454.7 | Hz |
| 22459-22459.7 | Hz |
| 22484-22484.7 | Hz |
| 22491-22491.7 | Hz |
| 22519-22519.7 | Hz |
| 22535-22535.7 | Hz |
| 22538-22538.7 | Hz |
| 22548-22548.7 | Hz |
| 22559.3-22560 | Hz |
| 22582-22582.7 | Hz |
| 22597-22597.7 | Hz |
| 22709-22709.7 | Hz |
| 22730-22730.6 | Hz |
| 22742-22742.7 | Hz |
| 22743-22743.7 | Hz |
| 22763-22763.7 | Hz |
| 22822-22822.7 | Hz |
| 22832-22832.7 | Hz |
| 22840.3-22841 | Hz |
| 22846-22846.7 | Hz |
| 22859-22859.7 | Hz |
| 22863-22863.7 | Hz |
| 22867-22867.7 | Hz |
| 22879-22879.7 | Hz |
| 22932-22932.7 | Hz |
| 22973-22973.7 | Hz |
| 22983-22983.7 | Hz |
| 23012-23012.7 | Hz |
| 23044-23044.7 | Hz |
| 23111-23111.7 | Hz |
| 23117-23117.7 | Hz |
| 23137-23137.7 | Hz |
| 23216-23216.7 | Hz |
| 23237-23237.7 | Hz |
| 23246-23246.7 | Hz |
| 23249-23249.7 | Hz |
| 23258-23258.7 | Hz |
| 23259-23259.7 | Hz |
| 23261-23261.7 | Hz |
| 23284-23284.7 | Hz |
| 23344-23344.7 | Hz |
| 23412-23412.7 | Hz |
| 23444-23444.7 | Hz |
| 23509-23509.7 | Hz |
| 23526-23526.7 | Hz |
| 23548-23548.7 | Hz |
| 23560-23560.7 | Hz |
| 23589-23589.7 | Hz |
| 23624-23624.7 | Hz |
| 23652-23652.7 | Hz |
| 23659-23659.7 | Hz |
| 23667-23667.7 | Hz |
| 23686-23686.7 | Hz |
| 23730-23730.7 | Hz |
| 23921-23921.7 | Hz |
| 23946-23946.7 | Hz |
| 23957-23957.7 | Hz |
| 24014-24014.7 | Hz |
| 24060-24060.7 | Hz |
| 24073-24073.7 | Hz |
| 24085-24085.7 | Hz |
| 24112-24112.7 | Hz |
| 24117-24117.7 | Hz |
| 24142-24142.7 | Hz |
| 24146-24146.7 | Hz |
| 24174-24174.7 | Hz |
| 24196-24196.7 | Hz |
| 24198-24198.7 | Hz |
| 24228-24228.7 | Hz |
| 24275-24275.7 | Hz |
| 24413-24413.7 | Hz |
| 24508-24508.7 | Hz |
| 24609.3-24610 | Hz |
| 24789-24789.7 | Hz |
| 24811-24811.7 | Hz |
| 24841-24841.7 | Hz |
| 24869-24869.7 | Hz |
| 25199-25199.7 | Hz |
| 25449-25449.7 | Hz |
| 25454-25454.7 | Hz |
| 26044-26044.7 | Hz |
| 26073-26073.7 | Hz |
| 26113-26113.7 | Hz |
| 26142-26142.7 | Hz |
| 26163-26163.7 | Hz |
| 26164-26164.7 | Hz |
| 26212-26212.7 | Hz |
| 26343-26343.7 | Hz |
| 26403-26403.7 | Hz |
| 26465-26465.7 | Hz |
| 26534-26534.7 | Hz |
| 26722-26722.7 | Hz |
| 26861-26861.7 | Hz |
| 26954-26954.7 | Hz |
| 27114-27114.7 | Hz |
| 27214-27214.7 | Hz |
| 27291-27291.7 | Hz |
| 27328-27328.6 | Hz |
| 27478-27478.7 | Hz; |
| 27713-27713.7 | Hz |
| 28212-28212.7 | Hz |
| 28222-28222.7 | Hz |
| 28272-28272.7 | Hz |
| 28361-28361.7 | Hz |
| 28537-28537.7 | Hz |
| 28653-28653.7 | Hz |
| 28744-28744.7 | Hz |
| 30075-30075.7 | Hz |
| 30304-30304.7 | Hz |
| 30373-30373.7 | Hz |
| 30463.3-30464 | Hz |
| 30666-30666.7 | Hz |
| 30713-30713.7 | Hz |
| 30759-30759.7 | Hz |
| 30942-30942.7 | Hz |
| 31051-31051.7 | Hz |
| 31572-31572.7 | Hz |
| 31633-31633.7 | Hz |
| 31685-31685.7 | Hz |
| 31713-31713.7 | Hz |
| 31847-31847.7 | Hz |
| 32143-32143.7 | Hz |
| 32493-32493.7 | Hz |
| 32591.3-32592 | Hz |
| 32866-32866.7 | Hz |
| 32870-32870.7 | Hz |
| 33366-33366.7 | Hz |
| 33414-33414.7 | Hz |
| 33536-33536.7 | Hz |
| 33546-33546.7 | Hz |
| 33553-33553.7 | Hz |
| 33765-33765.7 | Hz |
| 34405-34405.7 | Hz |
| 34557.3-34558 | Hz |
| 34562-34562.7 | Hz |
| 34614-34614.7 | Hz |
| 34854-34854.7 | Hz |
| 34984-34984.6 | Hz |
| 35296-35296.7 | Hz |
| 35324-35324.7 | Hz |
| 35543-35543.7 | Hz |
| 35826-35826.7 | Hz |
| 36343-36343.7 | Hz |
| 36410-36410.7 | Hz |
| 37527-37527.7 | Hz |
| 37546-37546.7 | Hz |
| 37680-37680.7 | Hz |
| 38144-38144.7 | Hz |
| 38216-38216.7 | Hz |
| 38483-38483.7 | Hz |
| 38659-38659.7 | Hz |
| 38682-38682.7 | Hz |
| 39035-39035.7 | Hz |
| 39053-39053.7 | Hz |
| 39153-39153.7 | Hz |
| 39183-39183.7 | Hz |
| 39223-39223.7 | Hz |
| 39423-39423.7 | Hz |
| 39510-39510.7 | Hz |
| 39586-39586.7 | Hz |
| 39729-39729.7 | Hz |
| 40111-40111.7 | Hz |
| 40133-40133.7 | Hz |
| 40394-40394.7 | Hz |
| 40489-40489.7 | Hz |
| 40595-40595.7 | Hz |
| 40693-40693.7 | Hz |
| 40734-40734.7 | Hz |
| 40762-40762.7 | Hz |
| 40896-40896.7 | Hz |
| 40899-40899.7 | Hz |
| 41062-41062.7 | Hz |
| 41172-41172.7 | Hz |
| 41244-41244.7 | Hz |
| 41279-41279.7 | Hz |
| 41325-41325.7 | Hz |
| 41424-41424.7 | Hz |
| 41473-41473.7 | Hz |
| 41534-41534.7 | Hz |
| 41557-41557.7 | Hz |
| 41561-41561.7 | Hz |
| 41574-41574.7 | Hz |
| 41623-41623.7 | Hz |
| 41735-41735.7 | Hz |
| 41859-41859.7 | Hz |
| 41861-41861.7 | Hz |
| 41883-41883.7 | Hz |
| 42146-42146.7 | Hz |
| 42293-42293.7 | Hz |
| 42402-42402.7 | Hz |
| 42544-42544.7 | Hz |
| 42692.3-42693 | Hz |
| 42713-42713.7 | Hz |
| 42812-42812.7 | Hz |
| 42813-42813.7 | Hz |
| 42913-42913.7 | Hz |
| 42923-42923.7 | Hz |
| 43162-43162.7 | Hz |
| 43166-43166.7 | Hz |
| 43172-43172.7 | Hz |
| 43243-43243.7 | Hz |
| 43325-43325.7 | Hz |
| 43379-43379.7 | Hz |
| 43413-43413.7 | Hz |
| 43542-43542.7 | Hz |
| 43603-43603.7 | Hz |
| 43633-43633.7 | Hz |
| 43692-43692.7 | Hz |
| 43851-43851.7 | Hz |
| 44162-44162.7 | Hz |
| 44517-44517.7 | Hz |
| 44531-44531.7 | Hz |
| 44575.3-44576 | Hz |
| 44652-44652.7 | Hz |
| 44692-44692.7 | Hz |
| 44742-44742.7 | Hz |
| 44773-44773.7 | Hz |
| 44855-44855.7 | Hz |
| 44913-44913.7 | Hz |
| 45163-45163.7 | Hz |
| 45362-45362.7 | Hz |
| 45393-45393.7 | Hz |
| 45474-45474.7 | Hz |
| 45490-45490.7 | Hz |
| 45526-45526.7 | Hz |
| 46112-46112.7 | Hz |
| 46143-46143.7 | Hz |
| 46282-46282.7 | Hz |
| 46285-46285.7 | Hz |
| 46340-46340.7 | Hz |
| 46662-46662.7 | Hz |
| 46666-46666.7 | Hz |
| 46672.3-46673 | Hz |
| 46673-46673.7 | Hz |
| 46689-46689.6 | Hz |
| 46746-46746.7 | Hz |
| 46793-46793.7 | Hz |
| 47036-47036.7 | Hz |
| 47297-47297.7 | Hz |
| 47693.4-47694 | Hz |
| 48103-48103.7 | Hz |
| 48173-48173.7 | Hz |
| 48204-48204.7 | Hz |
| 48314-48314.7 | Hz |
| 48535-48535.7 | Hz |
| 48583-48583.7 | Hz |
| 48653-48653.7 | Hz |
| 48683-48683.7 | Hz |
| 48793-48793.7 | Hz |
| 49434-49434.7 | Hz |
| 50072-50072.7 | Hz |
| 50229-50229.7 | Hz |
| 50243-50243.7 | Hz |
| 50254-50254.7 | Hz |
| 50292-50292.7 | Hz |
| 51008-51008.7 | Hz |
| 51592-51592.7 | Hz |
| 51946-51946.7 | Hz |
| 52063-52063.7 | Hz |
| 52097-52097.7 | Hz |
| 52752-52752.7 | Hz |
| 52786-52786.7 | Hz |
| 52812-52812.7 | Hz |
| 52912-52912.7 | Hz |
| 53079-53079.7 | Hz |
| 53947-53947.7 | Hz |
| 54293-54293.7 | Hz |
| 54297-54297.7 | Hz |
| 55072-55072.7 | Hz |
| 55592-55592.7 | Hz |
| 55723-55723.7 | Hz |
| 56093-56093.7 | Hz |
| 57927-57927.7 | Hz |
| 58094-58094.7 | Hz |
| 59089-59089.7 | Hz |
| 59288-59288.7 | Hz |
| 59907-59907.7 | Hz |
| 59997-59997.7 | Hz |
| 61287-61287.7 | Hz |
| 61789-61789.7 | Hz |
| 65586-65586.7 | Hz |
| 66553-66553.7 | Hz |
| 66969-66969.7 | Hz |
| 67683-67683.7 | Hz |
| 67697-67697.6 | Hz |
| 69481-69481.7 | Hz |
| 71204-71204.7 | Hz |
| 71447-71447.7 | Hz |
| 71635-71635.7 | Hz |
| 74017-74017.7 | Hz |
| 76227-76227.7 | Hz |
| 76537-76537.7 | Hz |
| 76659-76659.7 | Hz |
| 78229-78229.7 | Hz |
| 78477.4-78478 | Hz |
| 79658-79658.7 | Hz |
| 79867-79867.7 | Hz |
| 80715-80715.7 | Hz |
| 80993.3-80994 | Hz |
| 81999-81999.7 | Hz |
| 82024-82024.7 | Hz |
| 82845-82845.7 | Hz |
| 83612-83612.7 | Hz |
| 84086-84086.7 | Hz |
| 85810-85810.7 | Hz |
| 86521-86521.7 | Hz |
| 87507-87507.7 | Hz |
| 87798.3-87799 | Hz |
| 88281-88281.7 | Hz |
| 88898-88898.7 | Hz |
| 89748-89748.7 | Hz |
| 91245-91245.7 | Hz |
| 91271-91271.7 | Hz |
| 91877-91877.7 | Hz |
| 92182-92182.7 | Hz |
| 92361.3-92362 | Hz |
| 92787-92787.7 | Hz |
| 93332-93332.7 | Hz |
| 93530-93530.7 | Hz |
| 94494-94494.7 | Hz |
| 94968.3-94969 | Hz |
| 95245-95245.7 | Hz |
| 95742-95742.7 | Hz |
| 99281-99281.7 | Hz |
| 99846-99846.7 | Hz |

30. The apparatus of claim 29, wherein the cancer is selected from the group consisting of: breast cancer, neuroendocrine tumors, non-Hodgkin lymphoma, adenocarcinoma, head and neck cancer, gastric cancer, glioblastoma, squamous cell carcinoma, hepatocellular carcinoma, cholangiocarcinoma, mesothelioma, thyroid cancer, prostate cancer, rhabdomyosarcoma, lung cancer, kidney cancer, ovarian cancer, bladder cancer, leiomyosarcoma, myeloma, lymphoma, leukemia, chronic lymphoid cancer, brain cancer, and colorectal cancer.

31. The apparatus of claim 29, wherein the carrier frequency is from about 0.1-1000 MHz.

32. The apparatus according to claim 29, wherein the frequency synthesizer is a digital frequency synthesizer comprising:
a digital carrier frequency synthesizer configured to output the carrier frequency signal;
a digital modulation frequency synthesizer configured to output the one or more amplitude modulation frequency signals; and
an arithmetic logic unit (ALU) configured to numerically compute one or more digital modulated signals from the carrier frequency signal and a modulation frequency digital signal in real-time; and
a digital to analog converter (DAC) configured to convert the one or more digital modulated signals to the one or more amplitude-modulated output signals.

33. An apparatus for treating a subject suffering from cancer, the apparatus comprising:
a conductive applicator configured to apply to the subject low energy high frequency radiation, wherein the low energy high frequency radiation comprises one or more amplitude-modulated output signals; and
a frequency synthesizer coupled to the conductive applicator and configured to generate the one or more amplitude-modulated output signals by generating:
a carrier frequency signal having a carrier frequency from about 1 KHz to 5000 MHz; and
amplitude modulation frequency signals having amplitude modulation frequencies from about 0.1 Hz to about 150,000 Hz, wherein the amplitude modulation frequencies are selected to be cancer-specific frequencies;
wherein the frequency synthesizer is a digital frequency synthesizer comprising:
a digital carrier frequency synthesizer configured to output the carrier frequency signal;
a digital modulation frequency synthesizer configured to output the one or more amplitude modulation frequency signals;
an arithmetic logic unit (ALU) configured to numerically compute one or more digital modulated signals from the carrier frequency signal and a modulation frequency digital signal in real-time; and
a digital to analog converter (DAC) configured to convert the one or more digital modulated signals to the one or more amplitude-modulated output signals.

34. The apparatus of claim 33, wherein the cancer is selected from the group consisting of: breast cancer, neuroendocrine tumors, non-Hodgkin lymphoma, adenocarcinoma, head and neck cancer, gastric cancer, glioblastoma, squamous cell carcinoma, hepatocellular carcinoma, cholangiocarcinoma, mesothelioma, thyroid cancer, prostate cancer, rhabdomyosarcoma, lung cancer, kidney cancer, ovarian cancer, bladder cancer, leiomyosarcoma, myeloma, lymphoma, leukemia, chronic lymphoid cancer, brain cancer, and colorectal cancer.

35. The apparatus of claim 33, wherein the carrier frequency is from about 0.1-1000 MHz.

36. The apparatus according to claim 33, wherein the modulation frequency is from 100 Hz to 99,000 Hz.

37. The apparatus according to claim 33, wherein the digital frequency synthesizer further comprises a controller configured to control a frequency and a power associated with the one or more amplitude-modulated output signals.

38. The apparatus according to claim 33, further comprising a directional coupler for providing, to the controller, power and absorption information relating to each of the one or more amplitude-modulated output signals.

39. The apparatus according to claim 33, wherein the digital frequency synthesizer further comprises a phase-locked loop (PLL) frequency multiplier configured to set a system clock for one or more of the following: the digital carrier frequency synthesizer or the digital modulation frequency synthesizer.

40. The apparatus according to claim 33, wherein the digital carrier frequency synthesizer is a direct digital synthesizer (DDS).

41. The apparatus according to claim 33, wherein the digital modulation frequency synthesizer is a direct digital synthesizer (DDS).

42. The apparatus according to claim 33, wherein the frequency synthesizer is configured to generate the one or more amplitude-modulated output signals, either sequentially or simultaneously.

43. The apparatus according to claim 33, wherein the conductive applicator is configured for insertion into an oral cavity of the subject undergoing treatment.

44. The apparatus according to claim 33, wherein the amplitude modulation frequencies are determined or predetermined by a bio-feedback process involving observations or measurements of physiological reactions by the subject during a time that cellular functions of the subject are excited by exposing the subject to emission of a modulated output signal.
